(19) United States
(12) Patent Application Publication
(10) Pub. No.: US 2024/0368565 A1
(43) Pub. Date: Nov. 7, 2024

(54) COMPLEMENTATION-BASED TAGS AND REPORTERS FOR DUAL-MODALITY LABELING

(71) Applicant: Promega Corporation, Madison, WI (US)

(72) Inventors: Michael KILLORAN, Madison, WI (US); Rahele Esmatpour, Madison, WI (US); Lance P. Encell, Madison, WI (US); Christopher Todd Eggers, Madison, WI (US); Nina J. Bonde, Madison, WI (US)

(21) Appl. No.: 18/655,005

(22) Filed: May 3, 2024

Related U.S. Application Data
(60) Provisional application No. 63/500,118, filed on May 4, 2023.

(51) Int. Cl. C12N 9/02; C12N 9/14; G01N 33/58

(52) U.S. Cl. CPC C12N 9/0069; C12N 9/14; G01N 33/582; G01N 33/583; C12Y 113/12007

(57) ABSTRACT

Provided herein are compositions and systems comprising complementation-based tags and reporters for labeling and detection of targets by luminescence and a second modality (e.g., fluorescence), and methods of use thereof. In particular, tags are provided comprising the fusion of a first component of bioluminescent complex and a first component of modified dehalogenase complex, reporters are provided comprising the second component of bioluminescent complex and the second component of modified dehalogenase complex, and systems and methods are provided comprising the tags and reporters herein for dual-modality labeling and detection of targets.

### CROSS-REFERENCE TO RELATED APPLICATIONS

The present invention claims the priority benefit of U.S. Provisional Patent Application 63/500,118, filed May 4, 2023, which is incorporated by reference in its entirety.

### SEQUENCE LISTING

The text of the computer readable sequence listing filed herewith, titled “PRMG_41898_202_SequenceListing.xml,” created on May 3, 2024, having a file size of 4,666,369 bytes, is hereby incorporated by reference in its entirety.

### FIELD

Provided herein are compositions and systems comprising complementation-based tags and reporters for labeling and detection of targets by luminescence and a second modality (e.g., fluorescence), and methods of use thereof. In particular, tags are provided comprising the fusion of a first component of bioluminescent complex and a first component of modified dehalogenase complex, reporters are provided comprising the second component of bioluminescent complex and the second component of modified dehalogenase complex, and systems and methods are provided comprising the tags and reporters herein for dual-modality labeling and detection of targets.

### BACKGROUND

Reporter technologies based on complementation of split protein sequences have proven utility across a broad range of biomedical applications. In their most common configuration, these reporters function by initially providing little or no signal generation under conditions where their complementary sequences are kept separate, or their interaction is unfavorable. Upon changing conditions that enable interaction, such as through chemical induction, physical proximity, altered concentration, or affinity, the sequences interact with one another and fold into an active protein complex that generates a measurable signal, for example, in the form of luminescence or fluorescence.

The utility of split protein complementation systems as research tools has been demonstrated in their application for biosensing, diagnostics, drug discovery, targeted molecular degradation, cell and molecular imaging, and detection of energy transfer (BRET, FRET), among other examples. The capabilities of these research tools can be targeted to specific molecular events through fusion of the split protein sequences to intracellular proteins, providing simple yet robust versatility in the methods and the design of their application. The emergence of CRISPR genome editing technology has further increased the value small-sized (<35 amino acids) split protein sequences for use as fusion tags, since their small size enables more efficient integration into the genome and makes them less likely to disrupt the function of the targeted protein fusion partner.

Several examples of asymmetric split protein reporters with small sequences have been reported, including fluorescent proteins (GFP, RFP), luciferases (NanoLuc), and self-labeling proteins (HaloTag, SNAP-tag). Introduction of these reporters as fusions to endogenous genes using CRISPR-based genome editing has been demonstrated to result in many of the functional capabilities outlined above. However, despite the utility and versatility of these split protein tags, users have to select a single detection method (e.g., fluorescence or luminescence) and subsequently commit to generating assays or engineered cell lines that are limited to the advantages and disadvantages of the chosen detection type. For example, while luminescence-based detection, for example, with NanoLuc Binary Technology (NANOBIT) has exceptional sensitivity and linearity, it has not been widely adopted for cellular imaging or flow cytometry due to the difficulty of luminescence detection with typical microscopy or cytometry detection instrumentation. Additional methods where fluorescence is utilized preferentially over bioluminescence for imaging include the following: single molecule tracking, localization, fixation, single cell/sub-cellular resolution, and colocalization experiments using multiple colors.

Conversely, while small, split fluorescent protein tags can be used for fluorescence microscopy or cytometry, they are often limited in their dynamic range and available emission wavelengths for measurement.

What is needed is a single, split protein technology that provides use of both luminescence and fluorescence for detection of complementation between a tag and a complementary reporter sequence. Such a system would enable users to measure the functional dynamics of a protein target of interest under minimally disruptive conditions while realizing the benefits of both detection methods.

### SUMMARY

Provided herein are compositions and systems comprising complementation-based tags and reporters for labeling and detection of targets by luminescence and a second modality (e.g., fluorescence), and methods of use thereof. In particular, tags are provided comprising the fusion of a first component of bioluminescent complex and a first component of modified dehalogenase complex, reporters are provided comprising the second component of bioluminescent complex and the second component of modified dehalogenase complex, and systems and methods are provided comprising the tags and reporters herein for dual-modality labeling and detection of targets. In some embodiments, provided herein is a dual luminescence-fluorescence detection technology based on the fusion of split NanoLuc® (LgBit and SmBiT/HiBiT) and split HaloTag® (LgHT and SmHT) sequences. Embodiments of such configurations maintain: (1) a single, small-sized tag comprising a fusion between SmHT and SmBiT/HiBiT sequences and (2) a complementary reporter polypeptide comprising a fusion between LgHT and LgBiT sequences. The advantages provided such embodiments are initially realized through introduction of the small sized fusion tag, for example the SmHT-HiBiT tag, to a protein target of interest. Once tagged, detection of the protein target through its high affinity, spontaneous interaction with a LgHT-LgBiT reporter polypeptide reconstitutes a “detection complex” comprised of reconstituted and active NanoLuc® and HaloTag® complexes. In the non-complemented/non-complexed state, the dual peptide tag is not capable of generating a signal, providing a zero-background state not attainable when using full length fluorescence proteins. The higher binding affinity between LgBiT and HiBiT drives the interaction between LgHT and SmHT via induced proximity. Introduction of luciferase substrate and a fluorescent HaloTag ligand, respectively, results in separate luminescent and fluorescent signals that can be configured to enable multiple functional measurements or assay types. The high affinity complementation with the LgHT-LgBiT detection polypeptide provides the advantage of choosing which detection modality is the most beneficial under the circumstances without the need to change the tag fused to the protein target.

While selection of detection modality is an example of the simplicity and versatility offered by the technology, another advantage is the potential to sequentially or simultaneously measure both signals in the same reaction or within the same cells, contained in a single tube or microplate well, since the fluorescence and luminescence signals can be detected separately and do not interfere with each other. For example, tagged target protein expression levels can be sensitively quantitated with the luminescence of the active split NanoLuc® component of the complex, while its sub-cellular localization measured with fluorescence microscopy of the active split HaloTag component of the complex. These measurements can be configured in multiplex, homogeneous, live cell assays without the need for lysis, washing, or purification steps. In these examples, LgBiT or LgHT, or a fusion between the two, is produced inside of the cell where the tags or dual tag is being detected. However, this system could also be used for in vitro biochemical or cell lytic formats where the dual peptide tag is produced as a fusion to a target gene and detected in lytic format by recombinant LgBiT, LgHT, or a LgBiT-LgHT fusion.

Various embodiments herein include different substrates for the detection complexes, particularly for HaloTag® ligands, where many modified chloroalkanes have been described with dyes that fluoresce (in fluorogenic or non-fluorogenic fashion) at wavelengths across the visible and near IR spectrum as well as non-fluorescent ligands with a host of functionalities (e.g., mechanical and biological sensors, chemically-induced proximity, targeted degradation and post-translational modification, energy transfer, etc.). This provides a distinct advantage over a similar competitive technology with a small tag, split fluorescent proteins (GFP, RFP), since split FPs can only emit at one wavelength and do not offer the non-fluorescent labeling functionalities of HaloTag. Together, the advantages provided by dual luminescence and fluorescence detection using a small fusion tag and a single complementary detection polypeptide, along with the versatility offered by their substrates, imparts a broad set of advantages underlying individual technologies while providing new capabilities that can only be realized by configuring them together.

In some embodiments, provided herein are dual-reporter systems comprising: (a) a tandem peptide tag comprising (i) a peptide component of bioluminescent complex fused to (ii) a peptide component of modified dehalogenase complex; and (b) a tandem polypeptide reporter comprising (i) a polypeptide component of the bioluminescent complex and (ii) a polypeptide component of the modified dehalogenase complex; wherein the peptide component and the polypeptide component of the bioluminescent complex are capable of interacting to form the bioluminescent complex, and wherein the bioluminescent complex is capable of producing bioluminescence in the presence of a substrate for the bioluminescent complex; and wherein the peptide component and the polypeptide component of the modified dehalogenase complex are capable of interacting to form the modified dehalogenase complex, and wherein the modified dehalogenase complex is capable of forming a covalent bond with a haloalkyl ligand. In some embodiments, systems further comprise a substrate for the bioluminescent complex. In some embodiments, systems further comprise a haloalkyl ligand. In some embodiments, the haloalkyl ligand comprises a haloalkane moiety linked to a fluorophore. In some embodiments, the tandem peptide tag is linked to a target element (e.g., cellular target, protein, peptide, etc.). In some embodiments, the tandem peptide tag and the target element are expressed within a cell as a fusion.

In some embodiments, provided herein are tandem peptide tags comprising a peptide component of bioluminescent complex fused to a peptide component of modified dehalogenase complex; wherein the peptide component of the bioluminescent complex is capable of interacting with a polypeptide component of the bioluminescent complex to form the bioluminescent complex, and wherein the bioluminescent complex is capable of producing bioluminescence in the presence of a substrate for the bioluminescent complex; and wherein the peptide component of the modified dehalogenase complex is capable of interacting with a polypeptide component of the modified dehalogenase complex to form the modified dehalogenase complex, and wherein the modified dehalogenase complex is capable of forming a covalent bond with a haloalkyl ligand.

In some embodiments provided herein are systems comprising the tandem peptide tags herein and a polypeptide component of the bioluminescent complex. In some embodiments, systems further comprise a substrate for the bioluminescent complex. In some embodiments, systems further comprise a polypeptide component of the modified dehalogenase complex. In some embodiments, systems further comprise a haloalkyl ligand. In some embodiments, the polypeptide component of the modified dehalogenase complex and the polypeptide component of the bioluminescent complex are present in the system as independent reporter polypeptides. In some embodiments, the polypeptide component of the modified dehalogenase complex and the polypeptide component of the bioluminescent complex are present in the system as a tandem polypeptide reporter.

In some embodiments, provided herein are systems comprising a tandem peptide tag herein and a polypeptide component of the modified dehalogenase complex. In some embodiments, systems further comprise a substrate for the bioluminescent complex.

In some embodiments, provided herein are tandem polypeptide reporters comprising a polypeptide component of the bioluminescent complex and a polypeptide component of the modified dehalogenase complex; wherein the polypeptide component of the bioluminescent complex is capable of interacting with a peptide component of the bioluminescent complex to form the bioluminescent complex, and wherein the bioluminescent complex is capable of producing bioluminescence in the presence of a substrate for the bioluminescent complex; and wherein the polypeptide component of the modified dehalogenase complex is capable of interacting with a peptide component of the modified dehalogenase complex to form the modified dehalogenase complex, and wherein the modified dehalogenase complex is capable of forming a covalent bond with a haloalkyl ligand.

In some embodiments, provided herein are systems comprising a tandem peptide reporter herein and a peptide component of the bioluminescent complex. In some embodiments, systems further comprise a substrate for the bioluminescent complex. In some embodiments, systems further comprise a peptide component of the modified dehalogenase complex. In some embodiments, systems further comprise a haloalkyl ligand. In some embodiments, the peptide component of the modified dehalogenase complex and the peptide component of the bioluminescent complex are present in the system as independent reporter polypeptides. In some embodiments, the peptide component of the modified dehalogenase complex and the peptide component of the bioluminescent complex are present in the system as a tandem polypeptide reporter.

In some embodiments, provided herein are systems comprising a tandem polypeptide reporter herein and a peptide component of the modified dehalogenase complex. In some embodiments, systems further comprise a substrate for the bioluminescent complex.

In some embodiments, provided herein are methods of detecting a component of interest in a system, the method comprising: (a) linking the component of interest to a tandem peptide tag comprising a peptide component of bioluminescent complex fused to a peptide component of modified dehalogenase complex, wherein the peptide component of the bioluminescent complex is capable of interacting with a polypeptide component of the bioluminescent complex to form the bioluminescent complex, wherein the bioluminescent complex is capable of producing bioluminescence in the presence of a substrate for the bioluminescent complex, wherein the peptide component of the modified dehalogenase complex is capable of interacting with a polypeptide component of the modified dehalogenase complex to form the modified dehalogenase complex, and wherein the modified dehalogenase complex is capable of forming a covalent bond with a haloalkyl ligand; (b) contacting the component of interest linked to the tandem peptide tag with a polypeptide component of a modified dehalogenase complex under condition such that the modified dehalogenase complex is formed; (c) contacting the modified dehalogenase complex with a haloalkyl ligand comprising a haloalkane linked to a fluorophore; and (d) detecting fluorescence.

In some embodiments, provided herein are methods of detecting a component of interest in a system, the method comprising: (a) linking the component of interest to a tandem peptide tag comprising a peptide component of bioluminescent complex fused to a peptide component of modified dehalogenase complex, wherein the peptide component of the bioluminescent complex is capable of interacting with a polypeptide component of the bioluminescent complex to form the bioluminescent complex, wherein the bioluminescent complex is capable of producing bioluminescence in the presence of a substrate for the bioluminescent complex, wherein the peptide component of the modified dehalogenase complex is capable of interacting with a polypeptide component of the modified dehalogenase complex to form the modified dehalogenase complex, and wherein the modified dehalogenase complex is capable of forming a covalent bond with a haloalkyl ligand; (b) contacting the component of interest linked to the tandem peptide tag with a polypeptide component of a bioluminescent complex under condition such that the bioluminescent complex is formed; (c) contacting the bioluminescent complex with a substrate for the bioluminescent complex; and (d) detecting bioluminescence.

In some embodiments, provided herein are methods of detecting a component of interest in a system, the method comprising: (a) linking the component of interest to a tandem peptide tag comprising a peptide component of bioluminescent complex fused to a peptide component of modified dehalogenase complex, wherein the peptide component of the bioluminescent complex is capable of interacting with a polypeptide component of the bioluminescent complex to form the bioluminescent complex, wherein the bioluminescent complex is capable of producing bioluminescence in the presence of a substrate for the bioluminescent complex, wherein the peptide component of the modified dehalogenase complex is capable of interacting with a polypeptide component of the modified dehalogenase complex to form the modified dehalogenase complex, and wherein the modified dehalogenase complex is capable of forming a covalent bond with a haloalkyl ligand; (b) contacting the component of interest linked to the tandem peptide tag with a tandem polypeptide reporter comprising (i) the polypeptide component of a bioluminescent complex and (ii) the polypeptide component of a modified dehalogenase complex under condition such that the bioluminescent complex and modified dehalogenase complexes are formed; (c) contacting the bioluminescent complex with a substrate for the bioluminescent complex and/or a haloalkyl ligand comprising a haloalkane linked to a fluorophore; and (d) detecting bioluminescence and/or fluorescence.

In some embodiments, provided herein are methods of detecting a component of interest in a system, the method comprising: (a) linking the component of interest to a tandem peptide tag comprising a peptide component of bioluminescent complex fused to a peptide component of modified dehalogenase complex, wherein the peptide component of the bioluminescent complex is capable of interacting with a polypeptide component of the bioluminescent complex to form the bioluminescent complex, wherein the bioluminescent complex is capable of producing bioluminescence in the presence of a substrate for the bioluminescent complex, wherein the peptide component of the modified dehalogenase complex is capable of interacting with a polypeptide component of the modified dehalogenase complex to form the modified dehalogenase complex, and wherein the modified dehalogenase complex is capable of forming a covalent bond with a haloalkyl ligand; (b) contacting the component of interest linked to the tandem peptide tag with independent polypeptides comprising (i) the polypeptide component of a bioluminescent complex and (ii) the polypeptide component of a modified dehalogenase complex under condition such that the bioluminescent complex and modified dehalogenase complexes are formed; (c) contacting the bioluminescent complex with a substrate for the bioluminescent complex and/or a haloalkyl ligand comprising a haloalkane linked to a fluorophore; and (d) detecting bioluminescence and/or fluorescence.

### BRIEF DESCRIPTION OF THE DRAWINGS

FIG. 1. Cartoon depiction of an exemplary embodiment of the dual tag/reporter technology described herein.

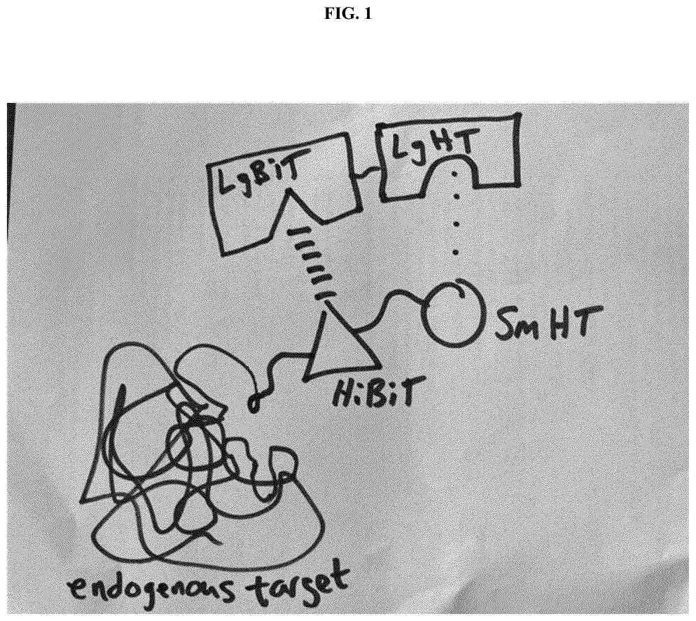

FIG. 2. Complementation of split HaloTag using the SmHT-HiBiT fusion. E. coli lysates expressing HaloTag[23-297]—FRB were combined with lysates expressing FKBP-SmHT with the indicated modifications (+/−HiBiT tag) and incubated with or without Rapamycin for 2 hours. Fluorescence activity of each combination was measured following addition JF646 HaloTag ligand to the mixture and incubation for 1 hour. SmHT refers to the HaloTag[3-19] fragment.

FIG. 3. Complementation of split HaloTag using the SmHT-HiBiT fusion. E. coli lysates expressing HaloTag[22-297](Q145H+P154)-FRB was combined with lysates expressing FKBP-SmHT with the indicated modifications (+/−HiBiT tag) and incubated with or without Rapamycin for 2 hours. Fluorescence activity of each combination was measured following addition JF646 HaloTag ligand to the mixture and incubation for 1 hour. SmHT refers to the HaloTag[3-19] fragment.

FIG. 4. Complementation of split HaloTag using the SmHT-HiBiT fusion. E. coli lysates expressing HaloTag[23-297]—FRB were combined with lysates expressing FKBP-SmHT with the indicated modifications (+/−HiBiT tag) and incubated with or without Rapamycin for 2 hours. Fluorescence activity of each combination was measured following addition JF646 HaloTag ligand to the mixture and incubation for 1 hour. Fold response was calculated as the fluorescence with Rapamycin divided by the fluorescence without Rapamycin. SmHT refers to the HaloTag[3-19] fragment.

FIG. 5. Complementation of split HaloTag using the SmHT-HiBiT fusion. E. coli lysates expressing HaloTag[22-297](Q145H+P154)-FRB was combined with lysates expressing FKBP-SmHT with the indicated modifications (+/−HiBiT tag) and incubated with or without Rapamycin for 2 hours. Fluorescence activity of each combination was measured following addition JF646 HaloTag ligand to the mixture and incubation for 1 hour. Fold response was calculated as the fluorescence without Rapamycin subtracted from the fluorescence with Rapamycin. SmHT refers to the HaloTag[3-19] fragment.

FIG. 6. Luminescence activity of the SmHT-HiBiT fusion tags when complemented with LgBiT. Purified LgBiT protein and Furimazine substrate was added to E. coli lysates expressing FKBP-SmHT with the indicated modifications (+/−HiBiT tag) following incubation with or without Rapamycin for 2 hours. Luminescence activity of each combination was measured after 20 minute incubation. SmHT refers to the HaloTag[3-19] fragment.

FIG. 7. Detection of the SmHT-HiBiT fusion tag through facilitated complementation and the LgHT-LgBiT detection polypeptide. E. coli lysates expressing HaloTag[22-297(M2F)]—FRB or HaloTag[22-297(M2F)]-LgBiT were combined with lysates expressing FKBP-SmHT with the indicated modifications (+/−HiBiT tag) and incubated with or without Rapamycin for 2 hours. Fluorescence activity of each combination was measured following addition JF646 HaloTag ligand to the mixture and incubation for 1 hour. SmHT refers to the HaloTag[3-19] fragment.

FIG. 8. Detection of the SmHT-HiBiT fusion tag through facilitated complementation and the LgHT-LgBiT detection polypeptide. E. coli lysates expressing HaloTag[22-297(Q145H+P154R)]—FRB or HaloTag[22-297](Q145H+P154R)-LgBiT was combined with lysates expressing FKBP-SmHT with the indicated modifications (+/−HiBiT tag) and incubated with or without Rapamycin for 2 hours. Fluorescence activity of each combination was measured following addition JF646 HaloTag ligand to the mixture and incubation for 1 hour. SmHT refers to the HaloTag[3-19] fragment.

FIG. 9. Detection of the SmHT-HiBiT fusion tag with luminescence following facilitated complementation and the LgHT-LgBiT detection polypeptide. E. coli lysates expressing the indicated LgHT-FRB or LgHT-LgBiT fusions were combined with lysates expressing FKBP-SmHT with the indicated modifications (+/−HiBiT tag) and incubated with or without Rapamycin for 2 hours. Following measurement for fluorescence activity by addition JF646 HaloTag ligand to the mixture and incubation for 1 hour, the mixture was diluted 1:10, Furimazine was added, and the luminescence activity measured. SmHT refers to the HaloTag[3-19] fragment.

FIG. 10. Detection of synthetic SmHT-HiBiT peptide with the LgHT-LgBiT detection polypeptide. E. coli lysates expressing HaloTag[22-297(M2F)]-LgBiT or HaloTag[22-297(Q145H+P174R)]-LgBiT were combined with a concentration range of synthetic SmHT-HiBiT peptide and incubated for 2 hours. Fluorescence activity of each combination was measured following addition JF646 HaloTag ligand to the mixture and incubation for 1 hour. For each combination, the lowest fluorescence intensity was used as the baseline for subtraction for all datapoints in order to enable nonlinear curve fitting and calculation of affinity. SmHT refers to the HaloTag[3-19] fragment.

FIG. 11. Detection of synthetic SmHT-HiBiT peptide with the LgHT-LgBiT detection polypeptide. E. coli lysates expressing HaloTag[22-297(M2F)]-LgBiT or HaloTag[22-297(Q145H+P174R)]-LgBiT were combined with a concentration range of synthetic SmHT-HiBiT peptide and incubated for 2 hours. Luminescence activity of each combination was measured following the addition of furimazine to the mixture and incubation for 20 minutes. SmHT refers to the HaloTag[3-19] fragment.

FIG. 12. Effects of varying HaloTag[3-19]-HiBiT internal linker lengths as C-terminal fusions to FKBP on luminescence activity in E. coli lysates. Constructs with varying internal linker lengths separating the HaloTag[3-19], and HiBiT sequences were expressed as C-terminal FKBP fusions in E. coli lysates. FKBP fusion-expressing lysates were combined with E. coli lysates expressing LgBiT, HaloTag[22-297](M2F)-12×Gly/Ser-LgBiT, or LgBiT-12×Gly/Ser-HaloTag[22-297](M2F). Reactions were incubated for 30 minutes at room temperature. Fluorofurimazine substrate was added to the reaction 5 minutes prior to luminescence detection.

FIG. 13. Effects of varying HaloTag[3-19]-HiBiT internal linker length on fluorescence activity in E. coli lysates. Constructs with varying internal linker lengths separating the HaloTag[3-19], and HiBiT sequences were expressed as C-terminal FKBP fusions in E. coli lysates. FKBP fusion-expressing lysates were combined with E. coli lysates expressing HaloTag[22-297](M2F)-12×Gly/Ser-LgBiT or LgBiT-12×Gly/Ser-HaloTag[22-297](M2F). Reactions were incubated for 30 minutes at room temperature. JF646 HaloTag ligand was added to the lysate mixture, and the reaction was incubated for an additional 2 hours at room temperature prior to fluorescence detection.

FIG. 14. Effects of varying HaloTag[3-19]-HiBiT internal linker length on fold response in fluorescence activity in E. coli lysates. Constructs with varying internal linker lengths separating the HaloTag[3-19], and HiBiT sequences were expressed as C-terminal FKBP fusions in E. coli lysates. FKBP fusion-expressing lysates were combined with E. coli lysates expressing HaloTag[22-297](M2F)-12×Gly/Ser-LgBiT or LgBiT-12×Gly/Ser-HaloTag[22-297](M2F). Reactions were incubated for 30 minutes at room temperature. JF646 HaloTag ligand was added to the lysate mixture, and the reaction was incubated for an additional 2 hours at room temperature prior to fluorescence detection. Fold response was calculated by dividing raw fluorescence readings for each reaction containing FKBP fusion and polypeptide reporter lysate by the fluorescence reading of polypeptide reporter lysate alone.

FIG. 15. Effects of varying internal HaloTag[3-19]-HiBiT linker lengths for N-terminal tags on luminescence activity in E. coli lysates. Constructs with varying internal linker lengths separating the HaloTag[3-19], and HiBiT sequences were expressed as N-terminal FKBP fusions in E. coli lysates. FKBP fusion-expressing lysates were combined with E. coli lysates expressing LgBiT, HaloTag[22-297](M2F)-12×Gly/Ser-LgBiT, or LgBiT-12×Gly/Ser-HaloTag[22-297](M2F). Reactions were incubated for 30 minutes at room temperature. Reactions were diluted 10-fold into buffer, and fluorofurimazine substrate was added to the diluted reaction 5 minutes prior to luminescence detection.

FIG. 16. Effects of varying internal HaloTag[3-19]-HiBiT linker lengths for N-terminal fusions to FKBP on fluorescence activity in E. coli lysates. Constructs with varying internal linker lengths separating the HaloTag[3-19], and HiBiT sequences were expressed as N-terminal FKBP fusions in E. coli lysates. FKBP fusion-expressing lysates were combined with E. coli lysates expressing HaloTag[22-297](M2F)-12×Gly/Ser-LgBiT or LgBiT-12×Gly/Ser-HaloTag[22-297](M2F). Reactions were incubated for 30 minutes at room temperature. JF646 HaloTag ligand was added to the lysate mixture, and the reaction was incubated for an additional 2 hours at room temperature prior to fluorescence detection.

FIG. 17. Effects of varying internal HaloTag[3-19]-HiBiT linker lengths for N-terminal fusions to FKBP on fold response in fluorescence activity in E. coli lysates. Constructs with varying internal linker lengths separating the HaloTag[3-19], and HiBiT sequences were expressed as N-terminal FKBP fusions in E. coli lysates. FKBP fusion-expressing lysates were combined with E. coli lysates expressing HaloTag[22-297](M2F)-12×Gly/Ser-LgBiT or LgBiT-12×Gly/Ser-HaloTag[22-297](M2F). Reactions were incubated for 30 minutes at room temperature. JF646 HaloTag ligand was added to the lysate mixture, and the reaction was incubated for an additional 2 hours at room temperature prior to fluorescence detection. Fold response was calculated by dividing raw fluorescence readings for each reaction containing FKBP fusion and polypeptide reporter lysate by the fluorescence reading of polypeptide reporter lysate alone.

FIG. 18. Effects of varying HaloTag[3-19]-HiBiT internal linker length and composition on luminescence activity in E. coli lysates. Constructs with varying internal linker lengths and compositions separating the HaloTag[3-19], and HiBiT sequences were expressed as C-terminal FKBP fusions in E. coli lysates. FKBP fusion-expressing lysates were combined with E. coli lysates expressing LgBiT, HaloTag[22-297](M2F)-12×Gly/Ser-LgBiT, or LgBiT-12×Gly/Ser-HaloTag[22-297](M2F). Reactions were incubated for 30 minutes at room temperature. Fluorofurimazine substrate was added to the reaction 5 minutes prior to luminescence detection.

FIG. 19. Effects of varying HaloTag[3-19]-HiBiT internal linker length and composition on fluorescence activity in E. coli lysates. Constructs with varying internal linker lengths separating the HaloTag[3-19], and HiBiT sequences were expressed as C-terminal FKBP fusions in E. coli lysates. FKBP fusion-expressing lysates were combined with E. coli lysates expressing HaloTag[22-297](M2F)-12×Gly/Ser-LgBiT or LgBiT-12×Gly/Ser-HaloTag[22-297](M2F). Reactions were incubated for 30 minutes at room temperature. JF646 HaloTag ligand was added to the lysate mixture, and the reaction was incubated for an additional 2 hours at room temperature prior to fluorescence detection.

FIG. 20. Effects of varying linker length between FKBP and HaloTag[3-19]-HiBiT C-terminal fusions on luminescence activity in E. coli lysates. Constructs with varying linker lengths separating FKBP and HaloTag[3-19]-HiBiT were expressed in E. coli lysates. FKBP fusion-expressing lysates were combined with E. coli lysates expressing LgBiT, HaloTag[22-297](M2F)-12×Gly/Ser-LgBiT, or LgBiT-12×Gly/Ser-HaloTag[22-297](M2F). Reactions were incubated for 30 minutes at room temperature. Fluorofurimazine substrate was added to the reaction 5 minutes prior to luminescence detection.

FIG. 21. Effects of varying linker length between FKBP and HaloTag[3-19]-HiBiT C-terminal fusions on fluorescence activity in E. coli lysates. Constructs with varying linker lengths separating FKBP and HaloTag[3-19]-HiBiT were expressed in E. coli lysates. FKBP fusion-expressing lysates were combined with E. coli lysates expressing LgBiT, HaloTag[22-297](M2F)-12×Gly/Ser-LgBiT, or LgBiT-12×Gly/Ser-HaloTag[22-297](M2F). Reactions were incubated for 30 minutes at room temperature. JF646 HaloTag ligand was added to the lysate mixture, and the reaction was incubated for an additional 2 hours at room temperature prior to fluorescence detection.

FIG. 22. Effects of varying linker length between N-terminal HaloTag[3-19]-HiBiT fusions to FKBP on luminescence activity in E. coli lysates. Constructs with varying linker lengths separating FKBP and HaloTag[3-19]-HiBiT were expressed in E. coli lysates. FKBP fusion-expressing lysates were combined with E. coli lysates expressing LgBiT, HaloTag[22-297](M2F)-12×Gly/Ser-LgBiT, or LgBiT-12×Gly/Ser-HaloTag[22-297](M2F). Reactions were incubated for 30 minutes at room temperature. Reactions were diluted 10-fold in buffer, and fluorofurimazine substrate was added to the diluted reaction 5 minutes prior to luminescence detection.

FIG. 23. Effects of varying linker length between N-terminal HaloTag[3-19]-HiBiT fusions to FKBP on fluorescence activity in E. coli lysates. Constructs with varying linker lengths separating FKBP and HaloTag[3-19]-HiBiT were expressed in E. coli lysates. FKBP fusion-expressing lysates were combined with E. coli lysates expressing LgBiT, HaloTag[22-297](M2F)-12×Gly/Ser-LgBiT, or LgBiT-12×Gly/Ser-HaloTag[22-297](M2F). Reactions were incubated for 30 minutes at room temperature. JF646 HaloTag ligand was added to the lysate mixture, and the reaction was incubated for an additional 2 hours at room temperature prior to fluorescence detection.

FIG. 24. Effects of HaloTag[3-19] truncation on luminescence activity in E. coli lysates. HaloTag[3-19]-HiBiT constructs containing HaloTag[3-19] truncations were expressed in E. coli lysates as C-terminal fusions to FKBP. FKBP fusion-expressing lysates were combined with E. coli lysates expressing LgBiT, HaloTag[22-297](M2F)-12×Gly/Ser-LgBiT, or LgBiT-12×Gly/Ser-HaloTag[22-297](M2F). Reactions were incubated for 30 minutes at room temperature. Fluorofurimazine substrate was added to the reaction 5 minutes prior to luminescence detection.

FIG. 25. Effects of HaloTag[3-19] truncation on fluorescence activity of HaloTag[3-19]-HiBiT in E. coli lysates. HaloTag[3-19]-HiBiT constructs containing HaloTag[3-19] truncations were expressed in E. coli lysates as C-terminal fusions to FKBP. FKBP fusion-expressing lysates were combined with E. coli lysates expressing HaloTag[22-297](M2F)-12×Gly/Ser-LgBiT or LgBiT-12×Gly/Ser-HaloTag[22-297](M2F). Reactions were incubated for 30 minutes at room temperature. JF646 HaloTag ligand was added to the lysate mixture, and the reaction was incubated for an additional 2 hours at room temperature prior to fluorescence detection.

FIG. 26. Effects of HiBiT mutations on luminescence activity of HaloTag[3-19]-HiBiT in E. coli lysates. HaloTag[3-19]-HiBiT constructs containing HiBiT mutations were expressed in E. coli lysates as FKBP fusions. FKBP fusion-expressing lysates were combined with E. coli lysates expressing LgBiT, HaloTag[22-297](M2F)-12×Gly/Ser-LgBiT, or LgBiT-12×Gly/Ser-HaloTag[22-297](M2F). Reactions were incubated for 30 minutes at room temperature. Fluorofurimazine substrate was added to the reaction 5 minutes prior to luminescence detection.

FIG. 27 Effects of HiBiT mutations on fluorescence activity of HaloTag[3-19]-HiBiT in E. coli lysates. HaloTag[3-19]-HiBiT constructs containing HiBiT mutations were expressed in E. coli lysates as C-terminal fusions to FKBP. FKBP fusion-expressing lysates were combined with E. coli lysates expressing HaloTag[22-297](M2F)-12×Gly/Ser-LgBiT or LgBiT-12×Gly/Ser-HaloTag[22-297](M2F). Reactions were incubated for 30 minutes at room temperature. JF646 HaloTag ligand was added to the lysate mixture, and the reaction was incubated for an additional 2 hours at room temperature prior to fluorescence detection.

FIG. 28. Effects of HaloTag[3-19]-HiBiT variants on enhancement of LgBiT luminescence across a range of synthetic peptide concentrations. Titrations of HaloTag[3-19]-HiBiT synthetic peptide variants containing mutations (bolded) from the native sequence (EIGTGFPFDPHYVEVLG) were added to purified LgBiT-6×His protein. Reactions were incubated for 30 minutes at 4° C. Furimazine substrate was added to the reaction immediately prior to measuring luminescence. Peptides in this experiment are conjugated to biotin at the N-terminus.

FIG. 29. Effects of HaloTag[3-19]-HiBiT variants on enhancement of HaloTag[22-297](M2F+K140E)-12×Gly/Ser-LgBiT-6×His luminescence across a range of synthetic peptide concentrations. Titrations of HaloTag[3-19]-HiBiT variants synthetic peptides containing mutations (bolded) from the native sequence (EIGTGFPFDPHYVEVLG) were added to purified HaloTag[22-297](M2F+K140E)-12×Gly/Ser-LgBiT-6×His protein. Reactions were incubated for 30 minutes at 4° C. Furimazine substrate was added to the reaction immediately prior to measuring luminescence. Peptides in this experiment are conjugated to biotin at the N-terminus.

FIG. 30. Effects of HaloTag[3-19]-HiBiT variants on luminescence enhancement of HaloTag[22-297](M2F+K140E)-12×Gly/Ser-LgBiT-6×His across a range of peptide concentrations. Titrations of HaloTag[3-19]-HiBiT synthetic peptides containing mutations (bolded) from the native sequence (EIGTGFPFDPHYVEVLG) were added to purified HaloTag[22-297](M2F+K140E)-12×Gly/Ser-LgBiT-6×His protein. Reactions were incubated for 30 minutes at 4° C. Furimazine substrate was added to the reaction immediately prior to measuring luminescence. Peptides in this experiment are conjugated to biotin at the N-terminus.

FIG. 31. Effects of HaloTag[3-19]-HiBiT variants on enhancement of HaloTag[22-297](M2F)-12×Gly/Ser-LgBiT-6×His luminescence across a range of synthetic peptide concentrations. Titrations of HaloTag[3-19]-HiBiT variant synthetic peptides containing sequence mutations or additional linker residues (both bolded) from the native sequence (EIGTGFPFDPHYVEVLG) were added to purified HaloTag[22-297](M2F)-12×Gly/Ser-LgBiT-6×His protein. Reactions were incubated for 30 minutes at 4° C. Furimazine substrate was added to the reaction immediately prior to measuring luminescence.

FIG. 32. Effects of HaloTag[3-19]-HiBiT variants on luminescence enhancement of HaloTag[22-297](M2F)-12×Gly/Ser-LgBiT-6×His across a range of synthetic peptide concentrations. Titrations of HaloTag[3-19]-HiBiT variant synthetic peptides containing mutations (bolded) from the native sequence (EIGTGFPFDPHYVEVLG) were added to purified HaloTag[22-297](M2F)-12×Gly/Ser-LgBiT-6×His protein. Reactions were incubated for 30 minutes at 4° C. Furimazine substrate was added to the reaction immediately prior to measuring luminescence.

FIG. 33. Effects of HaloTag[3-19]-HiBiT variants on enhancement of HaloTag[22-297](M2F+K140E)-12×Gly/Ser-LgBiT-6×His fluorescence across a range of synthetic peptide concentrations. Titrations of HaloTag[3-19]-HiBiT variant synthetic peptides containing mutations (bolded) from the native sequence (EIGTGFPFDPHYVEVLG) were added to purified HaloTag[22-297](M2F+K140E)-12×Gly/Ser-LgBiT-6×His protein. Reactions were incubated for 30 minutes at 4° C. JF646 HaloTag ligand was added to each reaction, and fluorescence was measured after 30 minutes room temperature incubation with ligand. Peptides in this experiment are conjugated to biotin at the N-terminus.

FIG. 34. Effects of HaloTag[3-19]-HiBiT variants on enhancement of HaloTag[22-297](M2F+K140E)-12×Gly/Ser-LgBiT fluorescence of when complemented with synthetic peptides across a range of peptide concentrations. Titrations of HaloTag[3-19]-HiBiT variant synthetic peptides containing mutations (bolded) from the native sequence (EIGTGFPFDPHYVEVLG) were added to purified HaloTag[22-297](M2F+K140E)-12×Gly/Ser-LgBiT-6×His protein. Reactions were incubated for 30 minutes at 4° C. JF646 HaloTag ligand was added to each reaction, and fluorescence was measured after 30 minutes room temperature incubation with ligand. Peptides in this experiment are conjugated to biotin at the N-terminus.

FIG. 35. Effects of HaloTag[3-19]-HiBiT variants on enhancement of HaloTag[22-297](M2F+K140E)-12×Gly/Ser-LgBiT fluorescence across a range of synthetic peptide concentrations. Titrations of HaloTag[3-19]-HiBiT variant synthetic peptides containing mutations (bolded) from the native sequence (EIGTGFPFDPHYVEVLG) were added to purified HaloTag[22-297](M2F+K140E)-12×Gly/Ser-LgBiT-6×His protein. Reactions were incubated for 30 minutes at 4° C. JF646 HaloTag ligand was added to each reaction, and fluorescence was measured after 30 minutes room temperature incubation with ligand.

FIG. 36. Effects of HaloTag[3-19]-HiBiT variants on fluorescence of HaloTag[22-297](M2F+K140E)-12×Gly/Ser-LgBiT-6×His across a range of synthetic peptide concentrations. Titrations of HaloTag[3-19]-HiBiT variant synthetic peptides containing mutations (bolded) from the native sequence (EIGTGFPFDPHYVEVLG) were added to purified HaloTag[22-297](M2F+K140E)-12×Gly/Ser-LgBiT-6×His protein. Reactions were incubated for 30 minutes at 4° C. JF646 HaloTag ligand was added to each reaction, and fluorescence was measured after 30 minutes room temperature incubation with ligand.

FIG. 37. Effects of HaloTag[3-19] variants on fluorescence intensity of HaloTag[22-297](M2F)-12×Gly/Ser-LgBiT-6×His across a range of synthetic peptide concentrations. Titrations of HaloTag[3-19] synthetic peptides containing mutations (bolded) or deletions (denoted by a dash) from the native sequence (SEQ ID NO: 3061: EIGTGFPFDPHYVEVLG) were added to an E. coli lysate expressing HaloTag[22-297](M2F)-12×Gly/Ser-LgBiT. Reactions were incubated for 2 hours at room temperature. JF646 HaloTag ligand was added to the synthetic peptide/lysate mixtures, and the reaction was incubated for an additional 1.5 hours at room temperature prior to fluorescence detection.

FIG. 38. Effects of HaloTag[3-19] variants on fluorescence intensity of HaloTag[22-297](M2F)-12×Gly/Ser-LgBiT across a range of synthetic peptide concentrations. Titrations of HaloTag[3-19] synthetic peptides containing mutations (bolded) from the native sequence (SEQ ID NO: 3061: EIGTGFPFDPHYVEVLG) were added to an E. coli lysate expressing HaloTag[22-297](M2F)-12×Gly/Ser-LgBiT. Reactions were incubated for 2 hours at room temperature. JF646 HaloTag ligand was added to the synthetic peptide/lysate mixtures, and the reaction was incubated for an additional 1.5 hours at room temperature prior to fluorescence detection.

FIG. 39. Effects of HaloTag[3-19] variants on enhancement of HaloTag[22-297](M2F)-12×Gly/Ser-LgBiT fluorescence across a range of synthetic peptide concentrations. Titrations of HaloTag[3-19] synthetic peptides containing mutations (bolded) from the native sequence (SEQ ID NO: 3061: EIGTGFPFDPHYVEVLG) were added to an E. coli lysate expressing HaloTag[22-297](M2F)-12×Gly/Ser-LgBiT. Reactions were incubated for 2 hours at room temperature. JF646 HaloTag ligand was added to the synthetic peptide/lysate mixtures, and the reaction was incubated for an additional 1.5 hours at room temperature prior to fluorescence detection.

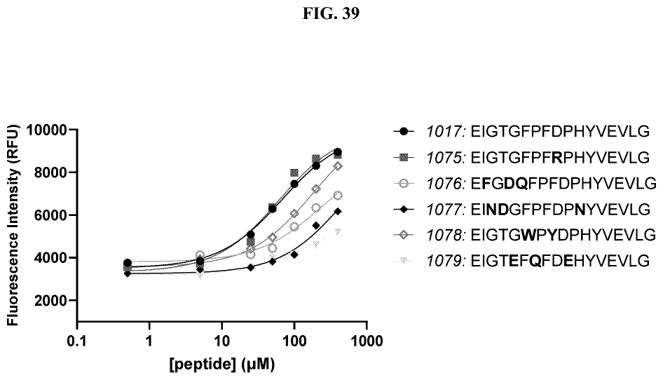

FIG. 40. Effects of HaloTag[3-19] variants on fluorescence intensity of HaloTag[22-297](M2F)-12×Gly/Ser-LgBiT across a range of synthetic peptide concentrations. Titrations of HaloTag[3-19] synthetic peptides containing mutations (bolded) from the native sequence (SEQ ID NO: 3061: EIGTGFPFDPHYVEVLG) were added to an E. coli lysate expressing HaloTag[22-297](M2F)-12×Gly/Ser-LgBiT. Reactions were incubated for 2 hours at room temperature. JF646 HaloTag ligand was added to the synthetic peptide/lysate mixtures, and the reaction was incubated for an additional 1.5 hours at room temperature prior to fluorescence detection.

FIG. 41. Binding of HaloTag[22-297](M2F+F148M+V177E)-12×Gly/Ser-LgBiT-6×His to Biotin-HaloTag[3-19](12R)-4×Gly/Ser-VS-HiBiT synthetic peptide. Association and dissociation (right of the dotted line) of polypeptide reporter HaloTag[22-297](M2F+F148M+V177E)-12×Gly/Ser-LgBiT-6×His across a concentration range of 0.62-150 nM to biotin-HaloTag[3-19](12R)-4×Gly/Ser-VS-HiBiT synthetic peptide immobilized on streptavidin probes was measured using biolayer interferometry in PBST at 25° C.

FIG. 42. Yeast surface display of HaloTag[3-19]-4×Gly/Ser-VS-HiBiT. HaloTag[3-19]-4×Gly/Ser-VS-HiBiT was fused to yeast cell mating factor Aga2 for surface display. The resulting construct was transformed and expressed in S. cerevisiae. Cells were incubated with HaloTag[22-297](M2F)-12×Gly/Ser-LgBiT-6×His for 45 minutes at room temperature. Unbound HaloTag[22-297](M2F)-12×Gly/Ser-LgBiT-6×His was washed, and cells were incubated with JF549 HaloTag ligand for 45 minutes at room temperature. Unbound ligand was washed away from cells, and cells were incubated with anti-HA tag antibody from mouse for 30 minutes at room temperature. Excess primary antibody was washed away, and cells were incubated with the secondary antibody, goat anti-mouse IgG conjugated to AlexaFluor488, for 30 minutes at room temperature. Cells were washed and analyzed on a Sony Cell Sorter. The histograms represent 10,192 recorded events from a singlet cell positive gate. Cells in the AF488+ gate represent the population of cells that are positive for surface display of HaloTag[3-19]-4×Gly/Ser-VS-HiBiT (left histogram, 51.23%). Cells in the JF549+ gate represent the population of cells that are expressing HaloTag[3-19]-4×Gly/Ser-VS-HiBiT and are complemented with JF549 HaloTag ligand-bound HaloTag[22-297](M2F)-12×Gly/Ser-LgBiT-6×His (right histogram, 48.88%).

FIG. 43. Yeast surface display of EGFP-GGSG-HaloTag[3-19]-GGSG-VS-HiBiT. EGFP-GGSG-HaloTag[3-19]-GGSG-VS-HiBiT was fused to yeast cell mating factor Aga2 for surface display. The resulting construct was transformed and expressed in S. cerevisiae. Cells were incubated with HaloTag[22-297](M2F)-12×Gly/Ser-LgBiT-6×His for 45 minutes at room temperature. Unbound HaloTag[22-297](M2F)-12×Gly/Ser-LgBiT-6×His was washed, and cells were incubated with JF549 HaloTag ligand for 45 minutes at room temperature. Cells were washed and analyzed on a Sony Cell Sorter. The histograms represent 10,569 recorded events. Cells in the EGFP+ gate represent the population of cells that are positive for surface display of EGFP-GGSG-HaloTag[3-19]-GGSG-VS-HiBiT (left histogram, 48.07%). Cells in the JF549+ gate represent the population of cells that are complemented with JF549 HaloTag ligand-bound HaloTag[22-297](M2F)-12×Gly/Ser-LgBiT-6×His (right histogram, 46.41%).

FIG. 44. Yeast surface display of EGFP-GGSG-HaloTag[3-19]-GGSG-NVSGWRLFKKISN. The Aga2-EGFP-GGSG-HaloTag[3-19]-GGSG-VS-HiBiT surface display construct was altered so that the VS-HiBiT sequence was replaced by the sequence NVSGWRLFKKISN, which complements LgBiT with a lower affinity than HiBiT. The resulting construct was transformed and expressed in S. cerevisiae. Cells were incubated with HaloTag[22-297](M2F)-12×Gly/Ser-LgBiT-6×His for 45 minutes at room temperature. Unbound HaloTag[22-297](M2F)-12×Gly/Ser-LgBiT-6×His was washed, and cells were incubated with JF549 HaloTag ligand for 45 minutes at room temperature. Cells were washed and analyzed on a Sony Cell Sorter. The histograms represent 11,216 recorded events. Cells in the EGFP+ gate represent the population of cells that are positive for surface display of EGFP-GGSG-HaloTag[3-19]-NVSGWRLFKKISN (left histogram, 44.57%). Cells in the JF549+ gate represent the population of cells that are complemented with JF549 HaloTag ligand-bound HaloTag[22-297](M2F)-12×Gly/Ser-LgBiT-6×His (right histogram, 44.24%).

FIG. 45. Yeast surface display of EGFP-GGSG-HaloTag[3-19]-GGSG-NVTGYRLFKKISN. The Aga2-EGFP-GGSG-HaloTag[3-19]-GGSG-VS-HiBiT surface display construct was altered so that the VS-HiBiT sequence was replaced by the sequence NVTGYRLFKKISN, which complements LgBiT with a lower affinity than HiBiT. The resulting construct was transformed and expressed in S. cerevisiae. Cells were incubated with HaloTag[22-297](M2F)-12×Gly/Ser-LgBiT-6×His for 45 minutes at room temperature. Unbound HaloTag[22-297](M2F)-12×Gly/Ser-LgBiT-6×His was washed, and cells were incubated with JF549 HaloTag ligand for 45 minutes at room temperature. Cells were washed and analyzed on a Sony Cell Sorter. The histograms represent 11,203 recorded events. Cells in the EGFP+ gate represent the population of cells that are positive for surface display of EGFP-GGSG-HaloTag[3-19]-GGSG-NVTGYRLFKKISN (left histogram, 46.47%). Cells in the JF549+ gate represent the population of cells that are complemented with JF549 HaloTag ligand-bound HaloTag[22-297](M2F)-12×Gly/Ser-LgBiT-6×His (right histogram, 44.84%).

FIG. 46. Yeast surface display of EGFP-GGSG-HaloTag[3-19]-GGSG-VTGYRLFEKIS. The Aga2-EGFP-GGSG-HaloTag[3-19]-GGSG-VS-HiBiT surface display construct was altered so that the VS-HiBiT sequence was replaced by the sequence VTGYRLFEKIS, which complements LgBiT with a lower affinity than HiBiT. The resulting construct was transformed and expressed in S. cerevisiae. Cells were incubated with HaloTag[22-297](M2F)-12×Gly/Ser-LgBiT-6×His for 45 minutes at room temperature. Unbound HaloTag[22-297](M2F)-12×Gly/Ser-LgBiT-6×His was washed, and cells were incubated with JF549 HaloTag ligand for 45 minutes at room temperature. Cells were washed and analyzed on a Sony Cell Sorter. The histograms represent 11,230 recorded events. Cells in the EGFP+ gate represent the population of cells that are positive for surface display of EGFP-GGSG-HaloTag[3-19]-GGSG-VTGYRLFEKIS (left histogram, 49.51%). Cells in the JF549+ gate represent the population of cells that are complemented with JF549 HaloTag ligand-bound HaloTag[22-297](M2F)-12×Gly/Ser-LgBiT-6×His (right histogram, 6.96%).

FIG. 47. Complementation of HaloTag[22-297](M2F)-12×Gly/Ser-LgBiT variant in E. coli lysates using a genetic fusion of FKBP to HaloTag[3-19] peptide. HaloTag[22-297](M2F)-12×Gly/Ser-LgBiT or variant with M49F+D53G mutations was expressed in E. coli lysates and combined with lysates expressing FKBP fused to different tag sequences. Reactions were incubated at room temperature for 2 hours and then labeled with 50 nM JF646 HaloTag ligand prior to fluorescence detection. Data points represent independent cultures of each construct combined during the experiment, with error bars representing one standard deviation from the mean.

FIG. 48. Complementation and stabilization of HaloTag[22-297](M2F+D53S+D56P)-12×Gly/Ser-LgBiT in E. coli lysates using a synthetic HaloTag[3-19] peptide. HaloTag[22-297](M2F+D53S+D56P)-12×Gly/Ser-LgBiT was expressed in E. coli lysates and combined with excess synthetic HaloTag[3-19] peptide. Reactions were incubated for indicated time and temperatures and then labeled with 50 nM JF646 HaloTag ligand prior to fluorescence detection.

FIG. 49. Stability of HaloTag[22-297](M2F)-12×Gly/Ser-LgBiT in E. coli lysates after complementation with different synthetic peptides. HaloTag[22-297](M2F)-12×Gly/Ser-LgBiT was expressed in E. coli lysates and combined with 10 micromolar or more of the indicated synthetic peptides. Reactions were incubated at indicated temperature for 10 minutes and then labeled with 50 nM JF646 HaloTag ligand prior to fluorescence detection.

FIG. 50. Stability of HaloTag[22-297](M2F+D53G+V177E)-12×Gly/Ser-LgBiT in E. coli lysates after complementation with different synthetic peptides. HaloTag[22-297](M2F+D53G+V177E)-12×Gly/Ser-LgBiT was expressed in E. coli lysates and combined with 10 micromolar or more of the indicated synthetic peptides. Reactions were incubated at indicated temperature for 10 minutes and then labeled with 50 nM JF646 HaloTag ligand prior to fluorescence detection.

FIG. 51. Stability of HaloTag[22-297](M2F+M49F+S89A)-12×Gly/Ser-LgBiT in E. coli lysates after complementation with different synthetic peptides. HaloTag[22-297](M2F+M49F+S89A)-12×Gly/Ser-LgBiT was expressed in E. coli lysates and combined with 10 micromolar or more of the indicated synthetic peptides. Reactions were incubated at indicated temperature for 10 minutes and then labeled with 50 nM JF646 HaloTag ligand prior to fluorescence detection.

FIG. 52. Stability of HaloTag[22-297](M2F+M49F+L57I)-12×Gly/Ser-LgBiT in E. coli lysates after complementation with different synthetic peptides. HaloTag[22-297](M2F+M49F+L57I)-12×Gly/Ser-LgBiT was expressed in E. coli lysates and combined with 10 micromolar or more of the indicated synthetic peptides. Reactions were incubated at indicated temperature for 10 minutes and then labeled with 50 nM JF646 HaloTag ligand prior to fluorescence detection.

FIG. 53. Labeling kinetics of HaloTag[22-297](M2F+D53G+V177E)-12×Gly/Ser-LgBiT-3×Gly/Ser-6×His variant in E. coli lysates after complementation with synthetic peptide. HaloTag[22-297](M2F)-12×Gly/Ser-LgBiT-3×Gly/Ser-6×His or its variant with D53G+V177E mutations were expressed in E. coli lysates and combined with 200 micromolar HaloTag[3-19] synthetic peptide. Reactions were incubated at room temperature for 3 hours and then added to 10 nM TMR HaloTag ligand, reading continuously for fluorescence polarization of the ligand to monitor labeling. Lysate was added at the 3 minute timepoint of the graph to initially measure the baseline fluorescence polarization of the ligand and subsequent increase in binding upon lysate addition.

FIG. 54. Labeling kinetics of HaloTag[22-297](M2F+K140E)-12×Gly/Ser-LgBiT-3×Gly/Ser-6×His variant in E. coli lysates after complementation with synthetic peptide. HaloTag[22-297](M2F)-12×Gly/Ser-LgBiT-3×Gly/Ser-6×His or its variant with K140E mutation was expressed in E. coli lysates and combined with 200 micromolar HaloTag[3-19] synthetic peptide. Reactions were incubated at room temperature for 3 hours and then added to 10 nM TMR HaloTag ligand, reading continuously for fluorescence polarization of the ligand to monitor labeling. Lysate was added at the 3 minute timepoint of the graph to initially measure the baseline fluorescence polarization of the ligand and subsequent increase in binding upon lysate addition.

FIG. 55. Labeling kinetics of HaloTag[22-297](M2F+D53G+K140E)-12×Gly/Ser-LgBiT-3×Gly/Ser-6×His variant in E. coli lysates after complementation with synthetic peptide. HaloTag[22-297](M2F)-12×Gly/Ser-LgBiT-3×Gly/Ser-6×His or variants with K140E or D53G+K140E were expressed in E. coli lysates and combined with 200 micromolar HaloTag[3-19] synthetic peptide. Reactions were incubated at room temperature for 3 hours and then added to 10 nM TMR HaloTag ligand, reading continuously for fluorescence polarization of the ligand to monitor labeling. Lysate was added at the 3 minute timepoint of the graph to initially measure the baseline fluorescence polarization of the ligand and subsequent increase in binding upon lysate addition.

FIG. 56. Endpoint labeling activity of HaloTag[22-297](M2F+D53G)-12×Gly/Ser-LgBiT-3×Gly/Ser-6×His variants in E. coli lysates after complementation with synthetic peptide. HaloTag[22-297](M2F+D53G)-12×Gly/Ser-LgBiT-3×Gly/Ser-6×His variants were expressed in E. coli lysates and combined with 200 micromolar HaloTag[3-19] synthetic peptide. Reactions were incubated at room temperature for 3 hours and then combined with 100 nM JF646 HaloTag ligand. Fluorescence intensity was measured after 60 minute incubation with the ligand.

FIG. 57. Stability and labeling kinetics of purified HaloTag[22-297](M2F)-12×Gly/Ser-LgBiT-3×Gly/Ser-6×His after complementation with synthetic peptide. HaloTag[22-297](M2F)-12×Gly/Ser-LgBiT-3×Gly/Ser-6×His at 10 nM was combined with 125 micromolar HaloTag[3-19] synthetic peptide and incubated at 4 C or 32 C for 10 minutes. After preincubation with peptide at the indicated temperature, 2.5 nM TMR HaloTag ligand was injected into the reaction, and fluorescence polarization monitored over time to observe labeling kinetics.

FIG. 58. Stability and labeling kinetics of purified HaloTag[22-297](M2F+K140E)-12×Gly/Ser-LgBiT-3×Gly/Ser-6×His after complementation with synthetic peptide. HaloTag[22-297](M2F+K140E)-12×Gly/Ser-LgBiT-3×Gly/Ser-6×His at 10 nM was combined with 125 micromolar HaloTag[3-19] synthetic peptide and incubated at 4° C. or 32° C. for 10 minutes. After preincubation with peptide at the indicated temperature, 2.5 nM TMR HaloTag® ligand was injected into the reaction, and fluorescence polarization monitored over time to observe labeling kinetics.

FIG. 59. Stability and labeling kinetics of purified HaloTag[22-297](M2F+D53G+K140E)-12×Gly/Ser-LgBiT-3×Gly/Ser-6×His after complementation with synthetic peptide. HaloTag[22-297](M2F+D53G+K140E)-12×Gly/Ser-LgBiT-3×Gly/Ser-6×His at 10 nM was combined with 125 micromolar HaloTag[3-19] synthetic peptide and incubated at 4° C. or 32° C. for 10 minutes. After preincubation with peptide at the indicated temperature, 2.5 nM TMR HaloTag® ligand was injected into the reaction, and fluorescence polarization monitored over time to observe labeling kinetics.

FIG. 60. Stability and labeling kinetics of purified HaloTag[22-297](M2F+M49F+D53S+V177E)-12×Gly/Ser-LgBiT-3×Gly/Ser-6×His after complementation with synthetic peptide. HaloTag[22-297](M2F+D53S+V177E)-12×Gly/Ser-LgBiT-3×Gly/Ser-6×His at 10 nM was combined with 125 micromolar HaloTag[3-19] synthetic peptide and incubated at 4° C. or 37° C. for 10 minutes. After preincubation with peptide at the indicated temperature, 2.5 nM TMR HaloTag ligand was injected into the reaction, and fluorescence polarization monitored over time to observe labeling kinetics.

FIG. 61. Stability and labeling kinetics of purified HaloTag[22-297](M2F+D53G+K140E)-12×Gly/Ser-LgBiT-3×Gly/Ser-6×His after complementation with synthetic peptide. HaloTag[22-297](M2F+D53G+K140E)-12×Gly/Ser-LgBiT-3×Gly/Ser-6×His at 10 nM was combined with 125 micromolar HaloTag[3-19] synthetic peptide and incubated at 4° C. or 32° C. for 10 minutes. After preincubation with peptide at the indicated temperature, 2.5 nM TMR HaloTag ligand was injected into the reaction, and fluorescence polarization monitored over time to observe labeling kinetics.

FIG. 62. Fluorescent Intensity of HaloTag[22-297](M2F)-12×Gly/Ser-LgBiT Variants co-expressing FKBP-HaloTag[3-19]-5×Gly/Ser-VS-HiBiT in Live Mammalian Cells. HeLa cells were transiently transfected with separate plasmids expressing the indicated HaloTag[22-297](M2F)-12×Gly/Ser-LgBiT variant and HaloTag[3-19]-5×Gly/Ser-VS-HiBiT fragment fused to FKBP. Approximately 48 hours post-transfection, the cells were then labeled with 100 nM JF646 HaloTag ligand prior to fluorescence detection at indicated time points. This figure illustrates the fluorescence intensity of JF646 HaloTag ligand in live cell assays over time, comparing the fluorescent activity of cells expressing both HaloTag[3-19]-5×Gly/Ser-VS-HiBiT and HaloTag[22-297](M2F)-12×Gly/Ser-LgBiT variants with cells expressing only HaloTag[22-297](M2F)-12×Gly/Ser-LgBiT. HT-7 refers to full-length HaloTag. A non-transfected cell (NTC) control is included for reference. Three technical replicates were measured for each sample. The error bars represent the standard deviation of the mean (SD) of the data.

FIG. 63. Fluorescent Intensity of HaloTag[22-297](M2F)-12×Gly/Ser-LgBiT Variants co-expressing FKBP-HaloTag[3-19]-5×Gly/Ser-VS-HiBiT in Live Mammalian Cells. HeLa cells were transiently transfected with separate plasmids expressing HaloTag[22-297](M2F)-12×Gly/Ser-LgBiT variants and HaloTag[3-19]-5×Gly/Ser-VS-HiBiT fragment fused to FKBP. Approximately 48 hours post-transfection, the cells were then labeled with 100 nM JF646 HaloTag ligand prior to fluorescence detection at indicated time points. This figure illustrates the fluorescence intensity of JF646 HaloTag ligand in live cell assays over time, comparing the fluorescent activity of cells expressing both HaloTag[3-19]-5×Gly/Ser-VS-HiBiT and HaloTag[22-297](M2F)-12×Gly/Ser-LgBiT variants with cells expressing only HaloTag[22-297](M2F)-12×Gly/Ser-LgBiT. HT-7 refers to full-length HaloTag. A non-transfected cell (NTC) control is included for reference. Three technical replicates were measured for each sample. The error bars represent the standard deviation of the mean (SD) of the data.

FIG. 64. Fold Response Comparison of HaloTag[22-297](M2F)-12×Gly/Ser-LgBiT Variants co-expressing FKBP-HaloTag[3-19]-5×Gly/Ser-VS-HiBiT in Live Mammalian Cells. HeLa cells were transiently transfected with separate plasmids expressing HaloTag[22-297](M2F)-12×Gly/Ser-LgBiT variants and HaloTag[3-19]-5×Gly/Ser-VS-HiBiT fragment fused to FKBP. Approximately 48 hours post-transfection, the cells were labeled with 100 nM JF646 HaloTag ligand, and fluorescence was detected at indicated time points. The fold response of each assay condition was calculated as the ratio of fluorescence signal for cells expressing both the HaloTag[3-19]-5×Gly/Ser-VS-HiBiT and HaloTag[22-297](M2F)-12×Gly/Ser-LgBiT variants to cells expressing only the HaloTag[22-297](M2F)-12×Gly/Ser-LgBiT variant. HT-7 refers to full-length HaloTag. A non-transfected cell (NTC) control is included for reference. The error bars represent the standard deviation of the mean (SD) of the data.

FIG. 65. Comparison of Fluorescence Intensity and Fluorescence Fold Response of HaloTag[22-297](M2F)-12×Gly/Ser-LgBiT Variants. HeLa cells were transiently transfected with separate plasmids expressing HaloTag[22-297](M2F)-12×Gly/Ser-LgBiT variants and HaloTag[3-19]-5×Gly/Ser-VS-HiBiT fused to FKBP. After approximately 48 hours, post-transfection, the cells were labeled with 100 nM JF646 HaloTag ligand. Fluorescence was detected after 5 hours of incubation with JF646 ligand. The fold response was calculated as the ratio of fluorescence signal for cells expressing both the FKBP-HaloTag[3-19]-5×Gly/Ser-VS-HiBiT and HaloTag[22-297](M2F)-12×Gly/Ser-LgBiT variant to cells expressing only the HaloTag[22-297](M2F)-12×Gly/Ser-LgBiT variant. The M2F in this graph represents the template HaloTag[22-297](M2F)-12×Gly/Ser-LgBiT construct. All double mutants are added to the template, but only the two added mutations are shown for each construct. A non-transfected cell (NTC) control is included as a reference. This plot demonstrates the comparison of fold responses versus the total fluorescence of each mutant, highlighting the improved performance of HaloTag[22-297](M2F)-12×Gly/Ser-LgBiT variants in the presence of co-expressed FKBP-HaloTag[3-19]-5×Gly/Ser-VS-HiBiT.

FIG. 66. Fluorescence Intensity of HaloTag[22-297](M2F)-12×Gly/Ser-LgBiT Variants in Live Cell Fluorescent Plate Assays. HeLa cells were transiently transfected with separate plasmids expressing HaloTag[22-297](M2F)-12×Gly/Ser-LgBiT variants and HaloTag[3-19]-5×Gly/Ser-VS-HiBiT fused to FKBP. Approximately 48 hours post-transfection, the cells were labeled with 100 nM JF646 HaloTag ligand. Fluorescence intensity was detected after 5 hours of incubation with the JF646 HaloTag ligand. This figure illustrates the fluorescence intensity of JF646 HaloTag ligand in live cell assays, comparing the fluorescent activity of cells expressing both HaloTag[3-19]-5×Gly/Ser-VS-HiBiT and HaloTag[22-297](M2F)-12×Gly/Ser-LgBiT variants with cells expressing only the HaloTag[22-297](M2F)-12×Gly/Ser-LgBiT variants. Three technical replicates were measured for each sample. The error bars represent the standard deviation of the mean (SD) of the data.

FIG. 67. Fluorescent Fold Response of HaloTag[22-297](M2F)-12×Gly/Ser-LgBiT Variants in Live Cell Fluorescent Plate Assays. HeLa cells were transiently transfected with separate plasmids expressing HaloTag[22-297](M2F)-12×Gly/Ser-LgBiT variants and HaloTag[3-19]-5×Gly/Ser-VS-HiBiT fragment fused to FKBP. Approximately 48 hours post-transfection, the cells were labeled with 100 nM JF646 HaloTag® ligand. Fluorescence activity was detected after 5 hours of incubation with the JF646 ligand. The fold response of each assay condition was calculated as the ratio of fluorescence signal for cells expressing both the HaloTag[3-19]-5×Gly/Ser-VS-HiBiT and HaloTag[22-297](M2F)-12×Gly/Ser-LgBiT variants to cells expressing only the HaloTag[22-297](M2F)-12×Gly/Ser-LgBiT variants, and then normalized to the fold response of the template HaloTag[22-297](M2F)-12×Gly/Ser-LgBiT. The error bars represent the standard deviation of the mean (SD) of the data.

FIG. 68. Luminescence Activity of HaloTag[22-297](M2F)-12×Gly/Ser-LgBiT Variants when co-expressed with FKBP-HaloTag[3-19]-5×Gly/Ser-VS-HiBiT in Live Cells. HeLa cells were transfected with separate plasmids expressing HaloTag[22-297](M2F)-12×Gly/Ser-LgBiT variants and HaloTag[3-19]-5×Gly/Ser-VS-HiBiT fragment fused to FKBP. After approximately 48 hours post-transfection, the cells were treated with luminescent live-cell substrate (Furimazine). A non-transfected cell (NTC) control shown was measured identically except without introduction of an expression plasmid. Three technical replicates were measured for each sample. The error bars represent the standard deviation of the mean (SD) of the data.

FIG. 69. Luminescence Activity of HaloTag[22-297](M2F)-12×Gly/Ser-LgBiT Variants when co-expressed with FKBP-HaloTag[3-19]-5×Gly/Ser-VS-HiBiT in Live Cells. HeLa cells were transfected with separate plasmids expressing HaloTag[22-297](M2F)-12×Gly/Ser-LgBiT variants fragment and HaloTag[3-19]-5×Gly/Ser-VS-HiBiT fused to FKBP. After approximately 48 hours post-transfection, the cells were treated with a luminescent live-cell substrate (Furimazine). Three technical replicates were measured for each sample. The error bars represent the standard deviation of the mean (SD) of the data.

FIG. 70. Expression of HaloTag[22-297](M2F)-12×Gly/Ser-LgBiT Variants. HeLa cells were transiently transfected with plasmids expressing HaloTag[22-297](M2F)-12×Gly/Ser-LgBiT variants. After approximately 48 hours post-transfection, luminescence signal intensity was measured following cell lysis by adding HiBiT peptide at a final concentration of 200 nM, along with the lytic luminescent substrate (Furimazine). To prevent signal saturation and enhance signal linearity, the initial cell samples were diluted 80× with Opti-MEM media. A non-transfected cell (NTC) control is included for reference. Three technical replicates were measured for each sample. The error bars represent the standard deviation of the mean (SD) of the data.

FIG. 71. Comparison of Fold Response and Expression of LgHT-LgBiT HaloTag[22-297](M2F)-12×Gly/Ser-LgBiT Variants. To measure the fold response, HeLa cells were transiently transfected with separate plasmids expressing the HaloTag[22-297](M2F)-12×Gly/Ser-LgBiT variants fragment and HaloTag[3-19]-5×Gly/Ser-VS-HiBiT fragment fused to FKBP. After approximately 48 hours post-transfection, the cells were labeled with 100 nM JF646 HaloTag® ligand. Fluorescence was detected after 5 hours of incubation with JF646 HaloTag® ligand. The fold response was calculated as the ratio of fluorescence signal for cells expressing both the FKBP-HaloTag[3-19]-5×Gly/Ser-VS-HiBiT and HaloTag[22-297](M2F)-12×Gly/Ser-LgBiT variants to cells expressing only the HaloTag[22-297](M2F)-12×Gly/Ser-LgBiT variants. The M2F in this graph represents the template HaloTag[22-297](M2F)-12×Gly/Ser-LgBiT construct. All double mutants are added to the template, but only the two added mutations are shown for each construct for brevity. A non-transfected cell (NTC) control is included as a reference.

FIG. 72. Expression of HaloTag[22-297](M2F)-12×Gly/Ser-LgBiT Variants. HeLa cells were transiently transfected with plasmids expressing HaloTag[22-297](M2F)-12×Gly/Ser-LgBiT variants. After approximately 48 hours post-transfection, luminescence signal intensity was measured following cell lysis by adding HiBiT peptide at a final concentration of 200 nM, along with the lytic luminescent substrate (Furimazine). To prevent signal saturation and enhance signal linearity, the initial cell samples were diluted 80× with Opti-MEM media. A non-transfected cell (NTC) control is included for reference. Three technical replicates were measured for each sample. The error bars represent the standard deviation of the mean (SD) of the data.

FIG. 73. Expression of HaloTag[22-297](M2F)-12×Gly/Ser-LgBiT Variants. HeLa cells were transiently transfected with plasmids expressing the HaloTag[22-297](M2F)-12×Gly/Ser-LgBiT variants fragment. After approximately 48 hours post-transfection, luminescence signal intensity was measured following cell lysis by adding HiBiT peptide at a final concentration of 200 nM, along with the lytic luminescent substrate (Furimazine). To prevent signal saturation and enhance signal linearity, the initial cell samples were diluted 80× with Opti-MEM media. Four technical replicates were measured for each sample. The error bars represent the standard deviation of the mean (SD) of the data.

FIG. 74. Expression Variability of HaloTag[22-297](M2F)-12×Gly/Ser-LgBiT Variants across Transfection Replicates. HeLa cells were transiently transfected with plasmids expressing the HaloTag[22-297](M2F)-12×Gly/Ser-LgBiT variants fragment. Luminescence signal intensity was measured approximately 48 hours post-transfection after cell lysis by adding HiBiT peptide at a final concentration of 200 nM, along with the lytic luminescent substrate (Furimazine). To prevent signal saturation and enhance signal linearity, the initial cell samples were diluted 80× with Opti-MEM media. Three technical replicates were measured for each sample. The error bars represent the standard deviation of the mean (SD) of the data.

FIG. 75. Expression Variability of HaloTag[22-297](M2F)-12×Gly/Ser-LgBiT Variants across Transfection Replicates. HeLa cells were transiently transfected with the plasmid expressing HaloTag[22-297](M2F)-12×Gly/Ser-LgBiT. Luminescence signal intensity was measured approximately 48 hours post-transfection after cell lysis by adding HiBiT peptide at a final concentration of 200 nM, along with the lytic luminescent substrate (Furimazine). To prevent signal saturation and enhance signal linearity, the initial cell samples were diluted 80× with Opti-MEM media. Several technical replicates were measured for each transfection replicate. The error bars represent the standard deviation of the mean (SD) of the data. This graph demonstrates the observed variability of the lytic luminescence plate assays. The expression levels of the template across multiple transfection replicates enable the normalization of experimental results obtained from different replicates to the template.

FIG. 76A-B. Density Plots of Non-Transfected Cells in Cell Cytometry Assay for Filtering Dead Cells, Cell Debris, Doublets, and Cell Aggregates in Cell Cytometry Assay. Non-transfected HeLa cells were labeled with 100 nM JF646 HaloTag® ligand after approximately 48 hours post-seeding. Following a 1-hour incubation with the ligand, the cells were washed with DPBS media for 30 minutes (2× for 15 minutes each). Subsequently, the cells were lifted, and their fluorescence activity was measured using a live cell cytometry assay. The density plots of non-transfected cells were utilized in the cell cytometry assay to filter out dead cells, cell debris, doublets, and cell aggregates from the population of singlet, live HeLa cells. FIG. 76A. The Side Scatter Area (SSC-A) of non-transfected cells plotted against the Forward Scatter Area (FSC-A), which effectively filters out dead cells and cell debris. FIG. 76B. Density plot of the Forward Scatter Height (FSC—H) against the Forward Scatter Area (FSC-A) of non-transfected cells, which effectively filters out doublets and cell aggregates. The same gating strategies were applied to every single sample in the cell cytometry assay to effectively remove dead cells, cell debris, and doublets, ensuring accurate analysis of the singlet live HeLa cell population. In every cytometry experiment, listed in this document, the total number of events for the ungated population is 10,000-12,000 cells. The numbers on the graph indicate the percentage of cell populations within each depicted gate.

FIG. 77. Gating Strategy of Non-Transfected HeLa Cells for Detection of JF646 Positive Cells. Non-transfected HeLa cells were labeled with 100 nM JF646 HaloTag® ligand after approximately 48 hours post-seeding. Following a 1-hour incubation with the ligand, the cells were washed with DPBS media for 30 minutes (2× for 15 minutes each). Subsequently, the cells were lifted, and their fluorescence activity was measured using a live cell cytometry assay. The non-transfected sample was utilized for gating and measuring the activity of transfected cells in comparison to the non-transfected or non-fluorescently active cells. The histogram depicts the number of cells on the Y-axis against their activity with JF646 ligand on the X-axis. A consistent gating strategy, based on the baseline activity detected with non-transfected cells, was applied to every transfected sample to accurately detect their fluorescence activity. The initial number of events ranged from 10,000 to 12,000 cells. Specifically, for the analysis of JF646 activity, the number of events being analyzed is usually between 2,000 and 4,000. The cells being analyzed in the JF646+ gate is dependent on the transfection efficiency of the mutant and based on the gate settings for the JF646 activity.

FIG. 78. Fluorescent Intensity of HaloTag[22-297](M2F)-12×Gly/Ser-LgBiT Variants co-expressed with HaloTag[3-19]-5×Gly/Ser-VS-HiBiT in the JF646+ Gate Measured by Flow cytometry. HeLa cells were transiently transfected with separate plasmids expressing HaloTag[22-297](M2F)-12×Gly/Ser-LgBiT variants and HaloTag[3-19]-5×Gly/Ser-VS-HiBiT fragment fused to FKBP. After approximately 48 hours post-transfection, the cells were labeled with 100 nM JF646 HaloTag® ligand. Following a 1-hour incubation with the ligand, the cells were washed with DPBS media for 30 minutes (2× for 15 minutes each). Subsequently, the cells were lifted, and their fluorescence activity in the JF646+ gate was measured using the cell cytometry assay. Three technical replicates were measured for each sample. The error bars represent the standard deviation of the mean (SD) of the data.

FIG. 79. Fluorescence Fold Response Comparison of HaloTag[22-297](M2F)-12×Gly/Ser-LgBiT Variants co-expressing FKBP-HaloTag[3-19]-5×Gly/Ser-VS-HiBiT Using Flow Cytometry. HeLa cells were transiently transfected with separate plasmids expressing the HaloTag[22-297](M2F)-12×Gly/Ser-LgBiT variants fragment and HaloTag[3-19]-5×Gly/Ser-VS-HiBiT fragment fused to FKBP. After approximately 48 hours post-transfection, the cells were labeled with 100 nM JF646 HaloTag® ligand. Following a 1-hour incubation with the ligand, the cells were washed with DPBS media for 30 minutes (2× for 15 minutes each). Subsequently, the cells were lifted, and their fluorescence activity in the JF646+ gate was measured using the cell cytometry assay. The fold response of each assay condition was calculated as the ratio of mean fluorescence signal in the JF646+ gate for cells expressing both FKBP-HaloTag[3-19]-5×Gly/Ser-VS-HiBiT and HaloTag[22-297](M2F)-12×Gly/Ser-LgBiT variant to the mean fluorescence intensity of cells expressing only the HaloTag[22-297](M2F)-12×Gly/Ser-LgBiT variants in the same JF646+ gate. The error bars represent the standard deviation of the mean (SD) of the data.

FIG. 80. Comparison of Fluorescence Intensity and Fold Response of HaloTag[22-297](M2F)-12×Gly/Ser-LgBiT Variants Measured using Flow Cytometry. HeLa cells were transiently transfected with separate plasmids expressing HaloTag[22-297](M2F)-12×Gly/Ser-LgBiT variants and HaloTag[3-19]-5×Gly/Ser-VS-HiBiT fragment fused to FKBP. After approximately 48 hours post-transfection, the cells were labeled with 100 nM JF646 HaloTag® ligand. Following a 1-hour incubation with the ligand, the cells were washed with DPBS media for 30 minutes (2× for 15 minutes each). Subsequently, the cells were lifted, and their fluorescence activity in the JF646+ gate was measured using the cell cytometry assay. The fold response of each assay condition was calculated as the ratio of mean fluorescence signal in the JF646+ gate for cells expressing both the FKBP-HaloTag[3-19]-5×Gly/Ser-VS-HiBiT and HaloTag[22-297](M2F)-12×Gly/Ser-LgBiT variants to the mean fluorescence signal of cells expressing only the HaloTag[22-297](M2F)-12×Gly/Ser-LgBiT variants in the same JF646+ gate. The M2F in this graph represents the template, HaloTag[22-297](M2F)-12×Gly/Ser-LgBiT. All double mutants are added to the template, but only the two added mutations are shown for each construct to fit the data. A non-transfected cell (NTC) control is included as a reference. This plot demonstrates the comparison of fold responses versus the total fluorescent activity of each mutant, highlighting the improved performance of HaloTag[22-297](M2F)-12×Gly/Ser-LgBiT variants when co-expressed with FKBP-HaloTag[3-19]-5×Gly/Ser-VS-HiBiT.

FIG. 81A-C. Fluorescence Intensity During Co-expression of FKBP-HaloTag[3-19]-5×Gly/Ser-VS-HiBiT and HaloTag[22-297](M2F)-12×Gly/Ser-LgBiT Variants Using Flow Cytometry. The histograms of variants representing higher fold response FIG. 81(A) and lower fold response FIG. 81(B) phenotypes demonstrate how mutations can change the performance of the template, HaloTag[22-297](M2F)-12×Gly/Ser-LgBiT, measured using flow cytometry. Cells expressing only HaloTag[22-297](M2F)-12×Gly/Ser-LgBiT Variants are shown in light grey and those also co-expressing FKBP-HaloTag[3-19]-5×Gly/Ser-VS-HiBiT are shown in dark grey. HeLa cells were transiently transfected with separate plasmids expressing HaloTag[22-297](M2F)-12×Gly/Ser-LgBiT variants and HaloTag[3-19]-5×Gly/Ser-VS-HiBiT fragment fused to FKBP. After approximately 48 hours post-transfection, the cells were labeled with 100 nM JF646 HaloTag® ligand. Following a 1-hour incubation with the ligand, the cells were washed with DPBS media for 30 minutes (2× for 15 minutes each). Subsequently, the cells were lifted, and their fluorescence activity was measured using the cell cytometry assay. The M2F in this graph represents the template, HaloTag[22-297](M2F)-12×Gly/Ser-LgBiT construct. All double mutants are added to the template, but only the two added mutations are shown for each construct to represent the data. “Normalized to mode” on the Y-axis scales the number of events values based on the most frequently occurring value, in this graph the number of events in the JF646 negative gate. This enables standardized comparisons and highlights relative differences between populations.

FIG. 82. Fluorescent Intensity of HaloTag[22-297](M2F)-12×Gly/Ser-LgBiT Variants Co-expressed with FKBP-HaloTag[3-19]-5×Gly/Ser-VS-HiBiT in the JF646+ Gate Measured by Flow Cytometry. HeLa cells were transiently transfected with separate plasmids expressing the HaloTag[22-297](M2F)-12×Gly/Ser-LgBiT variants fragment and HaloTag[3-19]-5×Gly/Ser-VS-HiBiT fragment fused to FKBP. After approximately 48 hours post-transfection, the cells were labeled with 100 nM JF646 HaloTag® ligand. Following a 1-hour incubation with the ligand, the cells were washed with DPBS media for 30 minutes (2× for 15 minutes each). Subsequently, the cells were lifted, and their fluorescence activity in the JF646+ gate was measured using flow cytometry. Three technical replicates were measured for each sample. The error bars represent the standard deviation of the mean (SD) of the data.

FIG. 83. Fluorescence Fold Response Comparison of HaloTag[22-297](M2F)-12×Gly/Ser-LgBiT Variants Co-expressed with FKBP-HaloTag[3-19]-5×Gly/Ser-VS-HiBiT in Live Mammalian Cells Measured Using Flow Cytometry. HeLa cells were transiently transfected with separate plasmids expressing the HaloTag[22-297](M2F)-12×Gly/Ser-LgBiT variants fragment and HaloTag[3-19]-5×Gly/Ser-VS-HiBiT fragment fused to FKBP. After approximately 48 hours post-transfection, the cells were labeled with 100 nM JF646 HaloTag® ligand. Following a 1-hour incubation with the ligand, the cells were washed with DPBS media for 30 minutes (2× for 15 minutes each). Subsequently, the cells were lifted, and their fluorescence activity in the JF646+ gate was measured using the cell cytometry assay. The fold response of each assay condition was calculated as the ratio of mean fluorescence signal in the JF646+ gate for cells expressing both the FKBP-HaloTag[3-19]-5×Gly/Ser-VS-HiBiT and HaloTag[22-297](M2F)-12×Gly/Ser-LgBiT variants to the mean fluorescence signal of cells expressing only the HaloTag[22-297](M2F)-12×Gly/Ser-LgBiT variants in the same JF646+ gate. The error bars represent the standard deviation of the mean (SD) of the data.

FIG. 84. Fluorescent Intensity of HaloTag[22-297](M2F)-12×Gly/Ser-LgBiT Variants Co-expressed with FKBP-HaloTag[3-19]-5×Gly/Ser-VS-HiBiT in the JF646+ Gate Measured using Flow Cytometry. HeLa cells were transiently transfected with separate plasmids expressing the HaloTag[22-297](M2F)-12×Gly/Ser-LgBiT variants fragment and HaloTag[3-19]-5×Gly/Ser-VS-HiBiT fragment fused to FKBP. After approximately 48 hours post-transfection, the cells were labeled with 100 nM JF646 HaloTag® ligand. Following a 1-hour incubation with the ligand, the cells were washed with DPBS media for 30 minutes (2× for 15 minutes each). Subsequently, the cells were lifted, and their fluorescence activity in the JF646+ gate was measured using the cell cytometry assay. Two technical replicates were measured for each sample. The error bars represent the standard deviation of the mean (SD) of the data.

FIG. 85. Fluorescence Fold Response Comparison of HaloTag[22-297](M2F)-12×Gly/Ser-LgBiT Variants Co-expressed with FKBP-HaloTag[3-19]-5×Gly/Ser-VS-HiBiT in Live Mammalian Cells Measured Using Flow Cytometry. HeLa cells were transiently transfected with separate plasmids expressing the HaloTag[22-297](M2F)-12×Gly/Ser-LgBiT variants fragment and HaloTag[3-19]-5×Gly/Ser-VS-HiBiT fragment fused to FKBP. After approximately 48 hours post-transfection, the cells were labeled with 100 nM JF646 HaloTag® ligand. Following a 1-hour incubation with the ligand, the cells were washed with DPBS media for 30 minutes (2× for 15 minutes each). Subsequently, the cells were lifted, and their fluorescence activity in the JF646+ gate was measured using the cell cytometry assay. The fold response of each assay condition was calculated as the ratio of mean fluorescence signal in the JF646+ gate for cells expressing both the HaloTag[3-19]-5×Gly/Ser-VS-HiBiT and FKBP-HaloTag[22-297](M2F)-12×Gly/Ser-LgBiT variants to the mean fluorescence signal of cells expressing only the HaloTag[22-297](M2F)-12×Gly/Ser-LgBiT variants in the same JF646+ gate. The error bars represent the standard deviation of the mean (SD) of the data.

FIG. 86. Fluorescent Intensity of HaloTag[22-297](M2F)-12×Gly/Ser-LgBiT Variants Co-expressed with FKBP-HaloTag[3-19]-5×Gly/Ser-VS-HiBiT in the JF646+ Gate upon Prolonged Washing Period Measured by Flow Cytometry. HeLa cells were transiently transfected with separate plasmids expressing the HaloTag[22-297](M2F)-12×Gly/Ser-LgBiT variants fragment and HaloTag[3-19]-5×Gly/Ser-VS-HiBiT fragment fused to FKBP. Approximately 48 hours post-transfection, the cells were labeled with 100 nM JF646 HaloTag® ligand. Following a 1-hour incubation with the ligand, the cells were washed with DPBS media for 2 hours and a half (2× for 75 minutes each). Subsequently, the cells were lifted, and their fluorescence activity in the JF646+ gate was measured using flow cytometry. Two technical replicates were measured for each sample. The error bars represent the standard deviation of the mean (SD) of the data.

FIG. 87. Fluorescence Fold Response Comparison of HaloTag[22-297](M2F)-12×Gly/Ser-LgBiT Variants Co-expressed with FKBP-HaloTag[3-19]-5×Gly/Ser-VS-HiBiT upon Prolonged Washing Period in Live Mammalian Cells Measured Using Flow Cytometry. HeLa cells were transiently transfected with separate plasmids expressing HaloTag[22-297](M2F)-12×Gly/Ser-LgBiT variants and HaloTag[3-19]-5×Gly/Ser-VS-HiBiT fragment fused to FKBP. After approximately 48 hours post-transfection, the cells were labeled with 100 nM JF646 HaloTag® ligand. Following a 1-hour incubation with the ligand, the cells were washed with DPBS media for 2 hours and a half (2× for 75 minutes each). Subsequently, the cells were lifted, and their fluorescence activity in the JF646+ gate was measured using the cell cytometry assay. The fold response of each assay condition was calculated as the ratio of mean fluorescence signal in the JF646+ gate for cells expressing both the HaloTag[3-19]-5×Gly/Ser-VS-HiBiT and HaloTag[22-297](M2F)-12×Gly/Ser-LgBiT variants to the mean fluorescence signal of cells expressing only the HaloTag[22-297](M2F)-12×Gly/Ser-LgBiT variants in the same JF646+ gate. The error bars represent the standard deviation of the mean (SD) of the data.

FIG. 88. Fluorescent Activity of HaloTag[22-297](M2F)-12×Gly/Ser-LgBiT Variants Co-expressed with FKBP-HaloTag[3-19]-5×Gly/Ser-VS-HiBiT in the JF646+ Gate Measured by Flow Cytometry. HeLa cells were transiently transfected with separate plasmids expressing HaloTag[22-297](M2F)-12×Gly/Ser-LgBiT variants and HaloTag[3-19]-5×Gly/Ser-VS-HiBiT fragment fused to FKBP. Approximately 48 hours post-transfection, the cells were labeled with 100 nM JF646 HaloTag® ligand. Following a 1-hour incubation with the ligand, the cells were washed with DPBS media for 30 minutes (2× for 15 minutes each). Subsequently, the cells were lifted, and their fluorescence activity in the JF646+ gate was measured using the cell cytometry assay. The live cell cytometry assay revealed a relatively consistent level of fluorescent activity, normalized to the template, HaloTag[22-297](M2F)-12×Gly/Ser-LgBiT, between the first and second transfection replicates for the complemented FKBP-HaloTag[3-19]-5×Gly/Ser-VS-HiBiT and HaloTag[22-297](M2F)-12×Gly/Ser-LgBiT variants.

FIG. 89. Fluorescence Fold Response of HaloTag[22-297](M2F)-12×Gly/Ser-LgBiT Variants Co-expressed with FKBP-HaloTag[3-19]-5×Gly/Ser-VS-HiBiT in Live Mammalian Cells Measured Using Flow Cytometry. HeLa cells were transiently transfected with separate plasmids expressing HaloTag[22-297](M2F)-12×Gly/Ser-LgBiT variants and HaloTag[3-19]-5×Gly/Ser-VS-HiBiT fragment fused to FKBP. After approximately 48 hours post-transfection, the cells were labeled with 100 nM JF646 HaloTag® ligand. Following a 1-hour incubation with the ligand, the cells were washed with DPBS media for 1 hour (2× for 15 minutes each). Subsequently, the cells were lifted, and their fluorescence activity in the JF646+ gate was measured using the cell cytometry assay. The fold response of each assay condition was calculated as the ratio of mean fluorescence signal in the JF646+ gate for cells expressing both the FKBP-HaloTag[3-19]-5×Gly/Ser-VS-HiBiT and HaloTag[22-297](M2F)-12×Gly/Ser-LgBiT variants to the mean fluorescence signal of cells expressing only the HaloTag[22-297](M2F)-12×Gly/Ser-LgBiT variants in the same JF646+ gate. Flow cytometry revealed a relatively consistent level of fluorescent fold response, normalized to the template, HaloTag[22-297](M2F)-12×Gly/Ser-LgBiT construct, between the first and second transfection replicates.

FIG. 90. Variability of Fluorescent Activity of HaloTag[22-297](M2F)-12×Gly/Ser-LgBiT Variants Co-expressed with FKBP-HaloTag[3-19]-5×Gly/Ser-VS-HiBiT in the JF646+ Gate Measured by Flow Cytometry across Transfection Replicates. HeLa cells were transiently transfected with separate plasmids expressing HaloTag[22-297](M2F)-12×Gly/Ser-LgBiT and HaloTag[3-19]-5×Gly/Ser-VS-HiBiT fragment fused to FKBP. Approximately 48 hours post-transfection, the cells were labeled with 100 nM JF646 HaloTag® ligand. Following a 1-hour incubation with the ligand, the cells were washed with DPBS media for 30 minutes (2× for 15 minutes each). Subsequently, the cells were lifted, and their fluorescence activity in the JF646+ gate was measured using flow cytometry. The expression levels of the template across multiple transfection replicates enable the normalization of experimental results obtained from different replicates to the template.

FIG. 91. Comparison of Fluorescence Intensity and Fold Response of HaloTag[22-297](M2F)-12×Gly/Ser-LgBiT Variants Co-expressed with FKBP-HaloTag[3-19]-5×Gly/Ser-VS-HiBiT Using Flow Cytometry. HeLa cells were transiently transfected with separate plasmids expressing HaloTag[22-297](M2F)-12×Gly/Ser-LgBiT variants and HaloTag[3-19]-5×Gly/Ser-VS-HiBiT fragment fused to FKBP. Approximately 48 hours post-transfection, the cells were labeled with 100 nM JF646 HaloTag® ligand. Following a 1-hour incubation with the ligand, the cells were washed with DPBS media for 30 minutes (2× for 15 minutes each). Subsequently, the cells were lifted, and their fluorescence activity in the JF646+ gate was measured using flow cytometry. The fold response of each assay condition was calculated as the ratio of the mean fluorescence signal in the JF646+ gate for cells expressing both the FKBP-HaloTag[3-19]-5×Gly/Ser-VS-HiBiT and HaloTag[22-297](M2F)-12×Gly/Ser-LgBiT variants to the mean fluorescence signal of cells expressing only the HaloTag[22-297](M2F)-12×Gly/Ser-LgBiT variants in the same JF646+ gate. Four transfection replicates are depicted for the template, HaloTag[22-297](M2F)-12×Gly/Ser-LgBiT construct, shown in darker gray. All single and double mutants were added to the template, HaloTag[22-297](M2F)-12×Gly/Ser-LgBiT construct, but only the added mutations are shown for each construct. This plot highlights the comparison of fold responses versus the total fluorescent activity of each mutant, emphasizing the improved performance of HaloTag[22-297](M2F)-12×Gly/Ser-LgBiT variants mutations when Co-expressed with FKBP-HaloTag[3-19]-5×Gly/Ser-VS-HiBiT.

FIG. 92. Gating of Non-Transfected Cells in Flow Cytometry for Separating Single and Double Positive Cells of JF646 and EGFP Signal. Non-transfected HeLa cells were labeled with 100 nM JF646 HaloTag® ligand approximately 48 hours post-seeding. After a 1-hour incubation with the ligand, the cells were washed with DPBS media for 30 minutes (2× for 15 minutes each). Subsequently, the cells were lifted, and their fluorescence activity was measured using a live cell cytometry assay. The density plot of non-transfected cells was utilized to establish gating regions: JF646 negative (Q4) and JF646 positive (Q3), as well as negative EGFP expression (Q4) and positive EGFP expression (Q1). Consequently, double-negative cells were identified in Q4, single-positive cells for JF646 and EGFP were observed in Q3 and Q1, respectively. Double-positive cells were identified in Q2.

FIG. 93A-B. Comparison of Cells Expressing HaloTag[22-297](M2F)-12×Gly/Ser-LgBiT Co-expressing EGFP-HaloTag[3-19]-5×Gly/Ser-VS-HiBiT Using Flow Cytometry. HeLa cells were transiently transfected with separate plasmids expressing either FIG. 93(A) HaloTag[22-297](M2F)-12×Gly/Ser-LgBiT alone or FIG. 93(B) co-expressing HaloTag[22-297](M2F)-12×Gly/Ser-LgBiT and EGFP-HaloTag[3-19]-5×Gly/Ser-VS-HiBiT on separate plasmids. Approximately 48 hours post-transfection, the cells were labeled with 100 nM JF646 HaloTag® ligand. Following a 1-hour incubation with the ligand, the cells were washed with DPBS media for 30 minutes (2× for 15 minutes each). Subsequently, the cells were lifted, and their fluorescence activity was measured using flow cytometry. As depicted, As, a significant shift of cells is observed from the Q3 quadrant to the Q2 quadrant when both EGFP-HaloTag[3-19]-5×Gly/Ser-VS-HiBiT and HaloTag22-297-12×Gly/Ser-LgBiT variants are expressed, compared to when only the HaloTag22-297-12×Gly/Ser-LgBiT is expressed. This shift indicates the formation of a complex between the HaloTag[3-19]-5×Gly/Ser-VS-HiBiT and HaloTag22-297-12×Gly/Ser-LgBiT variants, or the co-expression of both EGFP-HaloTag[3-19]-5×Gly/Ser-VS-HiBiT and HaloTag22-297-12×Gly/Ser-LgBiT components in cells.

FIG. 94. Fluorescent Intensity of HaloTag[22-297](M2F)-12×Gly/Ser-LgBiT Variants Co-Expressed with EGFP-HaloTag[3-19]-5×Gly/Ser-VS-HiBiT Measured Using Flow Cytometry. HeLa cells were transiently transfected with separate plasmids expressing HaloTag[22-297](M2F)-12×Gly/Ser-LgBiT variants and HaloTag[3-19]-5×Gly/Ser-VS-HiBiT fragment fused to EGFP. Approximately 48 hours post-transfection, the cells were labeled with 100 nM JF646 HaloTag® ligand. Following a 1-hour incubation with the ligand, the cells were washed with DPBS media for 30 minutes (2× for 15 minutes each). Subsequently, the cells were lifted, and their fluorescence activity was measured using flow cytometry. The fluorescent intensity was determined based on the mean intensity in the second (double positive) quadrant, while the fluorescent intensity of the HaloTag[22-297](M2F)-12×Gly/Ser-LgBiT variant alone samples were measured based on the mean fluorescent intensity in the third (JF646+) quadrant. Two technical replicates were performed for each sample. The error bars represent the standard deviation of the mean (SD) of the data.

FIG. 95. Fluorescence Fold Response Comparison of HaloTag[22-297](M2F)-12×Gly/Ser-LgBiT Variants Co-expressed with EGFP-HaloTag[3-19]-5×Gly/Ser-VS-HiBiT in Live Mammalian Cells Measured Using Flow Cytometry with JF646 HaloTag ligand. HeLa cells were transiently transfected with separate plasmids expressing the HaloTag[22-297](M2F)-12×Gly/Ser-LgBiT variants and HaloTag[3-19]-5×Gly/Ser-VS-HiBiT fragment fused to EGFP. Approximately 48 hours post-transfection, the cells were labeled with 100 nM JF646 HaloTag® ligand. Following a 1-hour incubation with the ligand, the cells were washed with DPBS media for 30 minutes (2× for 15 minutes each). Subsequently, the cells were lifted, and their fluorescence activity was measured using flow cytometry. The fluorescent activity in the co-expression samples was determined based on the mean intensity in the second (double positive) quadrant, while the fluorescent activity of HaloTag[22-297](M2F)-12×Gly/Ser-LgBiT alone variants were measured based on the mean fluorescent intensity in the third (JF646+) quadrant. The fold response of each assay condition was calculated as the ratio of mean fluorescence signal in the second quadrant (double positive) by the mean fluorescence signal in the third (JF646+) quadrant.

FIG. 96. Fluorescence Fold Response Comparison of HaloTag[22-297](M2F)-12×Gly/Ser-LgBiT Variants Co-Expressed with EGFP-HaloTag[3-19]-5×Gly/Ser-VS-HiBiT in Live Mammalian Cells Measured Using Flow Cytometry with JF635 HaloTag Ligand. HeLa cells were transiently transfected with separate plasmids expressing the HaloTag[22-297](M2F)-12×Gly/Ser-LgBiT variants fragment and HaloTag[3-19]-5×Gly/Ser-VS-HiBiT fragment fused to EGFP. Approximately 48 hours post-transfection, the cells were labeled with 100 nM JF635 HaloTag® ligand. Following a 1-hour incubation with the ligand, the cells were washed with DPBS media for 30 minutes (2× for 15 minutes each). Subsequently, the cells were lifted, and their fluorescence activity was measured using flow cytometry. The fluorescent activity in the co-expression samples was determined based on the mean intensity in the second (double positive) quadrant, while the fluorescent activity of HaloTag[22-297](M2F)-12×Gly/Ser-LgBiT alone variants were measured based on the mean fluorescent intensity in the third (JF646+) quadrant. The fold response of each assay condition was calculated as the ratio of mean fluorescence signal in the second quadrant (double positive) by the mean fluorescence signal in the third (JF646+) quadrant. The results demonstrate that the fold responses are enhanced when using the more fluorogenic ligand, JF635, compared to the less fluorogenic ligand, JF646.

FIG. 97. Comparison of Fluorescence Intensity and Fold Response for Selected HaloTag[22-297](M2F)-12×Gly/Ser-LgBiT Variants Measured by Flow Cytometry. HeLa cells were transiently transfected with separate plasmids expressing HaloTag[22-297](M2F)-12×Gly/Ser-LgBiT variants and HaloTag[3-19]-5×Gly/Ser-VS-HiBiT fragment fused to EGFP. Approximately 48 hours post-transfection, the cells were labeled with 100 nM JF646 HaloTag® ligand. Following a 1-hour incubation with the ligand, the cells were washed with DPBS media for 30 minutes (2× for 15 minutes each). Subsequently, the cells were lifted, and their fluorescence activity in the JF646+ gate was measured using flow cytometry. The fold response of each assay condition was calculated as the ratio of the mean fluorescence signal in the JF646+ gate for cells expressing both the EGFP-HaloTag[3-19]-5×Gly/Ser-VS-HiBiT and HaloTag[22-297](M2F)-12×Gly/Ser-LgBiT variants to the mean fluorescence signal of cells expressing only the HaloTag[22-297](M2F)-12×Gly/Ser-LgBiT variants in the same JF646+ gate. Two transfection replicates are depicted for the template, HaloTag[22-297](M2F)-12×Gly/Ser-LgBiT, shown in dark gray. This plot emphasizes the improved fluorescence intensity of HaloTag[22-297](M2F)-12×Gly/Ser-LgBiT variants mutations when co-expressed with EGFP-HaloTag[3-19]-5×Gly/Ser-VS-HiBiT.

FIG. 98A-C. Fluorescence of HeLa cells Co-Expressing HaloTag[22-297](M2F)-12×Gly/Ser-LgBiT Variants with EGFP-HaloTag[3-19]-5×Gly/Ser-VS-HiBiT Using Confocal Imaging. HeLa cells were transiently transfected with separate plasmids expressing the HaloTag[22-297](M2F)-12×Gly/Ser-LgBiT variants and HaloTag[3-19]-5×Gly/Ser-VS-HiBiT fragment fused to EGFP. For comparison, cells were transfected HaloTag[22-297](M2F)-12×Gly/Ser-LgBiT variants alone. Approximately 48 hours post-transfection, the cells were labeled with 100 nM JF646 HaloTag® ligand. Following a 1-hour incubation, the cells were imaged using a confocal microscope in both the green channel (Ex. 488 nm) and the far-red channel (Ex. 640 nm). The laser and gain settings were optimized for each construct to minimize saturated pixels. Rows show the co-expressed and single plasmid control signals for FIG. 98(A) HaloTag[22-297](M2F)-12×Gly/Ser-LgBiT, FIG. 98(B) HaloTag[22-297](M2F+D53G+F148M)-12×Gly/Ser-LgBiT and FIG. 98(C) HaloTag[22-297](M2F+D53G+V177E)-12×Gly/Ser-LgBiT.

FIG. 99. Quantitated Fluorescence of HeLa cells Co-Expressing HaloTag[22-297](M2F)-12×Gly/Ser-LgBiT Variants with EGFP-HaloTag[3-19]-5×Gly/Ser-VS-HiBiT Using Confocal Imaging. HeLa cells were transiently transfected with separate plasmids expressing HaloTag[22-297](M2F)-12×Gly/Ser-LgBiT variants and HaloTag[3-19]-5×Gly/Ser-VS-HiBiT fused to EGFP. Additionally, cells were transfected with mutants of HaloTag[22-297](M2F)-12×Gly/Ser-LgBiT variants alone to measure signal from background labeling. Approximately 48 hours post-transfection, the cells were labeled with 100 nM JF646 HaloTag® ligand. Following a 1-hour incubation, the cells were imaged using a confocal microscope in both the green channel (Ex. 488 nm) and the far-red channel (Ex. 640 nm). The laser and gain settings were optimized for each construct to minimize saturated pixels. To quantify the confocal images, multiple fields of view were collected for each sample, and the intensity of individual cells was measured for both the co-expression and background signal. The bar represents the mean of the data. Fold response indicated above samples was calculated as the ratio of the mean specific signal for cells expressing both the EGFP-HaloTag[3-19]-5×Gly/Ser-VS-HiBiT and HaloTag[22-297](M2F)-12×Gly/Ser-LgBiT variants to the mean intensity of the signal for cells expressing only the HaloTag[22-297](M2F)-12×Gly/Ser-LgBiT variants. This graph illustrates how mutating the template has improved the performance of HaloTag[22-297](M2F)-12×Gly/Ser-LgBiT variants when co-expressed with EGFP-HaloTag[3-19]-5×Gly/Ser-VS-HiBiT in confocal imaging applications.

FIG. 100. CRISPR Knock-In Efficiency at Endogenous PARP1 and CTNNB1 Loci in DLD-1 Cell Pools During Introduction of HaloTag[3-19]—VS-HiBiT Variants. The variants include HaloTag[3-19]—VS-HiBiT without linkers and HaloTag[3-19]—VS-HiBiT variants with 4×Gly/Ser linkers (GGSG-HaloTag[3-19]-GGSG VS-HiBiT). Data was collected using Droplet Digital Polymerase Chain Reaction. Knock-in efficiency was calculated as the percentage of PCR amplicon copies per volume containing the tagged sequence versus non-tagged sequence at the target loci. The error bars represent the standard deviation of the mean (SD) of the data.

FIG. 101. Expression of PARP1 and CTNNB1 Tagged with HaloTag[3-19]—VS-HiBiT Variants or HiBiT in CRISPR Pools. The luminescence signal intensity of DLD-1 CRISPR cell pools (40,000 cells per pool replicate) was measured following cell lysis by adding LgBiT protein along with the lytic luminescent substrate (Furimazine). Six technical replicates were tested for each construct's CRISPR pool. This result confirms the integration and functionality of each of the tags. Additionally, it demonstrates that the addition of a linker in the HaloTag[3-19]-GGSG-VS-HiBiT variant improved detection. To ensure that the luminescence activities were not affected by the knock-in efficiencies of the insert within the CRISPR pools, pools that exhibited relatively similar knock-in efficiencies were selected for this experiment. The error bars represent the standard deviation of the mean (SD) of the data.

FIG. 102. Endogenous Expression of PARP1 fused to HaloTag[3-19]—VS-HiBiT Variants or HiBiT in CRISPR clones. Luminescence signal intensity was measured for DLD-1 CRISPR cell clones (40,000 cells per clone for each replicate) following cell lysis by adding LgBiT protein and the lytic luminescent substrate (Furimazine). Several technical replicates were performed for each construct's CRISPR clone. The results confirm successful integration and functionality of each of the tags. Additionally, it demonstrates that the addition of a linker in the HaloTag[3-19]-GGSG-VS-HiBiT variant improved detection at the CRISPR clone level. The error bars represent the standard deviation of the mean (SD) of the data.

FIG. 103A-C. Gating Strategy of Non-Transfected PARP1-GGSG-HaloTag[3-19]-GGSG-VS-HiBiT DLD-1 Cells for JF646 Positive Cells. DLD-1 cell line expressing PARP1-GGSG-HaloTag[3-19]-GGSG-VS-HiBiT was labeled with 100 nM JF646 HaloTag® ligand after approximately 48 hours post-seeding. Following a 1-hour incubation with the ligand, the cells were washed with DPBS media for 30 minutes (2×15 minutes). Subsequently, the cells were detached, and their fluorescence intensity was measured using flow cytometry. The density plots of non-transfected cells were utilized in the cell cytometry assay to filter out dead cells, cell debris, doublets, and cell aggregates from the population of singlet DLD-1 cells. FIG. 103(A) Side Scatter Area (SSC-A) of non-transfected cells plotted against the Forward Scatter Area (FSC-A), which effectively filters out dead cells and cell debris. FIG. 103(B) Density plot of the Height of the Forward Scatter (FSC—H) against the Area of the Forward Scatter (FSC-A) of non-transfected cells, which effectively filters out doublets and cell aggregates. The same gating strategies were applied to every single sample in the cell cytometry assay to effectively remove dead cells, cell debris, and doublets, ensuring accurate analysis of the singlet live DLD-1 cell population. FIG. 103(C) The histogram represents the number of cells on the Y-axis and their intensity with JF646 ligand on the X-axis. A consistent gating strategy, established using the baseline activity detected with non-transfected cells, was applied to each transfected sample to accurately identify and quantify their fluorescence activity. Prior to this step, the gating strategy was consistent with previous cytometry experiments, excluding dead cells, cell debris, and cell aggregates. In every cytometry experiment, listed in this document, the total number of events for the ungated population is 10,000-12,000 cells. The numbers on the graph indicate the percentage of cell populations within each depicted gate. Specifically, for the analysis of JF646 activity, the number of events being analyzed is usually between 2,000 and 4,000. The cells being analyzed in the JF646+ gate depend on the transfection efficiency of the mutant and based on the gate settings for the JF646 activity.

FIG. 104. Comparison of Fluorescent Intensity of HaloTag[22-297](M2F)-12×Gly/Ser-LgBiT Transiently Expressed with Different Promoters in a PARP1-GGSG-HaloTag[3-19]-GGSG-VS-HiBiT DLD-1 cell line. DLD-1 cells expressing endogenous PARP1-GGSG-HaloTag[3-19]-GGSG-VS-HiBiT were transiently transfected plasmids expressing HaloTag[22-297](M2F)-12×Gly/Ser-LgBiT under control of a CMV or TK promoter. Approximately 48 hours post-transfection, the cells were labeled with 100 nM JF646 HaloTag® ligand. Following a 1-hour incubation, the cells were washed with DPBS media for 30 minutes (2×15 minutes each). Subsequently, the cells were detached, and their fluorescence activity was measured using flow cytometry. The gating strategy employed is consistent with previous cytometry experiments, excluding dead cells, cell debris, and cell aggregates. Additionally, labeled, and non-transfected CRISPR cells were used to discriminate JF646 positive and JF646 negative populations. The values for the x-axis corresponding to the amount of plasmid DNA expressing HaloTag[22-297](M2F)-12×Gly/Ser-LgBiT using for transient transfection in each well of a 96-well plate. The error bars represent the standard deviation of the mean (SD) of the data. This graph illustrates that under all plasmid DNA concentrations, the fluorescent signal intensity in the JF646 positive gate is approximately 10-fold higher when the HaloTag[22-297](M2F)-12×Gly/Ser-LgBiT variant is expressed under the CMV promoter compared to expression under the TK promoter.

FIG. 105. Comparison of Fold Response and Fluorescent Intensity of HaloTag[22-297](M2F)-12×Gly/Ser-LgBiT Transiently Expressed with Different Promoters in a PARP1-GGSG-HaloTag[3-19]-GGSG-VS-HiBiT DLD-1 cell line. DLD-1 cells expressing endogenous PARP1-GGSG-HaloTag[3-19]-GGSG-VS-HiBiT were transiently transfected plasmids expressing HaloTag[22-297](M2F)-12×Gly/Ser-LgBiT under control of a CMV or TK promoter. Approximately 48 hours post-transfection, the cells were labeled with 100 nM JF646 HaloTag® ligand. Following a 1-hour incubation, the cells were washed with DPBS media for 30 minutes (2× at 15 minutes each). Subsequently, the cells were detached, and their fluorescence activity was measured using flow cytometry. The fold response of each assay condition was calculated as the ratio of mean fluorescence signal in the JF646+ gate for PARP1-GGSG-HaloTag[3-19]-GGSG-VS-HiBiT DLD-1 cells transiently expressing HaloTag[22-297](M2F)-12×Gly/Ser-LgBiT to the mean fluorescence signal of parental DLD-1 cells transiently expressing HaloTag[22-297](M2F)-12×Gly/Ser-LgBiT in the same JF646+ gate. The values on the x-axis correspond to the amount of plasmid DNA expressing HaloTag[22-297](M2F)-12×Gly/Ser-LgBiT used to transiently transfect cells in each well of the 96-well plate. The error bars represent the standard deviation of the mean (SD) of the data. These results demonstrate that the fold response can be improved by adjusting the concentration of plasmid DNA expressing HaloTag22-297-12×Gly/Ser-LgBiT, particularly under a promoter that expresses a lower amount of HaloTag22-297-12×Gly/Ser-LgBiT such as a TK promoter.

FIG. 106A-C. Comparison of Promoter Influence on Percentage of Cells Labeled with HaloTag Ligand in Endogenous tagged PARP1-GGSG-HaloTag[3-19]-GGSG-VS-HiBiT DLD-1 Cells and Parental DLD-1 Cells Transiently Expressing HaloTag[22-297](M2F)-12×Gly/Ser-LgBiT. FIG. 106(A) and FIG. 106(B) Parental DLD-1 Cells and DLD-1 cells expressing endogenous PARP1-GGSG-HaloTag[3-19]-GGSG-VS-HiBiT were transiently transfected with different concentrations of plasmids expressing HaloTag[22-297](M2F)-12×Gly/Ser-LgBiT under control of a CMV (A) and TK (B) promoters. FIG. 106(C) DLD-1 cells expressing endogenous PARP1-GGSG-HaloTag[3-19]-GGSG-VS-HiBiT were transiently transfected with different concentrations of plasmids expressing HaloTag[22-297](M2F)-12×Gly/Ser-LgBiT under control of a CMV or TK promoter. Approximately 48 hours post-transfection, the cells were labeled with 100 nM JF646 HaloTag® ligand. Following a 1-hour incubation, the cells were washed with DPBS media for 30 minutes (2× at 15 minutes each). Subsequently, the cells were detached, and their fluorescence activity was measured using flow cytometry. The values on the x-axis correspond to the amount of plasmid DNA encoding HaloTag[22-297](M2F)-12×Gly/Ser-LgBiT used for transient transfection in each well of a 96-well plate. The error bars represent the standard deviation of the mean (SD) of the data. This graph illustrates that the percentage of DLD-1 cells overexpressing the HaloTag[22-297](M2F)-12×Gly/Ser-LgBiT variants under the CMV promoter in the JF646+ gate is generally higher than the corresponding percentage for CRISPR cells overexpressing the HaloTag[22-297](M2F)-12×Gly/Ser-LgBiT variant under the TK promoter.

FIG. 107. Fold Difference in the Percentage of Cells Labeled with HaloTag Ligand in Endogenous tagged PARP1-GGSG-HaloTag[3-19]-GGSG-VS-HiBiT DLD-1 Cells Transiently Expressing HaloTag[22-297](M2F)-12×Gly/Ser-LgBiT Under Different Promoters. DLD-1 cells expressing endogenous PARP1-GGSG-HaloTag[3-19]-GGSG-VS-HiBiT were transiently transfected plasmids expressing HaloTag[22-297](M2F)-12×Gly/Ser-LgBiT under control of a CMV or TK promoter. Approximately 48 hours post-transfection, the cells were labeled with 100 nM JF646 HaloTag® ligand. Following a 1-hour incubation, the cells were washed with DPBS media for 30 minutes (2× at 15 minutes each). Subsequently, the cells were detached, and their fluorescence activity was measured using flow cytometry. The fold response of each assay condition was calculated as the ratio of the frequency of the parent in the JF646+ gate for PARP1-GGSG-HaloTag[3-19]-GGSG-VS-HiBiT DLD-1 cells expressing HaloTag[22-297](M2F)-12×Gly/Ser-LgBiT to the frequency of the parent of parental DLD-1 cells transiently expressing HaloTag[22-297](M2F)-12×Gly/Ser-LgBiT in the same JF646+ gate. The values on the x-axis correspond to the amount of plasmid DNA encoding HaloTag[22-297](M2F)-12×Gly/Ser-LgBiT used for transient transfection in each well of a 96-well plate. The error bars represent the standard deviation of the mean (SD) of the data. This graph illustrates that although the overall fluorescent intensity with the TK promoter is approximately one log lower compared to the expression with the CMV promoter, resulting in a lower percentage of cells in the JF646+ gate (lower frequency of parent values), the fold difference of tagged CRISPR cells over parental cells in the JF646+ gate is higher with the TK promoter compared to the CMV promoter. The fold response is maximized around 10-40 ng/well of HaloTag [22-297](M2F)-12×Gly/Ser-LgBiT plasmid DNA concentration for expression with the TK promoter.

FIG. 108A-B. Comparison of Fluorescence Intensity between HaloTag[22-297](M2F)-12×Gly/Ser-LgBiT and HaloTag[22-297](M2F+K140E)-12×Gly/Ser-LgBiT in Flow Cytometry. FIG. 108(A) Overlaid histograms of PARP1-GGSG-HaloTag[3-19]-GGSG-VS-HiBiT DLD-1 cells transiently expressing either HaloTag[22-297](M2F)-12×Gly/Ser-LgBiT (light grey) or its variant HaloTag[22-297](M2F+K140E)-12×Gly/Ser-LgBiT (dark grey). “Normalized to mode” on the Y-axis scales the number of events values based on the most frequently occurring value, in this graph the number of events in the JF646 negative gate. This enables standardized comparisons and highlights relative differences between populations. FIG. 108(B) Mean fluorescent intensity of HaloTag[22-297](M2F)-12×Gly/Ser-LgBiT or its variant containing the K140E mutation in the JF646 positive gate in PARP1-GGSG-HaloTag[3-19]-GGSG-VS-HiBiT DLD-1 cells. Cells expressing endogenous PARP1-GGSG-HaloTag[3-19]-GGSG-VS-HiBiT were transiently transfected with HaloTag[22-297](M2F)-12×Gly/Ser-LgBiT or variant plasmids at 20 ng/well in a 96 well plate. Approximately 48 hours post-transfection, the cells were labeled with 100 nM JF646 HaloTag® ligand. Following a 1-hour incubation, the cells were washed with DPBS media for 30 minutes (2× at 15 minutes each). Subsequently, the cells were detached, and their fluorescence activity was measured using flow cytometry. Two technical replicates were tested for each construct. The gating strategy employed to exclude dead cells, cell debris, and cell aggregates is consistent with previous cytometry experiments. This graph illustrates that HaloTag[22-297](M2F)-12×Gly/Ser-LgBiT variant exhibits higher mean fluorescent intensity within the applied gating for JF646 activity. The error bars represent the standard deviation of the mean (SD) of the data.

FIG. 109A-B. Comparison of Fluorescence Fold Response in the JF646 Positive Gate between HaloTag[22-297](M2F)-12×Gly/Ser-LgBiT and HaloTag[22-297](M2F+K140E)-12×Gly/Ser-LgBiT Variants using Flow Cytometry. FIG. 109(A) Overlaid histograms of PARP1-GGSG-HaloTag[3-19]-GGSG-VS-HiBiT DLD-1 cells transiently expressing either HaloTag[22-297](M2F)-12×Gly/Ser-LgBiT (left) or its variant HaloTag[22-297](M2F+K140E)-12×Gly/Ser-LgBiT (right). For each variant histograms, the light grey histograms are for of CRIPSR cell transiently expressing the HaloTag[22-297](M2F)-12×Gly/Ser-LgBiT variants and darker grey histograms are for parental DLD-1 cells transiently expressing HaloTag[22-297](M2F)-12×Gly/Ser-LgBiT variants. “Normalized to mode” on the Y-axis scales the number of events values based on the most frequently occurring value, in this graph the number of events in the JF646 negative gate. This enables standardized comparisons and highlights relative differences between populations. FIG. 109(B) Fold Response of HaloTag[22-297](M2F+K140E)-12×Gly/Ser-LgBiT variants in the JF646 positive gate. Cells expressing endogenous PARP1-GGSG-HaloTag[3-19]-GGSG-VS-HiBiT were transiently transfected with HaloTag[22-297](M2F)-12×Gly/Ser-LgBiT or variant plasmids at 20 ng/well into wells of a 96-well plate. Approximately 48 hours post-transfection, the cells were labeled with 100 nM JF646 HaloTag® ligand. Following a 1-hour incubation, the cells were washed with DPBS media for 30 minutes (2× at 15 minutes each). Subsequently, the cells were detached, and their fluorescence activity was measured using flow cytometry. The fold response of each assay condition was calculated as the ratio of mean fluorescence signal in the JF646+ gate for PARP1-GGSG-HaloTag[3-19]-GGSG-VS-HiBiT DLD-1 cells transiently expressing HaloTag[22-297](M2F)-12×Gly/Ser-LgBiT to the mean fluorescence signal of parental DLD-1 cells expressing HaloTag[22-297](M2F)-12×Gly/Ser-LgBiT in the same JF646+ gate. The gating strategy employed to exclude dead cells, cell debris, and cell aggregates is consistent with previous cytometry experiments. The error bars represent the standard deviation of the mean (SD) of the data.

FIG. 110A-B. Influence of the K140E Mutation on Fold Difference of JF646 Positive Cells in Endogenous tagged PARP1-GGSG-HaloTag[3-19]-GGSG-VS-HiBiT DLD-1 Cells Transiently Expressing HaloTag[22-297](M2F)-12×Gly/Ser-LgBiT. DLD-1 cells expressing endogenous PARP1-GGSG-HaloTag[3-19]-GGSG-VS-HiBiT were transiently transfected plasmids expressing either FIG. 110(A) 20 ng/well or FIG. 110(B) 40 ng/well of HaloTag[22-297](M2F)-12×Gly/Ser-LgBiT or HaloTag[22-297](M2F+K140E)-12×Gly/Ser-LgBiT. Approximately 48 hours post-transfection, the cells were labeled with 100 nM JF646 HaloTag® ligand. Following a 1-hour incubation, the cells were washed with DPBS media for 30 minutes (2× at 15 minutes each). Subsequently, the cells were detached, and their fluorescence activity was measured using flow cytometry. The fold response of each assay condition was calculated as the ratio of frequency of parent in the JF646+ gate for PARP1-GGSG-HaloTag[3-19]-GGSG-VS-HiBiT DLD-1 cells expressing the HaloTag[22-297](M2F)-12×Gly/Ser-LgBiT variants to the frequency of parent of DLD-1 parental cells expressing the HaloTag[22-297](M2F)-12×Gly/Ser-LgBiT variants in the same JF646+ gate. The gating strategy employed to exclude dead cells, cell debris, and cell aggregates is consistent with previous cytometry experiments. Two technical replicates were tested for each construct. The error bars represent the standard deviation of the mean (SD) of the data. It also demonstrates that the frequency of parent and the fold response for the frequency of parent change with different amounts of plasmid DNA expressing HaloTag[22-297](M2F)-12×Gly/Ser-LgBiT being used to transfect cells.

FIG. 111. Comparison of Fluorescent Intensity of HaloTag[22-297](M2F)-12×Gly/Ser-LgBiT Transiently Expressed using mRNA in Parental and a PARP1-GGSG-HaloTag[3-19]-GGSG-VS-HiBiT DLD-1 cell lines. DLD-1 cells expressing endogenous PARP1-GGSG-HaloTag[3-19]-GGSG-VS-HiBiT were transfected with mRNA encoding HaloTag[22-297](M2F)-12×Gly/Ser-LgBiT. After approximately 24 hours post-transfection, the cells were labeled with 100 nM JF646 HaloTag® ligand. Following a 1-hour incubation, the cells underwent two 15-minute washes with DPBS media, totaling 30 minutes. Subsequently, the cells were detached, and their fluorescence activity was quantified using flow cytometry. The gating strategy employed for excluding dead cells, cell debris, and cell aggregates remained consistent with previous cytometry experiments. For each sample, the JF646 positive gate setting was optimized to maximize differentiation between tagged and untagged cells that varied for each mRNA concentration, in order to compare optimal performance at each mRNA concentration. The values on the x-axis correspond to the mRNA concentration used for transfection in each well of a 96-well plate. Two technical replicates were performed for each construct. The error bars represent the standard deviation of the mean (SD) of the data. The fluorescence intensity value for the no-mRNA control is zero in the JF646 positive gate.

FIG. 112. Fold Response Comparison of HaloTag[22-297](M2F)-12×Gly/Ser-LgBiT Transiently Expressed using mRNA in Parental and a PARP1-GGSG-HaloTag[3-19]-GGSG-VS-HiBiT DLD-1 cell lines. DLD-1 cells expressing endogenous PARP1-GGSG-HaloTag[3-19]-GGSG-VS-HiBiT were transfected with mRNA encoding HaloTag[22-297](M2F)-12×Gly/Ser-LgBiT. After approximately 24 hours post-transfection, the cells were labeled with 100 nM JF646 HaloTag® ligand. Following a 1-hour incubation, the cells underwent two 15-minute washes with DPBS media, totaling 30 minutes. Subsequently, the cells were detached, and their fluorescence activity was quantified using flow cytometry. The gating strategy employed for excluding dead cells, cell debris, and cell aggregates remained consistent with previous cytometry experiments. However, the gate setting to distinguish between JF646-positive and negative populations varied for each mRNA concentration. The setting was optimized to achieve maximum separation between the tagged cells and parental cells. The fold response of each assay condition was calculated as the ratio of mean fluorescence signal in the JF646+ gate for PARP1-GGSG-HaloTag[3-19]-GGSG-VS-HiBiT DLD-1 cells expressing HaloTag[22-297](M2F)-12×Gly/Ser-LgBiT to the mean fluorescence intensity of DLD-1 parental cells expressing HaloTag[22-297](M2F)-12×Gly/Ser-LgBiT in the same JF646+ gate. The values on the x-axis correspond to the mRNA concentration used for transfection in each well of a 96-well plate. This graph illustrates that the fold responses are higher at lower concentrations of HaloTag[22-297](M2F)-12×Gly/Ser-LgBiT variant mRNA. The error bars represent the standard deviation of the mean (SD) of the data. This observation suggests that at higher concentrations of HaloTag[22-297](M2F)-12×Gly/Ser-LgBiT variant mRNA, the abundance of HaloTag[22-297](M2F)-12×Gly/Ser-LgBiT variant exceeds the endogenously expressed PARP1-GGSG-HaloTag[3-19]-GGSG-VS-HiBiT, resulting in non-specific signal accumulation and reduced fold response.

FIG. 113A-B. Comparison of Fluorescence Intensity of HaloTag[22-297](M2F)-12×Gly/Ser-LgBiT transiently expressed using mRNA in PARP1-GGSG-HaloTag[3-19]-GGSG-VS-HiBiT DLD-1 Cells using Flow Cytometry. FIG. 113(A) Comparison of Fluorescence Intensity between DLD-1 cells expressing endogenous PARP1-GGSG-HaloTag[3-19]-GGSG-VS-HiBiT (Right) and Parental DLD-1 cells (Left) which were transiently transfected with varying concentrations of mRNA expressing HaloTag[22-297](M2F)-12×Gly/Ser-LgBiT. FIG. 113(B). PARP1-GGSG-HaloTag[3-19]-GGSG-VS-HiBiT DLD-1 cells (light grey) were transiently transfected with mRNA encoding HaloTag[22-297](M2F)-12×Gly/Ser-LgBiT (5 ng/well for a 96 well plate). As a control, the same mRNA was used to transfect parental DLD-1 cells (dark grey). After approximately 24 hours post-transfection, the cells were labeled with 100 nM JF646 HaloTag® ligand. Following a 1-hour incubation, the cells underwent two 15-minute washes with DPBS media, totaling 30 minutes. Subsequently, the cells were detached, and their fluorescence intensity was measured using flow cytometry. The gating strategy employed for excluding dead cells, cell debris, and cell aggregates remained consistent with previous cytometry experiments. The JF646 positive gate is shown for samples where 5 ng/well of mRNA was used for transfection. “Normalized to mode” on the Y-axis scales the number of events values based on the most frequently occurring value, in this graph the number of events in the JF646 negative gate. This enables standardized comparisons and highlights relative differences between populations.

FIG. 114. Fold Difference in the Percentage of Cells Labeled with HaloTag Ligand when transiently expressing HaloTag[22-297](M2F)-12×Gly/Ser-LgBiT using mRNA in PARP1-GGSG-HaloTag[3-19]-GGSG-VS-HiBiT DLD-1 Cells using Flow Cytometry. DLD-1 cells expressing endogenous PARP1-GGSG-HaloTag[3-19]-GGSG-VS-HiBiT were transfected with indicated amounts of mRNA encoding HaloTag[22-297](M2F)-12×Gly/Ser-LgBiT. After approximately 24 hours post-transfection, the cells were labeled with 100 nM HaloTag® ligands, JF646 and TMR. Following a 1-hour incubation, the cells underwent two 15-minute washes with DPBS media, totaling 30 minutes. Subsequently, the cells were detached, and their fluorescence activity was quantified using flow cytometry. The frequency of the parent for each assay condition was measured using the exact same gate setting for fluorescent intensity measurements. The fold response of each assay condition was calculated as the ratio of the frequency of the parent in the JF646+ gate for PARP1-GGSG-HaloTag[3-19]-GGSG-VS-HiBiT DLD-1 cells expressing HaloTag[22-297](M2F)-12×Gly/Ser-LgBiT to the frequency of the parent of parental DLD-1 cells expressing HaloTag[22-297](M2F)-12×Gly/Ser-LgBiT in the same JF646+ gate. The values on the x-axis correspond to the mRNA concentration used for transfection in each well of a 96-well plate. The error bars represent the standard deviation of the mean (SD) of the data.

FIG. 115. Comparison of Fluorescence Intensity of HaloTag[22-297](M2F)-12×Gly/Ser-LgBiT transiently expressed using plasmid DNA or mRNA in PARP1-GGSG-HaloTag[3-19]-GGSG-VS-HiBiT DLD-1 Cells using Flow Cytometry. DLD-1 cells expressing endogenous PARP1-GGSG-HaloTag[3-19]-GGSG-VS-HiBiT were transfected with 5 ng/well of either plasmid DNA (dark grey) or mRNA (light grey) encoding HaloTag[22-297](M2F)-12×Gly/Ser-LgBiT. After approximately 24 hours post-transfection, the cells were labeled with 100 nM JF646 HaloTag® ligand. Following a 1-hour incubation, the cells underwent two 15-minute washes with DPBS media, totaling 30 minutes. Subsequently, the cells were detached, and their fluorescence activity was measured using flow cytometry. The gating strategy employed for excluding dead cells, cell debris, and cell aggregates remained consistent with previous cytometry experiments. This graph demonstrates the impact of HaloTag[22-297](M2F)-12×Gly/Ser-LgBiT expression method on fluorescence intensity across the cell population. When expressed via mRNA, there is lower expression variability, leading to a more distinct peak for JF646 positive cells. In contrast, when comparing the same amount of plasmid DNA expression, a broad peak is observed, indicating a wider range of HaloTag[22-297](M2F)-12×Gly/Ser-LgBiT expression. “Normalized to mode” on the Y-axis scales the number of events values based on the most frequently occurring value, in this graph the number of events in the JF646 negative gate. This enables standardized comparisons and highlights relative differences between populations.

FIG. 116. Confocal Imaging of Live PARP1-HaloTag[3-19]-GGSG-VS-HiBiT DLD-1 Cells transiently transfected with HaloTag[22-297](M2F)-12×Gly/Ser-LgBiT plasmid DNA. DLD-1 cells expressing endogenous PARP1-GGSG-HaloTag[3-19]-GGSG-VS-HiBiT (top panels) or parental DLD-1 cells (bottom panels) were transfected with plasmid DNA encoding HaloTag[22-297](M2F)-12×Gly/Ser-LgBiT. Approximately 48 hours post-transfection, the cells were labeled with 100 nM JF646 HaloTag® ligand. Following a 1-hour incubation, the cells were imaged using a confocal microscope in the blue channel (Ex. 405 nm), the far-red channel (Ex. 640 nm), along with DIC (Differential interference contrast). The laser and gain settings were optimized to minimize saturated pixels in each channel. The PARP1 protein naturally localizes in the cell nucleus, and the images of the PARP1-HaloTag[3-19]-GGSG-VS-HiBiT DLD-1 cells show the localization of the far-red signal in the nucleus, along with a clear colocalization of DAPI and JF646. These images provide evidence that tagging the endogenous target, PARP1, with GGSG-HaloTag[3-19]-GGSG-VS-HiBiT will not affect the tagged protein localization to the nucleus.

FIG. 117. Quantification of Confocal Imaging of Live PARP1-HaloTag[3-19]-GGSG-VS-HiBiT DLD-1 Cells transiently transfected with HaloTag[22-297](M2F)-12×Gly/Ser-LgBiT plasmid DNA. DLD-1 cells expressing endogenous PARP1-GGSG-HaloTag[3-19]-GGSG-VS-HiBiT or parental DLD-1 cells were transfected with plasmid DNA encoding HaloTag[22-297](M2F)-12×Gly/Ser-LgBiT. Approximately 48 hours post-transfection, the cells were labeled with 100 nM JF646 HaloTag® ligand. Confocal microscopy was performed using a blue channel (Ex. 405 nm), far-red channel (Ex. 640 nm), and DIC (Differential Interference Contrast). The laser and gain settings were carefully optimized to minimize saturated pixels, ensuring optimal image quality. These settings were consistently applied for collecting both the specific and background signals. For quantification, a single field of view (FOV) of PARP1-GGSG-HaloTag[3-19]-GGSG-VS-HiBiT DLD-1 cells was analyzed to measure the nuclear-localized fluorescence intensity for PARP1, while a separate FOV of parental cells was assessed to quantify the whole cell background fluorescence intensity. The intensity of individual cells analyzed by this strategy was used to generate datapoints on the plot. The bar represents the Mean of the data. The graph illustrates the heterogeneity of expression with plasmid DNA and highlights outliers resulting from higher expression that can influence the mean of the data. The imaging analysis correlates with cytometry assay data analysis, as the fold response of fluorescence in CRISPR cells does not significantly differ from the fluorescent intensity of parental cells. However, the distinguishing factor lies in the number of fluorescent cells within a defined gate in cytometry or with a threshold setting in imaging.

FIG. 118. Confocal Imaging of Live PARP1-HaloTag[3-19]-GGSG-VS-HiBiT DLD-1 Cells transiently transfected with HaloTag[22-297](M2F)-12×Gly/Ser-LgBiT mRNA. DLD-1 cells expressing endogenous PARP1-GGSG-HaloTag[3-19]-GGSG-VS-HiBiT (top row) or parental DLD-1 cells (bottom row) were transfected with 5 ng/well mRNA encoding HaloTag[22-297](M2F)-12×Gly/Ser-LgBiT. Approximately 24 hours post-transfection, the cells were labeled with 100 nM JF646 HaloTag® ligand. Following a 1-hour incubation, the cells were imaged using a confocal microscope in the blue channel (Ex. 405 nm), the far-red channel (Ex. 640 nm), along with DIC (Differential Interference Contrast). The laser and gain settings were optimized to minimize saturated pixels in each channel. The PARP1 protein naturally localizes within the cell nucleus. The images of the PARP1-GGSG-HaloTag[3-19]-GGSG-VS-HiBiT DLD-1 cells illustrates the far-red signal's localization in the nucleus, along with a noticeable colocalization of DAPI and JF646. The background signal from the non-complemented HaloTag[22-297](M2F)-12×Gly/Ser-LgBiT labeling exhibits whole-cell localization since excess HaloTag[22-297](M2F)-12×Gly/Ser-LgBiT expression also occurs in the cell cytoplasm. A comparison of confocal images depicting the expression of HaloTag[22-297](M2F)-12×Gly/Ser-LgBiT using plasmid DNA versus mRNA shows significantly more homogeneous expression with mRNA. These images also provide evidence that tagging the endogenous target, PARP1 protein, with GGSG-HaloTag[3-19]-GGSG-VS-HiBiT does not affect its localization.

FIG. 119. Quantification of Confocal Imaging of Live PARP1-HaloTag[3-19]-GGSG-VS-HiBiT DLD-1 Cells transiently transfected with HaloTag[22-297](M2F)-12×Gly/Ser-LgBiT mRNA. DLD-1 cells expressing endogenous PARP1-GGSG-HaloTag[3-19]-GGSG-VS-HiBiT or parental DLD-1 cells were transfected with 5 ng/well mRNA encoding HaloTag[22-297](M2F)-12×Gly/Ser-LgBiT. Approximately 24 hours post-transfection, cells were labeled with 100 nM JF646 HaloTag® ligand and incubated for 1 hour. Confocal microscopy was performed using a blue channel (Ex. 405 nm), far-red channel (Ex. 640 nm), and DIC (Differential Interference Contrast). The laser and gain settings were carefully optimized to minimize saturated pixels, ensuring optimal image quality. These settings were consistently applied for collecting both the specific and background signals. For quantification, a single field of view (FOV) of PARP1-GGSG-HaloTag[3-19]-GGSG-VS-HiBiT DLD-1 cells was analyzed to measure the nuclear-localized fluorescence intensity for PARP1, while a separate FOV of parental cells was assessed to quantify the whole cell background fluorescence intensity. The intensity of individual cells analyzed by this strategy was used to generate datapoints on the plot. The bar represents the Mean of the data. The graph demonstrates a higher level of homogeneity in expression with mRNA, with only a few outliers present. As a result, the mean (1215.003) and median (1192.750) values of the data are similar.

FIG. 120A-B. Dissociation kinetics of purified HaloTag[22-297](M2F)-12×Gly/Ser-LgBiT-3×Gly/Ser-6×His or LgBiT after complementation with synthetic peptide. FIG. 120(A) Purified HaloTag[22-297](M2F)-12×Gly/Ser-LgBiT-3×Gly/Ser-6×His (Reporter) or FIG. 120(B) LgBiT-6×His at 100 nM was combined with 200 nM HaloTag[3-19](I2R)-4×Gly/Ser-VS-HiBiT (Tag) or HiBiT synthetic peptides and incubated at 4° C. for 5 minutes. After preincubation with peptide, samples were diluted to 1 pM in either buffer alone or buffer 100 nM Tag peptide. At various time points, samples were transferred into luminescent substrate (Furimazine) to monitor dissociation of the complex. All measurements were performed in triplicate, and error bars represent one standard deviation from the mean.

FIG. 121A-C. Bioluminescence Resonance Energy Transfer (BRET) Enables Measurement of Purified HaloTag[22-297](M2F)-12×Gly/Ser-LgBiT-3×Gly/Ser-6×His Ligand Occupancy Across a Broad Range of Concentrations. Purified HaloTag[22-297](M2F)-12×Gly/Ser-LgBiT-3×Gly/Ser-6×His at 200 nM was combined with 2 μM HaloTag[3-19](I2R)-4×Gly/Ser-VS-HiBiT or VS-HiBiT synthetic peptide. After preincubation with peptide, complexes were serially diluted by half logs into buffer to give concentrations between 0.1 pM and 10 nM, plus buffer alone. These dilutions were added 1:1 to furimazine substrate in the presence or absence of 100 nM final concentration of HaloTag® NanoBRET® 618 Ligand. Luminescence was measured after about 1 min incubation for both FIG. 121(A) the luminescent donor signal using a 450 nm bandpass filter and FIG. 121(B) the luminescent acceptor signal using a 600 nm longpass filter. The BRET ratio FIG. 121(C) is determined by dividing the acceptor signal by the donor signal for each sample. All samples were performed in triplicate and error bars represent one standard deviation from the mean.

FIG. 122A-C. Bioluminescence Resonance Energy Transfer (BRET) Enables Measurement of Purified HaloTag[22-297](M2F)-12×Gly/Ser-LgBiT-3×Gly/Ser-6×His stabilization by synthetic peptide. Purified HaloTag[22-297](M2F)-12×Gly/Ser-LgBiT-3×Gly/Ser-6×His (Reporter) at 200 nM was combined with 2 μM HaloTag[3-19](12R)-4×Gly/Ser-VS-HiBiT (Tag) or VS-HiBiT synthetic peptide. After preincubation with peptide at 4° C., solutions were diluted 100-fold into buffer and incubated at room temperature. At time points, samples were added 1:1 to buffer either containing or lacking 10 nM final HaloTag® NanoBRET® 618 Ligand. For the reporter alone condition, samples were added 1:1 to a solution containing 10 nM final VS-HiBiT peptide with or without the 618 ligand in order to complement the LgBiT to create the BRET donor. After the last time point, furimazine substrate was added, followed by measuring the FIG. 122(A) luminescent donor signal using a 450 nm bandpass filter and FIG. 122(B) luminescent acceptor signal using a 600 nm longpass filter. The BRET ratio FIG. 122(C) is determined by dividing the acceptor signal by the donor signal for each sample.

FIG. 123A-B. Stabilization of HaloTag[22-297](M2F)-12×Gly/Ser-LgBiT Activity in the Presence of PARP1 Endogenously Tagged with HaloTag[3-19]-4×Gly/Ser-HiBiT in Live Cells Using Flow Cytometry. Separate mRNAs encoding FIG. 123(A) HaloTag[22-297](M2F)-12×Gly/Ser-LgBiT and EGFP or FIG. 123(B) HaloTag-LgBiT and EGFP were co-transfected into either parental DLD-1 (light grey) or PARP1-HaloTag[3-19]-4×Gly/Ser-VS-HiBiT DLD-1 (dark grey) cells. After 48 hours of expression, cells were labeled with 100 nM JF646 HaloTag® ligand for 60 minutes and then analyzed by flow cytometry. Stabilization of HaloTag[22-297](M2F)-12×Gly/Ser-LgBiT in the presence of PARP1-HaloTag[3-19]-4×Gly/Ser-VS-HiBiT results in increased JF646 HaloTag® ligand labeling of cells expressing both fragments relative to parental DLD-1 cells. Because the expression level and activity of HaloTag-LgBiT is not affected by the presence or absence of DualTag, its HaloTag® activity shows excellent proportionality with co-transfected EGFP, with a very similar distribution in both cell lines. The EGFP signal therefore represents a good indicator of the expression level in each cell of the co-transfected reporter.

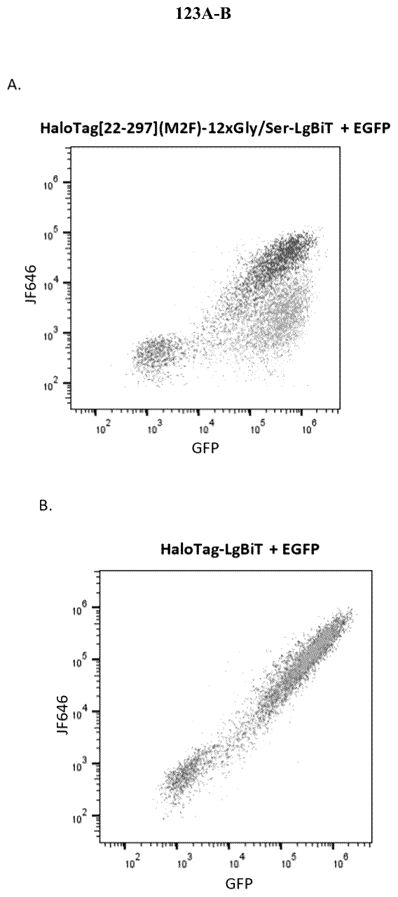

FIG. 124. Stabilization of HaloTag[22-297](M2F)-12×Gly/Ser-LgBiT Activity in the Presence of PARP1 Endogenously Tagged with HaloTag[3-19]-4×Gly/Ser-HiBiT in Cells using Luminescence. mRNA encoding HaloTag[22-297](M2F)-12×Gly/Ser-LgBiT was transfected into either parental DLD-1 or PARP1-HaloTag[3-19]-4×Gly/Ser-VS-HiBiT DLD-1 cells. After 24 hours of expression, cells were treated with 50 μg/ml Cycloheximide (0 hour timepoint) and at subsequent timepoints replicate samples were lysed. To measure changes in total reporter levels, a lytic reagent containing excess HiBiT peptide and furimazine substrate was added at various time points. The ratio of luminescence signal for +/−Cycloheximide treated samples for each condition was calculated and then normalized to the initial ratio at 0 hours post-cycloheximide treatment. Half-life was calculated using a one phase exponential decay model. Five replicates were measured for each condition, and error bars represent one standard deviation from the mean.

FIG. 125. Gate Settings for Distinguishing JF646 Positive and Negative Cell Populations. The histogram displays the number of cells (Y-axis) versus their intensity with JF646 ligand (X-axis). A consistent gating strategy, developed using baseline activity from non-transfected cells, was applied across all transfected samples to accurately identify and quantify fluorescence activity. This strategy aligns with previous cytometry experiments, effectively excluding dead cells, cell debris, and aggregates.

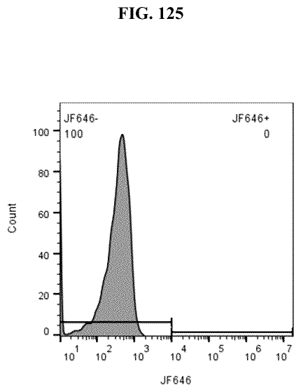

FIG. 126A-B. Comparison of Fluorescent Intensity for HaloTag[22-297](M2F)-12×Gly/Ser-LgBiT and HaloTag[22-297](M2F+D53G+K140E)-12×Gly/Ser-LgBiT Variants Transiently Expressed using mRNA in Parental and a PARP1-GGSG-HaloTag[3-19]-GGSG-VS-HiBiT DLD-1 cell lines at 24 and 48 Hours Post-Transfection. Parental DLD-1 cells and DLD-1 cells expressing endogenous PARP1-GGSG-HaloTag[3-19]-GGSG-VS-HiBiT were transfected with mRNA encoding HaloTag[22-297](M2F)-12×Gly/Ser-LgBiT and HaloTag[22-297](M2F+D53G+K140E)-12×Gly/Ser-LgBiT variants. At approximately 24 hours post-transfection (A), and 48 hours post-transfection (B), the cells were labeled with 100 nM JF646 HaloTag® ligand. Following a 1-hour incubation, the cells underwent two 15-minute washes with DPBS media, totaling 30 minutes. Subsequently, the cells were detached, and their fluorescence activity was quantified using flow cytometry. A consistent gating strategy was used across all experiments to exclude dead cells, debris, and aggregates and identical gate settings were employed to distinguish between JF646 positive and negative populations. The values on the x-axis correspond to the mRNA concentration used for transfection in each well of a 96-well plate. Two technical replicates were performed for each construct. The error bars represent the standard deviation of the mean (SD) of the data. The absence of a bar in any assay condition indicates no detectable signal in the JF646 positive gate for the parental DLD-1 cells transfected with mRNA encoding HaloTag[22-297](M2F)-12×Gly/Ser-LgBiT and HaloTag[22-297](M2F+D53G+K140E)-12×Gly/Ser-LgBiT variants. The fluorescence intensity value for the no-mRNA control is zero in the JF646 positive gate. These graphs illustrate that fluorescent intensity approximately halves at 48 hours post-transfection compared to 24 hours.

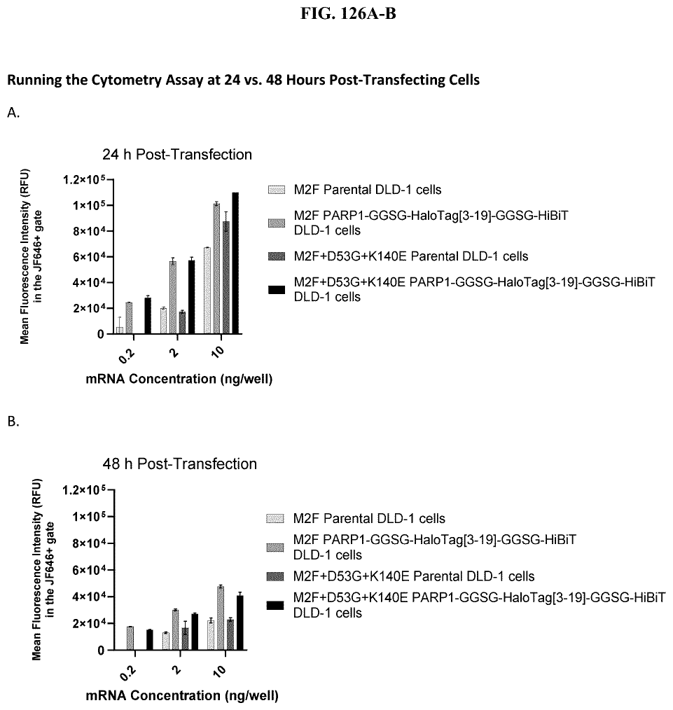

FIG. 127A-B. Fold Response Comparison of HaloTag[22-297](M2F)-12×Gly/Ser-LgBiT and HaloTag[22-297](M2F+d53g+K140E)-12×Gly/Ser-LgBiT Variants Transiently Expressed Using mRNA in Parental and PARP1-GGSG-HaloTag[3-19]-GGSG-VS-HiBiT DLD-1 Cell Lines at 24 and 48 Hours Post-Transfection. Parental DLD-1 cells and DLD-1 cells expressing endogenous PARP1-GGSG-HaloTag[3-19]-GGSG-VS-HiBiT were transfected with mRNA encoding HaloTag[22-297](M2F)-12×Gly/Ser-LgBiT and HaloTag[22-297](M2F+D53G+K140E)-12×Gly/Ser-LgBiT variants. At approximately 24 hours post-transfection (A), and 48 hours post-transfection (B), the cells were labeled with 100 nM JF646 HaloTag® ligand. Following a 1-hour incubation, the cells underwent two 15-minute washes with DPBS media, totaling 30 minutes. Subsequently, the cells were detached, and their fluorescence activity was quantified using flow cytometry. A consistent gating strategy was used across all experiments to exclude dead cells, debris, and aggregates, and identical gate settings were employed to distinguish between JF646 positive and negative populations. The fold response of each assay condition was calculated as the ratio of mean fluorescence signal in the JF646+ gate for PARP1-GGSG-HaloTag[3-19]-GGSG-VS-HiBiT DLD-1 cells expressing HaloTag[22-297](M2F)-12×Gly/Ser-LgBiT and HaloTag[22-297](M2F+D53G+K140E)-12×Gly/Ser-LgBiT variants to the mean fluorescence intensity of DLD-1 parental cells expressing HaloTag[22-297](M2F)-12×Gly/Ser-LgBiT and HaloTag[22-297](M2F+D53G+K140E)-12×Gly/Ser-LgBiT variants in the same JF646+ gate. The values on the x-axis correspond to the mRNA concentration used for transfection in each well of a 96-well plate. The error bars represent the standard deviation of the mean (SD) of the data. The absence of a bar in any assay condition indicates no detectable signal in the JF646 positive gate for the parental DLD-1 cells transfected with mRNA encoding HaloTag[22-297](M2F)-12×Gly/Ser-LgBiT and HaloTag[22-297](M2F+D53G+K140E)-12×Gly/Ser-LgBiT variants. These results suggest no significant difference in fold response between assays run at 24- and 48-hours post-transfection. Additionally, it indicates higher fold responses at lower mRNA concentrations.

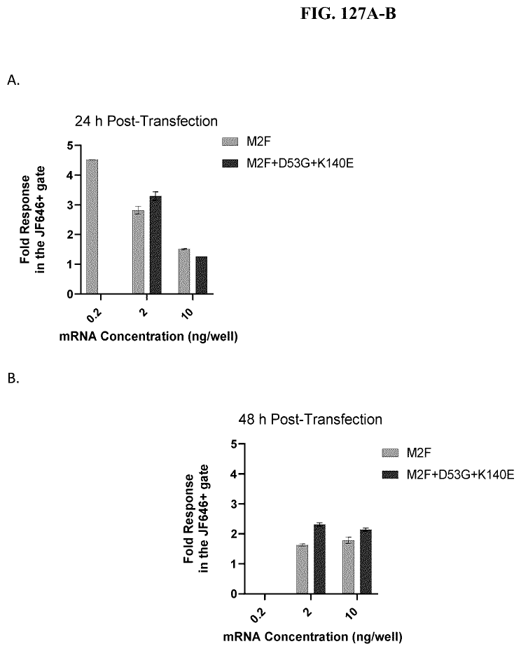

FIG. 128A-B. Comparison of Percentage of Cells Labeled with HaloTag[22-297](M2F)-12×Gly/Ser-LgBiT and HaloTag[22-297](M2F+D53G+K140E)-12×Gly/Ser-LgBiT Variants When Transiently Expressed Using mRNA in Parental and a PARP1-GGSG-HaloTag[3-19]-GGSG-VS-HiBiT DLD-1 Cell Lines at 24 and 48 Hours Post-Transfection. Parental DLD-1 cells and DLD-1 cells expressing endogenous PARP1-GGSG-HaloTag[3-19]-GGSG-VS-HiBiT were transfected with mRNA encoding HaloTag[22-297](M2F)-12×Gly/Ser-LgBiT and HaloTag[22-297](M2F+D53G+K140E)-12×Gly/Ser-LgBiT variants. After approximately 24 hours post-transfection (A) and 48 hours post-transfection (B), the cells were labeled with 100 nM JF646 HaloTag® ligand. Following a 1-hour incubation, the cells underwent two 15-minute washes with DPBS media, totaling 30 minutes. Subsequently, the cells were detached, and their fluorescence activity was quantified using flow cytometry. A consistent gating strategy was used across all experiments to exclude dead cells, debris, and aggregates and identical gate settings were employed to distinguish between JF646 positive and negative populations. The frequency of the parent for each assay condition was measured using the exact same gate setting for fluorescent intensity measurements. The values on the x-axis correspond to the mRNA concentration used for transfection in each well of a 96-well plate. Two technical replicates were performed for each construct. The error bars represent the standard deviation of the mean (SD) of the data. The absence of a bar in any assay condition indicates no detectable labeled cells in the JF646 positive gate for the parental DLD-1 cells transfected with mRNA encoding HaloTag[22-297](M2F)-12×Gly/Ser-LgBiT and HaloTag[22-297](M2F+D53G+K140E)-12×Gly/Ser-LgBiT variants. The fluorescence intensity value for the no-mRNA control is zero in the JF646 positive gate. These graphs illustrate that although fluorescent intensity decreases by approximately 50% at 48 hours post-transfection compared to 24 hours, there is a significant increase in the percentage of labeled PARP1-GGSG-HaloTag[3-19]-GGSG-VS-HiBiT DLD-1 cells expressing HaloTag® variants compared to labeled parental DLD-1 cells expressing the same variants at the JF646 positive gate. At mRNA concentrations of 0.2 ng/well, no labeled parental cells were detected, and less than 1% were detected at 2 ng/well. In contrast, on average 15% and 40% of the PARP1-GGSG-HaloTag[3-19]-GGSG-VS-HiBiT DLD-1 cells were labeled under these conditions, respectively. Therefore, for fluorescent sorting applications, it is recommended to conduct the assay 48 hours post-transfection. These results indicate that both HaloTag[22-297](M2F)-12×Gly/Ser-LgBiT and HaloTag[22-297](M2F+D53G+K140E)-12×Gly/Ser-LgBiT variants perform similarly in the cell cytometry assay. The latter variant demonstrates a slight improvement in fluorescence and the percentage of labeled cells' fold response.

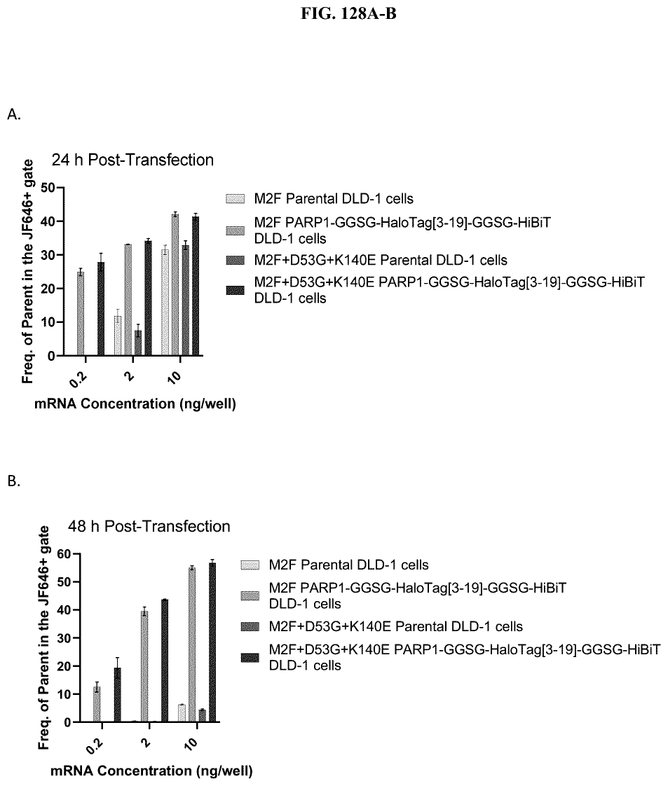

FIG. 129A-B. Fluorescence Intensity Comparison of HaloTag[22-297](M2F)-12×Gly/Ser-LgBiT Variants in a Time-Lapse Ligand Incubation Cytometry Assay in DLD-1 Cell Lines. Panel A depicts the fluorescent intensity for HaloTag[22-297](M2F)-12×Gly/Ser-LgBiT, HaloTag[22-297](M2F+K140E)-12×Gly/Ser-LgBiT, and HaloTag[22-297](M2F+D53G+K140E)-12×Gly/Ser-LgBiT variants expressed in both parental and PARP1-GGSG-HaloTag[3-19]-GGSG-VS-HiBiT DLD-1 cell lines. These variants were transiently expressed using mRNA in both parental and PARP1-GGSG-HaloTag[3-19]-GGSG-VS-HiBiT DLD-1 cell lines. Approximately 48 hours post-transfection, cells were labeled with 100 nM JF646 HaloTag® ligand and incubated for varying durations (0.5, 2, 8, and 24 hours) before detachment and fluorescence quantification via flow cytometry. A consistent gating strategy was used across all experiments to exclude dead cells, debris, and aggregates and identical gate settings were employed to distinguish between JF646 positive and negative populations. Panel B illiterates fluorescence fold response of each variant over these time points. Fold response was calculated by comparing the mean fluorescence in the JF646+ gate of transfected PARP1-GGSG-HaloTag[3-19]-GGSG-VS-HiBiT DLD-1 cell line to that of the parental cells control in the same gate. Two technical replicates were performed for each construct. The error bars represent the standard deviation of the mean (SD) of the data. The fluorescence intensity value for the no-mRNA control is zero in the JF646 positive gate. These graphs illustrate that the HaloTag[22-297](M2F)-12×Gly/Ser-LgBiT, and HaloTag[22-297](M2F+D53G+K140E)-12×Gly/Ser-LgBiT variants exhibit similar fluorescent intensities, which are slightly higher than the fluorescent intensities of HaloTag[22-297](M2F+K140E)-12×Gly/Ser-LgBiT variant. The fluorescent intensities peak around two hours post-ligand addition. While the maximum fluorescent fold response is achieved approximately two hours after ligand incubation, the fold response for HaloTag[22-297](M2F+D53G+K140E)-12×Gly/Ser-LgBiT variant reaches its peaks at about 8-hour post ligand addition.

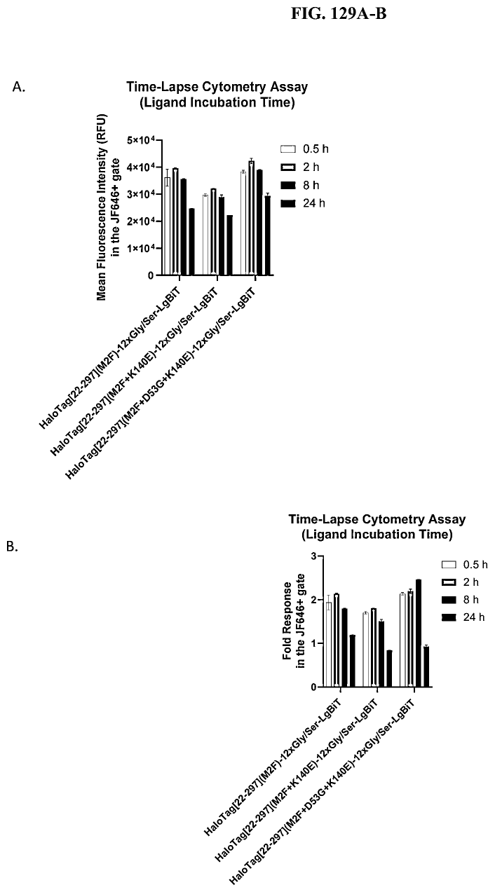

FIG. 130A-B. Comparison of Labeled Cell Percentages Among HaloTag[22-297](M2F)-12×Gly/Ser-LgBiT Variants in a Time-Lapse Ligand Incubation Cytometry Assay in DLD-1 Cell Lines. Panel A shows the percentage of cells labeled with HaloTag[22-297](M2F)-12×Gly/Ser-LgBiT, HaloTag[22-297](M2F+K140E)-12×Gly/Ser-LgBiT, and HaloTag[22-297](M2F+D53G+K140E)-12×Gly/Ser-LgBiT variants expressed in both parental and PARP1-GGSG-HaloTag[3-19]-GGSG-VS-HiBiT DLD-1 cell lines. These variants were transiently expressed using mRNA in both parental and PARP1-GGSG-HaloTag[3-19]-GGSG-VS-HiBiT DLD-1 cell lines. Approximately 48 hours post-transfection, cells were labeled with 100 nM JF646 HaloTag® ligand and incubated for varying durations (0.5, 2, 8, and 24 hours) before detachment and fluorescence quantification via flow cytometry. A consistent gating strategy was used across all experiments to exclude dead cells, debris, and aggregates and identical gate settings were employed to distinguish between JF646 positive and negative populations. Panel B illiterates the fold response of percentage of labeled cells (Frequency of Parent) of each variant over these time points. The fold response of each assay condition was calculated as the ratio of frequency of parent in the JF646+ gate for PARP1-GGSG-HaloTag[3-19]-GGSG-VS-HiBiT DLD-1 cells expressing the HaloTag[22-297](M2F)-12×Gly/Ser-LgBiT variants to the frequency of parent of DLD-1 parental cells expressing the HaloTag[22-297](M2F)-12×Gly/Ser-LgBiT variants in the same JF646+ gate. Two technical replicates were performed for each construct. The error bars represent the standard deviation of the mean (SD) of the data. The fluorescence intensity value for the no-mRNA control is zero in the JF646 positive gate. These graphs demonstrate that a longer ligand incubation time results in a better fold response for the frequency of parent. Approximately across all time points, the HaloTag[22-297](M2F+D53G+K140E)-12×Gly/Ser-LgBiT variant exhibits a slightly higher fold response compared to the other two HaloTag[22-297](M2F)-12×Gly/Ser-LgBiT, and HaloTag[22-297](M2F+K140E)-12×Gly/Ser-LgBiT variants.

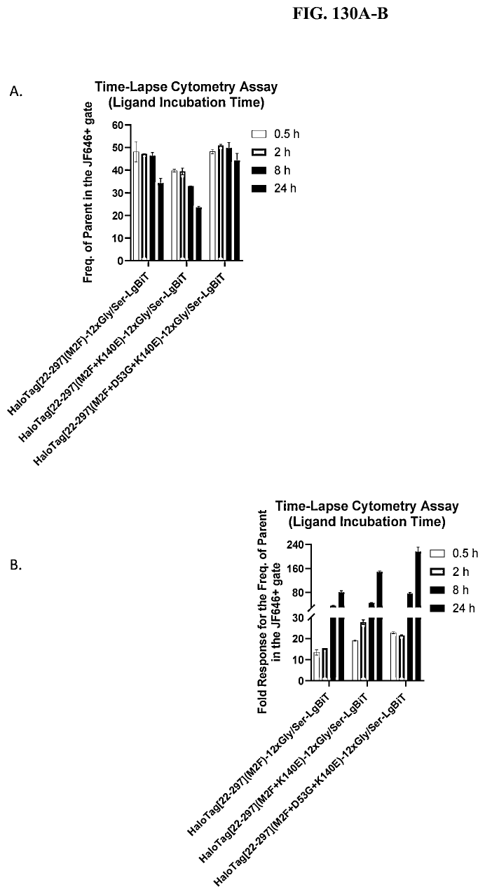

FIG. 131. Quantification of Confocal Imaging of Live PARP1-HaloTag[3-19]-GGSG-VS-HiBiT DLD-1 Cells transiently transfected with HaloTag[22-297](M2F)-12×Gly/Ser-LgBiT, HaloTag[22-297](M2F+K140E)-12×Gly/Ser-LgBiT, and HaloTag[22-297](M2F+D53G+K140E)-12×Gly/Ser-LgBiT Variants' mRNA. Parental DLD-1 cells and DLD-1 cells expressing endogenous PARP1-GGSG-HaloTag[3-19]-GGSG-VS-HiBiT were transfected with 0.5 ng/well mRNA encoding HaloTag[22-297](M2F)-12×Gly/Ser-LgBiT, HaloTag[22-297](M2F+K140E)-12×Gly/Ser-LgBiT, and HaloTag[22-297](M2F+D53G+K140E)-12×Gly/Ser-LgBiT variants. Approximately 24 hours post-transfection, cells were labeled with 100 nM JF646 HaloTag ligand and incubated for 1 hour. Confocal microscopy was performed using a blue channel (Ex. 405 nm), far-red channel (Ex. 640 nm), and DIC (Differential Interference Contrast). Laser and gain settings were optimized to avoid pixel saturation, maintaining uniform intensity across all variants to capture specific and background signals. For quantification of each variant, a single field of view (FOV) of PARP1-GGSG-HaloTag[3-19]-GGSG-VS-HiBiT DLD-1 cells was analyzed to measure the nuclear-localized fluorescence intensity for PARP1, while a separate FOV of parental cells was assessed to quantify the whole cell background fluorescence intensity. The intensity of individual cells analyzed by this strategy was used to generate datapoints on the plot. The bar represents the Mean of the data. The graph demonstrates a higher level of homogeneity in expression with mRNA, with only a few outliers present. The results suggest similar fluorescence fold responses among the variants.

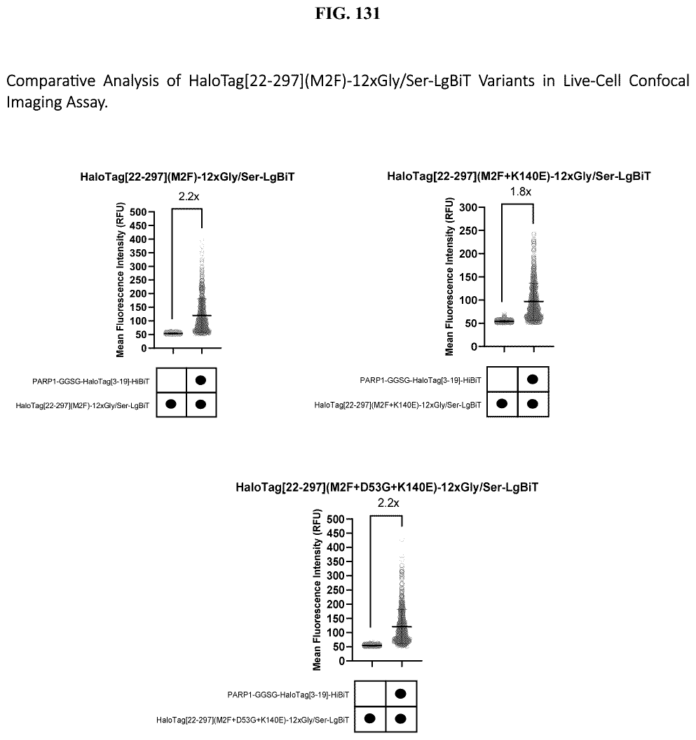

FIG. 132. Labeling kinetics of HaloTag7 and HaloTag[22-297](M2F+D53G+K140E)-12×Gly/Ser-LgBiT-3×Gly/Ser-6×His with HaloTag ligand JF549. Fluorescence polarization was monitored over time to observe labeling kinetics of JF549 HaloTag ligand by HaloTag standard protein or HaloTag[22-297](M2F+D53G+K140E)-12×Gly/Ser-LgBiT-3×Gly/Ser-6×His. The experiment was completed in Tris-buffered saline+0.01% CHAPS with proteins at 10 nM and ligand at 2.5 nM.

FIG. 133. Labeling kinetics of HaloTag7 and HaloTag[22-297](M2F+D53G+K140E)-12×Gly/Ser-LgBiT-3×Gly/Ser-6×His with HaloTag ligand JF503. Fluorescence polarization was monitored over time to observe labeling kinetics of JF503 HaloTag ligand by HaloTag standard protein or HaloTag[22-297](M2F+D53G+K140E)-12×Gly/Ser-LgBiT-3×Gly/Ser-6×His. The experiment was completed in Tris-buffered saline+0.01% CHAPS with proteins at 10 nM and ligand at 2.5 nM.

FIG. 134. Labeling kinetics of HaloTag7 and HaloTag[22-297](M2F+D53G+K140E)-12×Gly/Ser-LgBiT-3×Gly/Ser-6×His with HaloTag ligand JFX554. Fluorescence polarization was monitored over time to observe labeling kinetics of JFX554 HaloTag ligand by HaloTag standard protein or HaloTag[22-297](M2F+D53G+K140E)-12×Gly/Ser-LgBiT-3×Gly/Ser-6×His. The experiment was completed in Tris-buffered saline+0.01% CHAPS with proteins at 10 nM and ligand at 2.5 nM.

FIG. 135. Labeling kinetics of HaloTag7 and HaloTag[22-297](M2F+D53G+K140E)-12×Gly/Ser-LgBiT-3×Gly/Ser-6×His with HaloTag ligand Oregon Green. Fluorescence polarization was monitored over time to observe labeling kinetics of Oregon Green HaloTag ligand by HaloTag standard protein or HaloTag[22-297](M2F+D53G+K140E)-12×Gly/Ser-LgBiT-3×Gly/Ser-6×His. The experiment was completed in Tris-buffered saline+0.01% CHAPS with proteins at 10 nM and ligand at 2.5 nM.

FIG. 136. Labeling kinetics of HaloTag7 and HaloTag[22-297](M2F+D53G+K140E)-12×Gly/Ser-LgBiT-3×Gly/Ser-6×His with a FAM HaloTag ligand. Fluorescence polarization was monitored over time to observe labeling kinetics of FAM HaloTag ligand by HaloTag standard protein or HaloTag[22-297](M2F+D53G+K140E)-12×Gly/Ser-LgBiT-3×Gly/Ser-6×His. The experiment was completed in Tris-buffered saline+0.01% CHAPS with proteins at 10 nM and ligand at 2.5 nM.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies, or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a,” “an,” and “the” include plural reference unless the context clearly dictates otherwise. Thus, for example, reference to “a polypeptide” is a reference to one or more polypeptides and equivalents thereof known to those skilled in the art, and so forth.

As used herein, the term “and/or” includes any and all combinations of listed items, including any of the listed items individually. For example, “A, B, and/or C” encompasses A, B, C, AB, AC, BC, and ABC, each of which is to be considered separately described by the statement “A, B, and/or C.”

As used herein, the term “comprise” and linguistic variations thereof denote the presence of recited feature(s), element(s), method step(s), etc., without the exclusion of the presence of additional feature(s), element(s), method step(s), etc. Conversely, the term “consisting of” and linguistic variations thereof, denotes the presence of recited feature(s), element(s), method step(s), etc., and excludes any unrecited feature(s), element(s), method step(s), etc., except for ordinarily-associated impurities. The phrase “consisting essentially of” denotes the recited feature(s), element(s), method step(s), etc., and any additional feature(s), element(s), method step(s), etc., that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “substantially” means that the recited characteristic, parameter, and/or value need not be achieved exactly, but that deviations or variations, including for example, tolerances, measurement error, measurement accuracy limitations and other factors known to skill in the art, may occur in amounts that do not preclude the effect the characteristic was intended to provide. A characteristic or feature that is substantially absent (e.g., substantially non-fluorescent) may be one that is within the noise, beneath background, below the detection capabilities of the assay being used, or a small fraction (e.g., <1%, <0.1%, <0.01%, <0.001%, <0.00001%, <0.000001%, <0.0000001%) of the significant characteristic (e.g., fluorescent intensity of an active fluorophore).

As used herein, when referring to amino acid sequences or positions within an amino acid sequence, the phrase “corresponding to” refers to the relative position of an amino acid residue or an amino acid segment with the sequence being referred to, not necessarily the specific identity of the amino acids at that position. For example, a “peptide corresponding to positions 36 through 48 of SEQ ID NO: 1” may comprise less than 100% sequence identity with positions 36 through 48 of SEQ ID NO: 1 (e.g., >70% sequence identity), but within the context of the composition or system being described the peptide relates to those positions.

As used herein, the term “system” refers to multiple components (e.g., devices, compositions, etc.) that find use for a particular purpose. For example, two separate biological molecules, whether present in the same composition or not, may comprise a system if they are useful together for a shared purpose.

As used herein, the term “complementary” refers to the characteristic of two or more structural elements (e.g., peptide, polypeptide, nucleic acid, small molecule, etc.) of being able to hybridize, dimerize, or otherwise form a complex with each other. For example, a “complementary peptide and polypeptide” are capable of coming together to form a complex. Complementary elements may require assistance (facilitation) to form a complex (e.g., from interaction elements), for example, to place the elements in the proper conformation for complementarity, to place the elements in the proper proximity for complementarity, to co-localize complementary elements, to lower interaction energy for complementary, to overcome insufficient affinity for one another, etc.

As used herein, the term “complex” refers to an assemblage or aggregate of molecules (e.g., peptides, polypeptides, etc.) in direct and/or indirect contact with one another. In one aspect, “contact,” or more particularly “direct contact,” means two or more molecules are close enough so that attractive noncovalent interactions, such as Van der Waal forces, hydrogen bonding, ionic and hydrophobic interactions, and the like, dominate the interaction of the molecules. In such an aspect, a complex of molecules (e.g., peptides, polypeptides, etc.) is formed under assay conditions such that the complex is thermodynamically favored (e.g., compared to a non-aggregated, or non-complexed, state of its component molecules). As used herein the term “complex,” unless described as otherwise, refers to the assemblage of two or more molecules (e.g., peptides, polypeptides, etc.).

As used herein, the term “interaction element” refers to a moiety that assists or facilitates the bringing together of two or more structural elements (e.g., peptides, polypeptides, etc.) to form a complex. In some embodiments, a pair of interaction elements (a.k.a. “interaction pair”) is attached to a pair of structural elements (e.g., peptides, polypeptides, etc.), and the attractive interaction between the two interaction elements facilitate formation of a complex of the structural elements. Interaction elements may facilitate formation of a complex by any suitable mechanism (e.g., bringing structural elements into proximity, placing structural elements in proper conformation for stable interaction, reducing activation energy for complex formation, combinations thereof, etc.). An interaction element may be a protein, polypeptide, peptide, small molecule, cofactor, nucleic acid, lipid, carbohydrate, antibody, etc. An interaction pair may be made of two of the same interaction elements (i.e., homopair) or two different interaction elements (i.e., heteropair). In the case of a heteropair, the interaction elements may be the same type of moiety (e.g., polypeptides) or may be two different types of moieties (e.g., polypeptide and small molecule). In some embodiments, in which complex formation by the interaction pair is studied, an interaction pair may be referred to as a “target pair” or a “pair of interest,” and the individual interaction elements are referred to as “target elements” (e.g., “target peptide,” “target polypeptide,” etc.) or “elements of interest” (e.g., “peptide of interest,” “polypeptide or interest,” etc.).

As used herein, the term “low affinity” describes an intermolecular interaction between two or more entities that is too weak to result in significant complex formation between the entities, except at concentrations substantially higher (e.g., 2-fold, 5-fold, 10-fold, 100-fold, 1000-fold, or more) than physiologic or assay conditions, or with facilitation from the formation of a second complex of attached elements (e.g., interaction elements).

As used herein, the term “high affinity” describes an intermolecular interaction between two or more (e.g., three) entities that is of sufficient strength to produce detectable complex formation under physiologic or assay conditions without facilitation from the formation of a second complex of attached elements (e.g., interaction elements).

As used herein, the term “preexisting protein” refers to an amino acid sequence that was in physical existence prior to a certain event or date. A “peptide that is not a fragment of a preexisting protein” is a short amino acid chain that is not a fragment or sub-sequence of a protein (e.g., synthetic or naturally-occurring) that was in physical existence prior to the design and/or synthesis of the peptide.

As used herein, the term “fragment” refers to a peptide or polypeptide that results from dissection or “fragmentation” of a larger whole entity (e.g., protein, polypeptide, enzyme, etc.), or a peptide or polypeptide prepared to have the same sequence as such. Therefore, a fragment is a subsequence of the whole entity (e.g., protein, polypeptide, enzyme, etc.) from which it is made and/or designed. A peptide or polypeptide that is not a subsequence of a preexisting whole protein is not a fragment (e.g., not a fragment of a preexisting protein). A peptide or polypeptide that is “not a fragment of a preexisting protein” is an amino acid chain that is not a subsequence of a protein (e.g., natural or synthetic) that was in physical existence prior to design and/or synthesis of the peptide or polypeptide. A fragment of a hydrolase or dehalogenase, as used herein, is a sequence which is less than the full-length sequence, but which alone cannot form a substrate binding site, and/or has substantially reduced or no substrate binding activity but which, in close proximity to a second fragment of a hydrolase or dehalogenase, exhibits substantially increased substrate binding activity. In one embodiment, a fragment of a hydrolase or dehalogenase is at least 5, e.g., at least 10, at least 20, at least 30, at least 40, or at least 50, contiguous residues of a wild-type hydrolase or a mutated hydrolase, or a sequence with at least 70% sequence identity thereto, and may not necessarily include the N-terminal or C-terminal residue or N-terminal or C-terminal sequences of the corresponding full length protein.

As used herein, the term “subsequence” refers to peptide or polypeptide that has 100% sequence identify with a portion of another, larger peptide, or polypeptide. The subsequence is a perfect sequence match for a portion of the larger amino acid chain.

The term “amino acid” refers to natural amino acids, unnatural amino acids, and amino acid analogs, all in their D and L stereoisomers, unless otherwise indicated, if their structures allow such stereoisomeric forms.

The term “proteinogenic amino acids” refers to the 20 amino acids coded for in the human genetic code, and includes alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), Lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V). Selenocysteine and pyrrolysine may also be considered proteinogenic amino acids

The term “non-proteinogenic amino acid” refers to an amino acid that is not naturally-encoded or found in the genetic code of any organism, and is not incorporated biosynthetically into proteins during translation. Non-proteinogenic amino acids may be “unnatural amino acids” (amino acids that do not occur in nature) or “naturally-occurring non-proteinogenic amino acids” (e.g., norvaline, ornithine, homocysteine, etc.). Examples of non-proteinogenic amino acids include, but are not limited to, azetidinecarboxylic acid, 2-aminoadipic acid, 3-aminoadipic acid, beta-alanine, naphthylalanine, aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine, 2,4-diaminoisobutyric acid, desmosine, 2,2′-diaminopimelic acid, 2,3-diaminopropionic acid, N-ethylglycine, N-ethylasparagine, homoproline, hydroxylysine, allo-hydroxylysine, 3-hydroxyproline, 4-hydroxyproline, isodesmosine, allo-isoleucine, N-methylalanine, N-alkylglycine including N-methylglycine, N-methylisoleucine, N-alkylpentylglycine including N-methylpentylglycine. N-methylvaline, naphthylalanine, norvaline, norleucine (“Norleu”), octylglycine, ornithine, pentylglycine, pipecolic acid, thioproline, homolysine, and homoarginine. Non-proteinogenic also include D-amino acid forms of any of the amino acids herein, as well as non-alpha amino acid forms of any of the amino acids herein (beta-amino acids, gamma-amino acids, delta-amino acids, etc.), all of which are in the scope herein and may be included in peptides herein.

The term “amino acid analog” refers to an amino acid (e.g., natural or unnatural, proteinogenic or non-proteinogenic) where one or more of the C-terminal carboxy group, the N-terminal amino group and side-chain bioactive group has been chemically blocked, reversibly or irreversibly, or otherwise modified to another bioactive group. For example, aspartic acid-(beta-methyl ester) is an amino acid analog of aspartic acid; N-ethylglycine is an amino acid analog of glycine; or alanine carboxamide is an amino acid analog of alanine. Other amino acid analogs include methionine sulfoxide, methionine sulfone, S-(carboxymethyl)-cysteine, S-(carboxymethyl)-cysteine sulfoxide, and S-(carboxymethyl)-cysteine sulfone.

As used herein, unless otherwise specified, the terms “peptide” and “polypeptide” refer to polymer compounds of two or more amino acids joined through the main chain by peptide amide bonds (—C(O)NH—). The term “peptide” typically refers to short amino acid polymers (e.g., chains having fewer than 30 amino acids), whereas the term “polypeptide” typically refers to longer amino acid polymers (e.g., chains having more than 30 amino acids).

As used herein, the terms “artificial” or “synthetic” refer to compositions and systems that are designed or prepared by man and are not naturally occurring. For example, an artificial or synthetic peptide, peptoid, or nucleic acid is one comprising a non-natural sequence (e.g., a peptide without 100% identity with a naturally-occurring protein or a fragment thereof).

As used herein, a “conservative” amino acid substitution refers to the substitution of an amino acid in a peptide or polypeptide with another amino acid having similar chemical properties such as size or charge. For purposes of the present disclosure, each of the following eight groups contains amino acids that are conservative substitutions for one another:
1) Alanine (A) and Glycine (G);
2) Aspartic acid (D) and Glutamic acid (E);
3) Asparagine (N) and Glutamine (Q);
4) Arginine (R) and Lysine (K);
5) Isoleucine (I), Leucine (L), Methionine (M), and Valine (V);
6) Phenylalanine (F), Tyrosine (Y), and Tryptophan (W);
7) Serine (S) and Threonine (T); and
8) Cysteine (C) and Methionine (M).

Amino acid residues may be divided into classes based on common side chain properties, for example: polar positive (or basic) (e.g., histidine (H), lysine (K), and arginine (R)); polar negative (or acidic) (e.g., aspartic acid (D), glutamic acid (E)); polar neutral (e.g., serine (S), threonine (T), asparagine (N), glutamine (Q)); non-polar aliphatic (e.g., alanine (A), valine (V), leucine (L), isoleucine (I), methionine (M)); non-polar aromatic (e.g., phenylalanine (F), tyrosine (Y), tryptophan (W)); proline and glycine; and cysteine. As used herein, a “semi-conservative” amino acid substitution refers to the substitution of an amino acid in a peptide or polypeptide with another amino acid within the same class.

In some embodiments, unless otherwise specified, a conservative or semi-conservative amino acid substitution may also encompass non-naturally occurring amino acid residues that have similar chemical properties to the natural residue. These non-natural residues are typically incorporated by chemical peptide synthesis rather than by synthesis in biological systems. These include, but are not limited to, peptidomimetics and other reversed or inverted forms of amino acid moieties. Embodiments herein may, in some embodiments, be limited to natural amino acids, non-natural amino acids, and/or amino acid analogs.

Non-conservative substitutions may involve the exchange of a member of one class for a member from another class.

As used herein, the term “sequence identity” refers to the degree two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) have similar polymer sequences. For example, similar amino acids are those that share the same biophysical characteristics and can be grouped into the families, e.g., acidic (e.g., aspartate, glutamate), basic (e.g., lysine, arginine, histidine), non-polar (e.g., alanine, valine, leucine, isoleucine, proline, phenylalanine, methionine, tryptophan) and uncharged polar (e.g., glycine, asparagine, glutamine, cysteine, serine, threonine, tyrosine). The “percent sequence identity” (or “percent sequence similarity”) is calculated by: (1) comparing two optimally aligned sequences over a window of comparison (e.g., the length of the longer sequence, the length of the shorter sequence, a specified window), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

Any peptide/polypeptides described herein as having a particular percent sequence identity or similarity (e.g., at least 70%) with a reference sequence ID number, may also be expressed as having a maximum number of substitutions (or terminal deletions) with respect to that reference sequence. For example, a sequence having at least Y % sequence identity (e.g., 90%) with SEQ ID NO:Z (e.g., 100 amino acids) may have up to X substitutions (e.g., 10) relative to SEQ ID NO:Z, and may therefore also be expressed as “having X (e.g., 10) or fewer substitutions relative to SEQ ID NO:Z.”

As used herein, the term “wild-type” refers to a gene or gene product (e.g., protein, polypeptide, peptide, etc.) that has the characteristics (e.g., sequence) of that gene or gene product isolated from a naturally occurring source, and is most frequently observed in a population. In contrast, the term “mutant” or “variant” refers to a gene or gene product that displays modifications in sequence when compared to the wild-type gene or gene product. It is noted that “naturally-occurring variants” are genes or gene products that occur in nature, but have altered sequences when compared to the wild-type gene or gene product; they are not the most commonly occurring sequence. “Artificial variants” or “synthetic variants” are genes or gene products that have altered sequences when compared to the wild-type gene or gene product and do not occur in nature. Variant genes or gene products may be naturally occurring sequences that are present in nature, but not the most common variant of the gene or gene product, or “synthetic,” produced by human or experimental intervention.

As used herein, the term “physiological conditions” encompasses any conditions compatible with living cells, e.g., predominantly aqueous conditions of a temperature, pH, salinity, chemical makeup, etc. that are compatible with living cells.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum, and the like. Sample may also refer to cell lysates or purified forms of the enzymes, peptides, and/or polypeptides described herein. Cell lysates may include cells that have been lysed with a lysing agent or lysates such as rabbit reticulocyte or wheat germ lysates. Sample may also include cell-free expression systems. Environmental samples include environmental material such as surface matter, soil, water, crystals, and industrial samples. Such examples are not however to be construed as limiting the sample types applicable to the present invention.

As used herein, the terms “fusion,” “fusion polypeptide,” and “fusion protein” refer to a chimeric protein containing a first protein or polypeptide of interest joined to a second different peptide, polypeptide, or protein (e.g., interaction element).

As used herein, the terms “conjugated” and “conjugation” refer to the covalent attachment of two molecular entities (e.g., post-synthesis and/or during synthetic production). The attachment of a peptide or small molecule tag to a protein or small molecule, chemically (e.g., “chemically” conjugated) or enzymatically, is an example of conjugation.

As used herein, the terms “polypeptide component” or “peptide component” are used synonymously with the terms “polypeptide component of a [mutant dehalogenase] complex” or “peptide component of a [mutant dehalogenase] complex.” Typically, as used herein, a polypeptide component or peptide component is capable of forming a complex with a second component to form a desired complex, under appropriate conditions.

As used herein, the term “dehalogenase” refers to an enzyme that catalyzes the removal of a halogen atom from a substrate. The term “haloalkane dehalogenase” refers to an enzyme that catalyzes the removal of a halogen from a haloalkane substrate to produce an alcohol and a halide. Dehalogenases and haloalkyl dehalogenases belong to the hydrolase enzyme family, and may be referred to herein or elsewhere as such.

As used herein, the term “modified dehalogenase” refers to a dehalogenase variant (artificial variant) that has mutations that prevent the release of the substrate from the protein following removal of the halogen, resulting in a covalent bond between the substrate and the modified dehalogenase. Because the modified dehalogenase does not release the substrate, it is not capable of turnover, and is not a classical enzyme. The HALOTAG system (Promega) is a commercially available modified dehalogenase and substrate system.

As used herein, the term “bioluminescence” refers to production and emission of light by a chemical reaction catalyzed by, or enabled by, an enzyme, protein, protein complex, or other biomolecule (e.g., bioluminescent complex). In typical embodiments, a substrate for a bioluminescent entity (e.g., bioluminescent protein or bioluminescent complex) is converted into an unstable form by the bioluminescent entity; the substrate subsequently emits light.

As used herein, the term “non-luminescent” refers to an entity (e.g., peptide, polypeptide, complex, protein, etc.) that exhibits the characteristic of not emitting a detectable amount of light in the visible spectrum (e.g., in the presence of a substrate). For example, an entity may be referred to as non-luminescent if it does not exhibit detectable luminescence in a given assay. As used herein, the term “non-luminescent” is synonymous with the term “substantially non-luminescent. In some embodiments, an entity is considered “non-luminescent” if any light emission is sufficiently minimal so as not to create interfering background for a particular assay.

As used herein, the terms “non-luminescent peptide” and “non-luminescent polypeptide” refer to peptides and polypeptides that exhibit substantially no luminescence (e.g., in the presence of a substrate), or an amount that is beneath the noise (e.g., 100-fold, 200-fold, 500-fold, 1×10³-fold, 1×10⁴-fold, 1×10⁵-fold, 1×10⁶-fold, 1×10⁷-fold, etc.), when compared to a significant signal (e.g., a bioluminescent complex) under standard conditions (e.g., physiological conditions, assay conditions, etc.) and with typical instrumentation (e.g., luminometer, etc.). In some embodiments, such non-luminescent peptides and polypeptides assemble, according to the criteria described herein, to form a bioluminescent complex.

As used herein, the term “an Oplophorus luciferase” (“an OgLuc”) refers to a luminescent polypeptide having significant sequence identity, structural conservation, and/or the functional activity of the luciferase produce by and derived from the deep-sea shrimp Oplophorus gracilirostris. In particular, an OgLuc polypeptide refers to a luminescent polypeptide having significant sequence identity, structural conservation, and/or the functional activity of the mature 19 kDa subunit of the Oplophorus luciferase protein complex (e.g., without a signal sequence) such as SEQ ID NOs: 3034 (NanoLuc), which comprises 10 β strands (β1, β2, β3, β4, β5, β6, β7, β8, β9, β10) and utilize substrates such as coelenterazine or a coelenterazine derivative or analog to produce luminescence.

As used herein, the term “β9-like peptide” refers to a peptide (or peptide tag) comprising significant sequence identity, structural conservation, and/or the functional activity of the β (beta) 9 strand of an OgLuc polypeptide. In particular, a β9-like peptide is a peptide capable of structurally complementing an OgLuc polypeptide lacking a 39 strand resulting in enhanced luminescence of the complex compared to the OgLuc polypeptide in the absence of the β9-like peptide. Other “βX-like peptides” may be similarly named (e.g., β1-like, β2-like, β3-like, β4-like, β5-like, β6-like, β7-like, β8-like, β9-like).

As used herein, the term “β10-like peptide” refers to a peptide (or peptide tag) comprising significant sequence identity, structural conservation, and/or the functional activity of the β (beta) 10 strand of an OgLuc polypeptide. In particular, a β10-like peptide is a peptide capable of structurally complementing an OgLuc polypeptide lacking a 010 strand resulting in enhanced luminescence of the complex compared to the OgLuc polypeptide in the absence of the β10-like peptide. Other “βX-like peptides” may be similarly named (e.g., β1-like, β2-like, β3-like, β4-like, β5-like, β6-like, β7-like, β8-like, β9-like).

As used herein, the term “β₁₋₈-like polypeptide” refers to a polypeptide bearing sequence and structural similarity to R (beta) strands 1-8 of an OgLuc polypeptide, but lacking β (beta) strands 9 and 10. Other “β_{Y-Z}-like polypeptides” may be similarly named (e.g., β₁₋₄-like polypeptide, β2-8-like polypeptide, β₅₋₁₀-like polypeptide, etc.).

As used herein, the term “NANOLUC” refers to an artificial luciferase or bioluminescent polypeptide produced commercially by the Promega Corporation.

As used herein, the term “LgBiT” refers to a polypeptide corresponding to β₁₋₉-like polypeptide that finds use in, for example, binary complementation to form a bioluminescent complex and corresponds to SEQ ID NO: 3037.

As used herein, the term “SmBiT” refers to a peptide corresponding to β₁₀-like peptide that finds use in, for example, binary complementation to form a bioluminescent complex, but has low affinity for LgBiT (e.g., requires facilitation for complex formation) and corresponds to SEQ ID NO: 3039.

As used herein, the term “HiBiT” refers to a peptide corresponding to β₁₀-like peptide that finds use in, for example, binary complementation to form a bioluminescent complex, but has high affinity for LgBiT (e.g., does not require facilitation for complex formation). An exemplary HiBiT peptide corresponds to SEQ ID NO: 3038.

As used herein, the term “LgTrip” refers to a polypeptide corresponding to β₁₋₈-like polypeptide. An exemplary LgTrip corresponds to SEQ ID NO: 3045 and finds use in, for example, tripartite complementation with β₉-like and β₁₀-like peptides to form a bioluminescent complex, or binary complementation, with a β₉₋₁₀-like peptide to form a bioluminescent complex.

As used herein, the term “SmTrip10” refers to a peptide corresponding to β₁₀-like peptide that finds use in, for example, tripartite complementation to form a bioluminescent complex.

As used herein, the term “SmTrip9” refers to a peptide corresponding to β₉-like peptide that finds use in, for example, tripartite complementation to form a bioluminescent complex. As used herein, the term “split” (“sp”) refers to refers to a polypeptide that has been divided into two fragments at an interior site of the original polypeptide. The fragments of a sp polypeptide may reconstitute activity of the original polypeptide if they are structurally complementary and able to form an active complex. Nomenclature herein for referring to split components of a polypeptide recites a position number from the full polypeptide that corresponds to the last residue in the N-terminal component of the split polypeptide. For example, if a polypeptide is 100 residues in length, a sp52 version of that polypeptide comprises a first fragment corresponding to positions 1-52 of the parent polypeptide and a second fragment corresponding to positions 53-100 of the parent polypeptide. As another example, spHT(45) refers to a split variant of the commercially-available HALOTAG protein in which the first fragment comprises residues 1-45 of the HALOTAG polypeptide sequence and the second fragment comprises residues 46-297 of the HALOTAG polypeptide sequence.

Alternatively, a component of a split polypeptide may be expressed herein by referring to the name of the polypeptide from which it is derived, the residues within the source polypeptide that are present in the component (in brackets), followed by any substitutions in the component relative to the source polypeptide (in parenthesis). For example, a split component of the commercially-available HALOTAG protein corresponding to position 22-297 of the HALOTAG sequence could be written HaloTag[22-297]. If the second position of the component contained a M to F substitution, the components could be referred to as HaloTag[22-297](M2F). Components may contain an N-terminal methionine residues not present in the source sequence; such residues are counted in refereeing to the location of substitutions but not in the numbering of the fragment within the source polypeptide.

As used herein, the term “gapped” refers to split variant of a polypeptide that is missing a segment of the original polypeptide. For example, a “gapped sp polypeptide” is one that is missing a segment of the original sequence that occurs at the site of the split.

As used herein, the term “overlapped” refers to split variant of a polypeptide that contains a duplication of a segment of the original polypeptide. For example, an “overlapped sp polypeptide” is one in which a segment of the original sequence adjacent to the split site is present (duplicated) at the C-terminus of a first fragment and the N-terminus of the second fragment.

The term “binding moiety” refers to a domain that specifically binds an antigen or epitope independently of a different epitope or antigen binding domain. A binding moiety may be an antibody, antibody fragment, a receptor domain that binds a target ligand, proteins that bind to immunoglobulins (e.g., protein A, protein G, protein A/G, protein L, protein M), a binding domain of a proteins that bind to immunoglobulins (e.g., protein A, protein G, protein A/G, protein L, protein M), oligonucleotide probe, peptide nucleic acid, DARPin, aptamer, affimer, a purified protein (either the analyte itself or a protein that binds to the analyte), and analyte binding domain(s) of proteins etc. Table A provides a list of exemplary binding moieties that could be used singly or in various combinations in methods, systems, and assays (e.g., immunoassays) herein.

TABLE A
Exemplary binding moieties

Protein A
Ig Binding domain of protein A
Protein G
Ig Binding domain of protein G
Protein L
Ig Binding domain of protein L
Protein M
Ig Binding domain of protein M
polyclonal antibody against analyte X
monoclonal antibody
recombinant antibody
scFv
variable light chain (V_{L}) of antibody
(monoclonal, recombinant, polyclonal)
recognizing target analyte X
protein (e.g., receptor) binding domain that
binds to analyte X
(Fab) fragment
Fab′ fragment
Fv fragment
F(ab′)2 fragment
oligonucleotide probe
DARPins and other synthetic binding
scaffolds (ex: Bicycles)
peptide nucleic acid
aptamer
affimer

### DETAILED DESCRIPTION

Provided herein are compositions and systems comprising complementation-based tags and reporters for labeling and detection of targets by luminescence and a second modality (e.g., fluorescence), and methods of use thereof. In particular, tags are provided comprising the fusion of a first component of bioluminescent complex and a first component of modified dehalogenase complex, reporters are provided comprising the second component of bioluminescent complex and the second component of modified dehalogenase complex, and systems and methods are provided comprising the tags and reporters herein for dual-modality labeling and detection of targets

In some embodiments, provided herein are systems comprising (a) a tandem peptide tag comprising (i) a peptide component of a bioluminescent complex fused to (ii) a peptide component of a modified dehalogenase complex; and (b) a tandem polypeptide reporter comprising a polypeptide component of a bioluminescent complex fused to a polypeptide component of a modified dehalogenase complex; wherein the bioluminescent complex and modified dehalogenase complex form upon interaction (facilitated or unfacilitated) of the tandem peptide tag and the tandem polypeptide reporter; wherein the bioluminescent complex is capable of generating bioluminescence in the presence of a substrate (e.g., coelenterazine, furimazine, etc.); and wherein the modified dehalogenase complex is capable of binding to a haloalkane ligand. In some embodiments, a high affinity interaction of one pair of components to form a complex (e.g., components of the bioluminescent complex or components of the modified dehalogenase complex) is sufficient to facilitate formation of the second complex (e.g., modified dehalogenase complex of bioluminescent complex).

### HaloTag

In some embodiments, provided herein are compositions (e.g., fusion peptides and polypeptides) and systems (e.g., multiple complementary fusion peptides and polypeptides, substrates, ligands, etc.) comprising complementary peptide/polypeptide fragments capable of interacting (e.g., facilitated or unfacilitated) to form an active modified dehalogenase complex capable of forming a covalent bond to a haloalkane ligand. In some embodiments, a first fusion is provided comprising a complementary peptide fragment of a modified dehalogenase, and a second fusion is provided comprising a complementary polypeptide fragment of the modified dehalogenase, wherein upon interacting (e.g., facilitated or unfacilitated), the complementary peptide and polypeptide form an active modified dehalogenase complex capable of forming a covalent bond to a haloalkane ligand. In some embodiments, a first fusion is provided comprising a complementary peptide fragment of a modified dehalogenase, and a complementary polypeptide fragment of the modified dehalogenase is provided (e.g., not as a fusion), wherein upon interacting (e.g., facilitated or unfacilitated), the complementary peptide and polypeptide form an active modified dehalogenase complex capable of forming a covalent bond to a haloalkane ligand. In some embodiments, the complementary peptide and polypeptide are fragments of a split mutant dehalogenase. In alternative embodiments, both fragments may be polypeptides.

Provided herein, as components of the compositions, systems, and methods herein are split mutated dehalogenases, such as those derived from the commercially available HALOTAG protein (Promega) and/or mutated dehalogenases disclosed in U.S. published application 20060024808, the disclosure of which is incorporated by reference herein.

Even though these mutant dehalogenases are not technically enzymes (no substrate turnover), the stable binding of a substrate thereto is dependent on proper protein structure. The consequence of re-associating the split fragments of a mutant dehalogenase differs from that of a split enzyme system because the labeling function of a mutant dehalogenase is retained on one of the fragments even after it has separated from its partner, whereas split enzymes are only active when they are brought together and bear no artifact of their prior activity after they are separated. In effect, the labeling reaction of a split mutant dehalogenase provides a molecular memory of a protein interaction. In the case of fluorogenic ligands, the label is retained on one of the fragments, but may not be detectable after complex dissociation (since the fluorogen-activating contacts with the protein may be disrupted/absent); therefore, the combination of split dehalogenase and fluorogenic ligands produce a unique situation of permanent labeling, but with dynamic (on/off) fluorescence detection of the retained label.

A mutant dehalogenase provides for efficient labeling within a living cell or lysate thereof. This labeling is only conditional on the presence or expression of the protein and the presence of the labeled hydrolase substrate. In contrast, the labeling of a split mutant dehalogenase is dependent on a specific protein interaction occurring within the cell and the presence of the labeled hydrolase substrate.

In some embodiments, provided as a component of the compositions, systems, and methods herein are split modified dehalogenases. In some embodiments, a first fragment of a mutant dehalogenase is fused to a first fragment of luminescent protein (e.g., and optionally a protein or molecule of interest), and a second fragment of the mutant dehalogenase is fused to a second fragment of the luminescent protein (e.g., and optionally a protein or molecule of interest)). In some embodiments, at least one of the mutant dehalogenase fragments has a substitution that if present in a full-length modified dehalogenase having the sequence of the two fragments, forms a bond with a haloalkane ligand. In some embodiments, the first fragment of the mutant dehalogenase and the second fragment of the mutant dehalogenase are capable of interacting (e.g., facilitated or unfacilitated) to form an active modified dehalogenase complex.

HALOTAG is a 297-residue self-labeling polypeptide (33 kDa) derived from a bacterial hydrolase (dehalogenase) enzyme, which has been modified to covalently bind to its ligand, a haloalkane moiety. The HALOTAG ligand can be linked to solid surfaces (e.g., beads) or functional groups (e.g., fluorophores), and the HALOTAG polypeptide can be fused to various proteins of interest, allowing covalent attachment of the protein of interest to the solid surface or functional group.

The HALOTAG polypeptide is a modified dehalogenase with a genetically modified active site, which specifically binds to the haloalkane ligand chloroalkane linker with an enhanced and increased rate of ligand binding (Pries et al. The Journal of Biological Chemistry, 270(18):10405-11; incorporated by reference in its entirety). The reaction that forms the bond between the protein tag and chloroalkane linker is fast and essentially irreversible under physiological conditions (Waugh DS (June 2005). Trends in Biotechnology. 23(6):316-20; incorporated by reference in its entirety). In the natural hydrolase enzyme, nucleophilic attack of the chloroalkane reactive linker causes displacement of the halogen with an amino acid residue, which results in the formation of a covalent alkyl-enzyme intermediate. This intermediate would then be hydrolyzed by an amino acid residue within the wild-type hydrolase (Chen et al. (February 2005) Current Opinion in Biotechnology. 16(1):35-40; incorporated by reference in its entirety). This would lead to regeneration of the enzyme following the reaction. However, with HALOTAG, the modified haloalkane dehalogenase, the reaction intermediate cannot proceed through the second reaction because it cannot be hydrolyzed due to the mutation in the enzyme. This causes the intermediate to persist as a stable covalent adduct with which there is no associated back reaction (Marks et al. (August 2006) Nature Methods. 3 (8): 591-6; incorporated by reference in its entirety).

HALOTAG fusion proteins can be expressed using standard recombinant protein expression techniques (Adams et al. (May 2002) Journal of the American Chemical Society. 124(21):6063-76; incorporated by reference in its entirety). Since the HALOTAG polypeptide is a relatively small protein, and the reactions are foreign to mammalian cells, there is no interference by endogenous mammalian metabolic reactions (Naested et al. The Plant Journal. 18(5):571-6; incorporated by reference in its entirety). Once the fusion protein has been expressed, there is a wide range of potential areas of experimentation including enzymatic assays, cellular imaging, protein arrays, determination of sub-cellular localization, and many additional possibilities (Janssen DB (April 2004). Current Opinion in Chemical Biology. 8(2):150-9; incorporated by reference in its entirety).

Various HALOTAG ligands, functional groups, fusions, assays, modifications, uses, etc. are described in U.S. Pat. Nos. 8,748,148; 9,593,316; 10,246,690; 8,742,086; 9,873,866; 10,604,745; U.S. Pat. App. 2009/0253131; U.S. Pat. App. 2010/0273186; 20130337539; U.S. Pat. App. 2012/0258470; U.S. Pat. App. 2012/0252048; U.S. Pat. App. 2011/0201024; U.S. 2014/0322794; each of which is incorporated by reference in their entireties.

In some embodiments, the fragments, complementary peptides, complementary polypeptides, etc., of a modified dehalogenase described herein are a HALOTAG-based complementation system. In some embodiments, the fragments, complementary peptides, complementary polypeptides, etc., of a modified dehalogenase described herein correspond (e.g., sequence identity, sequence similarity, 3D structure, etc.) to sequences within the HALOTAG protein. In some embodiments, a modified dehalogenase complex herein, comprising two or more peptide or polypeptide components corresponds to a HALOTAG protein and is capable of binding to a halkoalkyl ligand in a similar manner.

In some embodiments, as described in U.S. Prov. App. No. 63/338,323 and U.S. application Ser. No. 18/312,117, both of which are herein incorporated by reference in their entireties, extensive experiments have been conducted to demonstrate the feasibility of generating fragments of HALOTAG (and variants thereof) capable of interacting to form a modified dehalogenase complex capable of binding to a haloalkyl ligand, as well as optimizing variants of HALOTAG fragments for desired characteristics. As described herein, embodiments are not limited to the HALOTAG sequences. In some embodiments, provided herein are split modified dehalogenases that differ in sequence from HALOTAG (SEQ ID NO: 1).

In some embodiments, compositions and systems are provided comprising components of a split modified dehalogenase, such as a split HALOTAG (“spHT”) or variants thereof. In some embodiments, systems and compositions herein comprise spHT peptides and polypeptides (e.g., as a portion of the fusions described herein).

In some embodiments, compositions (e.g., fusions) and systems (e.g., multiple fusions with appropriate ligands and substrates) are provided comprising polypeptides, peptides, fragments, and combinations thereof derived from a modified dehalogenase sequence of SEQ ID NO: 1 (HALOTAG):

MAEIGTGFPFDPHYVEVLGERMHYVDVGPRDGTPVLFLHGNPTSSYVWR
NIIPHVAPTHRCIAPDLIGMGKSDKPDLGYFFDDHVRFMDAFIEALGLE
EVVLVIHDWGSALGFHWAKRNPERVKGIAFMEFIRPIPTWDEWPEFARE
TFQAFRTTDVGRKLIIDQNVFIEGTLPMGVVRPLTEVEMDHYREPFLNP
VDREPLWRFPNELPIAGEPANIVALVEEYMDWLHQSPVPKLLFWGTPGV
LIPPAEAARLAKSLPNCKAVDIGPGLNLLQEDNPDLIGSEIARWLSTLE
ISG.

In some embodiments, the spHT components herein lack the mutation(s) (e.g., 272 and/or 106) that produce covalent bonding to the haloalkane substrate. Such sp dehalogenases are true enzymes capable of substrate turnover, but otherwise comprising the sequences and characteristics of the embodiments described herein.

In some embodiments, spHT peptides and polypeptides herein (e.g., as portions of fusions herein, as standalone reporters or tags, etc.) comprise at least 70% sequence identity with a portion of SEQ ID NO: 1 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity). In some embodiments, spHT peptides and polypeptides herein (e.g., as portions of fusions herein, as standalone reporters or tags, etc.) comprise 100% sequence identity with all or a portion of SEQ ID NO: 1. In some embodiments, spHT peptides and polypeptides herein (e.g., as portions of fusions herein, as standalone reporters or tags, etc.) comprise at least 70% sequence similarity with all or a portion of SEQ ID NO: 1 (e.g., >70% sequence similarity, >75% sequence similarity, >80% sequence similarity, >85% sequence similarity, >90% sequence similarity, >95% sequence similarity, >96% sequence similarity, >97% sequence similarity, >98% sequence similarity, >99% sequence similarity). In some embodiments, spHT peptides and polypeptides herein (e.g., as portions of fusions herein, as standalone reporters or tags, etc.) comprise 100% sequence similarity with all or a portion of SEQ ID NO: 1.

In some embodiments, spHT peptides or polypeptides (e.g., as portions of fusions herein, as standalone reporters or tags, etc.) herein comprise an A at a position corresponding to position 2 of SEQ ID NO: 1. In other embodiments, spHT peptides or polypeptides (e.g., as portions of fusions herein, as standalone reporters or tags, etc.) herein comprise an S at a position corresponding to position 2 of SEQ ID NO: 1. In some embodiments, spHT peptides or polypeptides (e.g., as portions of fusions herein, as standalone reporters or tags, etc.) herein comprise a V at a position corresponding to position 47 of SEQ ID NO: 1. In some embodiments, spHT peptides or polypeptides (e.g., as portions of fusions herein, as standalone reporters or tags, etc.) herein comprise a T at a position corresponding to position 58 of SEQ ID NO: 1. In some embodiments, spHT peptides or polypeptides (e.g., as portions of fusions herein, as standalone reporters or tags, etc.) herein comprise a G at a position corresponding to position 78 of SEQ ID NO: 1. In some embodiments, spHT peptides or polypeptides (e.g., as portions of fusions herein, as standalone reporters or tags, etc.) herein comprise a F at a position corresponding to position 88 of SEQ ID NO: 1. In some embodiments, spHT peptides or polypeptides (e.g., as portions of fusions herein, as standalone reporters or tags, etc.) herein comprise a M at a position corresponding to position 89 of SEQ ID NO: 1. In some embodiments, spHT peptides or polypeptides (e.g., as portions of fusions herein, as standalone reporters or tags, etc.) herein comprise a F at a position corresponding to position 128 of SEQ ID NO: 1. In some embodiments, spHT peptides or polypeptides (e.g., as portions of fusions herein, as standalone reporters or tags, etc.) herein comprise a T at a position corresponding to position 155 of SEQ ID NO: 1. In some embodiments, spHT peptides or polypeptides (e.g., as portions of fusions herein, as standalone reporters or tags, etc.) herein comprise a K at a position corresponding to position 160 of SEQ ID NO: 1. In some embodiments, spHT peptides or polypeptides (e.g., as portions of fusions herein, as standalone reporters or tags, etc.) herein comprise a V at a position corresponding to position 167 of SEQ ID NO: 1. In some embodiments, spHT peptides or polypeptides (e.g., as portions of fusions herein, as standalone reporters or tags, etc.) herein comprise a T at a position corresponding to position 172 of SEQ ID NO: 1. In some embodiments, spHT peptides or polypeptides (e.g., as portions of fusions herein, as standalone reporters or tags, etc.) herein comprise a M at a position corresponding to position 175 of SEQ ID NO: 1. In some embodiments, spHT peptides or polypeptides (e.g., as portions of fusions herein, as standalone reporters or tags, etc.) herein comprise a G at a position corresponding to position 176 of SEQ ID NO: 1. In some embodiments, spHT peptides or polypeptides (e.g., as portions of fusions herein, as standalone reporters or tags, etc.) herein comprise a N at a position corresponding to position 195 of SEQ ID NO: 1. In some embodiments, spHT peptides or polypeptides (e.g., as portions of fusions herein, as standalone reporters or tags, etc.) herein comprise a E at a position corresponding to position 224 of SEQ ID NO: 1. In some embodiments, spHT peptides or polypeptides (e.g., as portions of fusions herein, as standalone reporters or tags, etc.) herein comprise a D at a position corresponding to position 227 of SEQ ID NO: 1. In some embodiments, spHT peptides or polypeptides (e.g., as portions of fusions herein, as standalone reporters or tags, etc.) herein comprise a K at a position corresponding to position 257 of SEQ ID NO: 1. In some embodiments, spHT peptides or polypeptides (e.g., as portions of fusions herein, as standalone reporters or tags, etc.) herein comprise an A at a position corresponding to position 264 of SEQ ID NO: 1. In some embodiments, peptides or polypeptides herein comprise a N at a position corresponding to position 272 of SEQ ID NO: 1. In some embodiments, spHT peptides or polypeptides (e.g., as portions of fusions herein, as standalone reporters or tags, etc.) herein comprise a L at a position corresponding to position 273 of SEQ ID NO: 1. In some embodiments, spHT peptides or polypeptides (e.g., as portions of fusions herein, as standalone reporters or tags, etc.) herein comprise a S at a position corresponding to position 291 of SEQ ID NO: 1. In some embodiments, spHT peptides or polypeptides (e.g., as portions of fusions herein, as standalone reporters or tags, etc.) herein comprise a T at a position corresponding to position 292 of SEQ ID NO: 1. In some embodiments, spHT peptides or polypeptides (e.g., as portions of fusions herein, as standalone reporters or tags, etc.) herein comprise a E at a position corresponding to position 294 of SEQ ID NO: 1. In some embodiments, spHT peptides or polypeptides (e.g., as portions of fusions herein, as standalone reporters or tags, etc.) herein comprise a I at a position corresponding to position 295 of SEQ ID NO: 1. In some embodiments, spHT peptides or polypeptides (e.g., as portions of fusions herein, as standalone reporters or tags, etc.) herein comprise a S at a position corresponding to position 296 of SEQ ID NO: 1. In some embodiments, spHT peptides or polypeptides (e.g., as portions of fusions herein, as standalone reporters or tags, etc.) herein comprise a G at a position corresponding to position 297 of SEQ ID NO: 1.

In some embodiments, spHT peptides or polypeptides (e.g., as portions of fusions herein, as standalone reporters or tags, etc.) herein do not have an S at a position corresponding to position 2 of SEQ ID NO: 1. In some embodiments, spHT peptides or polypeptides (e.g., as portions of fusions herein, as standalone reporters or tags, etc.) herein do not have a L at a position corresponding to position 47 of SEQ ID NO: 1. In some embodiments, spHT peptides or polypeptides (e.g., as portions of fusions herein, as standalone reporters or tags, etc.) herein do not have a S at a position corresponding to position 58 of SEQ ID NO: 1. In some embodiments, spHT peptides or polypeptides (e.g., as portions of fusions herein, as standalone reporters or tags, etc.) herein do not have a D at a position corresponding to position 78 of SEQ ID NO: 1. In some embodiments, spHT peptides or polypeptides (e.g., as portions of fusions herein, as standalone reporters or tags, etc.) herein do not have a Y at a position corresponding to position 88 of SEQ ID NO: 1. In some embodiments, spHT peptides or polypeptides (e.g., as portions of fusions herein, as standalone reporters or tags, etc.) herein do not have a L at a position corresponding to position 89 of SEQ ID NO: 1. In some embodiments, spHT peptides or polypeptides (e.g., as portions of fusions herein, as standalone reporters or tags, etc.) herein do not have a C at a position corresponding to position 128 of SEQ ID NO: 1. In some embodiments, spHT peptides or polypeptides (e.g., as portions of fusions herein, as standalone reporters or tags, etc.) herein do not have an A at a position corresponding to position 155 of SEQ ID NO: 1. In some embodiments, spHT peptides or polypeptides (e.g., as portions of fusions herein, as standalone reporters or tags, etc.) herein do not have a E at a position corresponding to position 160 of SEQ ID NO: 1. In some embodiments, spHT peptides or polypeptides (e.g., as portions of fusions herein, as standalone reporters or tags, etc.) herein do not have an A at a position corresponding to position 167 of SEQ ID NO: 1. In some embodiments, spHT peptides or polypeptides (e.g., as portions of fusions herein, as standalone reporters or tags, etc.) herein do not have an A at a position corresponding to position 172 of SEQ ID NO: 1. In some embodiments, spHT peptides or polypeptides (e.g., as portions of fusions herein, as standalone reporters or tags, etc.) herein do not have a K at a position corresponding to position 175 of SEQ ID NO: 1. In some embodiments, spHT peptides or polypeptides (e.g., as portions of fusions herein, as standalone reporters or tags, etc.) herein do not have a C at a position corresponding to position 176 of SEQ ID NO: 1. In some embodiments, spHT peptides or polypeptides (e.g., as portions of fusions herein, as standalone reporters or tags, etc.) herein do not have a K at a position corresponding to position 195 of SEQ ID NO: 1. In some embodiments, spHT peptides or polypeptides (e.g., as portions of fusions herein, as standalone reporters or tags, etc.) herein do not have an A at a position corresponding to position 224 of SEQ ID NO: 1. In some embodiments, spHT peptides or polypeptides (e.g., as portions of fusions herein, as standalone reporters or tags, etc.) herein do not have a N at a position corresponding to position 227 of SEQ ID NO: 1. In some embodiments, spHT peptides or polypeptides (e.g., as portions of fusions herein, as standalone reporters or tags, etc.) herein do not have a E at a position corresponding to position 257 of SEQ ID NO: 1. In some embodiments, spHT peptides or polypeptides (e.g., as portions of fusions herein, as standalone reporters or tags, etc.) herein do not have a T at a position corresponding to position 264 of SEQ ID NO: 1. In some embodiments, spHT peptides or polypeptides (e.g., as portions of fusions herein, as standalone reporters or tags, etc.) herein do not have a H at a position corresponding to position 272 of SEQ ID NO: 1. In some embodiments, spHT peptides or polypeptides (e.g., as portions of fusions herein, as standalone reporters or tags, etc.) herein do not have a Y at a position corresponding to position 273 of SEQ ID NO: 1. In some embodiments, spHT peptides or polypeptides (e.g., as portions of fusions herein, as standalone reporters or tags, etc.) herein do not have a P at a position corresponding to position 291 of SEQ ID NO: 1. In some embodiments, spHT peptides or polypeptides (e.g., as portions of fusions herein, as standalone reporters or tags, etc.) herein do not have an A at a position corresponding to position 292 of SEQ ID NO: 1. In some embodiments, spHT peptides or polypeptides (e.g., as portions of fusions herein, as standalone reporters or tags, etc.) herein do not have an amino acid at a position corresponding to position 294 of SEQ ID NO: 1. In some embodiments, spHT peptides or polypeptides (e.g., as portions of fusions herein, as standalone reporters or tags, etc.) herein do not have an amino acid at a position corresponding to position 295 of SEQ ID NO: 1.

In some embodiments, spHT peptides or polypeptides (e.g., as portions of fusions herein, as standalone reporters or tags, etc.) herein do not have an amino acid at a position corresponding to position 296 of SEQ ID NO: 1. In some embodiments, spHT peptides or polypeptides (e.g., as portions of fusions herein, as standalone reporters or tags, etc.) herein do not have an amino acid at a position corresponding to position 297 of SEQ ID NO: 1.

In some embodiments, a sp dehalogenase (e.g., spHT) comprises two peptide and/or polypeptide components that collectively comprise at least 70% sequence similarity or identity with all or a portion of SEQ ID NO: 1 (e.g., >70% sequence similarity or identity, >75% sequence similarity or identity, >80% sequence similarity or identity, >85% sequence similarity or identity, >90% sequence similarity or identity, >95% sequence similarity or identity, >96% sequence similarity or identity, >97% sequence similarity or identity, >98% sequence similarity or identity, >99% sequence similarity or identity). For example, the first peptide/polypeptide component of the sp polypeptide corresponds to a first portion of SEQ ID NO: 1 (e.g., at least 70% sequence similarity or identity to the first portion), and the second peptide/polypeptide component of the sp polypeptide corresponds to a second portion of SEQ ID NO: 1 (e.g., at least 70% sequence similarity or identity to the second portion). In some embodiments, a sp dehalogenase (e.g., spHT) comprises two fragments that collectively comprise 100% sequence similarity or identity with all or a portion of SEQ ID NO: 1. For example, the first fragment of the sp polypeptide has 100% sequence similarity or identity to a first portion of SEQ ID NO: 1, and the second fragment of the sp polypeptide has 100% sequence similarity or identity to a second portion SEQ ID NO: 1.

In some embodiments, a sp dehalogenase (e.g., present as a portion of a fusion herein, present as standalone reporter or tag, etc.) comprises a sp site. The sp site is an internal location in the parent sequence that defines the C-terminus of the first component or fragment and the N-terminus of the second component or fragment of the sp dehalogenase. For example, if a theoretical a 100 amino acid polypeptide were split with a sp site between residues 57 and 58 of the parent polypeptide (referred to herein as a sp site of 57), the first component polypeptide would correspond to positions 1-57, and the second component polypeptide would correspond to positions 58-100. In some embodiments herein, a sp site within SEQ ID NO: 1 may occur at any position from position 5 of SEQ ID NO:1 to position 290 of SEQ ID NO: 1. In some embodiments, SEQ ID NOS: 2-577 are exemplary components of spHT polypeptides having 100% sequence identity to SEQ ID NO: 1. In some embodiments, an active spHT complex is formed between two fragments that collectively comprise amino acids corresponding to each position in SEQ ID NO: 1. For example, a polypeptide having a sequence of SEQ ID NO: 26 and a peptide having a sequence of SEQ ID NO: 27 collectively comprise amino acids corresponding to each position in SEQ ID NO: 1. Any pairs of peptide and polypeptides (or two polypeptides) corresponding to two of SEQ ID NOS: 2-577 and together comprising amino acids corresponding to each position in SEQ ID NO: 1 (with or without deletion or duplication of positions) find use in embodiments herein. In some embodiments, a spHT dehalogenase (e.g., for use in the fusions herein or as a standalone reporter or tag) comprises any of the following pairs of fragments: SEQ ID NOS: 2 and 3, 4 and 5, 6 and 7, 8 and 9, 10 and 11, 12 and 13, 14 and 15, 16 and 17, 18 and 19, 20 and 21, 22 and 23, 24 and 25, 26 and 27, 28 and 29, 30 and 31, 32 and 33, 34 and 35, 36 and 37, 38 and 39, 40 and 41, 42 and 43, 44 and 45, 46 and 47, 48 and 49, 50 and 51, 52 and 53, 54 and 55, 56 and 57, 58 and 59, 60 and 61, 62 and 63, 64 and 65, 66 and 67, 68 and 69, 70 and 71, 72 and 73, 74 and 75, 76 and 77, 78 and 79, 80 and 81, 82 and 83, 84 and 85, 86 and 87, 88 and 89, 90 and 91, 92 and 93, 94 and 95, 96 and 97, 98 and 99, 100 and 101, 102 and 103, 104 and 105, 106 and 107, 108 and 109, 110 and 111, 112 and 113, 114 and 115, 116 and 117, 118 and 119, 120 and 121, 121, 122 and 123, 124 and 125, 126 and 127, 128 and 129, 130 and 131, 132 and 133, 134 and 135, 136 and 137, 138 and 139, 140 and 141, 142 and 143, 144 and 145, 146 and 147, 148 and 149, 150 and 151, 152 and 153, 154 and 155, 156 and 157, 158 and 159, 160 and 161, 172 and 173, 174 and 175, 176 and 177, 178 and 179, 180 and 181, 182 and 183, 184 and 185, 186 and 187, 188 and 189, 190 and 191, 192 and 193, 194 and 195, 196 and 197, 198 and 199, 200 and 201, 202 and 203, 204 and 205, 206 and 207, 208 and 209, 190 and 211, 212 and 213, 214 and 215, 216 and 217, 218 and 219, 220 and 221, 222 and 223, 224 and 225, 226 and 227, 228 and 229, 300 and 301, 302 and 303, 304 and 305, 306 and 307, 308 and 309, 310 and 311, 312 and 313, 314 and 315, 316 and 317, 318 and 319, 320 and 321, 322 and 323, 324 and 325, 326 and 327, 328 and 329, 330 and 331, 332 and 333, 334 and 335, 336 and 337, 338 and 339, 340 and 341, 342 and 343, 344 and 345, 346 and 347, 348 and 349, 350 and 351, 352 and 353, 354 and 355, 356 and 357, 358 and 359, 360 and 361, 362 and 363, 364 and 365, 366 and 367, 368 and 369, 370 and 371, 372 and 373, 374 and 375, 376 and 377, 378 and 379, 380 and 381, 382 and 383, 384 and 385, 386 and 387, 388 and 389, 390 and 391, 392 and 393, 394 and 395, 396 and 397, 398 and 399, 400 and 401, 402 and 403, 404 and 405, 406 and 407, 408 and 409, 410 and 411, 412 and 413, 414 and 415, 416 and 417, 418 and 419, 420 and 421, 422 and 423, 424 and 425, 426 and 427, 428 and 429, 430 and 431, 432 and 433, 434 and 435, 436 and 437, 438 and 439, 440 and 441, 442 and 443, 444 and 445, 446 and 447, 448 and 449, 450 and 451, 452 and 453, 454 and 455, 456 and 457, 458 and 459, 460 and 461, 462 and 463, 464 and 465, 466 and 467, 468 and 469, 470 and 471, 472 and 473, 474 and 475, 476 and 477, 478 and 479, 480 and 481, 482 and 483, 484 and 485, 486 and 487, 488 and 489, 490 and 491, 492 and 493, 494 and 495, 496 and 497, 498 and 499, 500 and 501, 502 and 503, 504 and 505, 506 and 507, 508 and 509, 510 and 511, 512 and 513, 514 and 515, 516 and 517, 518 and 519, 520 and 521, 522 and 523, 524 and 525, 526 and 527, 528 and 529, 530 and 531, 532 and 533, 534 and 535, 536 and 537, 538 and 539, 540 and 541, 542 and 543, 544 and 545, 546 and 547, 548 and 549, 550 and 551, 552 and 553, 554 and 555, 556 and 557, 558 and 559, 560 and 561, 562 and 563, 564 and 565, 566 and 567, 568 and 569, 570 and 571, 572 and 573, 574 and 575, and 576 and 577.

In some embodiments, a spHT comprises a peptide and polypeptide (or two polypeptides) pair corresponding to two of SEQ ID NOS: 2-577 together comprising amino acids corresponding to each position in SEQ ID NO: 1, but with a deletion of up to 40 amino acids in length (e.g., 1, 2, 3, 4, 5, 6, 7, 8, 9, 10, 15, 20, 25, 30, 35, 40, or ranges therebetween) at the C-terminus or N-terminus of one or both of fragments. For example, a pair corresponding to SEQ ID NOS: 7 and 28 together correspond to positions of SEQ ID NO: 1, but with an 11-residue deletion. In some embodiments, any pairs of SEQ ID NOS: 2-577, together corresponding to the sequence of SEQ ID NO: 1, but with deletions of up to 40 amino acids, are within the scope of spHTs herein. In some embodiments, the deletion is adjacent to the split site. In some embodiments, the deletion corresponds to the N- or C-terminus of SEQ ID NO: 1.

In some embodiments, a spHT comprises a peptide and polypeptide (or two polypeptides) pair corresponding to two of SEQ ID NOS: 2-577 together comprising amino acids corresponding to each position in SEQ ID NO: 1, but with a duplication of up to 40 amino acids in length (e.g., 1, 2, 3, 4, 5, 6, 7, 8, 9, 10, 15, 20, 25, 30, 35, 40, or ranges therebetween) at the C-terminus or N-terminus of one or both of fragments. For example, a pair corresponding to SEQ ID NOS: 6 and 29 together correspond to positions of SEQ ID NO: 1, but with an 11 residue duplication. In some embodiments, any pairs of SEQ ID NOS: 2-577, together corresponding to the sequence of SEQ ID NO: 1, but with duplications of up to 40 amino acids, are within the scope of spHTs herein. In some embodiments, the duplication is adjacent to the split site. In some embodiments, the duplication corresponds to the N- or C-terminus of SEQ ID NO: 1.

Fragments utilizing any sp sites, for example, corresponding to a position between position 5 and position 290 of SEQ ID NO: 1, are readily envisioned and within the scope herein.

In some embodiments, spHTs are provided with a sp site corresponding to position 5, 6, 7, 8, 9, 10, 11, 12, 13, 14, 15, 16, 17, 18, 19, 31, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 313, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 310, 311, 312, 313, 314, 315, 316, 317, 318, 319, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, or 290 of SEQ ID NO: 1.

In some embodiments, spHTs are provided with a sp site corresponding to a position between positions 5 and 13, 36 and 51, 63 and 72, 84 and 92, 104 and 130, 142 and 148, 160 and 174, 186 and 189, 311 and 313, 221 and 229, or 269 and 290 of SEQ ID NO: 1.

In some embodiments, the spHT peptides and polypeptides herein (e.g., of the fusions herein or as standalone reporters or tags) comprise one or more (e.g., 2, 3, 4, 5, 6, 7, 8, 9 10, 11, 12, 13, 14, 15, 16, 17, 18, 19, 20, 25, 30, 35, 40, 50, 75, or more) substitutions or deletions relative to one of SEQ ID NOS: 2-557. In some embodiments, sp peptides and polypeptides are provided (e.g., within the fusions herein or as standalone reporters or tags) having 70%-100% sequence identity to one of SEQ ID NOS: 2-557 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity). In some embodiments, sp peptides and polypeptides are provided having 70%-100% sequence similarity to one of SEQ ID NOS: 2-557 (e.g., >70% sequence similarity, >75% sequence similarity, >80% sequence similarity, >85% sequence similarity, >90% sequence similarity, >95% sequence similarity, >96% sequence similarity, >97% sequence similarity, >98% sequence similarity, >99% sequence similarity).

In some embodiments, pairs of sp peptides and/or polypeptides are provided (e.g., within the fusions herein or as standalone reporters or tags) that are capable of forming active sp dehalogenase complexes (active spHT complexes). In some embodiments, such pairs comprise at least 70% sequence identity or similarity to two of SEQ ID NOS: 2-557, and together comprise residues corresponding to 100% of the positions in SEQ ID NO: 1, allowing for up to 40 deletions or duplications at the C- or N-terminus of the peptides/polypeptides.

In some embodiments, the first fragment of a spHT complementary pair corresponds to position 1 through position 5, 6, 7, 8, 9, 10, 11, 12, 13, 14, 15, 16, 17, 18, 19, 31, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 313, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 310, 311, 312, 313, 314, 315, 316, 317, 318, 319, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, or 290 of SEQ ID NO: 1.

In some embodiments, the second fragment of a spHT complementary pair corresponds to position 7, 8, 9, 10, 11, 12, 13, 14, 15, 16, 17, 18, 19, 31, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 313, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 310, 311, 312, 313, 314, 315, 316, 317, 318, 319, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, or 290 through position 294 of SEQ ID NO: 1.

In some embodiments, the duplicated portion of a spHT complementary pair is 1-40 amino acids in length (e.g., 1, 2, 3, 4, 5, 6, 7, 8, 9, 10, 11, 12, 13, 14, 15, 16, 17, 18, 19, 31, 21, 22, 23, 24, 25, 26, 27, 28, 29, 30, 31, 32, 33, 34, 35, 36, 37, 38, 39, 40, or ranges therebetween).

In some embodiments, the deleted portion of a spHTs complementary pair is 1-40 amino acids in length (e.g., 1, 2, 3, 4, 5, 6, 7, 8, 9, 10, 11, 12, 13, 14, 15, 16, 17, 18, 19, 31, 21, 22, 23, 24, 25, 26, 27, 28, 29, 30, 31, 32, 33, 34, 35, 36, 37, 38, 39, 40, or ranges therebetween).

The exemplary spHT fragment sequences of SEQ ID NOS: 2-577 comprise 100% sequence identity to portions of SEQ ID NO: 1; there are no portions of these sequences that do not align with 100% sequence identity to SEQ ID NO: 1. However, as described herein, spHT peptides and polypeptides may have less than 100% sequence identity with SEQ ID NO: 1 (e.g., >70%, >75%, >80%, >85%, >90%, >95%, >96%, >97%, >98%, >99%, but less than 100% sequence identity). Therefore, peptides and polypeptide having less than 100% sequence identity with one or SEQ ID NOS: 2-577 (e.g., >70%, >75%, >80%, >85%, >90%, >95%, >96%, >97%, >98%, >99%, but less than 100% sequence identity) are provided herein and find use in the complementary pairs and complexes herein.

In some embodiments, a spHT complementary pair herein comprises a peptide corresponding to SEQ ID NO: 578 and a polypeptide corresponding to SEQ ID NO: 1188. SEQ NOS: 578 and 1188 are fragments of SEQ ID NO: 1 and have 100% sequence identity to portions of SEQ ID NO: 1. In some embodiments, a spHT complementary pair comprises a peptide having 100% sequence identity to SEQ ID NO: 578; such a peptide is referred to herein as “SmHT.” In some embodiments, a spHT complementary pair comprises a polypeptide having 100% sequence identity to SEQ ID NO: 1188; such a polypeptide is referred to herein as “LgHT.” Extensive experiments were conducted during development of embodiments herein to analyze variants of SmHT and LgHT. SEQ ID NOS: 579-1187 correspond to peptide variants having at least one and up to all positions of SEQ ID NO: 588 substituted. A peptide of each of SEQ ID NOS: 578-1187 was synthesized and tested for various characteristics, including the ability to form an active complex with a complementary LgHT variant polypeptide. SEQ ID NOS: 1189-3033 correspond to polypeptide variants having one or more substitutions relative to SEQ ID NO: 1188. A polypeptide of each of SEQ ID NOS: 1188-3033 was synthesized and tested for various characteristics, including the ability to form an active complex with a complementary SmHT variant peptide.

In some embodiments, provided herein are variant SmHT peptides having one or more substitutions relative to a reference SmHT peptide sequence, for example, peptides of SEQ ID NOS: 3061, 3064-3066, and 3079-3091.

In some embodiments, provided herein (e.g., within a fusion herein or as a standalone reporter or tag, etc.) is a SmHT peptide or SmHT variant peptide having at least 70% (e.g., 70%, 75%, 80%, 85%, 90%, 95%, 100%, or ranges therebetween) sequence similarity (e.g., conservative or semi-conservative similarity) with one of SEQ ID NOS: 578-1187, 3061, 3064-3066, and 3079-3091. In some embodiments, a peptide (e.g., within a fusion herein or as a standalone reporter or tag, etc.) corresponds to SmHT (SEQ ID NO: 578), but with one or more (e.g., 2, 3, 4, 5, 6, 7, 8, 9, 10, 11, 12, 13, 14, 15, 16, 17, or ranges therebetween) of the substitutions of one or more of SEQ ID NOS: 588-1187 relative to SEQ ID NO: 578. In some embodiments, a SmHT variant (e.g., within a fusion herein or as a standalone reporter or tag, etc.) has 1-8 (e.g., 1, 2, 3, 4, 5, 6, 7, 8, or ranges therebetween) non-conservative substitutions relative to one of SEQ ID NOS: 578-1187.

In some embodiments, a peptide component of a modified dehalogenase (e.g., within a fusion herein or as a standalone reporter or tag, etc.) corresponds to HT[3-19] (SEQ ID NO: 3061), or a variant with one or more (e.g., 2, 3, 4, 5, 6, 7, 8, 9, 10, 11, 12, 13, 14, 15, 16, 17, or ranges therebetween) of the substitutions of one or more of SEQ ID NOS: 3061, 3064-3066, and 3079-3091 relative to SEQ ID NO: 3061. In some embodiments, a HT[3-19] variant (e.g., within a fusion herein or as a standalone reporter or tag, etc.) has 1-8 (e.g., 1, 2, 3, 4, 5, 6, 7, 8, or ranges therebetween) non-conservative substitutions relative to one of SEQ ID NOS: 3061, 3064-3066, and 3079-3091.

In some embodiments, provided herein (e.g., within a fusion herein or as a standalone reporter or tag, etc.) is a SmHT peptide or SmHT variant peptide comprising:

(SEQ ID NO: 3034)
X₁X₂X₃X₄X₅(F/W/Y/M/H)X₇(F/W/Y/D/R)X₉X₁₀X₁₁(F/W/Y/M/H/
R)(V/I/L/M/A/C)X₁₄(V/I/L/A/C/MI/L/F/W)X₁₆X₁₇;
and/or
(SEQ ID NO: 3035)
X₁X₂X₃X₄X₅(F/W/Y)X₇(F/W/Y)X₉X₁₀X₁₁(F/W/Y)(V/I/L/M)X₁₄
(V/I/L X₁₆X₁₇;

wherein each X is any amino acid (e.g., proteinogenic amino acid).

In some embodiments, provided herein is a LgHT polypeptide or LgHT variant polypeptide (e.g., within a fusion herein or as a standalone reporter or tag, etc.) having at least 70% (e.g., 70%, 75%, 80%, 85%, 90%, 95%, 100%, or ranges therebetween) sequence similarity (e.g., conservative or semi-conservative similarity) with one of SEQ ID NOS: 1188-3033. In some embodiments, a polypeptide (e.g., within a fusion herein or as a standalone reporter or tag, etc.) corresponds to LgHT (SEQ ID NO: 1188), but with one or more (e.g., 2, 3, 4, 5, 6, 7, 8, 9, 10, 11, 12, 13, 14, 15, 16, 17, 18, 19, 20, 25, 30, or more, or ranges therebetween) of the substitutions of one or more of SEQ ID NOS: 1189-3033 relative to SEQ ID NO: 1188. In some embodiments, a LgHT variant (e.g., within a fusion herein or as a standalone reporter or tag, etc.) has at least 70% (e.g., 70%, 75%, 80%, 85%, 90%, 95%, 100%, or ranges therebetween) sequence identity with one of SEQ ID NOS: 1188-3033. In some embodiments, a LgHT polypeptide of LgHT variant polypeptide comprises a substitution relative to a reference LgHT sequence (SEQ ID NO: 1188) corresponding to a substitution present in the LgHT portion of a construct of one or SEQ ID NOS: 3110-4064.

In some embodiments, provided herein is a spHT complementary pair (e.g., each component of the pair present within a separate fusion) comprising (a) a SmHT peptide or SmHT variant peptide having (1) at least 70% (e.g., 70%, 75%, 80%, 85%, 90%, 95%, 100%, or ranges therebetween) sequence similarity (e.g., conservative or semi-conservative similarity) with one of SEQ ID NOS: 578-1187, 3061, 3064-3066, and 3079-3091, (2) one or more (e.g., 2, 3, 4, 5, 6, 7, 8, 9, 10, 11, 12, 13, 14, 15, 16, 17, or ranges therebetween) substitutions relative to SEQ ID NO: 578, and/or (3) 1-8 (e.g., 1, 2, 3, 4, 5, 6, 7, 8, or ranges therebetween) non-conservative substitutions relative to one of SEQ ID NOS: 578-1187, 3061, 3064-3066, and 3079-3091; and (b) a LgHT polypeptide or LgHT variant polypeptide having (1) at least 70% (e.g., 70%, 75%, 80%, 85%, 90%, 95%, 100%, or ranges therebetween) sequence similarity (e.g., conservative or semi-conservative similarity) with one of SEQ ID NOS: 1188-3033, (2) one or more (e.g., 2, 3, 4, 5, 6, 7, 8, 9, 10, 11, 12, 13, 14, 15, 16, 17, 18, 19, 20, 25, 30, or more, or ranges therebetween) substitutions relative to SEQ ID NO: 1188, and/or (3) at least 70% (e.g., 70%, 75%, 80%, 85%, 90%, 95%, 100%, or ranges therebetween) sequence identity with one of SEQ ID NOS: 1188-3033.

The formation of a spHT complex from two complementary fragments may be reversible or irreversible. In some embodiments, a spHT complex is capable of being denatured, renatured, and having its activity reconstituted. In some embodiments, such spHTs find use in methods that comprise exposing samples containing the spHTs to denaturing conditions (e.g., manufacturing conditions, storage conditions, etc.) prior to substrate binding.

### NanoLuc

In some embodiments, provided herein are compositions (e.g., fusion peptides and polypeptides) and systems (e.g., multiple complementary fusion peptides and polypeptides, substrates, ligands, etc.) comprising peptide/polypeptide fragments capable of interacting (e.g., facilitated or unfacilitated) to form an active luminescent protein capable of utilizing an appropriate substrate to generate luminescence.

In some embodiments, provided herein are compositions (e.g., fusion peptides and polypeptides) and systems (e.g., multiple complementary fusion peptides and polypeptides, substrates, ligands, etc.) comprising complementary peptide/polypeptide fragments capable of interacting (e.g., facilitated or unfacilitated) to form an active bioluminescent complex capable of generating luminescence upon interaction with an appropriate luminescent substrate. In some embodiments, a first fusion is provided comprising a complementary peptide fragment of a luminescent protein, and a second fusion is provided comprising a complementary polypeptide fragment of the luminescent protein, wherein upon interacting (e.g., facilitated or unfacilitated), the complementary peptide and polypeptide form an active bioluminescent complex capable of generating luminescence upon interaction with an appropriate luminescent substrate. In some embodiments, the complementary peptide and polypeptide are fragments of a split luminescent protein (e.g., luciferase). In alternative embodiments, both fragments may be polypeptides.

Provided herein, as components of the compositions, systems, and methods herein are bipartite or multipartite bioluminescent complexes, such as those derived from the commercially available NANOLUC protein (Promega), and/or the NANOBIT (Promega) or NANOTRIP structural complementation systems.

The native Oplophorus gracilirostris luciferase (OgLuc) and commercially-available NANOLUC luciferase (Promega Corporation) each comprise polypeptides of 10 β (beta) strands (β1, β2, β3, β4, β5, β6, β7, β8, β9, β10). U.S. Pat. No. 9,797,889 (herein incorporated by reference in its entirety) describes development and use of a complementation system comprising a β1-9-like polypeptide and a β10-like peptide (certain OgLuc/NANOLUC-based polypeptide and peptide sequences in polypeptide and peptide sequences in U.S. Pat. No. 9,797,889 differ from the corresponding sequences in NANOLUC and wild-type native OgLuc). Similarly, U.S. application Ser. No. 16/439,565 (herein incorporated by reference in its entirety) describes the development and use of a complementation systems comprising two or more OgLuc/NANOLUC peptides and/or polypeptides (certain OgLuc/NANOLUC-based polypeptide and peptide sequences in U.S. patent Ser. No. 16/439,565 differ from the corresponding sequences in NANOLUC and wild-type native OgLuc).

In some embodiments, provided herein is a peptide component of a binary bioluminescent complex (e.g., within a fusion herein or as a standalone reporter or tag, etc.) having greater than 40% (e.g., >40%, >45%, >50%, >55%, >60%, >65%, >70%, >75%, >80%, >85%, >90%, >95%, >98%, >99%, 100%) sequence identity with SEQ ID NO: 3036, wherein a detectable bioluminescent signal is produced when the peptide component of a binary bioluminescent complex contacts a polypeptide consisting of SEQ ID NO: 3037 (e.g., within a fusion herein or as a standalone reporter or tag, etc.) in the presence of a substrate for the bioluminescent complex (e.g., greater luminescence that the components of the complex in the presence of the substrate). In some embodiments, the peptide has less than 100% sequence identity with SEQ ID NO: 3036. In some embodiments, a detectable bioluminescent signal is produced when the peptide component of a binary bioluminescent complex contacts a polypeptide component of the binary bioluminescent complex having greater than 40% (e.g., >40%, >45%, >50%, >55%, >60%, >65%, >70%, >75%, >80%, >85%, >90%, >95%, >98%, >99%, 100%) sequence identity with SEQ ID NO: 3037. In certain embodiments, the detectable bioluminescent signal is produced, or is substantially increased, when the peptide associates with the polypeptide comprising or consisting of SEQ ID NO: 3037. In preferred embodiments, the peptide exhibits alteration (e.g., enhancement) of one or more traits compared to a peptide of SEQ ID NO: 3038 or 3039, wherein the traits are selected from: affinity for the polypeptide consisting of SEQ ID NO: 3037, expression, intracellular solubility, intracellular stability, and bioluminescent activity when combined with the polypeptide consisting of SEQ ID NO: 3037.

Exemplary sequences of peptide components of binary bioluminescent complexes that find use in the embodiments herein are described, for example, in U.S. Pat. No. 9,797,889 (incorporated by reference in its entirety). Although the peptide components of binary bioluminescent complexes herein are not limited to these sequences, in some embodiments, the peptide component of a binary bioluminescent complex herein may be selected from amino acid sequences of SEQ ID NOS: 3-438 and 2162-2365 of U.S. Pat. No. 9,797,889 (incorporated by reference in its entirety).

In some embodiments, provided herein is a peptide component of a binary bioluminescent complex (e.g., within a fusion herein or as a standalone reporter or tag, etc.) having greater than 40% (e.g., >40%, >45%, >50%, >55%, >60%, >65%, >70%, >75%, >80%, >85%, >90%, >95%, >98%, >99%, 100%) sequence identity with SEQ ID NO: 3038, wherein a detectable bioluminescent signal is produced when the peptide component of a binary bioluminescent complex contacts a polypeptide consisting of SEQ ID NO: 3037 (e.g., within a fusion herein or as a standalone reporter or tag, etc.) in the presence of a substrate for the bioluminescent complex (e.g., greater luminescence that the components of the complex in the presence of the substrate). In some embodiments, the peptide has less than 100% sequence identity with SEQ ID NO: 3036. In some embodiments, a detectable bioluminescent signal is produced when the peptide component of a binary bioluminescent complex contacts a polypeptide component of the binary bioluminescent complex having greater than 40% (e.g., >40%, >45%, >50%, >55%, >60%, >65%, >70%, >75%, >80%, >85%, >90%, >95%, >98%, >99%, 100%) sequence identity with SEQ ID NO: 3037. In certain embodiments, the detectable bioluminescent signal is produced, or is substantially increased, when the peptide associates with the polypeptide comprising or consisting of SEQ ID NO: 3037. In some embodiments, the peptide exhibits high affinity for a polypeptide of SEQ ID NO: 3037 and is capable of forming a bioluminescent complex without facilitation. In preferred embodiments, the peptide exhibits alteration (e.g., enhancement) of one or more traits compared to a peptide of SEQ ID NO: 3036, wherein the traits are selected from: affinity for the polypeptide consisting of SEQ ID NO: 3037, expression, intracellular solubility, intracellular stability, and bioluminescent activity when combined with the polypeptide consisting of SEQ ID NO: 3037.

In some embodiments, provided herein is a peptide component of a binary bioluminescent complex (e.g., within a fusion herein or as a standalone reporter or tag, etc.) having greater than 40% (e.g., >40%, >45%, >50%, >55%, >60%, >65%, >70%, >75%, >80%, >85%, >90%, >95%, >98%, >99%, 100%) sequence identity with SEQ ID NO: 3039, wherein a detectable bioluminescent signal is produced when the peptide component of a binary bioluminescent complex contacts a polypeptide consisting of SEQ ID NO: 3037 (e.g., within a fusion herein or as a standalone reporter or tag, etc.) in the presence of a substrate for the bioluminescent complex (e.g., greater luminescence that the components of the complex in the presence of the substrate). In some embodiments, the peptide has less than 100% sequence identity with SEQ ID NO: 3036. In some embodiments, a detectable bioluminescent signal is produced when the peptide component of a binary bioluminescent complex contacts a polypeptide component of the binary bioluminescent complex having greater than 40% (e.g., >40%, >45%, >50%, >55%, >60%, >65%, >70%, >75%, >80%, >85%, >90%, >95%, >98%, >99%, 100%) sequence identity with SEQ ID NO: 3037. In certain embodiments, the detectable bioluminescent signal is produced, or is substantially increased, when the peptide associates with the polypeptide comprising or consisting of SEQ ID NO: 3037. In some embodiments, the peptide exhibits low affinity for a polypeptide of SEQ ID NO: 3037 and does not form a stable bioluminescent complex without facilitation. In preferred embodiments, the peptide exhibits alteration (e.g., enhancement) of one or more traits compared to a peptide of SEQ ID NO: 3036, wherein the traits are selected from: affinity (or low affinity) for the polypeptide consisting of SEQ ID NO: 3037, expression, intracellular solubility, intracellular stability, and bioluminescent activity when combined with the polypeptide consisting of SEQ ID NO: 3037. In some embodiments, provided herein is a polypeptide component of a binary bioluminescent complex (e.g., within a fusion herein or as a standalone reporter or tag, etc.) having greater than 40% (e.g., >40%, >45%, >50%, >55%, >60%, >65%, >70%, >75%, >80%, >85%, >90%, >95%, >98%, >99%, 100%) sequence identity with SEQ ID NO: 3037, wherein a detectable bioluminescent signal is produced when the polypeptide contacts a peptide consisting of SEQ ID NO: 3036 (e.g., within a fusion herein or as a standalone reporter or tag, etc.) in the presence of a substrate for the bioluminescent complex (e.g., greater luminescence that the components of the complex in the presence of the substrate). In some embodiments, a polypeptide component of a binary bioluminescent complex (e.g., within a fusion herein or as a standalone reporter or tag, etc.) has less than 100% sequence identity with SEQ ID NO: 3037. In some embodiments, a detectable bioluminescent signal is produced when the polypeptide contacts a peptide having greater than 40% (e.g., >40%, >45%, >50%, >55%, >60%, >65%, >70%, >75%, >80%, >85%, >90%, >95%, >98%, >99%, 100%) sequence identity with SEQ ID NO: 3036. In some embodiments, the polypeptide exhibits alteration (e.g., enhancement) of one or more traits compared to a peptide of SEQ ID NO: 3037, wherein the traits are selected from: affinity for the peptide consisting of SEQ ID NO: 3036 or 3038, expression, intracellular solubility, intracellular stability, and bioluminescent activity when combined with the peptide consisting of SEQ ID NO: 3036, 3038, or 3039.

Exemplary sequences of polypeptide components of binary bioluminescent complexes that find use in the embodiments herein are described, for example, in U.S. Pat. No. 9,797,889 (incorporated by reference in its entirety). Although the peptide components of binary bioluminescent complexes herein are not limited to these sequences, in some embodiments, the polypeptide component of a binary bioluminescent complex herein may be selected from amino acid sequences of SEQ ID NOS: 441-2156 of U.S. Pat. No. 9,797,889 (incorporated by reference in its entirety).

In certain embodiments, the present invention provides bioluminescent complexes (formed between the fusions described herein) comprising: (a) a peptide comprising a peptide amino acid sequence having greater than 40% (e.g., >40%, >45%, >50%, >55%, >60%, >65%, >70%, >75%, >80%, >85%, >90%, >95%, >98%, >99%, 100%) sequence identity with SEQ ID NO: 3036, 3038, or 3039; and (b) a polypeptide comprising a polypeptide amino acid sequence having greater than 40% (e.g., >40%, >45%, >50%, >55%, >60%, >65%, >70%, >75%, >80%, >85%, >90%, >95%, >98%, >99%, 100%) sequence identity with SEQ ID NO: 3037, wherein the bioluminescent complex exhibits detectable luminescence in the presence of a substrate for the bioluminescent complex (e.g., greater luminescence that the components of the complex in the presence of the substrate).

In some embodiments, provided herein is a polypeptide component of a binary bioluminescent complex (e.g., within a fusion herein or as a standalone reporter or tag, etc.) having greater than 40% (e.g., >40%, >45%, >50%, >55%, >60%, >65%, >70%, >75%, >80%, >85%, >90%, >95%, >98%, >99%, 100%) sequence identity with SEQ ID NO: 3037, wherein a detectable bioluminescent signal is produced when the polypeptide contacts a peptide consisting of SEQ ID NO: 3036, 3038, or 3039 (e.g., within a fusion herein or as a standalone reporter or tag, etc.) in the presence of a substrate for the bioluminescent complex (e.g., greater luminescence that the components of the complex in the presence of the substrate).

In some embodiments, provided herein (alone and/or within fusions described herein) are components of a bioluminescent complex comprising a first component having 40% or greater (e.g., 40%, 45%, 50%, 55%, 60%, 65%, 70%, 75%, 80%, 85%, 90%, 95%, or more (e.g., 100%), or ranges therebetween) sequence identity to a first fragment of SEQ ID NO: 3040 or SEQ ID NO: 3041; and one or more complementary fragments collectively comprising 40% or greater (e.g., 40%, 45%, 50%, 55%, 60%, 65%, 70%, 75%, 80%, 85%, 90%, 95%, or more (e.g., 100%), or ranges therebetween) sequence identity to the complementary portion of SEQ ID NO: 3040 or SEQ ID NO: 3041; wherein a bioluminescent signal produced by a bioluminescent complex assembled of the components in the presence of a coelenterazine or a coelenterazine derivative substrate is substantially increased when compared to a bioluminescent signal produced by the coelenterazine substrate and individual components alone. In some embodiments, the first component comprises 40% or greater (e.g., 40%, 45%, 50%, 55%, 60%, 65%, 70%, 75%, 80%, 85%, 90%, 95%, or more (e.g., 100%), or ranges therebetween) sequence identity to SEQ ID NO: 3042, and the one or more complementary components collectively comprising 40% or greater (e.g., 40%, 45%, 50%, 55%, 60%, 65%, 70%, 75%, 80%, 85%, 90%, 95%, or more (e.g., 100%), or ranges therebetween) sequence identity to SEQ ID NO: 3046. In some embodiments, the first component comprises 40% or greater (e.g., 40%, 45%, 50%, 55%, 60%, 65%, 70%, 75%, 80%, 85%, 90%, 95%, or more (e.g., 100%), or ranges therebetween) sequence identity to SEQ ID NO: 3043, and the one or more complementary components collectively comprising 40% or greater (e.g., 40%, 45%, 50%, 55%, 60%, 65%, 70%, 75%, 80%, 85%, 90%, 95%, or more (e.g., 100%), or ranges therebetween) sequence identity to SEQ ID NO: 3047. In some embodiments, the first component comprises 40% or greater (e.g., 40%, 45%, 50%, 55%, 60%, 65%, 70%, 75%, 80%, 85%, 90%, 95%, or more (e.g., 100%), or ranges therebetween) sequence identity to SEQ ID NO: 3044, and the one or more complementary components collectively comprising 40% or greater (e.g., 40%, 45%, 50%, 55%, 60%, 65%, 70%, 75%, 80%, 85%, 90%, 95%, or more (e.g., 100%), or ranges therebetween) sequence identity to SEQ ID NO: 3048. In some embodiments, the first component comprises 40% or greater (e.g., 40%, 45%, 50%, 55%, 60%, 65%, 70%, 75%, 80%, 85%, 90%, 95%, or more (e.g., 100%), or ranges therebetween) sequence identity to SEQ ID NO: 3045, and the one or more complementary components collectively comprising 40% or greater (e.g., 40%, 45%, 50%, 55%, 60%, 65%, 70%, 75%, 80%, 85%, 90%, 95%, or more (e.g., 100%), or ranges therebetween) sequence identity to SEQ ID NO: 3049. In some embodiments, the first component comprises 40% or greater (e.g., 40%, 45%, 50%, 55%, 60%, 65%, 70%, 75%, 80%, 85%, 90%, 95%, or more (e.g., 100%), or ranges therebetween) sequence identity to SEQ ID NO: 3042, and the one or more complementary components collectively comprising 40% or greater (e.g., 40%, 45%, 50%, 55%, 60%, 65%, 70%, 75%, 80%, 85%, 90%, 95%, or more (e.g., 100%), or ranges therebetween) sequence identity to SEQ ID NO: 3050. In some embodiments, the first component comprises 40% or greater (e.g., 40%, 45%, 50%, 55%, 60%, 65%, 70%, 75%, 80%, 85%, 90%, 95%, or more (e.g., 100%), or ranges therebetween) sequence identity to SEQ ID NO: 3043, and the one or more complementary components collectively comprising 40% or greater (e.g., 40%, 45%, 50%, 55%, 60%, 65%, 70%, 75%, 80%, 85%, 90%, 95%, or more (e.g., 100%), or ranges therebetween) sequence identity to SEQ ID NO: 3051. In some embodiments, the first component comprises 40% or greater (e.g., 40%, 45%, 50%, 55%, 60%, 65%, 70%, 75%, 80%, 85%, 90%, 95%, or more (e.g., 100%), or ranges therebetween) sequence identity to SEQ ID NO: 3044, and the one or more complementary components collectively comprising 40% or greater (e.g., 40%, 45%, 50%, 55%, 60%, 65%, 70%, 75%, 80%, 85%, 90%, 95%, or more (e.g., 100%), or ranges therebetween) sequence identity to SEQ ID NO: 3052. In some embodiments, the first component comprises 40% or greater (e.g., 40%, 45%, 50%, 55%, 60%, 65%, 70%, 75%, 80%, 85%, 90%, 95%, or more (e.g., 100%), or ranges therebetween) sequence identity to SEQ ID NO: 3045, and the one or more complementary components collectively comprising 40% or greater (e.g., 40%, 45%, 50%, 55%, 60%, 65%, 70%, 75%, 80%, 85%, 90%, 95%, or more (e.g., 100%), or ranges therebetween) sequence identity to SEQ ID 3053. In some embodiments, the bioluminescent signal is substantially increased when the first component associates with the one or more complementary components.

Exemplary sequences of peptide and polypeptide components of binary or multipartite bioluminescent complexes that find use in the embodiments herein are described, for example, in U.S. application Ser. No. 16/439,565 (incorporated by reference in its entirety). Although the peptide and polypeptide components of binary or multipartite bioluminescent complexes herein are not limited to these sequences, in some embodiments, a peptide or polypeptide component of a binary or multipartite bioluminescent complex herein may be selected from amino acid sequences of SEQ ID NOS: 1-804 of U.S. application Ser. No. 16/439,565 (incorporated by reference in its entirety).

In some embodiments, provided herein are polypeptides (e.g., within a fusion described herein) comprising 40% or greater (e.g., 40%, 45%, 50%, 55%, 60%, 65%, 70%, 75%, 80%, 85%, 90%, 95%, or more (e.g., 100%), or ranges therebetween) sequence identity with one of SEQ ID NO: 790, 791, 792, or 793.

In some embodiments, provided herein are peptides polypeptides (e.g., within a fusion described herein) comprising SEQ ID NO: 3054-3060. In some embodiments, provided herein are peptides (e.g., within a fusion described herein) comprising 40% or greater (e.g., 40%, 45%, 50%, 55%, 60%, 65%, 70%, 75%, 80%, 85%, 90%, 95%, or more (e.g., 100%), or ranges therebetween) sequence identity with one of SEQ ID NO: 3054-3060.

In some embodiments, provided herein is a β6-7-like peptide (e.g., within a fusion described herein) comprising SEQ ID NOS: 3054 and 3055. In some embodiments, provided herein is a β6-7-like peptide having 40% or greater (e.g., 40%, 45%, 50%, 55%, 60%, 65%, 70%, 75%, 80%, 85%, 90%, 95%, or more (e.g., 100%), or ranges therebetween) sequence identity with SEQ ID NOS: 3054 and 3055.

In some embodiments, provided herein is a β7-8-like peptide (e.g., within a fusion described herein) comprising SEQ ID NOS: 3055 and 3056. In some embodiments, provided herein is a β7-8-like peptide (e.g., within a fusion described herein) having 40% or greater (e.g., 40%, 45%, 50%, 55%, 60%, 65%, 70%, 75%, 80%, 85%, 90%, 95%, or more (e.g., 100%), or ranges therebetween) sequence identity with SEQ ID NOS: 3055 and 3056.

In some embodiments, provided herein is a β8-9-like peptide (e.g., within a fusion described herein) comprising SEQ ID NOS: 3056/3059 or 3056/3060. In some embodiments, provided herein is a β8-9-like peptide (e.g., within a fusion described herein) having 40% or greater (e.g., 40%, 45%, 50%, 55%, 60%, 65%, 70%, 75%, 80%, 85%, 90%, 95%, or more (e.g., 100%), or ranges therebetween) sequence identity with SEQ ID NOS: 3056/3059 or 3056/3060.

In some embodiments, provided herein is a β9-10-like peptide (e.g., within a fusion described herein) comprising SEQ ID NOS: 3059/3057, 3059/3058, 3060/3057, or 3060/3058. In some embodiments, provided herein is a β8-9-like peptide (e.g., within a fusion described herein) having 40% or greater (e.g., 40%, 45%, 50%, 55%, 60%, 65%, 70%, 75%, 80%, 85%, 90%, 95%, or more (e.g., 100%), or ranges therebetween) sequence identity with SEQ ID NOS: 3059/3057, 3059/3058, 3060/3057, or 3060/3058.

In some embodiments, provided herein is a β6-8-like peptide or polypeptide (e.g., within a fusion described herein) comprising SEQ ID NOS: 3054-3056. In some embodiments, provided herein is a β6-8-like peptide or polypeptide (e.g., within a fusion described herein) having 40% or greater (e.g., 40%, 45%, 50%, 55%, 60%, 65%, 70%, 75%, 80%, 85%, 90%, 95%, or more (e.g., 100%), or ranges therebetween) sequence identity with SEQ ID NOS: 3054-3056.

In some embodiments, provided herein is a β7-9-like peptide or polypeptide (e.g., within a fusion described herein) comprising SEQ ID NOS: 3055/3056/3059 or 3055/3056/3060. In some embodiments, provided herein is a β7-9-like peptide or polypeptide (e.g., within a fusion described herein) having 40% or greater (e.g., 40%, 45%, 50%, 55%, 60%, 65%, 70%, 75%, 80%, 85%, 90%, 95%, or more (e.g., 100%), or ranges therebetween) sequence identity with SEQ ID NOS: 3055/3056/3059 or 3055/3056/3060.

In some embodiments, provided herein is a β8-10-like peptide or polypeptide (e.g., within a fusion described herein) comprising SEQ ID NOS: 3056/3059/3057, 3056/3059/3058, 3056/3060/3057, or 3056/3060/3058. In some embodiments, provided herein is a β7-9-like peptide or polypeptide (e.g., within a fusion described herein) having 40% or greater (e.g., 40%, 45%, 50%, 55%, 60%, 65%, 70%, 75%, 80%, 85%, 90%, 95%, or more (e.g., 100%), or ranges therebetween) sequence identity with NOS: SEQ ID NOS: 3056/3059/3057, 3056/3059/3058, 3056/3060/3057, or 3056/3060/3058.

### Tandem Peptide Tags

In some embodiments, provided herein are a tandem peptide tags comprising (i) a peptide component of a bioluminescent complex fused to (ii) a peptide component of a modified dehalogenase complex. In some embodiments, the peptide component of a bioluminescent complex is capable of forming a bioluminescent complex with a polypeptide component of the bioluminescent complex upon interaction therebetween (e.g., facilitated or unfacilitated). In some embodiments, the peptide component of a modified dehalogenase complex is capable of forming a modified dehalogenase complex with a polypeptide component of the modified dehalogenase complex upon interaction therebetween (e.g., facilitated or unfacilitated). In some embodiments, the bioluminescent complex and modified dehalogenase complex form upon interaction (facilitated or unfacilitated) of the tandem peptide tag and a tandem polypeptide reporter (e.g., comprising the polypeptide component of the bioluminescent complex fused to the polypeptide component of the modified dehalogenase complex). In some embodiments, the bioluminescent complex is capable of generating bioluminescence in the presence of a substrate (e.g., coelenterazine, furimazine, etc.), and the modified dehalogenase complex is capable of binding to a haloalkane ligand.

In some embodiments, a tandem peptide tag herein comprises a first component that is a component of a bioluminescent complex, as described herein and throughout U.S. Pat. No. 9,797,889 and U.S. application Ser. No. 16/439,565, both of which are herein incorporated by reference in their entireties. Any of the components of the bioluminescent complexes described herein or incorporated by reference may find use in the tandem peptide tags herein. In particular embodiments, a tandem peptide tag herein comprises a peptide component of a bioluminescent complex.

In some embodiments, a tandem peptide tag herein comprises a second component that is a component of a modified dehalogenase complex, as described herein and throughout U.S. Prov. App. No. 63/338,323, and U.S. application Ser. No. 18/312,117, both of which are herein incorporated by reference in their entireties. Any of the components of the modified dehalogenase complexes described herein or incorporated by reference may find use in the tandem peptide tags herein. In particular embodiments, a tandem peptide tag herein comprises a peptide component of a modified dehalogenase complex.

In some embodiments, a tandem peptide tag comprising a component of a bioluminescent complex and a component a modified dehalogenase complex is fused or otherwise linked to a peptide or protein of interest. In some embodiments, the tandem peptide tag is expressed as a fusion with the peptide or protein of interest. In some embodiments, the peptide or protein of interest is a cellular target to be detected, quantified, or otherwise characterized by using the systems and methods herein.

In some embodiments, a tandem peptide tag herein comprises at least 70% (e.g., 70%, 75%, 80%, 85%, 90%, 95%, 100%, or ranges therebetween) sequence identity to one or more of SEQ ID NOS: 3062-3063, 3067-3078, 3092, 3094-3109, and 4177-4181. In some embodiments, the peptide component of a modified dehalogenase portion (e.g., SmHT) of a tandem peptide tag herein comprises one or more substitutions relative to a reference SmHT peptide of any of the SmHT variant peptides herein and/or SmHT portions of one or more of SEQ ID NOS: 3062-3063, 3067-3078, 3092, and 3094-3109. In some embodiments, the peptide component of a bioluminescent complex portion (e.g., HiBiT) of a tandem peptide tag herein comprises one or more substitutions relative to a reference HiBiT peptide of any of the HiBiT variant peptides herein and/or HiBiT portions of one or more of SEQ ID NOS: 3062-3063, 3067-3078, 3092, 3094-3109, and 4177-4181.

### Tandem Polypeptide Reporters

In some embodiments, provided herein are a tandem polypeptide reporters comprising a polypeptide component of a bioluminescent complex fused to a polypeptide component of a modified dehalogenase complex. In some embodiments, the polypeptide component of a bioluminescent complex is capable of forming a bioluminescent complex with a peptide component of the bioluminescent complex upon interaction therebetween (e.g., facilitated or unfacilitated). In some embodiments, the polypeptide component of a modified dehalogenase complex is capable of forming a modified dehalogenase complex with a peptide component of the modified dehalogenase complex upon interaction therebetween (e.g., facilitated or unfacilitated). In some embodiments, the bioluminescent complex and modified dehalogenase complex form upon interaction (facilitated or unfacilitated) of the tandem polypeptide reporter and a tandem peptide tag (e.g., comprising the peptide component of the bioluminescent complex fused to the peptide component of the modified dehalogenase complex). In some embodiments, the bioluminescent complex can generate bioluminescence in the presence of a substrate (e.g., coelenterazine, furimazine, etc.), and the modified dehalogenase complex is capable of binding to a haloalkane ligand.

In some embodiments, a tandem polypeptide reporter herein comprises a first component that is a component of a bioluminescent complex, as described herein and throughout U.S. Pat. No. 9,797,889 and U.S. application Ser. No. 16/439,565, both of which are herein incorporated by reference in their entireties. Any of the components of the bioluminescent complexes described herein or incorporated by reference may find use in the tandem polypeptide reporters herein. In particular embodiments, a tandem polypeptide reporter herein comprises a polypeptide component of a bioluminescent complex.

In some embodiments, a tandem polypeptide reporter herein comprises a second component that is a component of a modified dehalogenase complex, as described herein and throughout U.S. Prov. App. No. 63/338,323, and U.S. application Ser. No. 18/312,117, both of which are herein incorporated by reference in their entireties. Any of the components of the modified dehalogenase complexes described herein or incorporated by reference may find use in the tandem polypeptide reporters herein. In particular embodiments, a tandem polypeptide reporter herein comprises a polypeptide component of a modified dehalogenase complex.

In some embodiments, a tandem polypeptide reporter herein comprises at least 70% (e.g., 70%, 75%, 80%, 85%, 90%, 95%, 100%, or ranges therebetween) sequence identity to one or more of SEQ ID NOS: 3110-4064. In some embodiments, the polypeptide component of a modified dehalogenase portion (e.g., LgHT) of a tandem polypeptide reporter herein comprises one or more substitutions relative to a reference LgHT peptide of any of the LgHT variant peptides herein and/or LgHT portions of one or more of SEQ ID NOS: 3110-4064. In some embodiments, the peptide component of a bioluminescent complex portion (e.g., HiBiT) of a tandem polypeptide reporter herein comprises one or more substitutions relative to a reference HiBiT peptide of any of the HiBiT variant peptides herein and/or HiBiT portions of one or more of SEQ ID NOS: 3110-4064.

### Other Tandem Tags and Reporters

Embodiments herein are predominantly described as tags comprising fused peptide components of bioluminescent and modified dehalogenase complexes and reporters comprising fused polypeptide components of the complexes. However, embodiments within the scope herein also include tags (e.g., for linking/fusing to a target) comprising: the polypeptide components of the bioluminescent and modified dehalogenase complexes, the polypeptide component of the bioluminescent complex and the peptide component of the modified dehalogenase complex, or the peptide component of the bioluminescent complex and the polypeptide component of the modified dehalogenase complex; and reporters comprising the peptide components of the bioluminescent and modified dehalogenase complexes, the polypeptide component of the bioluminescent complex and the peptide component of the modified dehalogenase complex, or the peptide component of the bioluminescent complex and the polypeptide component of the modified dehalogenase complex. Any suitable arrangement of the components described herein is within the scope.

### Linkers

Various fusions are described herein. In some embodiments, the components of the fusion are directly linked (e.g., C-terminus to N-terminus). In other embodiments, the fusions herein comprise peptide or polypeptide linkers between the components. Such linkers may be of any suitable sequence and up to 100 amino acids in length (e.g., 1, 2, 3, 4, 5, 6, 7, 8, 9, 10, 15, 20, 25, 30, 35, 40, 445, 50, 60, 70, 80, 90, 100, or ranges therebetween). In some embodiments, a tandem peptide tag herein comprises a linker sequence between the peptide component of the modified dehalogenase complex and the peptide component of the bioluminescent complex. In some embodiments, a tandem peptide tag is linked to a target element by a suitable linker sequence. In some embodiments, a tandem detector herein comprises a linker sequence between the polypeptide component of the modified dehalogenase complex and the polypeptide component of the bioluminescent complex. Exemplary linker sequences are provided in the tandem tags and tandem detectors exemplified herein, but other linker sequences are within the scope herein. Any peptide/polypeptide sequence capable of linking the components of a construct herein is within the scope herein, including but not limited to those specifically exemplified herein, both in terms of length and amino acid composition.

### Haloalkyl Ligands

As described herein, the spHT systems (e.g., within the fusions herein) utilize haloalkane ligands. In some embodiments, the ligand is of formula (I): R-linker-A-X, wherein R is a solid surface, one or more functional groups, or absent, wherein the linker is a multiatom straight or branched chain including C, N, S, or O, or a group that comprises one or more rings, e.g., saturated or unsaturated rings, such as one or more aryl rings, heteroaryl rings, or any combination thereof, wherein A-X is a ligand for a dehalogenase, hydrolase, HALOTAG, or a spHT system herein (e.g., wherein A is (CH₂)₄₋₂₀ and X is a halide (e.g., Cl or Br)). Suitable ligands are described, for example, in U.S. Pat. Nos. 11,072,812; 11,028,424; 10,618,907; and 10,101,332; incorporated by reference in their entireties. In certain embodiments, X of formula (I) is a methylsulfonamide or trifluoromethylsulfonamide, rather than a halide; such an embodiment results in an exchangeable ligand that reversibly binds to a modified dehalogenase (e.g., HALOTAG). Such ligands are described in, for example, Kompa et al. J. Am. Chem. Soc. 2023, 145, 5, 3075-3083; incorporated by reference in its entirety.

In some embodiments, R is one or more functional groups (such as a fluorophore, biotin, luminophore, or a fluorogenic or luminogenic molecule). Exemplary functional groups for use in the invention include, but are not limited to, an amino acid, protein, e.g., enzyme, antibody or other immunogenic protein, a radionuclide, a nucleic acid molecule, a drug, a lipid, biotin, avidin, streptavidin, a magnetic bead, a solid support, an electron opaque molecule, chromophore, MRI contrast agent, a dye, e.g., a xanthene dye, a calcium sensitive dye, e.g., 1-[2-amino-5-(2,7-dichloro-6-hydroxy-3-oxy-9-xanthenyl)-phenoxy]-2-(2′-am-ino-5′-methylphenoxy)ethane-N,N,N′,N′-tetraacetic acid (Fluo-3), a sodium sensitive dye, e.g., 1,3-benzenedicarboxylic acid, 4,4′-[1,4,10,13-tetraoxa-7,16-diazacyclooctadecane-7,16-diylbis(5-methoxy-6,2-benzofurandiyl)]bis (PBFI), a NO sensitive dye, e.g., 4-amino-5-methylamino-2′,7′-difluorescein, or other fluorophore. In one embodiment, the functional group is an immunogenic molecule, i.e., one which is bound by antibodies specific for that molecule.

In some embodiments, ligands of the invention are permeable to the plasma membranes of cells (i.e., capable of passing from the exterior of a cell (e.g., eukaryotic, prokaryotic) to the cellular interior without chemical, enzymatic, or mechanical disruption of the cell membrane).

In some embodiments, ligands herein comprise a cleavable linker, for example, those described in U.S. Pat. No. 10,618,907; incorporated by reference in its entirety.

In some embodiments, a ligand comprises a fluorescent functional group (R). Suitable fluorescent functional groups include, but are not limited to: stilbazolium derivatives (Marquesa et al. Mechanism-Based Strategy for Optimizing HaloTag Protein Labeling. ChemRxiv. Cambridge: Cambridge Open Engage; 2021; incorporated by reference in its entirety), xanthene derivatives (e.g., fluorescein, rhodamine, Oregon green, eosin, Texas red, etc.), cyanine derivatives (e.g., cyanine, indocarbocyanine, oxacarbocyanine, thiacarbocyanine, merocyanine, etc.), naphthalene derivatives (e.g., dansyl and prodan derivatives), oxadiazole derivatives (e.g., pyridyloxazole, nitrobenzoxadiazole, benzoxadiazole, etc.), pyrene derivatives (e.g., cascade blue), oxazine derivatives (e.g., Nile red, Nile blue, cresyl violet, oxazine 170, etc.), acridine derivatives (e.g., proflavin, acridine orange, acridine yellow, etc.), arylmethine derivatives (e.g., auramine, crystal violet, malachite green, etc.), tetrapyrrole derivatives (e.g., porphin, phtalocyanine, bilirubin, etc.), CF dye (Biotium), BODIPY (Invitrogen), ALEXA FLOUR (Invitrogen), DYLIGHT FLUOR (Thermo Scientific, Pierce), ATTO and TRACY (Sigma Aldrich), FluoProbes (Interchim), DY and MEGASTOKES (Dyomics), SULFO CY dyes (CYANDYE, LLC), SETAU AND SQUARE DYES (SETA BioMedicals), QUASAR and CAL FLUOR dyes (Biosearch Technologies), SURELIGHT DYES (APC, RPE, PerCP, Phycobilisomes)(Columbia Biosciences), APC, APCXL, RPE, BPE (Phyco-Biotech), autofluorescent proteins (e.g., YFP, RFP, mCherry, mKate), quantum dot nanocrystals, etc.

In some embodiments, a ligand comprises a fluorogenic functional group (R). A fluorogenic functional group is one that produces and enhanced fluorescent signal upon binding of the ligand to a target (e.g., binding of a haloalkane to a modified dehalogenase). By producing significantly increased fluorescence (e.g., 10×, 31×, 50×, 100×, 310×, 500×, 1000×, or more) upon target engagement, the problem of background signal is alleviated. Exemplary fluorogenic dyes for use in embodiments herein include the JANELIA FLUOR family of fluorophores, such as:

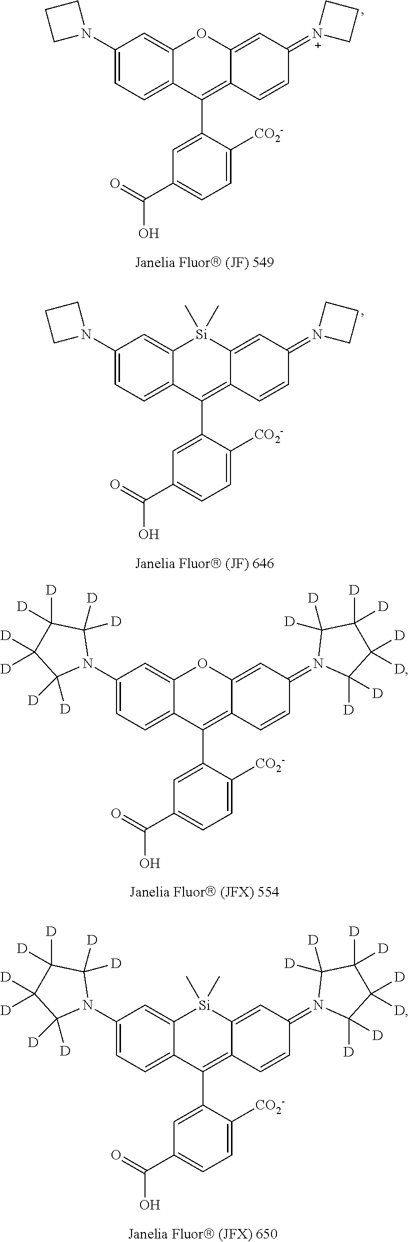

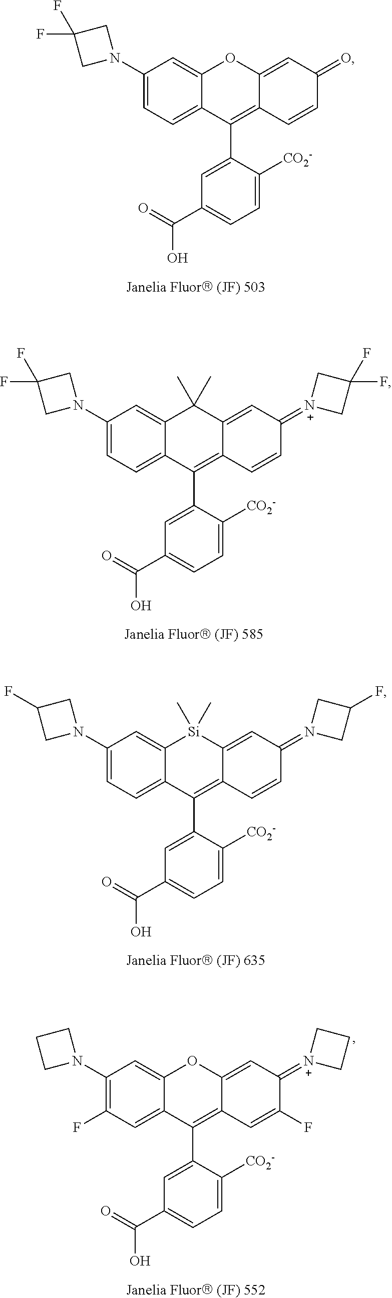

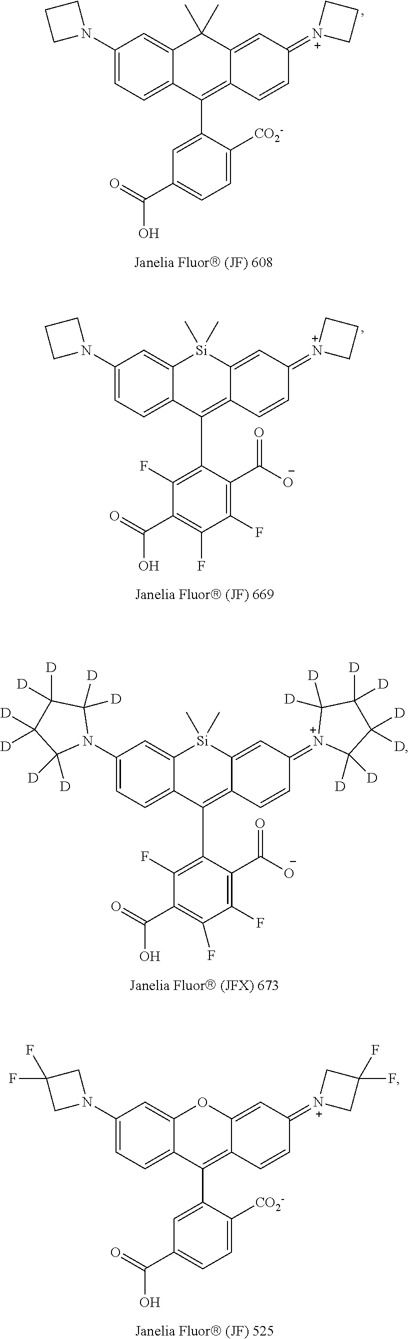

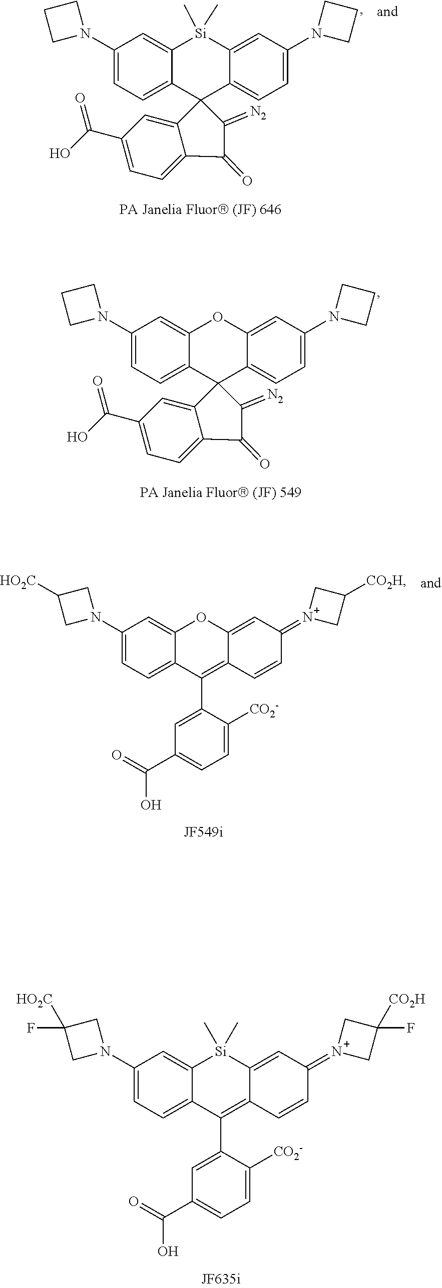

(see, e.g., U.S. Pat. Nos. 9,933,417; 10,018,624; 10,161,932; and 10,495,632; each of which is incorporated by reference in their entireties). In some embodiments, exemplary conjugates of JANELIA FLUOR 549 and JANELIA FLUOR 646 with haloalkane ligands for modified dehalogenase (e.g., HALOTAG) are commercially available (Promega Corp.). The use and design of fluorogenic functional groups, dyes, probes, and ligands is described in, for example, Grimm et al. Nat Methods. 3117 October; 14(10):987-994; Wang et al. Nat Chem. 3120 February; 12(2):165-172; incorporated by reference in their entireties.

In some embodiments a fluorophore or fluorogenic dye herein is a rhodamine and/or rhodol dye.

In some embodiments, the fluorophore (R) is of the structure:

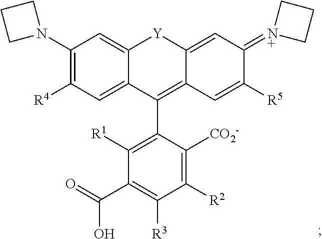

wherein Y is C, O, or Si, and wherein if Y is C or Si it is substituted with two CH₃ groups; wherein each of R¹, R², R³, R⁴, and R⁵ are independently H or F (e.g., R¹-R⁵ are all H; R¹-R⁵ are all F; R¹ and R² are F and R³ is H; R¹-R³ are F and R⁴-R⁵ are H, R¹ and R² are F and R³-R⁵ are H, etc.). In some embodiments, the azetidine are further substituted with one or two nonhydrogen substituents at the 3-position (e.g., CO²H, CH₃, F, etc.). In some embodiments, an exemplary compound herein is of the structure:

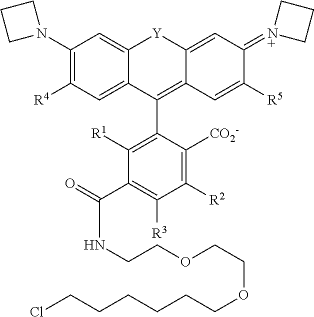

(wherein R¹⁻⁵ and Y are defined as above, Y and the azetidine are optionally substituted as above, and alternative linker and A-X groups are also within the scope).

In some embodiments, the fluorophore (R) is of the structure:

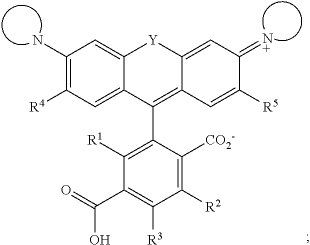

wherein Y is C, O, or Si, and wherein if Y is C or Si it is substituted with two CH₃ groups; wherein each of R¹, R², R³, R⁴, and R⁵ are independently H or F (e.g., R¹-R⁵ are all H; R¹-R⁵ are all F; R¹ and R² are F and R³ is H; R¹-R³ are F and R⁴-R⁵ are H, R¹ and R² are F and R³-R⁵ are H, etc.); and wherein each

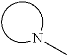

comprises an azetidine or a fully deuterated pyrrolidine. In some embodiments, the azetidine, when present are further substituted with one or two nonhydrogen substituents at the 3-position (e.g., CO²H, CH₃, F, etc.). In some embodiments, an exemplary compound herein is of the structure:

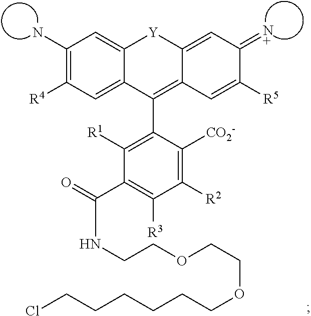

wherein R¹⁻⁵, Y, and

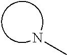

are defined as above, Y and the azetidine are optionally substituted as above, and alternative linker and A-X groups are also within the scope.

In some embodiments, the linker is a multiatom straight or branched chain including C, N, S, or O, or a group that comprises one or more rings, e.g., saturated or unsaturated rings, such as one or more aryl rings, heteroaryl rings, or any combination thereof. In some embodiments, the linker comprises a combination of —O(CH₂)₂— —(CH2)O—, —CH₂—, —NHC(O)O—, —OC(O)NH—, NHC(O)—, and —C(O)NH—. In some embodiments, the linker is 5 to 50 (e.g., 5, 10, 15, 20, 25, 30, 35, 40, 45, 50, or ranges therebetween) atoms in length. In some embodiments, the length of the linker for tethering the alkylhalide to R allows for optimization of proximity and geometry (e.g., for binding of a modified dehalogenase complex to the alkylhalide, for function of the functional moiety (e.g., fluorescence), etc.). The scope of embodiments herein is not limited by the types of linkers available. The fluorophore and A-X may be linked either directly (e.g., linker consists of a single covalent bond) or linked via a suitable linker. Embodiments are not limited to any particular linker group. A variety of linker groups are contemplated, and suitable linkers could comprise, but are not limited to, alkyl groups, methylene carbon chains, ether, polyether, alkyl amide linker, a peptide linker, a modified peptide linker, a Poly(ethylene glycol) (PEG) linker, a streptavidin-biotin or avidin-biotin linker, polyaminoacids (e.g., polylysine), functionalized PEG, polysaccharides, glycosaminoglycans, dendritic polymers (WO93/06868 and by Tomalia et al. in Angew. Chem. Int. Ed. Engl. 29:138-175 (1990), herein incorporated by reference in their entireties), PEG-chelant polymers (W94/08629, WO94/09056 and WO96/26754, herein incorporated by reference in their entireties), oligonucleotide linker, phospholipid derivatives, alkenyl chains, alkynyl chains, disulfide, or a combination thereof. In some embodiments, the linker is cleavable (e.g., enzymatically (e.g., TEV protease site), chemically, photoinduced, etc. In some embodiments, the cleavable linker comprises an allyl-heteroatom group or a propargyl-heteroatom group, such as the linkers described, for example, in U.S. application Ser. No. 16/813,295; incorporated by reference in its entirety.

The haloalkyl ligands herein comprise a haloalkyl group, a linker, and a functional group. Exemplary compounds comprising a rhodamine dye linked to a haloalkane include:

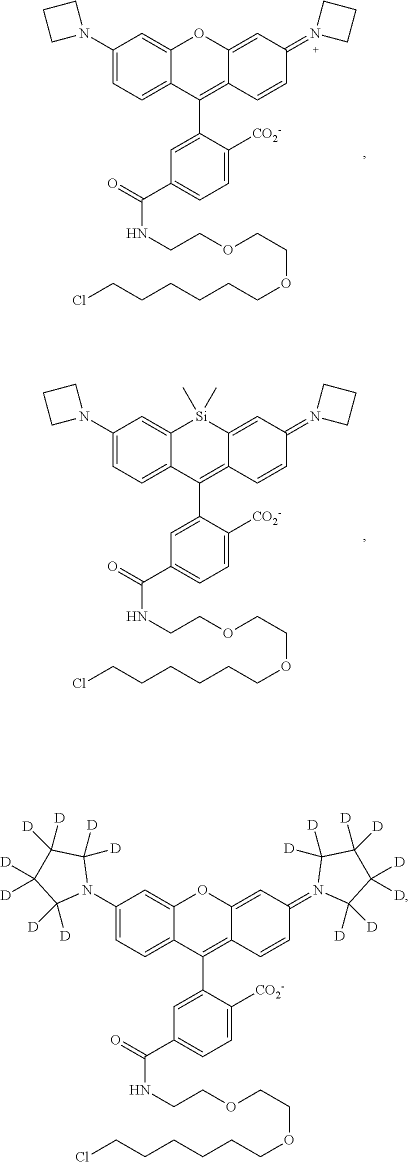

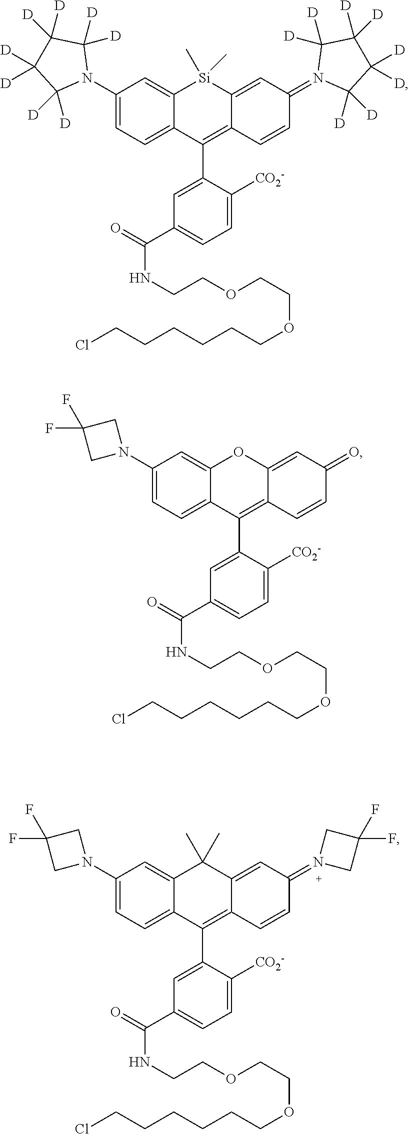

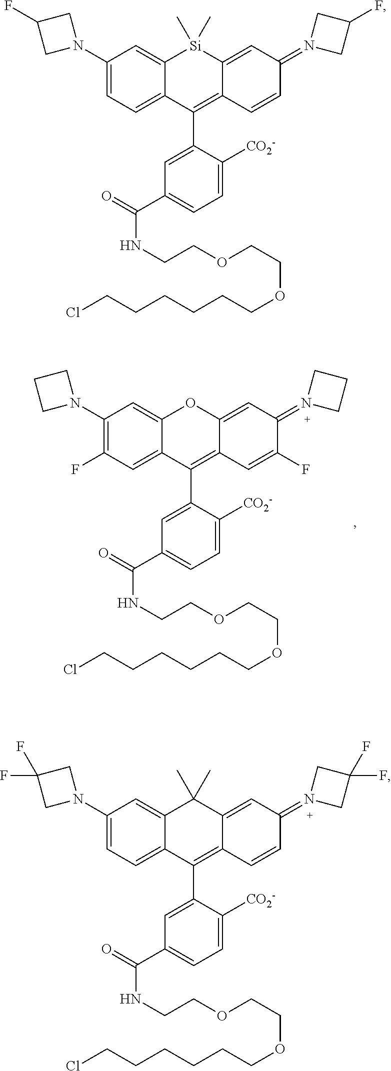

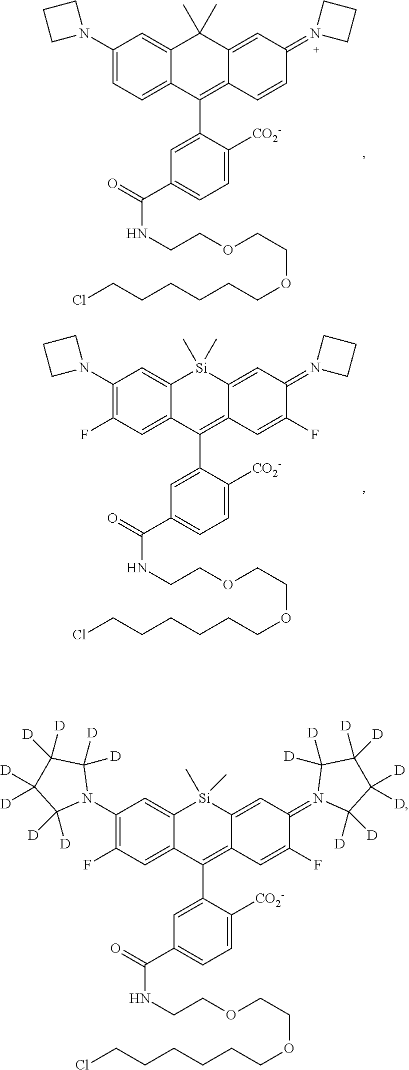

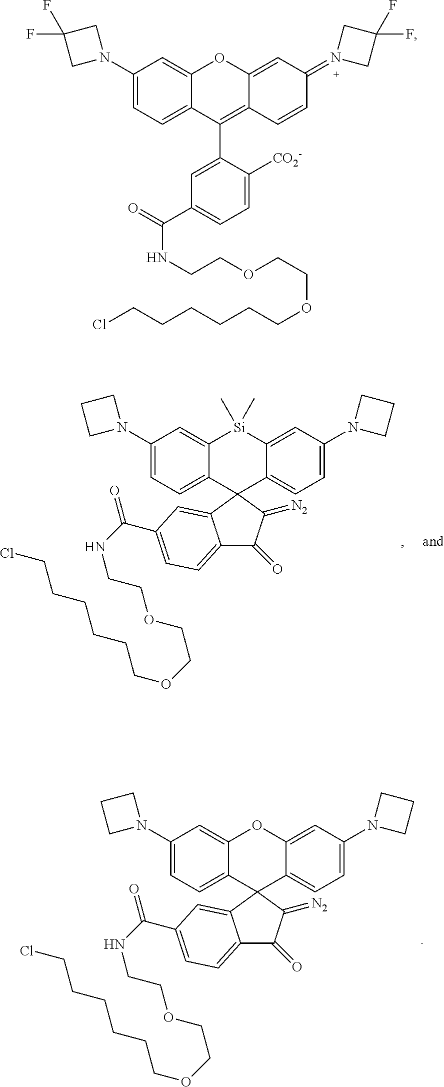

Other ligands comprising different halides (e.g., Br), different length of composition of A, and different R groups (e.g., biotin, other fluorophores, etc.) are within the scope herein. Other non-limiting examples of haloalkyl ligands that may find use in embodiments herein include:

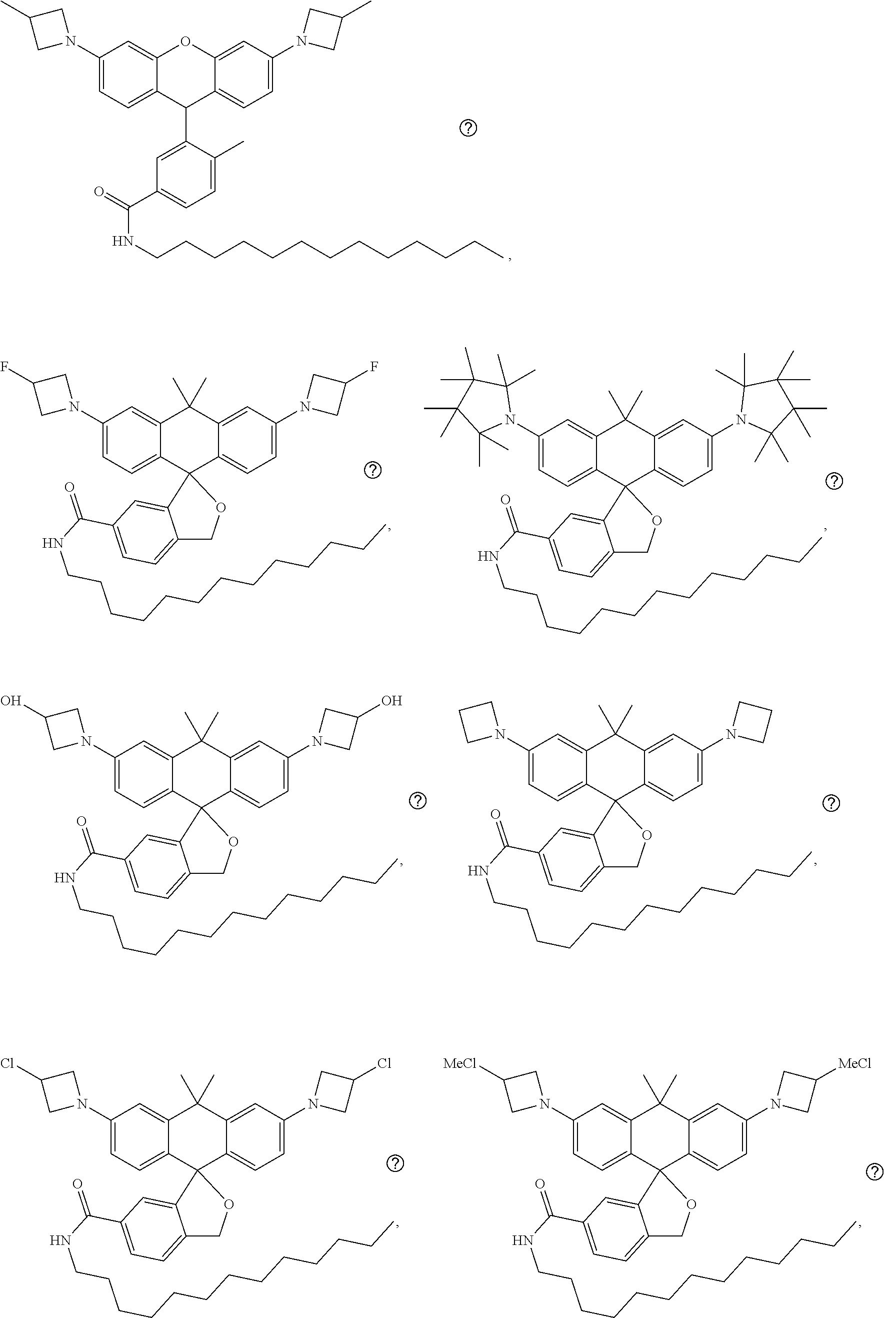

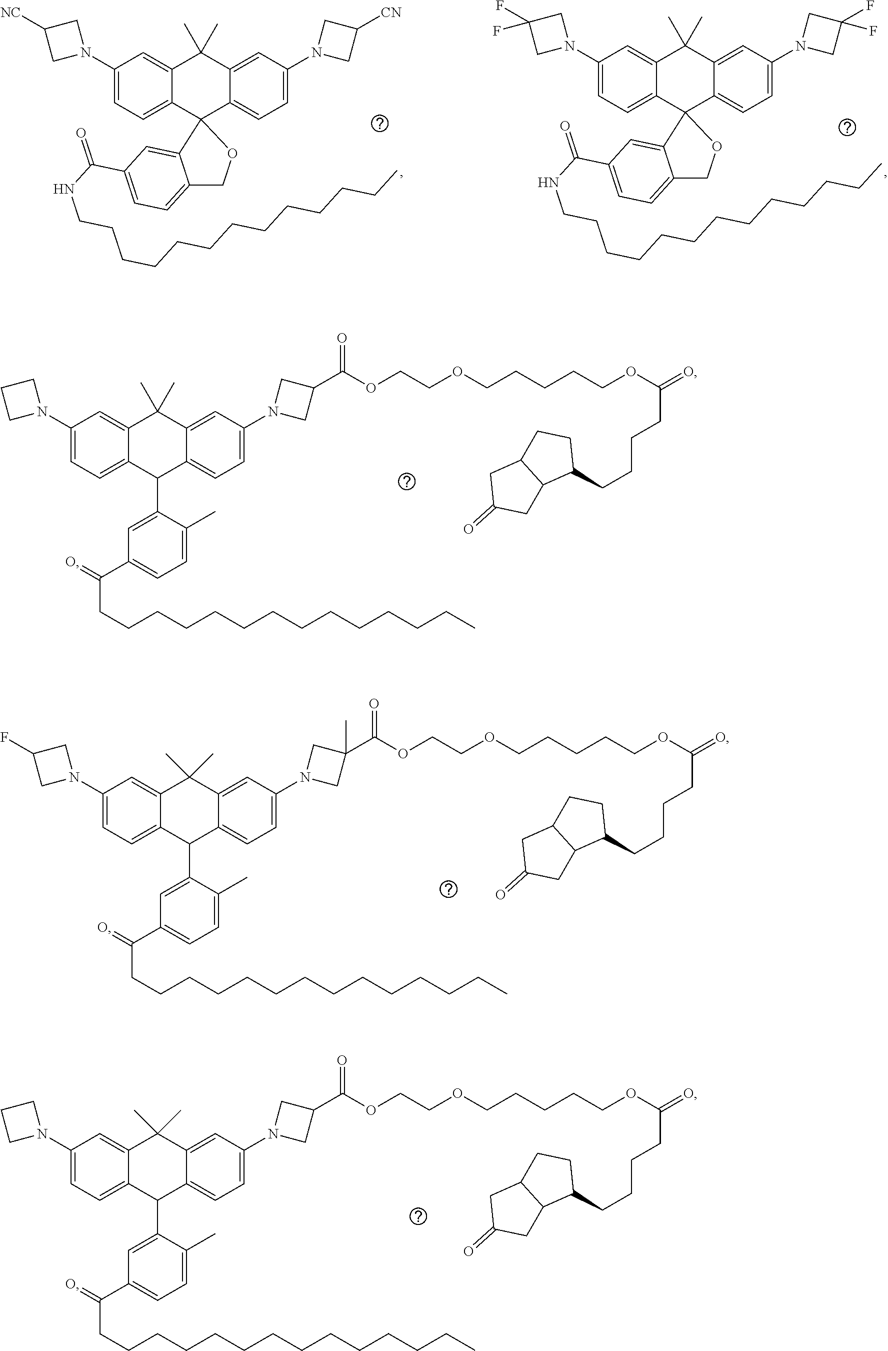

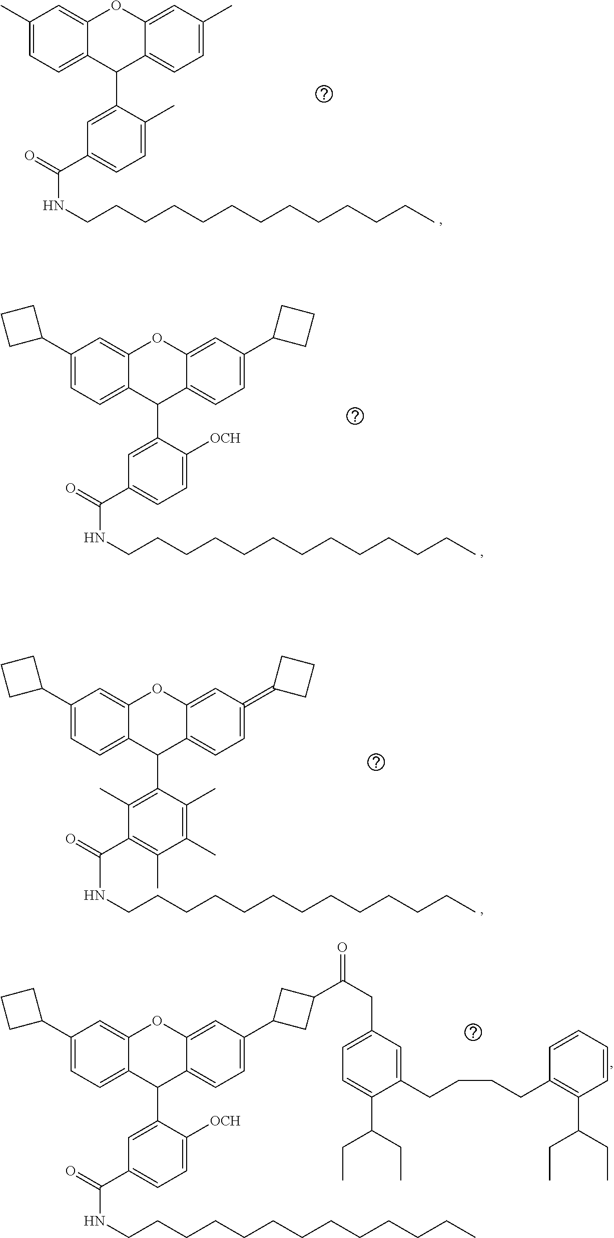

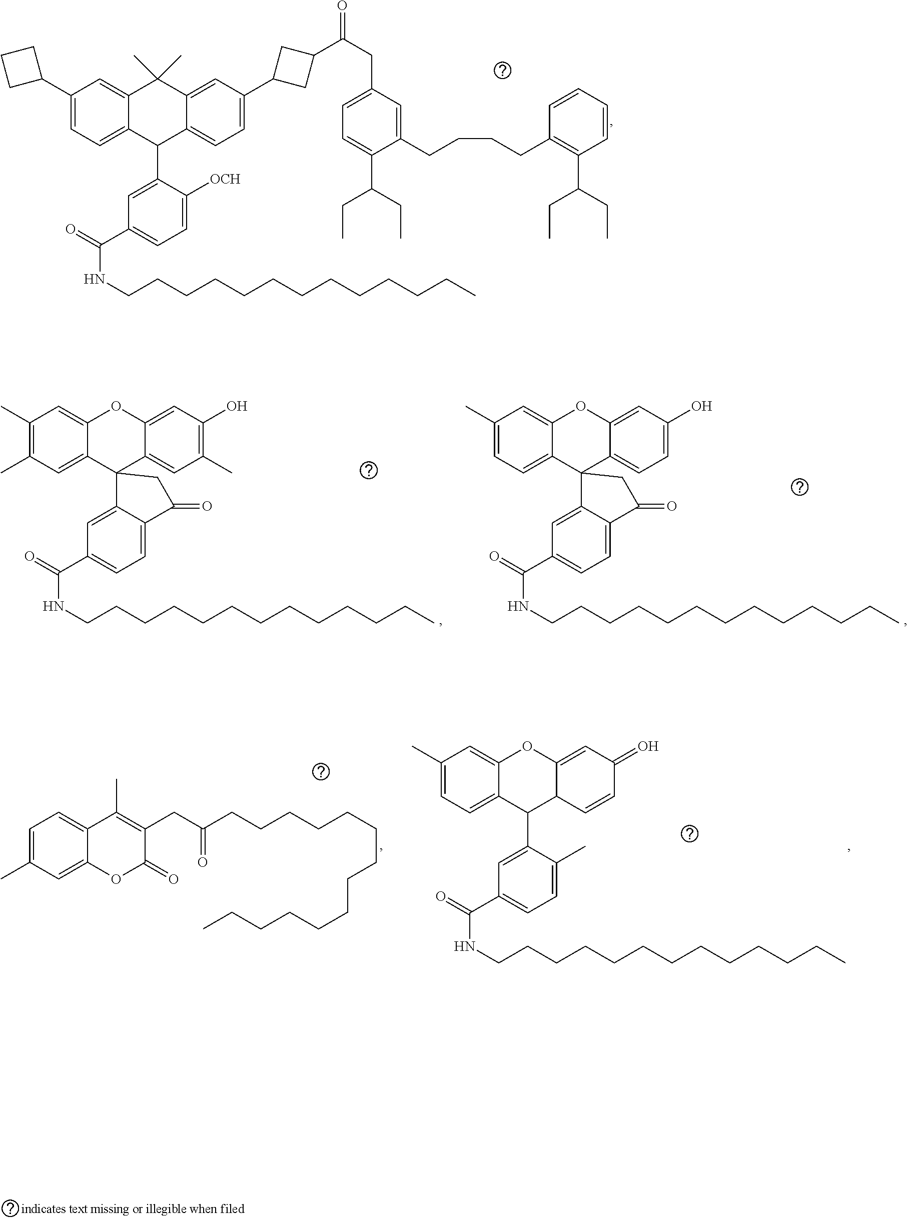

etc. (wherein alternative R, linker, and A-X groups are also within the scope).

### Bioluminescent Substrates

In some embodiments, the systems herein (comprising a bioluminescent complex and/or components thereof) utilize an imidazopyrazine luminophore substrate to generate bioluminescence. In some embodiments, the substrate is coelenterazine:

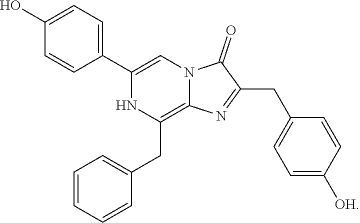 [SMILES: O=c1c(Cc2ccc(O)cc2)nc2c(Cc3ccccc3)[nH]c(-c3ccc(O)cc3)cn1-2]

In some embodiments, the substrate is a coelenterazine derivative, such as, furimazine, furimazine analogs (e.g., fluorofurimazine), coelenterazine-n, coelenterazine-f, coelenterazine-h, coelenterazine-hcp, coelenterazine-cp, coelenterazine-c, coelenterazine-e, coelenterazine-fcp, bis-deoxycoelenterazine (“coelenterazine-hh”), coelenterazine-i, coelenterazine-icp, coelenterazine-v, and 2-methyl coelenterazine, in addition to those disclosed in WO 2003/040100; U.S. application Ser. No. 12/056,073 (paragraph [0086]); U.S. Pat. No. 8,669,103; U.S. Prov. App. No. 63/379,573; the disclosures of which are incorporated by reference herein in their entireties.

In some embodiments, the substrate is furimazine:

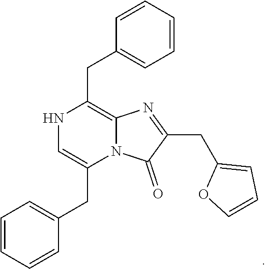 [SMILES: O=c1c(Cc2ccco2)nc2c(Cc3ccccc3)[nH]cc(Cc3ccccc3)n1-2]

In some embodiments the substrate is fluorofurimazine:

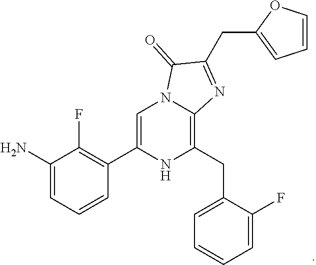 [SMILES: Nc1cccc(-c2cn3c(=O)c(Cc4ccco4)nc-3c(Cc3ccccc3F)[nH]2)c1F]

### Additional Reporter Peptides

In some embodiments, a bioluminescent and/or modified dehalogenase complex is a tripartite or multipartite complex (e.g., comprising three or more peptide/polypeptide components). In such embodiments, a first component of the complex may be present within the tandem peptide tag, a second component of the complex may be present within the tandem polypeptide reporter, and a third (or fourth or more) component of the complex may be present as an additional reporter. In some embodiments, interaction of the additional reporter, tandem polypeptide reporter, and tandem peptide tag (facilitated or unfacilitated) results in formation of the modified dehalogenase and bioluminescent complexes.

The additional reporter may be present as a fusion with one or more additional elements or amino acid sequences (e.g., interaction element, target protein, etc.). In some embodiments, the additional amino acid sequence is selected from the group consisting of a protein of interest, an interaction element, a co-localization element, and a binding moiety. In some embodiments, the additional amino acid sequence is a binding moiety selected from the group consisting of an antibody (polyclonal, monoclonal, and/or recombinant), antibody fragment, protein A, an Ig binding domain of protein A, protein G, an Ig binding domain of protein G, protein A/G, an Ig binding domain of protein A/G, protein L, a Ig binding domain of protein L, protein M, an Ig binding domain of protein M, peptide nucleic acid, DARPin, affimer, a purified protein (either the analyte itself or a protein that binds to the analyte), and analyte binding domain(s) of proteins. In some embodiments, the additional amino acid sequence is a first interaction polypeptide that is configured to form a complex with a second interaction polypeptide upon contact of the first interaction polypeptide and the second interaction polypeptide. In some embodiments, the additional amino acid sequence is a first co-localization polypeptide that is configured to co-localize within a cellular compartment, a cell, a tissue, or an organism within a with a second co-localization polypeptide. In some embodiments, the additional amino acid sequence is a protein of interest and is a candidate drug target. In some embodiments, the additional amino acid sequence is a fluorescent protein (e.g., GFP and variants thereof), a luciferase (e.g., Firefly or Renilla, etc.), SPYTAG, SPYCATCHER, SNAP-tag, CLIP-tag, etc.

### Description of the Systems

In some embodiments, systems are provided comprising bioluminescent and modified dehalogenase complexes and the components thereof. In some embodiments, provided herein are protein complementation systems (and components thereof) capable of both luminescence and fluorescence detection based on combining split NanoLuc® and split HaloTag® technologies. Certain embodiments are described in connection split NANOLUC-based systems and components (e.g., NanoBiT, NanoTrip, smBiT, HiBiT, LgBiT, etc., and variants thereof) and split HALOTAG-based systems and components (e.g., spHT, SmHT, LgHT, etc. and variants thereof). Experiments have been conducted combining two small peptide tags from each system in tandem (e.g., SmHT-HiBiT) demonstrating the tandem peptide tag can facilitate complementation with either technology by adding their cognate large sequence, LgHT or LgBiT, both separately and simultaneously. Additionally, experiments have demonstrated fusion of the reporter polypeptides into a single tandem reporter polypeptide (e.g., LgHT-LgBiT). An advantage of the LgHT-LgBiT reporter polypeptide is that the high affinity of the HiBiT:LgBiT interaction facilitates the SmHT:LgHT complementation in the complex, thereby providing the functionality of a high-affinity/spontaneous split HaloTag® system.

In some embodiments, the tags and reporters herein are not limited to combinations of the peptide components and polypeptide components. For example, a peptide component of a NanoLuc®-based complementation system can be fused with a polypeptide component of a HaloTag®-based complementation system for complementation with a fusion of a NanoLuc®-based polypeptide component and a HaloTag®-based peptide component. Additionally, the various components can be combined in different configurations or orientations, such as SmHT-LgBiT and HiBiT-LgHT. In some embodiments, the fusions herein comprise linkers to facilitate optimized geometries for complementation, ligand/substrate binding, etc. In some embodiments, the sequences of the HaloTag®-based components and NanoLuc®-based components are not limited to the commercially- or publicly-available sequences of the HaloTag, NanoLuc, or binary multipartite complementation systems thereof.

In some embodiments herein, a peptide component of a bioluminescent complex is fused to a peptide components of a modified dehalogenase complex to form a tandem peptide tag. In some systems, methods, assays, etc., there are certain advantages of fusion configurations in which the two peptide components are fused as a tandem tag and the two polypeptide components are fused as a tandem reporter. The small tag fusion is still a peptide-sized sequence (e.g., 28 amino acids (17 AA SmHT+11 AA HiBiT)). This is still within the size range (84 nucleotides) for convenient synthesis as a single-stranded donor DNA oligonucleotide and efficient delivery during CRISPR genome engineering to introduce the tandem tag on an endogenous protein target. In some embodiments, there is also the potential benefit of a small tag being minimally perturbing to its target protein fusion partner. Exemplary configurations of this tandem peptide tag include different versions including SmBiT, HiBiT, and other variants to those sequences that alter their affinity. Hundreds of sequence variants of the SmHT, all of which remain low affinity to LgHT and are functional in the complementation system. In some embodiments, any SmHT variants having desirable properties for both in vivo and in vitro use, alone and in tandem with the SmBiT/HiBiT (or variants thereof), such as altered charge and hydrophobicity or cysteine/arginine residues that enable chemical modification, find use in embodiments herein. Since HiBiT, for example, is positively charged, it may be preferable to have positively charged variants of the SmHT in the context of the SmHT-HiBiT fusion tag to maintain similar physical attributes across the length of the peptide tag. In some embodiments, there are advantages to introducing linkers between the SmHT and SmBiT/LgBiT in the fusion tag or changing their orientation, to minimize any potential stearic limitations on complementation, orientation, or flexibility. Alternative orientations include using each tag on a separate terminus (N- or C-) of the target protein, for example, in a protein where the termini are in close proximity, whereby the LgHT-LgBiT detector polypeptide could still form a complemented complex with both tags simultaneously.

In some embodiments, the user may only desire to use one technology, and the other is unused/noninterfering. For example, in some embodiments, the fluorescence component of embodiments of the present technology allows for fluorescent cell sorting after CRISPR editing to more effectively/rapidly isolate cells that contain the tag, whereafter a user may primarily be interested in using the HiBiT portion of the tag for downstream measurement of biological events inside the cells. Yet another example is the concept that the high affinity LgBiT:HiBiT interaction spontaneously facilitates interaction of the SmHT:LgHT in the complemented complex, effectively creating a novel high affinity version of split HaloTag. In this case, the luminescence technology is not used other than to bring the split HaloTag® pieces together if the user only desires to have a spontaneously completing split HaloTag® system for targeting chloroalkanes to a tagged protein target. In another embodiment the HiBiT component of the tandem peptide tag will be detected with LgBiT alone (i.e., not fused to LgHT). In situations where there is excess LgBiT-LgHT detector in the cell, it may be possible to amplify signal. This could happen if one molecule of detector binds and forms a complex to the dual peptide tag and the LgHT sequence becomes fluorescently-labeled but then dissociates from the dual peptide tag. This would allow for a previously non-labeled LgBiT-LgHT to bind to the dual peptide tag and become labeled.

In some embodiments of the present technology utilizing a LgHT-LgBiT polypeptide or variants thereof, since such a fusion imparts a versatile range of functionalities toward the detection and manipulation of the tagged target protein, the LgHT-LgBiT fusion is primarily envisioned in a configuration that fuses the two proteins at their termini. In some embodiments, the properties and performance of the LgHT-LgBiT fusion could be further optimized through modification of the linker between them and their relative orientation (LgHT-LgBiT versus LgBiT-LgHT). Other potential configurations include circular permutation of one or both proteins in the fusion or insertion of one sequence/permuted sequence inside the other. The latter is similar to our work creating “chimeras” between HaloTag and NanoLuc/LgBiT/LgTrip, where the luminescence protein is inserted within HaloTag, providing the benefit of a close proximity and specific geometry between their respective substrate binding sites resulting in exceptionally efficient energy transfer for BRET. A “split chimera” version using LgHT-LgBiT could be similarly configured, potentially providing the correct orientation to still form a complementation complex with the SmHT-HiBiT peptide tag.

Some advantages of the invention configuring the LgHT-LgBiT (or variants thereof) into a fusion polypeptide include its introduction as a single detection reagent, compatibility with all NanoLuc® and HaloTag® substrates/ligands, sequential or simultaneous luminescence/fluorescence measurement, and multiple different measurements of a tagged target (quantification, interaction, localization, etc.). As a single detection reagent, the LgHT-LgBiT fusion polypeptide (or variants thereof) can be delivered into an assay or cell as a purified protein, encoded on a plasmid or mRNA, or uniformly expressed from the chromosome in a stable or CRISPR cell line. As a purified protein, it can be fused or conjugated to a solid support/bead/surface or other protein, such as an antibody. The latter is advantageous for biochemical or immunoassays where the LgHT-LgBiT (or variants thereof) would be conjugated to an antibody and configured into a detection reagent that relies on recognition of the antibody target in an in vitro or diagnostic format.

The compatibility of the LgHT-LgBiT polypeptide (or variants thereof) with all NanoLuc® substrates and HaloTag® ligands provides exceptional versatility in the mode of detection (luminescence or fluorescence), emission wavelengths (UV to IR), and functionality (non-fluorescent HaloTag® ligands). An example application would be an animal model such as a mouse expressing the LgHT-LgBiT detection polypeptide (or variants thereof), in which introduction of a protein target tagged with the SmHT-HiBiT tag (e.g. through viral infection where small tags encoded on their genome are advantageous) enables the sensitive detection offered with low-background luminescence to quantify the target protein's abundance while the far-red, brain-permeable fluorescent HaloTag® ligands enable sophisticated neuronal imaging at another wavelength (with the potential for BRET). This example highlights the tradeoffs currently being made by researchers in animal imaging, where one detection technology has some but not all benefits that are solved by embodiments herein which enable both in a single model system without changing tags or reengineering cells/animals/hosts. For animal imaging, already existing studies showing targeted PET, SPECT, sensors, and MRI highlight the diversity of applications. Each of these can be combined with bioluminescence imaging with the tandem peptide tag.

In some embodiments, the systems herein find use with non-fluorescent HaloTag® ligands. For example, biotin chloroalkane ligands allow capture of dual-tagged target proteins. Another application is in the targeting of molecules for inducing protein proximity, where a particularly relevant example is targeted chimeras molecules (PROTACs, LyTACs, PhosTACs, AuTACs, etc.) and molecular glues. Since these molecules can be configured as chloroalkanes (i.e., HaloPROTACs), they offer a specific use case where this invention is novel. For example, a target protein tagged with SmHT-HiBiT can be monitored in real time for abundance with the luminescence activity of the LgHT-LgBiT detector, whereupon binding of a HaloPROTAC to the complex through the SmHT:LgHT interaction initiates recruitment of an E3 ligase, causing ubiquitination and degradation by the proteosome and subsequent decrease in the real-time luminescence signal. This highlights the simultaneous use of both split NanoLuc® and split HaloTag® capabilities of the technology in a way not currently possible with them separately. A HaloTag®-HiBiT tag could be used for this purpose, although there are examples of increased target protein expression and stability when the full-length HaloTag is used as fusion tag, which is particularly detrimental under conditions where the stability/degradation of the target is being studied, in addition to the need to add a much larger HaloTag®-HiBiT tag onto their target protein. Therefore, the capability of the SmHT-HiBiT to be minimally disruptive to the protein target while also enabling its manipulation through complementation with the LgHT-LgBiT detector is specific to this invention.

In some embodiments, the technologies herein, specifically the fluorescence output component, provides a more efficient workflow for engineering HiBiT-tagged endogenous gene cell lines through the use of cell sorting. The ability to sort positive cells (tagged gene of interest) in high-throughput could greatly diminish the amount of time and effort that goes into manually screening for positive pools or clones. This could be particularly impactful when working with targets that are difficult to modify using CRISPR editing or when working with difficult to propagate cells such as primary cells. A fluorescence ligand can be used for screening cells or pools of cells by fluorescence-activated cell sorting, and then when a clone or pool of clones is identified either the same ligand or a different ligand can be used for actual imaging experiments on cells. In some embodiments, the systems herein find use with various surface display technologies.

In certain embodiments, biomolecules, such as proteins/DNA/RNA, are immobilized to the surfaces of cells, microbeads, or other surfaces through a fusion to the DualTag or Dual Detector, taking advantage of the system's high affinity and very low rate of dissociation. In some embodiments, the displaying cells or microbeads are used as a platform for high-throughput methods of capture or measurement such as magnetic bead sorting or flow cytometry, respectively, to measure binding or modification of the fused target analyte of interest. Examples of target analytes include examples of various biomolecules such as protein variants, linker variants, small molecules, nucleic acids, or binding proteins. Such embodiments further allow for the concurrent employment of additional reporters, stains, tags, or markers on the surface of the cells or microbeads to achieve multiplex detection of signals on the surface, such as in the case of protein:protein interaction between two tagged/labeled species. The distinctive feature of the systems herein is provided not due to the high affinity interaction and slow dissociation of the tandem detector for the tandem peptide tag, but also in the capability to facilitate fluorescence detection via labeling with a fluorescent HaloTag® ligand, where a fluorescence signal is particularly useful for measurement with high-speed cell or bead cytometry or sorting applications.

In some embodiments, the capacity of the systems herein to facilitate cell surface display finds use in biological assays, for example, for monitoring cell surface receptors. Cell surface receptors, such as GPCRs, are valuable drug targets and heavily utilized for manipulating cell behavior and physiology. These receptors can be genetically modified to express the be linked to a tandem peptide tag herein. The small size and minimum expectation of perturbation of the tandem peptide tags herein allows for the display of the tandem tag externally on the cell surface or internally into the cytoplasm. Upon binding with a tandem detector herein, such systems enables both luminescence and fluorescence detection modalities. Depending on the orientation of the tag, either internal or external, various assays are enabled. If displayed externally, the systems herein can be used to measure receptor interactions, internalization, or dimerization when a tandem detector is provided exogenously as a purified protein, for example. If displayed internally, the systems herein can be used to measure post-translational modifications, protein-protein, and cell signaling pathways when, for example, a tandem detector is co-expressed inside the cell.

In some embodiments, the systems herein find use in monitoring dynamic cell entry. The entry into a cell of a tandem peptide tagged molecule and/or a tandem detector via a biological mechanism can be measured in the presence of the complementing fragment. An illustrative use involves viral infection, where the engineering of a tandem peptide tag onto a viral element would not compromise the formation of viral particles due to the tag's minimal size. This engineered virus, upon infecting a host cell that expresses a tandem detector, facilitates the release and subsequent detection of the tandem-tagged fusion protein as the tagged viral protein is released and associates with the tandem detector inside the cell. This technology provides for the real-time observation of viral entry into host cells using fluorescence imaging, including the application of HaloTag® ligands that specifically label the tandem peptide tag post-entry. Furthermore, such systems provide a drug discovery tool in the screening of neutralizing anti-viral drugs or antibodies by measuring the neutralization of viral entry, by reporting on the differential entry and localization of viral proteins inside the cell under treatment conditions. This use could also be further extended to study the mechanisms of action of such viral-neutralizing drugs, including their kinetic differences, potency effects, intracellular particle localization, and structural variances in virus-receptor complexes through the various fluorescence and luminescence detection assay formats available with the technology.

In some embodiments, the systems herein find use are molecular recorders through their self-labelling functionality. Molecular recorders are important tools for tracking transient cellular events over time. This capability is critical for understanding the dynamic processes that govern cell function, such as signaling pathways, gene expression changes, and physiological responses to environmental stimuli. Molecular recorders, like HaloTag and systems herein, offer advantages by converting transient cellular activities into permanent, detectable marks that can be analyzed subsequently. This separation of recording from analysis allows for the study of cellular activities with both high temporal resolution and across large populations of cells, providing a comprehensive view of cellular processes that were previously difficult or impossible to observe. Unlike enzymes that continuously convert substrates to generate a signal, molecular recorders offer a ‘write-once’ mechanism that marks the presence of a specific activity without the need for ongoing substrate turnover. This feature minimizes background noise and allows for the accumulation of signal only in the presence of the targeted activity, improving the specificity and sensitivity of detection. Furthermore, molecular recorders can be designed to respond to a variety of cellular events, providing flexibility that is not always possible with traditional enzymatic reporters. The use of distinguishable substrates with molecular recorders enables the sequential recording of multiple activities within the same cells or tissues, a feat difficult to achieve with conventional enzymatic reporters due to their continuous activity and signal production.

In the context of neuroscience, monitoring calcium levels in neurons is critical for understanding neuronal activity as calcium ions play a key role in transmitting signals within and between neurons. A specific application of systems herein in this area involves creating a fusion protein between a tandem peptide tag and a calcium-binding protein, such as calmodulin. Since the tandem peptide tag is a small tag, it can be introduced using CRISPR genome editing to the endogenous calmodulin locus. This fusion protein is then be expressed in neurons of interest along with a tandem detector, allowing labeling of the protein with a fluorescent HaloTag® ligand that is sensitive to calcium ions. Following the application of different stimuli (e.g., neurotransmitters or electrical stimulation) that induce calcium influx in neurons, live-cell imaging is used to monitor the fluorescence intensity of the HaloTag® ligand enabling quantitative measurement of intracellular calcium levels over time. This approach enables the mapping of calcium dynamics in response to specific stimuli, providing insights into the neural mechanisms underlying neuronal activation and communication.

Visualization and tracking of vesicle release in response to neuronal activation is an important aspect of neurotransmitter research. By creating a fusion between a tandem peptide tag and a protein involved in neurotransmitter vesicle docking or release, such as Synaptotagmin, labeling of vesicles responsible for neurotransmitter release is provided. Following the application of stimuli that trigger neuronal firing (such as pharmacological agents or optogenetic activation), time-lapse fluorescence microscopy can be used to observe the dynamics of vesicle fusion and neurotransmitter release in the presence of a tandem detector given its high affinity and labeling dependence for the tandem peptide tag. This method provides a direct way to study the temporal and spatial patterns of neurotransmitter release during different behavioral states or in response to various environmental cues, offering deeper understanding of the synaptic mechanisms contributing to behavior and neural circuit function.

Signal transduction pathways play pivotal roles in directing the differentiation of, for example, stem cells or organoids into specific tissue types. Utilizing a tandem peptide tag to monitor these pathways can reveal how signaling dynamics influence cell fate decisions during development under endogenous conditions where a small tag provides advantages in minimizing impact of its fusion partner. A specific application involves fusing a tandem peptide tag to key signaling molecules within a pathway, such as a receptor or transcription factor that is crucial for the differentiation of a particular cell type. For example, fusion of a tandem peptide tag to β-catenin, a central mediator of Wnt signaling, allows for monitoring the mechanisms underlying the differentiation of mesenchymal stem cells into osteoblasts, which is regulated by the Wnt signaling pathway. By introducing this construct into mesenchymal stem cells and using specific, fluorescently labeled HaloTag® ligands, the activation and nuclear localization of β-catenin is visualized in real-time using a fluorescence microscopy. This approach allows for the observation of how Wnt signaling fluctuates and orchestrates the differentiation process, providing insights into the molecular mechanisms driving tissue specification.

In some embodiments, systems herein find use in investigating tumor heterogeneity and metastatic potential. Tumor heterogeneity refers to the diversity of tumor cells within a single tumor, which can significantly influence its metastatic potential and response to treatment. Using the systems herein, researchers can label and track specific populations of tumor cells to study their proliferation, migration, and invasion abilities. A specific application involves tagging cancer stem cells (CSCs) known to play a pivotal role in tumor growth and metastasis. Tandem peptide tags herein are linked to surface markers unique to these cells. These CSCs are then introduced into a tumor model expressing a tandem detector, allowing researchers to track their behavior in vivo using fluorescent HaloTag® ligands. This approach enables the observation of how CSCs contribute to tumor heterogeneity, metastasis, and resistance to therapy, providing critical insights into the underlying mechanisms of cancer progression and identifying potential targets for intervention.

In some embodiments, the systems herein find use in monitoring dynamics of the tumor microenvironment. The tumor microenvironment plays a crucial role in cancer development and progression, consisting of a complex network of tumor cells, immune cells, stromal cells, and extracellular matrix components. A practical application includes tagging proteins involved in the crosstalk between tumor cells and immune cells within the microenvironment. For example, fusion of a tandem peptide tag herein with a cytokine or chemokine secreted by tumor cells that recruits T-regulatory cells, enables labeling and monitoring of the recruitment process of the T-regulatory cells to tumor cells displaying a tandem detector in real-time. Since T-regulatory cells can suppress anti-tumor immune responses, this method allows for the visualization of the mechanisms involved in tumor cell modulation of the immune environment to their advantage. Tracking these interactions could reveal new therapeutic targets aimed at disrupting the protective niche created by tumor cells in the tumor microenvironment, enhancing the efficacy of immunotherapies.

In some embodiments, the systems herein find use for protein tagging, capture, and analysis. A practical application of the systems herein includes a tandem peptide tag fused to a target protein of interest, maintaining the functional versatility of the original, monomeric HaloTag®, but requiring addition of a complementary sequence for active HaloTag® formation. In such a system, the tagged target protein can be overexpressed and captured in the presence of a dual detector reagent that includes the complementary component of the binary HaloTag (HT). Capture can then be accomplished using either a biotin HaloTag® ligand with a streptavidin surface or directly onto a chloroalkane surface. This method facilitates efficient purification of the target protein or pull-down of associated prey proteins, which could then identified by mass spectrometry. This application highlights the tandem tag/detector system's capability to provide detailed protein interaction studies and purification processes, similar to those possible with the full-length HaloTag®. However, the tandem peptide tag represents a much smaller tag, potentially reducing the risk of compromising the target protein's functionality. An additional advantage of this application is that it leaves the target protein tagged with HiBiT, enabling its detection, monitoring, and quantification during subsequent analyses.

### EXPERIMENTAL

### Example 1

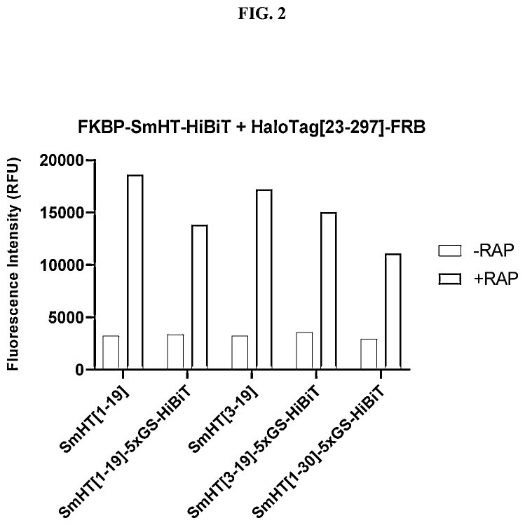

Experiments were conducted during development of embodiments herein to demonstrate that the SmHT and HiBiT tags were compatible with each other by fusing them together into a single peptide tag (e.g., SmHT-HiBiT) and measuring activity in facilitated complementation assays. The tags were fused to the C-terminus of FKBP and facilitated complementation with a FRB fusion to the C-terminus of a LgHT variant, HaloTag[23-297]. In the presence of Rapamycin, the FKBP-SmHT-HiBiT fusion was similarly capable of showing a complementation-dependent increase in JF646 HaloTag® ligand labeling as the control with only FKBP-SmHT (no HiBiT), indicating that the presence of the HiBiT tag in this manner did not interfere with split HaloTag® function. (FIG. 2).

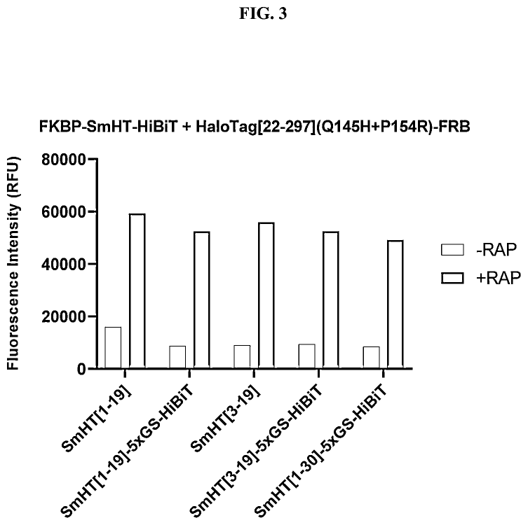

In the same system, a different LgHT variant, HaloTag[22-297](Q145H+P154) was used and exhibited similar performance with or without the HiBiT appended to the C-terminus of the FKBP-SmHT fusion (FIG. 3). This particular variant of LgHT has improved expression and stability and confirmed that the properties of the LgHT can be changed and maintain performance in this assay.

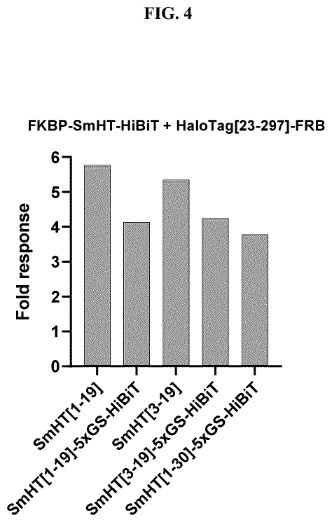

The fold response of the HaloTag[23-297]—FRB to Rapamycin was calculated with and without the HiBiT present on the FKBP-SmHT. The presence of the HiBiT tag did not interfere with complementation (FIG. 4).

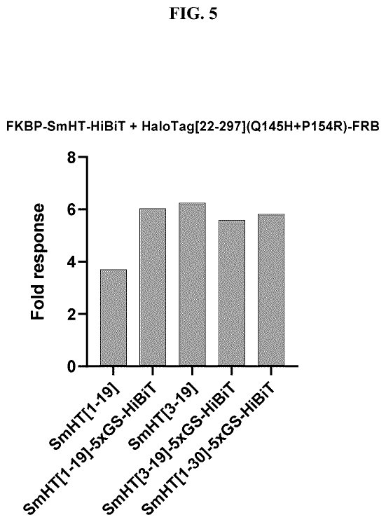

Similarly, the fold response was calculated for the more well expressed and stable HaloTag[22-297](Q145H+P154)-FRB to Rapamycin with and without the HiBiT present on the FKBP-SmHT. The presence of the HiBiT tag did not interfere with complementation (FIG. 5).

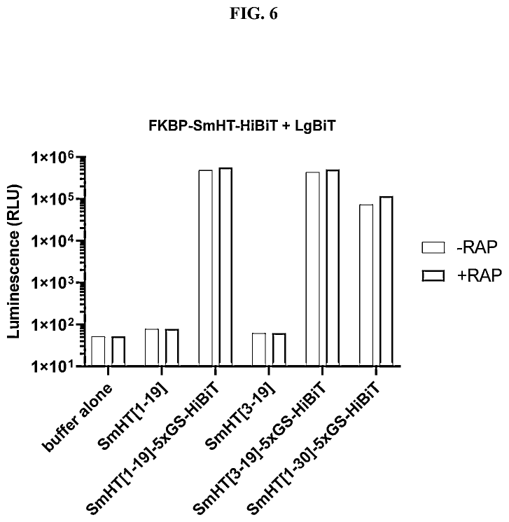

Experiments were conducted during development of embodiments herein to determine if the split NanoLuc® (NanoBiT) components retained function in the dual configuration as well. Purified LgBiT could be added to the lysate containing the FKBP-SmHT-HiBiT fusion and high levels of luminescence activity that was dependent on the presence of the HiBiT tag in the reaction was detected (FIG. 6). In this experiment, there is no LgHT present as it was used to confirm that the NanoBiT technology was still functional when the HiBiT was configured in the FKBP-SmHT-HiBiT fusion.

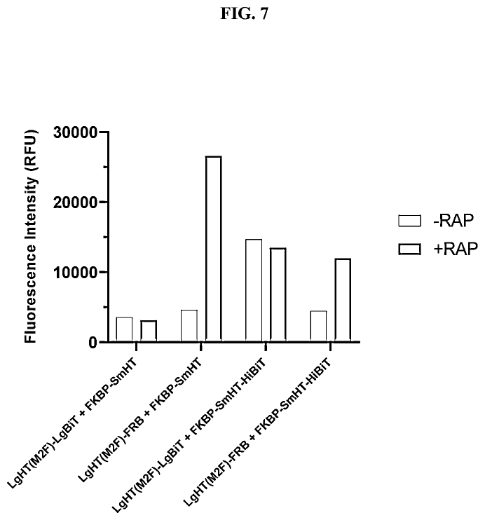

Experiments were conducted during development of embodiments herein to demonstrate the function of the LgHT-LgBiT reporter polypeptide for spontaneous complementation of the split HaloTag sequences and increase in activity. As a control, the LgHT-FRB fusions were compared to LgHT-LgBiT fusions in a facilitated complementation assay. For this experiment, LgHT variant HaloTag[22-297](M2F) was used, which has a high fold response in the assay due to its lower background (non-facilitated) activity levels. The FKBP-SmHT fusion was also compared to the FKBP-SmHT-HiBiT fusion. The data shows that when LgHT is fused to FRB, it requires the presence of Rapamycin to facilitate its complementation to FKBP-SmHT and FKBP-SmHT-HiBiT through the FRB:FKBP interaction (FIG. 7). When LgHT is fused to LgBiT, complementation of the split HaloTag® sequences occurs in a Rapamycin-independent manner, with the (−) Rapamycin conditions showing similar fluorescence to (+) Rapamycin conditions. The fluorescence response was also dependent on HiBiT fusion to the FKBP-SmHT, indicating the specificity of the configuration to both the LgBiT and HiBiT components.

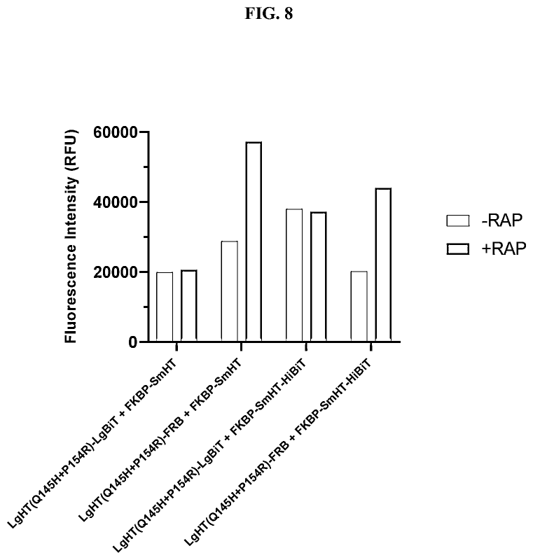

Experiments were conducted during development of embodiments herein to test another LgHT variant fused to LgBiT, HaloTag[22-297](Q145H+P154R), which typically shows higher expression and stability, but lower fold response in this assay due to its higher background activity. The results are similar to those of the previous LgHT(M2F) variant, in which we demonstrate the ability of the LgHT-LgBiT fusion to spontaneously complement (independent of Rapamycin) the split HaloTag® components of the configuration and that it is dependent on the presence of both LgBiT and HiBiT fusions (FIG. 8). Due to the high expression of this mutant in E. coli lysates, higher fluorescence intensity was achieved.

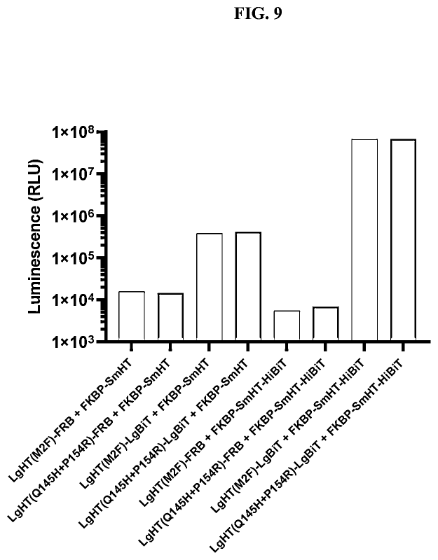

Experiments were conducted during development of embodiments herein to demonstrate the measurement of luminescence of the complemented LgHT-LgBiT polypeptide (two different LgHT variants) in complex with the FKBP-SmHT-HiBiT in the same reaction following the measurement of its fluorescence. Reactions from FIGS. 7 and 8, in which the split HaloTag® activity of the complex was measured using labeling with JF646 HaloTag® ligand, were diluted into a NanoGlo® assay buffer containing furimazine, and the luminescence measured. The data shows that the maximum luminescence is observed when the LgBiT and HiBiT are present in the configurations, about 100-fold higher than the same containing LgHT-LgBiT, but no HiBiT on the FKBP-SmHT fusion (FIG. 9). This supports the capability of this technology to measure both luminescence and fluorescence within the complementation complex simultaneously in the same homogeneous reaction mixture without any need for washing, enrichment, or purification.

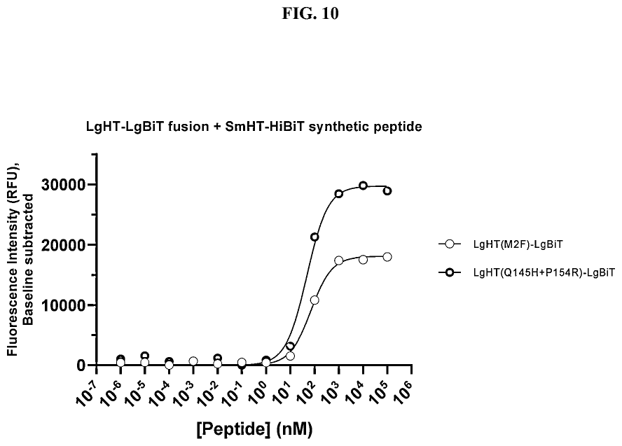

Experiments were conducted during development of embodiments herein to demonstrate that use of a synthetic peptide of the SmHT-HiBiT tag (without any other fusions) to form complementation complexes with two different LgHT-LgBiT variants in E. coli lysates. As the concentration of the synthetic SmHT-HiBiT peptide added to the reaction was increased, the high affinity interaction between the HiBiT:LgBiT facilitates the complementation of the split HaloTag components, resulting in increased labeling and fluorescence with JF646 HaloTag® ligand (FIG. 10). The data demonstrates the direct interaction between the two polypeptides is sufficient to observe increased split HaloTag® activity, without the need for fusion partners (such as FRB or FKBP).

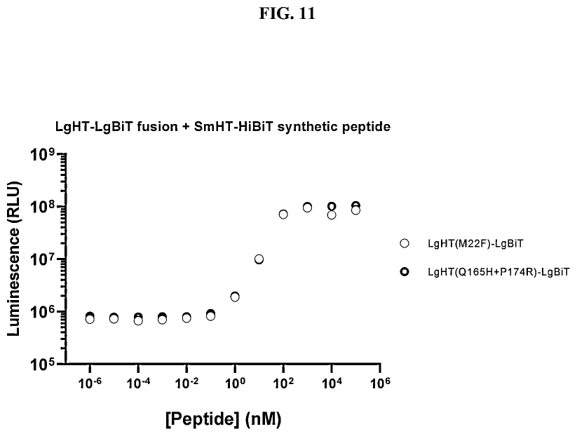

Experiments were conducted during development of embodiments herein to measure the luminescence activity of the complex at increasing concentrations of a synthetic SmHT-HiBiT peptide added to E. coli lysates expressing two different LgHT-LgBiT variant polypeptides (FIG. 11). The reactions show similar performance of the NanoBiT components of the complex regardless of their respective LgHT fusion partner. It also demonstrates that high luminescence is capable in an assay format using synthetic peptide and no other fusion partners (FRB or FKBP), indicating that the LgBiT:HiBiT interaction is sufficient for high affinity facilitation of the interaction and the presence of the LgHT or SmHT components of the complex do not interfere with NanoBiT performance.

### Example 2

### HaloTag[3-19]-HiBiT Configuration and Mutation Screening in E. coli Lysates

### Effects of Varying Internal Linker Length and Orientation of HaloTag[3-19]-HiBiT as C-Terminal FKBP Fusions on Activity in Lysates.

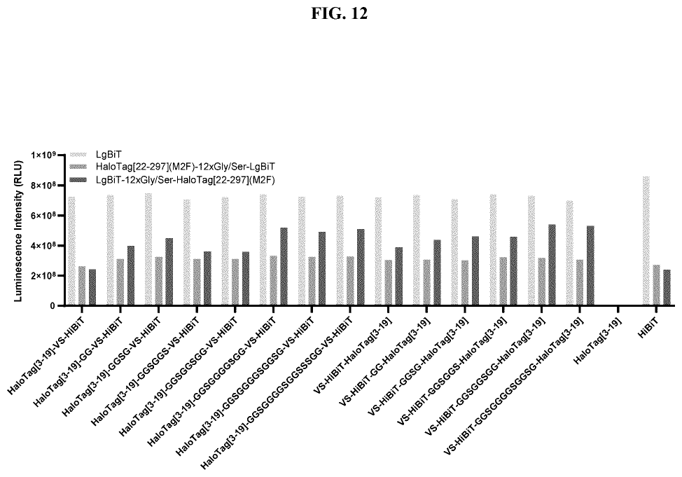

Experiments were conducted during development of embodiments herein demonstrating that HaloTag[3-19]-HiBiT variants are functional with varying Gly-Ser linker lengths between the HaloTag[3-19] and HiBiT sequences (FIG. 12). The variants are also functional in both orientations (HaloTag[3-19]-HiBiT and HiBiT-HaloTag[3-19]) as C-terminal fusions to FKBP, and when complemented with LgBiT alone or with either HaloTag[22-297](M2F)-12×Gly/Ser-LgBiT or LgBiT-12×Gly/Ser-HaloTag[22-297](M2F).

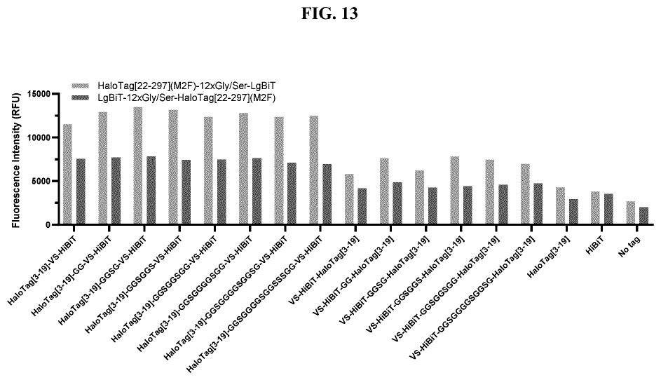

Experiments were conducted during development of embodiments herein demonstrating that HaloTag[3-19]-HiBiT variants are functional with various flexible linker lengths between the HaloTag[3-19] and HiBiT peptide sequences (FIG. 13). The variants are also functional in both orientations (HaloTag[3-19]-HiBiT and HiBiT-HaloTag[3-19]) as C-terminal fusions to FKBP and when complemented with HaloTag[22-297](M2F)-12×Gly/Ser-LgBiT or LgBiT-12×Gly/Ser-HaloTag[22-297](M2F).

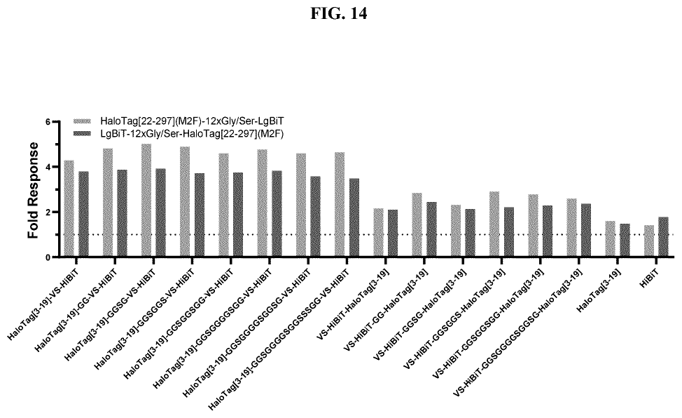

Experiments were conducted during development of embodiments herein demonstrating that HaloTag[3-19]-HiBiT variants are functional with varying Gly-Ser linker lengths between the HaloTag[3-19] and HiBiT sequences (FIG. 14). The HaloTag[3-19]-HiBiT variants are also functional in both orientations (HaloTag[3-19]-HiBiT and HiBiT-HaloTag[3-19]) as C-terminal fusions to FKBP and when complemented with HaloTag[22-297](M2F)-12×Gly/Ser-LgBiT or LgBiT-12×Gly/Ser-HaloTag[22-297](M2F).

### Effects of Varying Internal Linker Length and Orientation of HaloTag[3-19]-HiBiT as N-Terminal FKBP Fusions on Activity in Lysates.

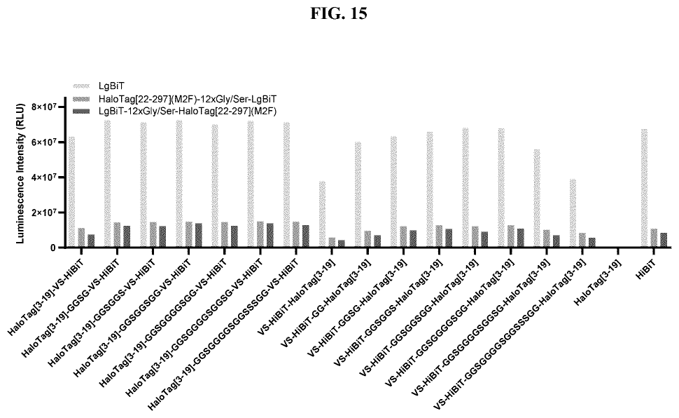

Experiments were conducted during development of embodiments herein demonstrating that HaloTag[3-19]-HiBiT variants are functional with varying Gly-Ser linker lengths between the HaloTag[3-19] and HiBiT sequences (FIG. 15). The variants are also functional in both orientations (HaloTag[3-19]-HiBiT and HiBiT-HaloTag[3-19]) as N-terminal fusions to FKBP, and when complemented with LgBiT alone, HaloTag[22-297](M2F)-12×Gly/Ser-LgBiT, or LgBiT-12×Gly/Ser-HaloTag[22-297](M2F).

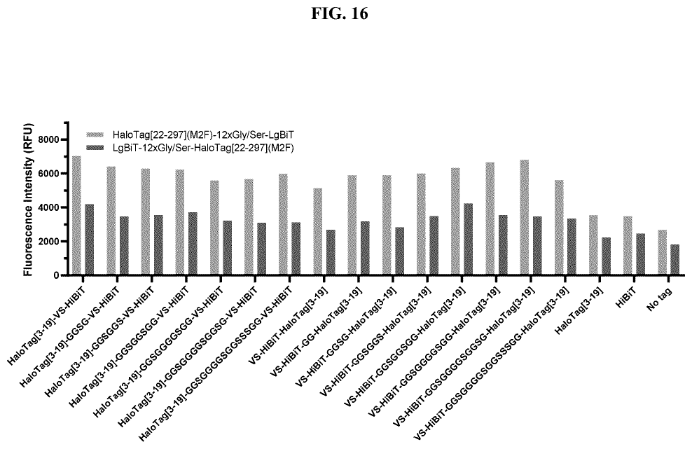

Experiments were conducted during development of embodiments herein demonstrating that HaloTag[3-19]-HiBiT variants are functional with varying Gly-Ser linker lengths between the HaloTag[3-19] and HiBiT sequences (FIG. 16). The variants are also functional in both orientations (HaloTag[3-19]-HiBiT and HiBiT-HaloTag[3-19]) as N-terminal fusions to FKBP and when complemented with HaloTag[22-297](M2F)-12×Gly/Ser-LgBiT or LgBiT-12×Gly/Ser-HaloTag[22-297](M2F).

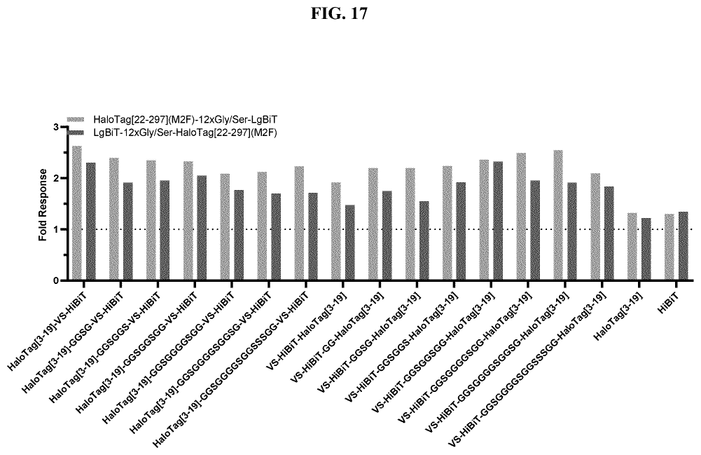

Experiments were conducted during development of embodiments herein demonstrating that HaloTag[3-19]-HiBiT variants are functional with varying Gly-Ser linker lengths between the HaloTag[3-19] and HiBiT sequences (FIG. 17). The HaloTag[3-19]-HiBiT variants are also functional in both orientations (HaloTag[3-19]-HiBiT and HiBiT-HaloTag[3-19]) as N-terminal fusions to FKBP and when complemented with HaloTag[22-297](M2F)-12×Gly/Ser-LgBiT or LgBiT-12×Gly/Ser-HaloTag[22-297](M2F).

### Effects of Varying Linker Length and Composition of HaloTag[3-19]-HiBiT as C-Terminal FKBP Fusions on Activity in Lysates.

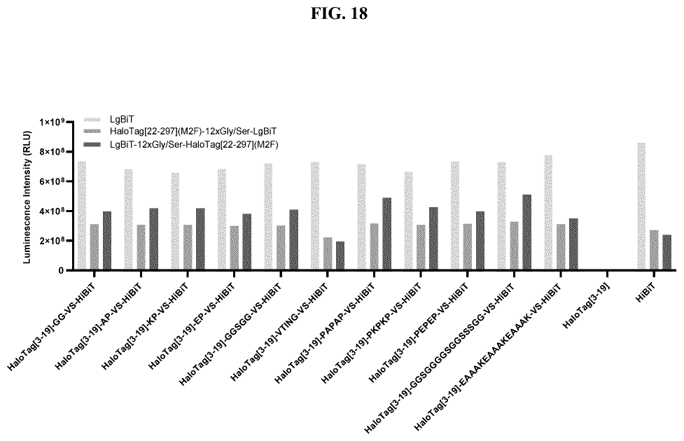

Experiments were conducted during development of embodiments herein demonstrating that HaloTag[3-19]-HiBiT variants are functional with varying Gly-Ser and other composition (rigid) linker lengths between the HaloTag[3-19] and HiBiT sequences (FIG. 18). The variants are also functional in both orientations (HaloTag[3-19]-HiBiT and HiBiT-HaloTag[3-19]) as C-terminal fusions to FKBP, and when complemented with LgBiT alone, HaloTag[22-297](M2F)-12×Gly/Ser-LgBiT, or LgBiT-12×Gly/Ser-HaloTag[22-297](M2F).

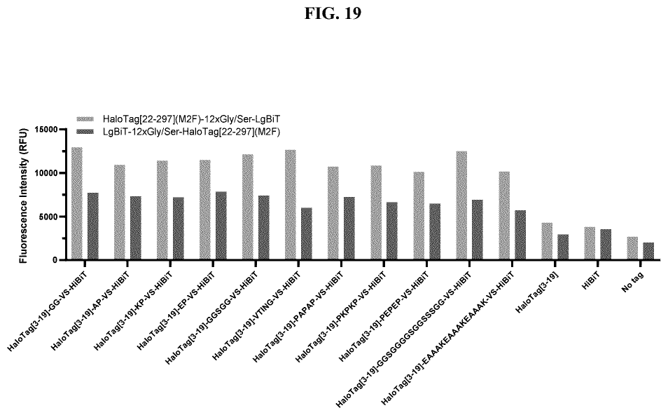

Experiments were conducted during development of embodiments herein demonstrating that HaloTag[3-19]-HiBiT variants are functional with varying flexible Gly-Ser and rigid linker lengths between the HaloTag[3-19] and HiBiT sequences (FIG. 19). The variants are also functional in both orientations (HaloTag[3-19]-HiBiT and HiBiT-HaloTag[3-19]) as C-terminal fusions to FKBP and when complemented with HaloTag[22-297](M2F)-12×Gly/Ser-LgBiT or LgBiT-12×Gly/Ser-HaloTag[22-297](M2F).

Effects of Varying Linker Length Between FKBP and HaloTag[3-19]-HiBiT C-Terminal Fusions on Activity in E. coli Lysates.

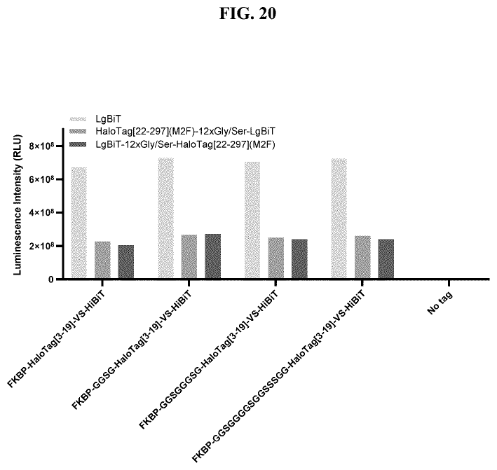

Experiments were conducted during development of embodiments herein demonstrating that HaloTag[3-19]-HiBiT variants are functional with varying Gly-Ser linkers separating the tandem peptide tag from its fusion partner, FKBP (FIG. 20). The variants are also functional when complemented with LgBiT alone, HaloTag[22-297](M2F)-12×Gly/Ser-LgBiT, or LgBiT-12×Gly/Ser-HaloTag[22-297](M2F).

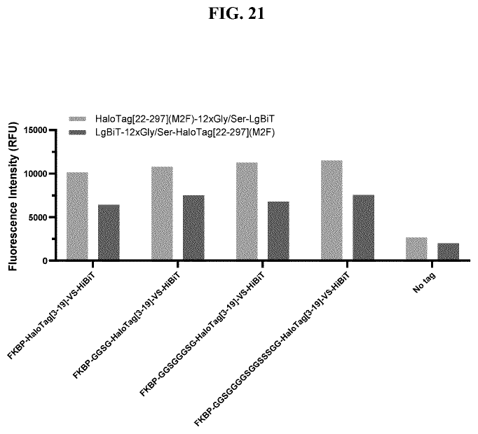

Experiments were conducted during development of embodiments herein demonstrating that HaloTag[3-19]-HiBiT variants are functional in fluorescence assays with varying Gly-Ser linkers separating the tandem peptide tag from its fusion partner, FKBP (FIG. 21). The variants are also functional when complemented with either HaloTag[22-297](M2F)-12×Gly/Ser-LgBiT or LgBiT-12×Gly/Ser-HaloTag[22-297](M2F).

### Effects of Varying Linker Length Between N-Terminal HaloTag[3-19]-HiBiT and FKBP Genetic Fusions on Activity in Lysates.

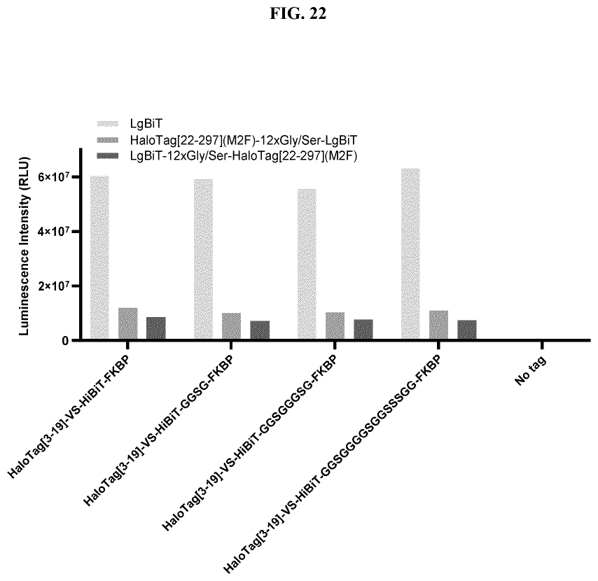

Experiments were conducted during development of embodiments herein demonstrating that HaloTag[3-19]-HiBiT variants are functional with varying Gly-Ser linkers separating the tandem peptide tag from its fusion partner, FKBP (FIG. 22). The variants are also functional when complemented with LgBiT alone, HaloTag[22-297](M2F)-12×Gly/Ser-LgBiT, or LgBiT-12×Gly/Ser-HaloTag[22-297](M2F).

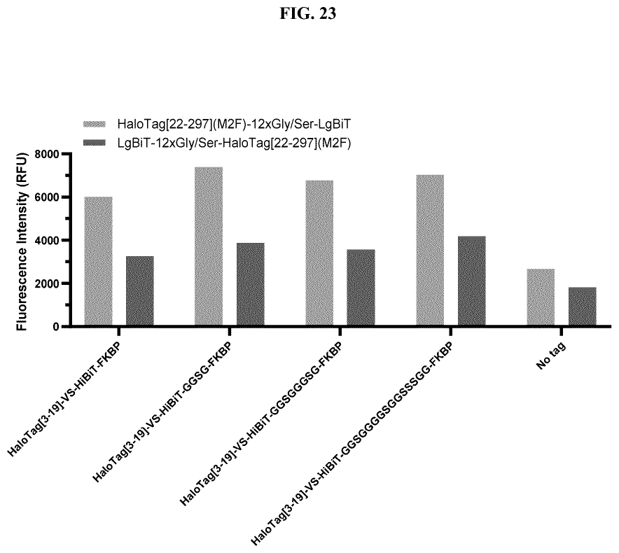

Experiments were conducted during development of embodiments herein demonstrating that HaloTag[3-19]-HiBiT variants are functional in fluorescence assays with varying Gly-Ser linkers separating the tandem peptide tag from its fusion partner, FKBP (FIG. 23). The variants are also functional when complemented with either HaloTag[22-297](M2F)-12×Gly/Ser-LgBiT or LgBiT-12×Gly/Ser-HaloTag[22-297](M2F).

### Effects of HaloTag[3-19] Truncations on HaloTag[3-19]-HiBiT Activity in Lysates.

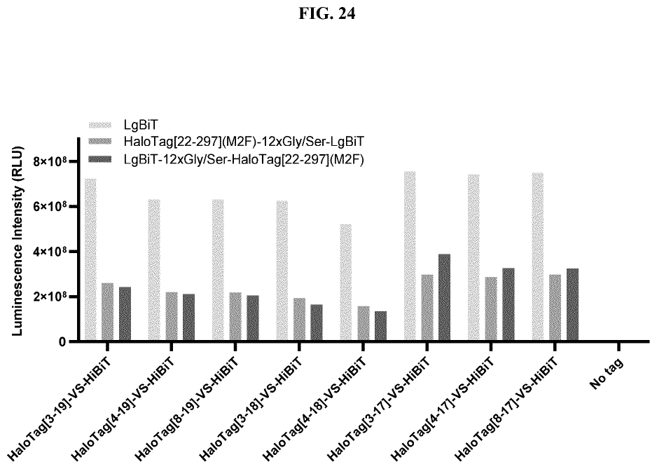

Experiments were conducted during development of embodiments herein demonstrating that HaloTag[3-19]-HiBiT variants are functional in luminescence assays with HaloTag[3-19] truncations (FIG. 24). The variants are also functional when complemented with LgBiT alone, HaloTag[22-297](M2F)-12×Gly/Ser-LgBiT, or LgBiT-12×Gly/Ser-HaloTag[22-297](M2F).

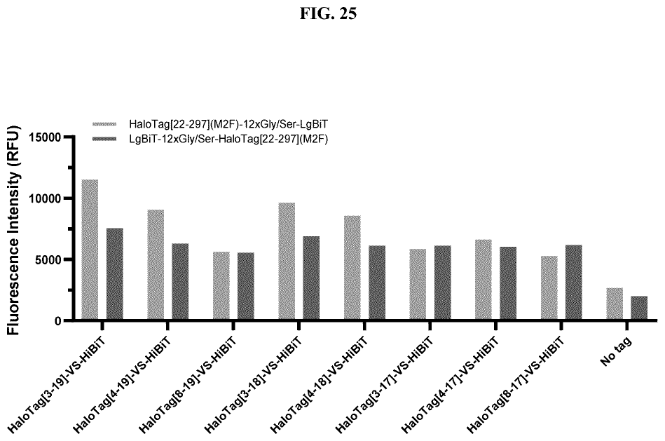

Experiments were conducted during development of embodiments herein demonstrating that HaloTag[3-19]-HiBiT variants are functional in fluorescence assays with HaloTag[3-19] truncations (FIG. 25). The variants are also functional when complemented with either HaloTag[22-297](M2F)-12×Gly/Ser-LgBiT or LgBiT-12×Gly/Ser-HaloTag[22-297](M2F).

### Effects of HiBiT Mutations on HaloTag[3-19]-HiBiT Activity in Lysates.

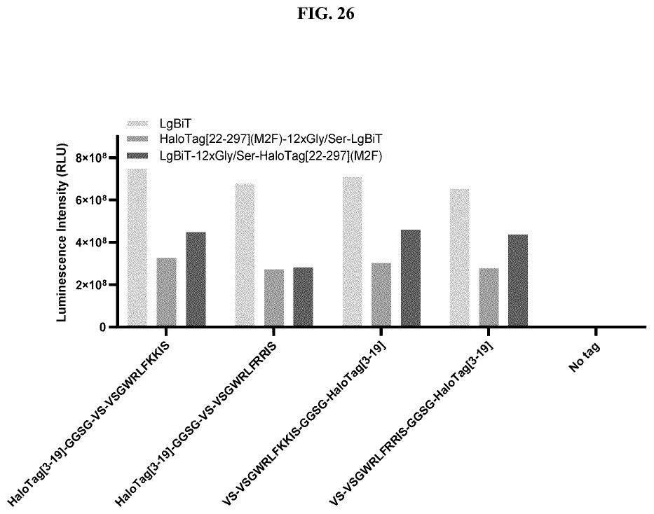

Experiments were conducted during development of embodiments herein demonstrating that HaloTag[3-19]-HiBiT variants with HiBiT mutations are functional in luminescence assays (FIG. 26). The variants are also functional when complemented with LgBiT alone, HaloTag[22-297](M2F)-12×Gly/Ser-LgBiT, or LgBiT-12×Gly/Ser-HaloTag[22-297](M2F).

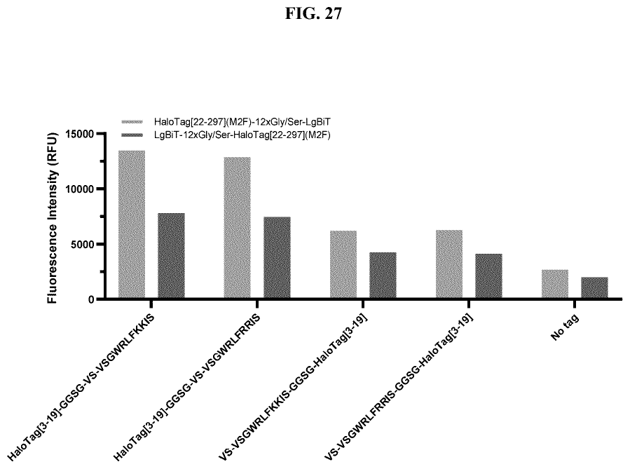

Experiments were conducted during development of embodiments herein demonstrating that HaloTag[3-19]-HiBiT variants with HiBiT mutations are functional in fluorescence assays (FIG. 27). The variants are also functional when complemented with either HaloTag[22-297](M2F)-12×Gly/Ser-LgBiT or LgBiT-12×Gly/Ser-HaloTag[22-297](M2F).

### Example 3

### HaloTag[3-19]-HiBiT Configuration and Mutation Screening with Synthetic Peptides

### Luminescence Activity of HaloTag[3-19]-HiBiT Synthetic Peptide Variants.

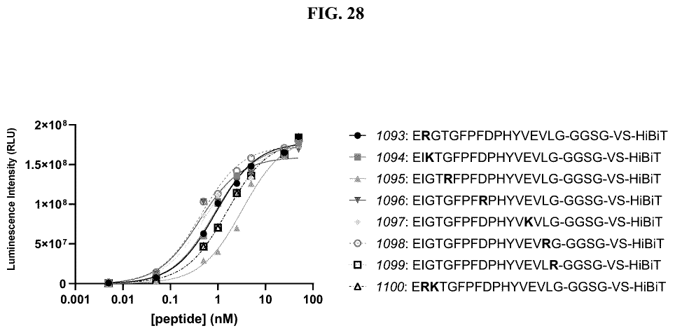

Experiments were conducted during development of embodiments herein demonstrating that HaloTag[3-19]-HiBiT variant synthetic peptides with one or more sequence changes are still functional in complementing LgBiT-6×His to enhance luminescence signal (FIG. 28).

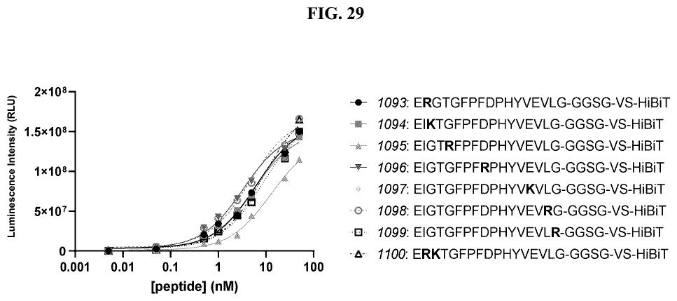

Experiments were conducted during development of embodiments herein demonstrating that HaloTag[3-19]-HiBiT synthetic peptides with one or more sequence changes are still functional in complementing HaloTag[22-297](M2F+K140E)-12×Gly/Ser-LgBiT-6×His to enhance luminescence signal (FIG. 29).

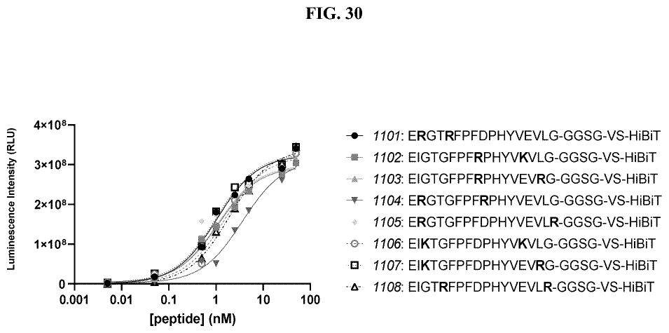

Experiments were conducted during development of embodiments herein demonstrating that HaloTag[3-19]-HiBiT synthetic peptides with one or more sequence changes are still functional in complementing HaloTag[22-297](M2F+K140E)-12×Gly/Ser-LgBiT-6×His to enhance luminescence signal (FIG. 30).

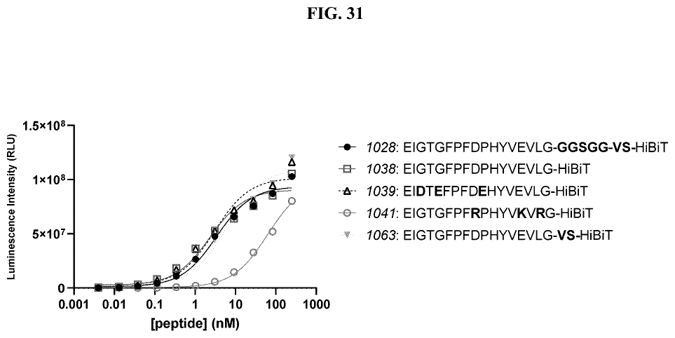

Experiments were conducted during development of embodiments herein demonstrating that HaloTag[3-19]-HiBiT variants synthetic peptides with sequence changes or additional linker residues between HaloTag[3-19] and HiBiT are still functional in complementing HaloTag[22-297](M2F)-12×Gly/Ser-LgBiT-6×His to enhance luminescence signal (FIG. 31).

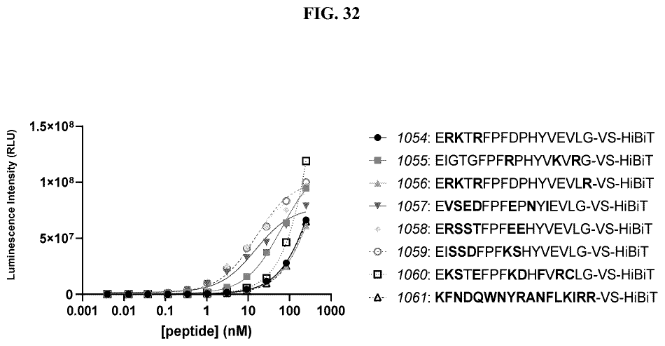

Experiments were conducted during development of embodiments herein demonstrating that HaloTag[3-19]-HiBiT variants synthetic peptides with one or more sequence changes are still functional in complementing HaloTag[22-297](M2F)-12×Gly/Ser-LgBiT-6×His to enhance luminescence signal (FIG. 32). Peptide 1061 contains a HaloTag[3-19] sequence in which all 17 residues have been mutated, and the peptide is still functional.

### Fluorescence Activity of HaloTag[3-19]-HiBiT Synthetic Peptide Variants.

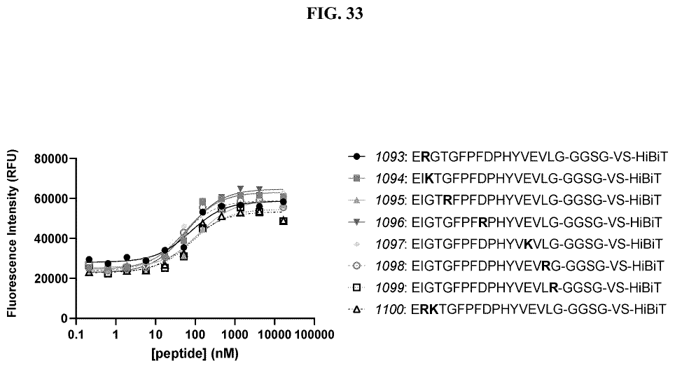

Experiments were conducted during development of embodiments herein demonstrating that HaloTag[3-19]-HiBiT variant synthetic peptides with one or more sequence changes are still functional in complementing HaloTag[22-297](M2F+K140E)-12×Gly/Ser-LgBiT-6×His to enhance fluorescence signal (FIG. 33).

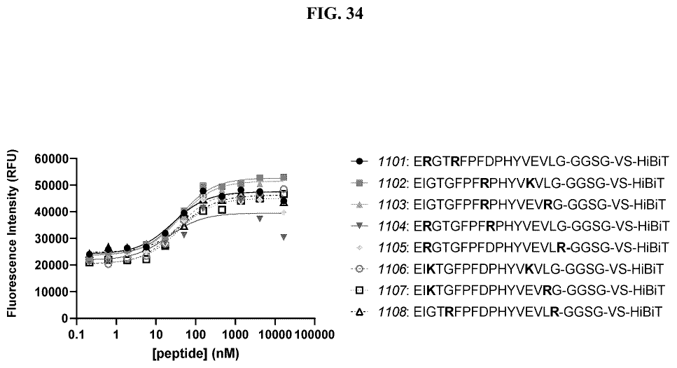

Experiments were conducted during development of embodiments herein demonstrating that HaloTag[3-19]-HiBiT variant synthetic peptides with one or more sequence changes are still functional in complementing HaloTag[22-297](M2F+K140E)-12×Gly/Ser-LgBiT-6×His to enhance fluorescence signal (FIG. 34).

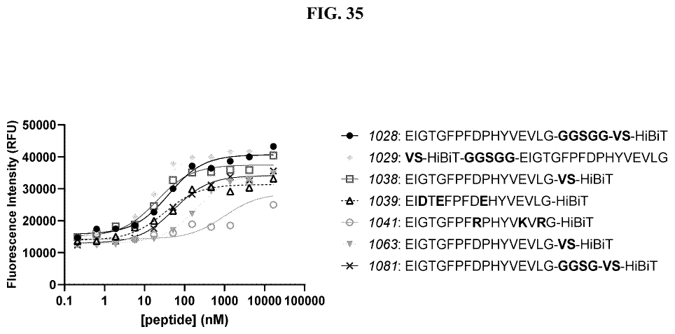

Experiments were conducted during development of embodiments herein demonstrating that HaloTag[3-19]-HiBiT variant synthetic peptides with one or more sequence changes are still functional in complementing HaloTag[22-297](M2F+K140E)-12×Gly/Ser-LgBiT-6×His to enhance fluorescence signal (FIG. 35).

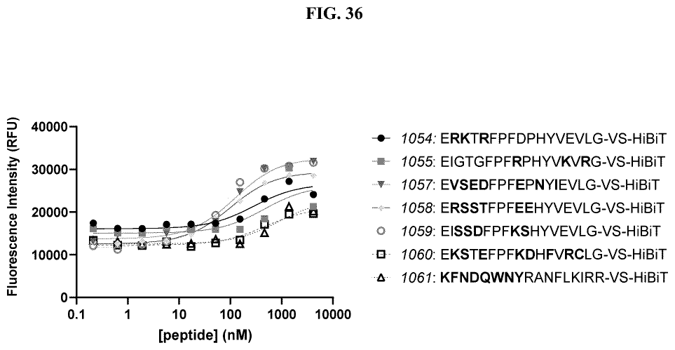

Experiments were conducted during development of embodiments herein demonstrating that HaloTag[3-19]-HiBiT variant synthetic peptides with one or more sequence changes are still functional in complementing HaloTag[22-297](M2F+K140E)-12×Gly/Ser-LgBiT-6×His to enhance fluorescence signal (FIG. 36).

### Fluorescence Activity of HaloTag[3-19] Synthetic Peptide Variants.

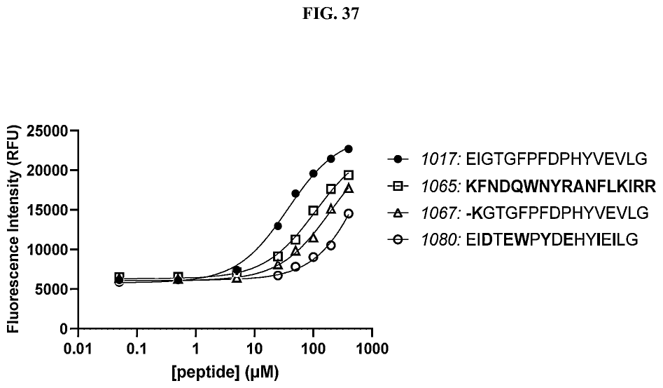

Experiments were conducted during development of embodiments herein demonstrating that HaloTag[3-19] synthetic peptides with a single residue deletion and/or one or more sequence changes are still functional in complementing HaloTag[22-297](M2F)-12×Gly/Ser-LgBiT and enhancing fluorescence intensity of HaloTag[22-297](M2F)-12×Gly/Ser-LgBiT bound to JF646 HaloTag ligand (FIG. 37). Every residue in peptide 1065 has been changed from the original HaloTag[3-19] sequence and is still a functional HaloTag[3-19].

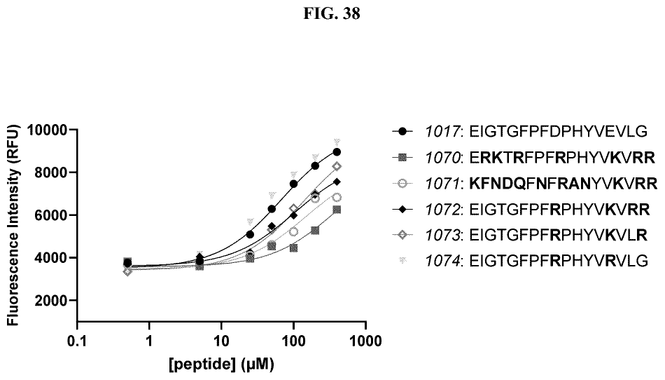

Experiments were conducted during development of embodiments herein demonstrating that HaloTag[3-19] synthetic peptides with two or more sequence changes are still functional in complementing HaloTag[22-297](M2F)-12×Gly/Ser-LgBiT and enhancing fluorescence intensity of HaloTag[22-297](M2F)-12×Gly/Ser-LgBiT bound to JF646 HaloTag® ligand (FIG. 38).

Experiments were conducted during development of embodiments herein demonstrating that HaloTag[3-19] synthetic peptides with one or more sequence changes are still functional in complementing HaloTag[22-297](M2F)-12×Gly/Ser-LgBiT and enhancing fluorescence intensity of HaloTag[22-297](M2F)-12×Gly/Ser-LgBiT bound to JF646 HaloTag® ligand.

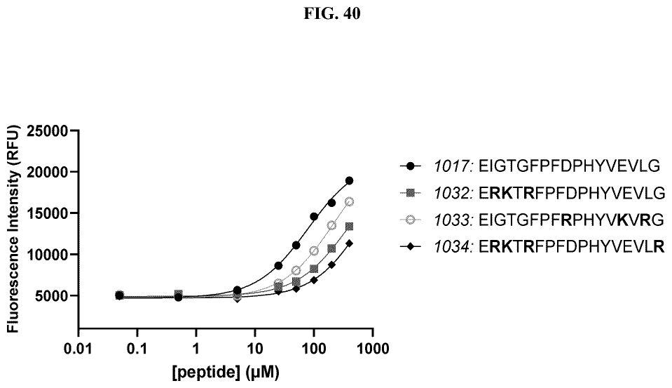

Experiments were conducted during development of embodiments herein demonstrating that HaloTag[3-19] synthetic peptides with one or more sequence changes are still functional in complementing HaloTag[22-297](M2F)-12×Gly/Ser-LgBiT and enhancing fluorescence intensity of HaloTag[22-297](M2F)-12×Gly/Ser-LgBiT bound to JF646 HaloTag® ligand (FIG. 40).

### Example 4

### Binding Kinetics of HaloTag[22-297]-12×Gly/Ser-LgBiT Variants with Synthetic Peptides

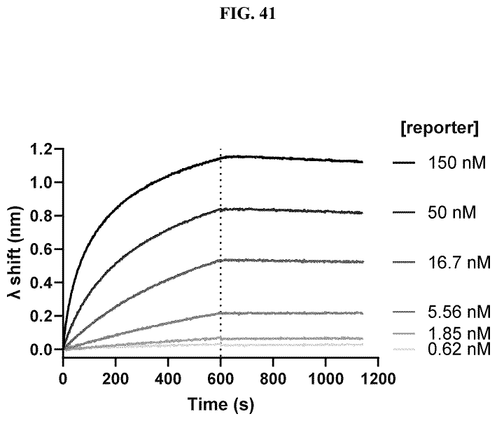

Experiments were conducted during development of embodiments herein demonstrating that the dissociation of HaloTag[3-19](12R)-4×Gly/Ser-VS-HiBiT from HaloTag[22-297](M2F+F148M+V177E)-12×Gly/Ser-LgBiT-6×His is very slow, as there is almost no decrease in shift during the dissociation phase of the experiment (FIG. 41).

Table 1 includes binding measurements for HaloTag[3-19]-HiBiT variant synthetic peptides and HaloTag[22-297]-12×Gly/Ser-LgBiT-6×His variants and includes a comparison to binding measurements for VS-HiBiT and LgBiT-6×His. The NA designation in the k_{off} column represents dissociation that is too slow for the instrument to measure (limit 10⁶ l/s), which also prohibits the calculation of a K_{D}. These results demonstrate that HaloTag[3-19]-HiBiT variants have very slow dissociation from HaloTag[22-297]-12×Gly/Ser-LgBiT-6×His variants and LgBiT-6×His alone.

TABLE 1
Binding measurements for polypeptide reporter/synthetic peptide complementation from biolayer
interferometry experiments. All peptides are biotinylated at the N-terminus for immobilization
onto streptavidin probes. Binding experiments were performed in PBST at 25° C.

| Peptide | Polypeptide reporter | K_{off} (1/s) | Kₒₙ (1/Ms) | K_{D} (M) |
|---|---|---|---|---|
| HaloTag[3-19](I2R)- | HaloTag[22-297](M2F)-12xGly/Ser- | 8.34E−005 | 5.19E+004 | 1.61E−009 |
| 4xGly/Ser-VSHiBiT | LgBiT-3xGly/Ser-6xHis | | | |
| HaloTag[3-19](I2R)- | HaloTag[22-297](M2F + K140E)- | 3.26E−005 | 7.00E+004 | 4.66E−010 |
| 4xGly/Ser-VSHiBiT | 12xGly/Ser-LgBiT-3xGly/Ser-6xHis | | | |
| HaloTag[3-19](I2R)- | HaloTag[22-297](M2F + D53G + V177E)- | 4.09E−005 | 6.13E+004 | 6.69E−010 |
| 4xGly/Ser-VSHiBiT | 12xGly/Ser-LgBiT-3xGly/Ser-6xHis | | | |
| HaloTag[3-19](I2R)- | HaloTag[22-297](M2F + K140E)- | 4.56E−005 | 6.67E+004 | 6.84E−010 |
| 4xGly/Ser-VSHiBiT | 12xGly/Ser-LgBiT-3xGly/Ser-6xHis | | | |
| HaloTag[3-19](G3K)- | HaloTag[22-297](M2F + K140E)- | 1.93E−005 | 7.30E+004 | 2.64E−010 |
| 4xGly/Ser-VSHiBiT | 12xGly/Ser-LgBiT-3xGly/Ser-6xHis | | | |
| HaloTag[3-19](G5R)- | HaloTag[22-297](M2F + K140E)- | NA | 8.53E+004 | NA |
| 4xGly/Ser-VSHiBiT | 12xGly/Ser-LgBiT-3xGly/Ser-6xHis | | | |
| HaloTag[3-19](D9R)- | HaloTag[22-297](M2F + K140E)- | 1.09E−005 | 8.66E+004 | 1.26E−010 |
| 4xGly/Ser-VSHiBiT | 12xGly/Ser-LgBiT-3xGly/Ser-6xHis | | | |
| HaloTag[3-19](E14K)- | HaloTag[22-297](M2F + K140E)- | NA | 1.08E+005 | NA |
| 4xGly/Ser-VSHiBiT | 12xGly/Ser-LgBiT-3xGly/Ser-6xHis | | | |
| HaloTag[3-19](L16R)- | HaloTag[22-297](M2F + K140E)- | NA | 7.79E+004 | NA |
| 4xGly/Ser-VSHiBiT | 12xGly/Ser-LgBiT-3xGly/Ser-6xHis | | | |
| VSHiBiT | HaloTag[22-297](M2F + F148M + V177E)- | 6.28E−004 | 2.61E+005 | 2.40E−009 |
| | 12xGly/Ser-LgBiT-3xGly/Ser-6xHis | | | |
| VSHiBiT | LgBiT-6xHis | 9.31E−004 | 1.84E+005 | 5.06E−009 |
| HaloTag[3-19](I2R)- | HaloTag[22-297](M2F + F148M + V177E)- | 2.75E−005 | 5.40E+004 | 5.09E−010 |
| 4xGly/Ser-VSHiBiT | 12xGly/Ser-LgBiT-3xGly/Ser-6xHis | | | |
| HaloTag[3-19](G3K)- | HaloTag[22-297](M2F + F148M + V177E)- | 5.03E−006 | 4.95E+004 | 1.02E−010 |
| 4xGly/Ser-VSHiBiT | 12xGly/Ser-LgBiT-3xGly/Ser-6xHis | | | |
| HaloTag[3-19](G5R)- | HaloTag[22-297](M2F + F148M + V177E)- | 2.23E−005 | 4.60E+004 | 4.85E−010 |
| 4xGly/Ser-VSHiBiT | 12xGly/Ser-LgBiT-3xGly/Ser-6xHis | | | |
| HaloTag[3-19](I2R + G3K)- | HaloTag[22-297](M2F + K140E)- | NA | 1.04E+005 | NA |
| 4xGS-VSHiBIT | 12xGly/Ser-LgBiT-3xGly/Ser-6xHis | | | |
| HaloTag[3-19](I2R + G5R)- | HaloTag[22-297](M2F + K140E)- | NA | 9.51E+004 | NA |
| 4xGS-VSHiBiT | 12xGly/Ser-LgBiT-3xGly/Ser-6xHis | | | |
| HaloTag[3-19](D9R + | HaloTag[22-297](M2F + K140E)- | 1.69E−005 | 1.52E+005 | 1.11E−010 |
| E14K)-4xGS-VSHiBiT | 12xGly/Ser-LgBiT-3xGly/Ser-6xHis | | | |
| HaloTag[3-19](D9R + | HaloTag[22-297](M2F + K140E)- | NA | 1.34E+005 | NA |
| L16R)-4xGS-VSHiBiT | 12xGly/Ser-LgBiT-3xGly/Ser-6xHis | | | |
| HaloTag[3-19](I2R + D9R)- | HaloTag[22-297](M2F + K140E)- | NA | 1.28E+005 | NA |
| 4xGS-VSHiBiT | 12xGly/Ser-LgBiT-3xGly/Ser-6xHis | | | |
| HaloTag[3-19](I2R + | HaloTag[22-297](M2F + K140E)- | NA | 9.33E+004 | NA |
| G17R)-4xGS-VSHiBiT | 12xGly/Ser-LgBiT-3xGly/Ser-6xHis | | | |
| HaloTag[3-19](G3K + | HaloTag[22-297](M2F + K140E)- | NA | 1.28E+005 | NA |
| E14K)-4xGS-VSHiBiT | 12xGly/Ser-LgBiT-3xGly/Ser-6xHis | | | |
| HaloTag[3-19](G3K + | HaloTag[22-297](M2F + K140E)- | NA | 9.71E+004 | NA |
| L16R)-4xGS-VSHiBiT | 12xGly/Ser-LgBiT-3xGly/Ser-6xHis | | | |
| HaloTag[3-19](I2R + | LgBiT-6xHis | 2.40E−05 | 7.45E+004 | 3.23E−010 |
| G3K)-4xGS-VSHiBiT | | | | |
| HaloTag[3-19](I2R + G5R)- | LgBiT-6xHis | 5.86E−06 | 5.32E+04 | 1.10E−010 |
| 4xGS-VSHiBiT | | | | |
| HaloTag[3-19](D9R + | LgBiT-6xHis | NA | 8.30E+04 | NA |
| E14K)-4xGS-VSHiBiT | | | | |
| HaloTag[3-19](D9R + | LgBiT-6xHis | NA | 6.48E+04 | NA |
| L16R)-4xGS-VSHiBiT | | | | |
| HaloTag[3-19](I2R)- | HaloTag[22-297](M2F + D53G + K140E)- | 4.82E−005 | 8.375E+004 | 5.755E−010 |
| 4xGly/Ser-VSHiBiT | 12xGly/Ser-LgBiT-3xGly/Ser-6xHis | | | |
| HaloTag[3-19](I2R)- | HaloTag[22-297](M2F + D53G + K140E)- | NA | 7.14E+004 | NA |
| 4xGly/Ser-VSHiBiT | 12xGly/Ser-LgBiT-3xGly/Ser-6xHis | | | |
| (K8R + K9R) | | | | |
| HaloTag[3-19](I2R)- | HaloTag[22-297](M2F + D53G + K140E)- | 6.05E−005 | 8.48E+004 | 7.13E−010 |
| VSHiBiT | 12xGly/Ser-LgBiT-3xGly/Ser-6xHis | | | |
| HaloTag[3-19](I2R)- | HaloTag[22-297](M2F + D53G + K140E)- | 1.98E−005 | 6.13E+004 | 3.23E−010 |
| 4xGly/Ser-HiBiT | 12xGly/Ser-LgBiT-3xGly/Ser-6xHis | | | |
| VSHiBiT-5xGly/Ser- | HaloTag[22-297](M2F + D53G + K140E)- | 6.94E−005 | 8.87E+004 | 7.82E−010 |
| HaloTag[3-19](I2R) | 12xGly/Ser-LgBiT-3xGly/Ser-6xHis | | | |
| VSHiBiT- | HaloTag[22-297](M2F + D53G + K140E)- | 6.77E−005 | 1.10E+005 | 6.13−010 |
| HaloTag[3-19](I2R) | 12xGly/Ser-LgBiT-3xGly/Ser-6xHis | | | |

### Example 5

### Surface Display and Complementation of HaloTag[3-19]-HiBiT Variants with HaloTag[22-297](M2F)-12×Gly/Ser-LgBiT

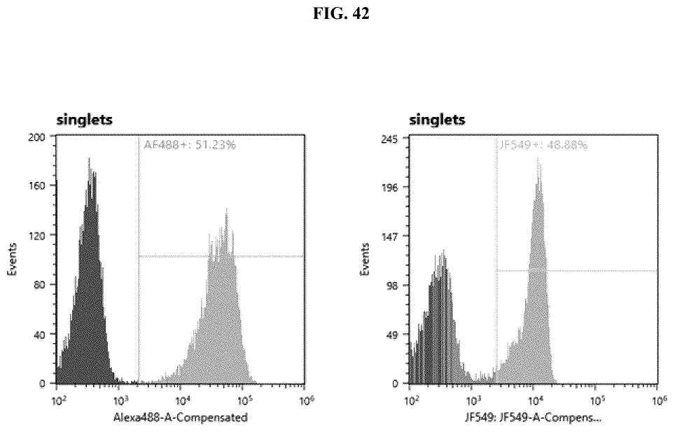

Experiments were conducted during development of embodiments herein demonstrating that HaloTag[3-19]-4×Gly/Ser-VS-HiBiT can be displayed on the surface of cells (FIG. 42). HaloTag[22-297](M2F)-12×Gly/Ser-LgBiT-6×His can complement surface displayed HaloTag[3-19]-4×Gly/Ser-VS-HiBiT, resulting in fluorescence signal upon addition of a fluorescent HaloTag® ligand (FIG. 42, right histogram).

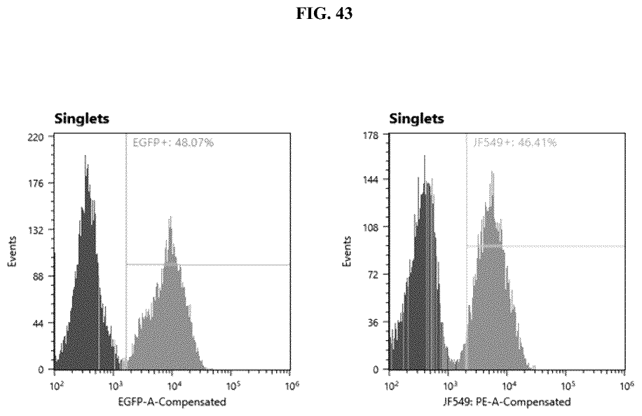

Experiments were conducted during development of embodiments herein demonstrating that HaloTag[3-19]-GGSG-VS-HiBiT can be displayed on the surface of cells as a genetic fusion to EGFP (FIG. 43). HaloTag[22-297](M2F)-12×Gly/Ser-LgBiT-6×His can complement surface displayed EGFP-GGSG-HaloTag[3-19]-GGSG-VS-HiBiT, resulting in fluorescence signal upon addition of a fluorescent HaloTag® ligand (FIG. 43, right histogram).

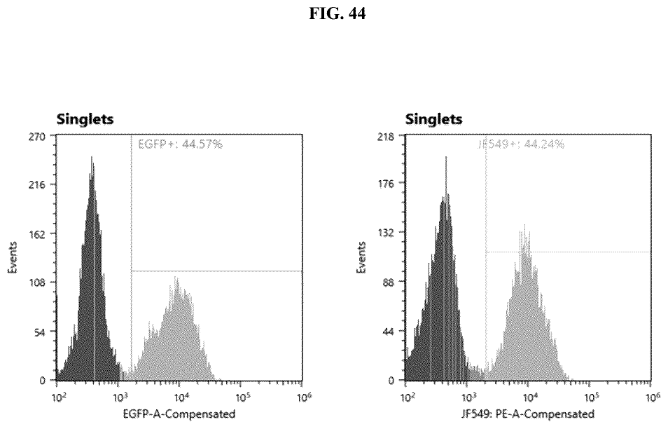

Experiments were conducted during development of embodiments herein demonstrating that HaloTag[3-19]-GGSG-VS-HiBiT variant constructs with changes to the HiBiT sequence can be displayed on the surface of cells (FIG. 44). HaloTag[22-297](M2F)-12×Gly/Ser-LgBiT-6×His can complement surface displayed EGFP-HaloTag[3-19]-HiBiT variants, resulting in fluorescence signal upon addition of a fluorescent HaloTag® ligand (FIG. 44, right histogram).

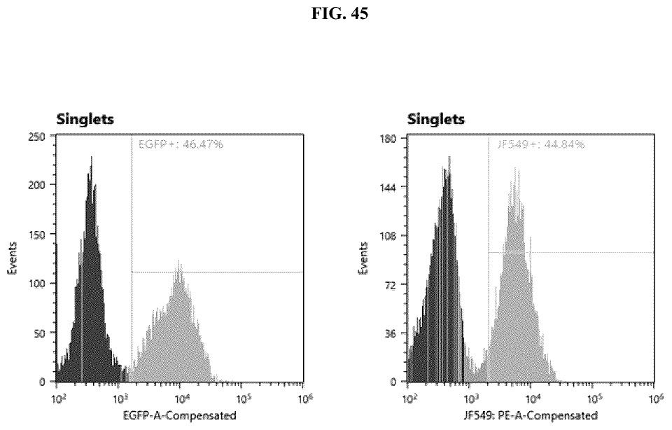

Experiments were conducted during development of embodiments herein demonstrating that HaloTag[3-19]-GGSG-VS-HiBiT variant constructs with changes to the HiBiT sequence can be displayed on the surface of cells (FIG. 45). HaloTag[22-297](M2F)-12×Gly/Ser-LgBiT-6×His can complement surface displayed EGFP-HaloTag[3-19]-HiBiT variants, resulting in fluorescence signal upon addition of a fluorescent HaloTag® ligand (FIG. 45, right histogram).

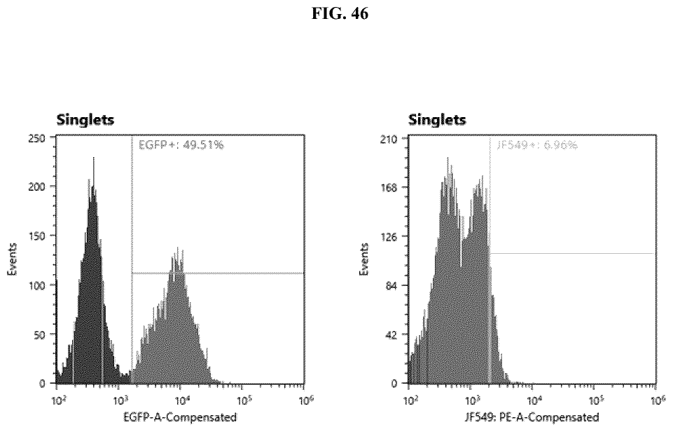

Experiments were conducted during development of embodiments herein demonstrating that HaloTag[3-19]-GGSG-VS-HiBiT variant constructs with changes to the HiBiT sequence can be displayed on the surface of cells (FIG. 46). HaloTag[22-297](M2F)-12×Gly/Ser-LgBiT-6×His can complement surface displayed EGFP-HaloTag[3-19]-HiBiT variants, resulting in fluorescence signal upon addition of a fluorescent HaloTag® ligand (FIG. 46, right histogram).

### Example 6

### Detection of Genetic Fusions to the HaloTag[3-19]-HiBiT Tag in E. coli Lysates

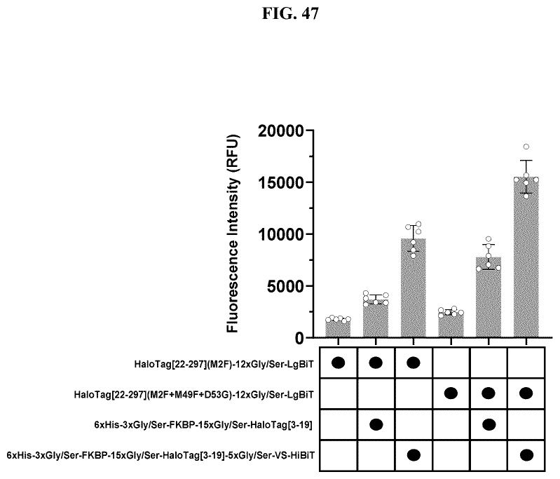

Experiments were conducted during development of embodiments herein demonstrating that we can genetically fuse either the HaloTag[3-19] or dual tag HaloTag[3-19]-HiBiT to FKBP as a target protein and observe complementation with HaloTag[22-297](M2F)-12×Gly/Ser-LgBiT or the more stable variant with M49F+D53G mutations (FIG. 47). The presence of the HiBiT in the dual tag fusion enhances the detection of the target protein, resulting in higher signals.

### Example 7

### Stabilization of Activity of LgHT-LgBiT Variants by Synthetic Peptides in E. coli Lysates

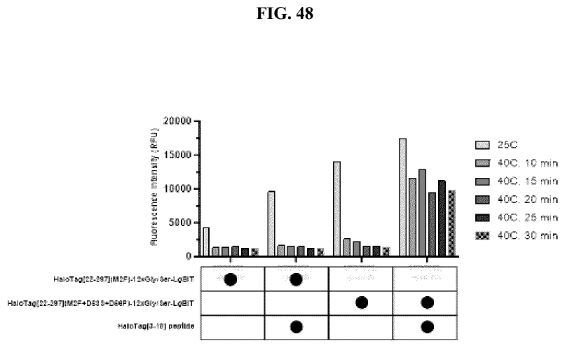

Experiments were conducted during development of embodiments herein demonstrating that we can use a synthetic peptide version of the HaloTag[3-19] fragment and observe stabilization of the more stable variant HaloTag[22-297](M2F+D53S+D56P)-12×Gly/Ser-LgBiT at higher temperature of 40 C, with no significant loss in activity after 30 minute incubation (FIG. 48). Without the D53G+D56S mutations, the parental protein loses stability and activity after incubation at 40 C.

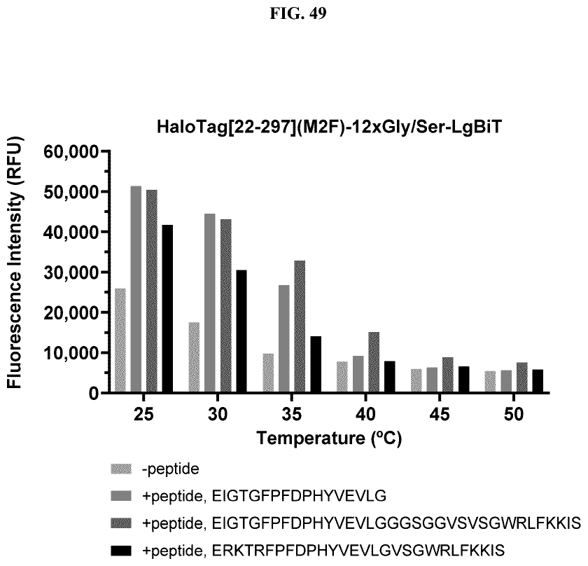

Experiments were conducted during development of embodiments herein demonstrating that differential activity and stabilization of the HaloTag[22-297](M2F)-12×Gly/Ser-LgBiT with different synthetic peptides (FIG. 49). The results show that adding the HiBiT to the dual tag peptide does not reduce stabilization of the HaloTag-[3-19] peptide. They also show that adding a Gly/Ser linker in the peptide between the two tag sequences, detection is improved with higher signals.

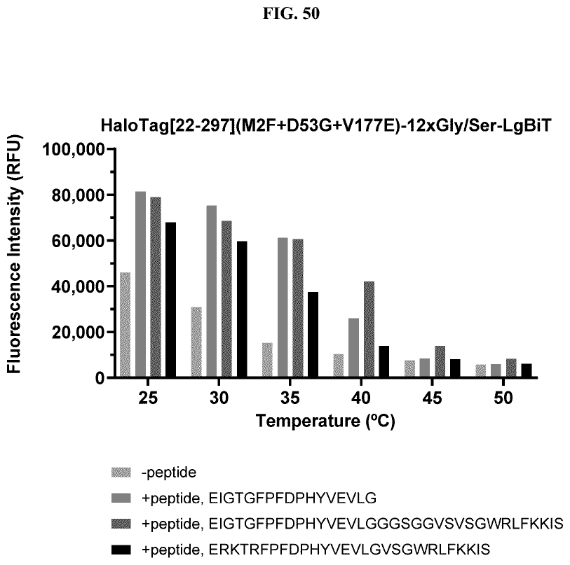

Similar to the experiment depicted in FIG. 49, except that with the mutations D53G+V177E, both expression and stabilization of the construct is increased while maintaining its response to peptide, the optimal temperature of peptide stabilization versus the non-peptide condition is at a higher temperature, closer to 35° C. (FIG. 50).

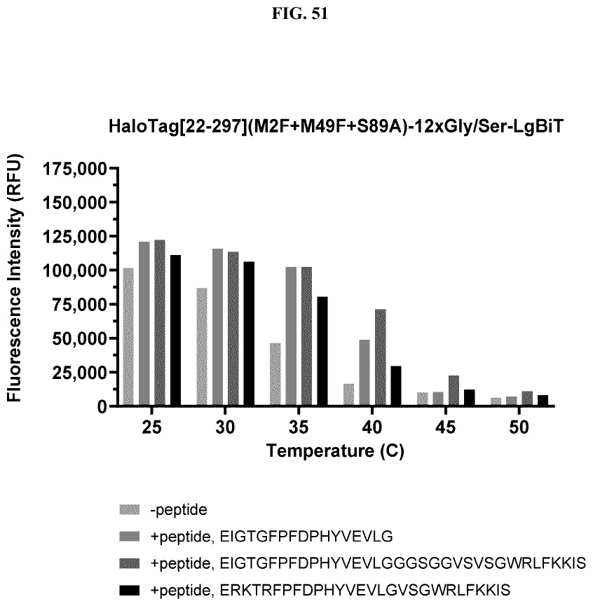

Similar to previous experiments, except that with the stabilizing mutations M49F+S89A, the optimal temperature of peptide stabilization versus the non-peptide condition is at a higher temperature, closer to 35° C. (FIG. 51).

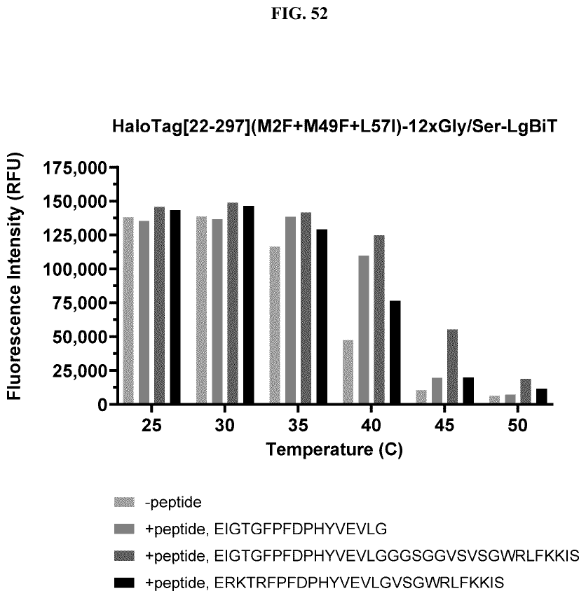

Similar to previous experiment except that with the stabilizing mutations M49F+L57I, the optimal temperature of peptide stabilization versus the non-peptide condition is at an even higher temperature, closer to 40° C. (FIG. 52).

### Example 8

### Labeling Kinetics of LgHT-LgBiT Variants with TMR HaloTag Ligand in E. coli Lysates

Experiments were conducted during development of embodiments herein to analyze the labeling kinetics of LgHT-LgBiT variants with TMR HaloTag® ligand in E. coli lysates.

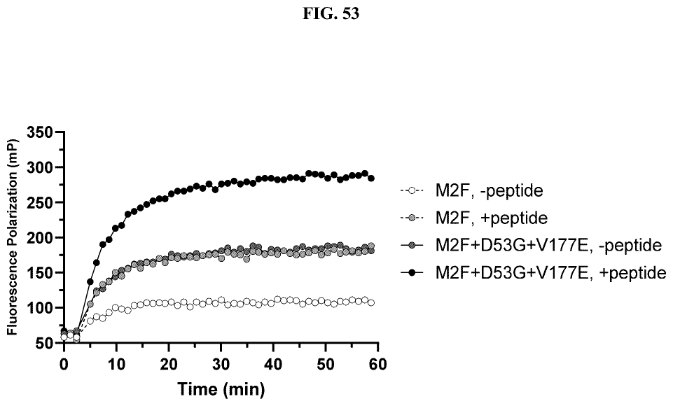

The higher plateau of the M2F+D53G+V177E variant over the M2F control is indicative of improved expression of this construct in E. coli lysates with simultaneous improvement in response to synthetic peptide complementation, measured in the experiments of FIG. 53 as faster ligand binding and labeling with the TMR chloroalkane HaloTag® ligand.

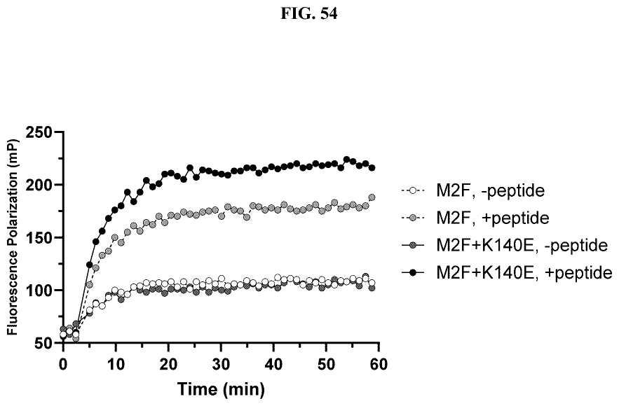

The higher plateau of the M2F+K140E variant over the M2F control is indicative of improved expression of this construct in E. coli lysates (FIG. 54). This variant shows that it has similar levels of non-complemented “background” labeling in the absence of peptide (-peptide) as the M2F control but with improved expression, resulting in better fold response to complementation with the peptide. Therefore, this is a high value variant since it improves the dynamic range of detection while also improving the expression and stability of the construct.

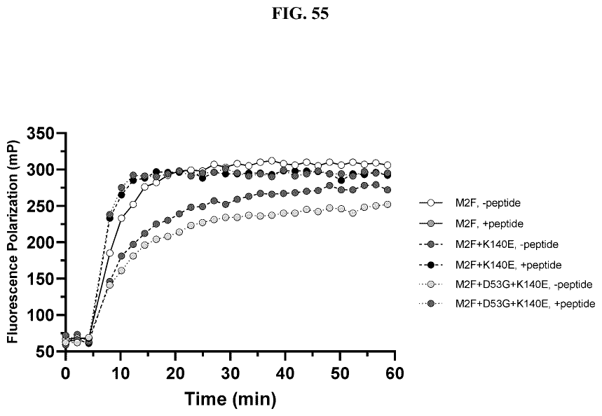

FIG. 55 demonstrates that the parental M2F variant is improved by adding the K140E mutation, and then further improved with the combined D53G+K140E mutations in terms of consecutively lower levels of “background” or peptide-independent labeling with the TMR HaloTag® ligand. Therefore, the triple mutant M2F+D53G+K140E has the best dynamic range of all combinations tested and is our best candidate protein.

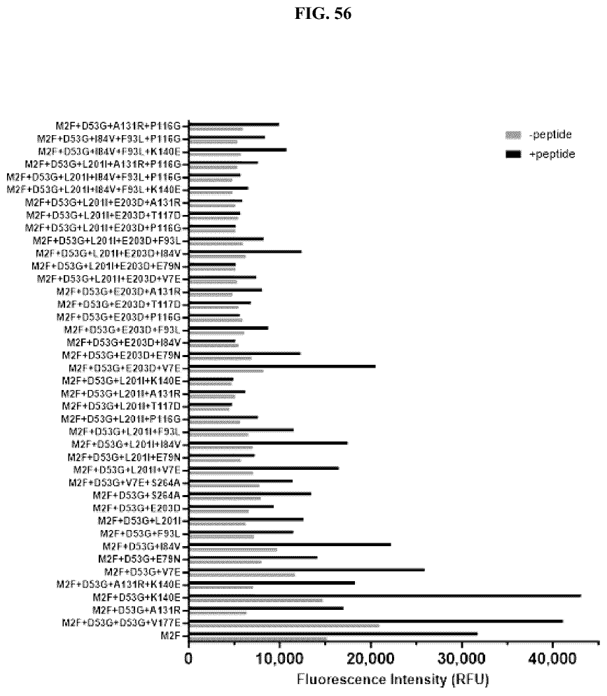

Several single and double mutant combinations that improve the fold response of the system to synthetic peptide over the M2F parental template (FIG. 56). Notably, the K140E, A131R, V7E, and V7E+E203D are some of the best performers, most of which are lower in expression except for K140E which improves both attributes.

### Example 9

### Stability and Labeling Kinetics of Purified LgHT-LgBiT Variants with TMR HaloTag Ligand

While experiments above were performed using E. coli lysates, experiments were also conducted during development of embodiments herein using a completely purified system with both purified HaloTag[22-297](M2F)-12×Gly/Ser-LgBiT-6×His combined with synthetic HaloTag[3-19] peptide.

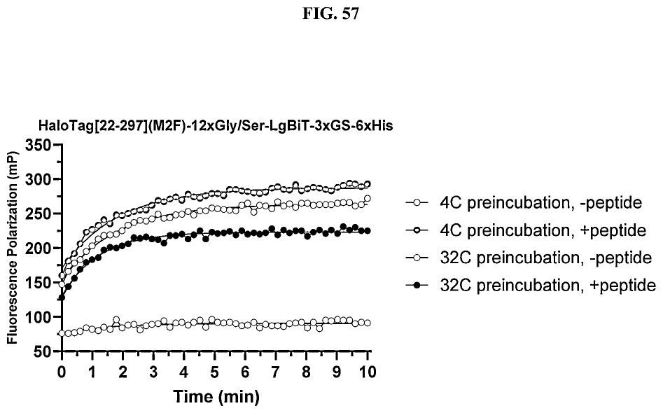

The peptide has a stabilization effect on the active protein, since a 32° C. incubation is enough to eliminate activity without peptide, whereas the presence of peptide retains a significant fraction of the original activity in non-heat treated (4 C) reactions (FIG. 57).

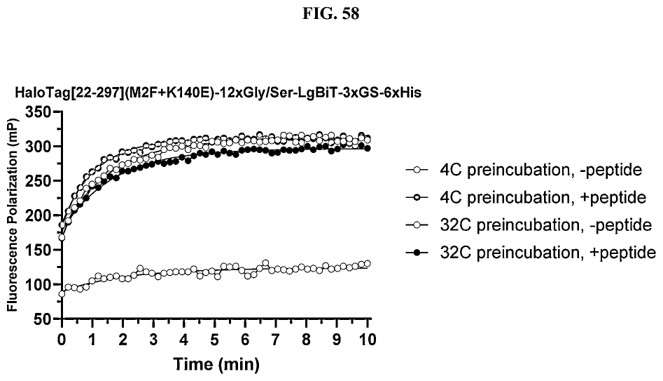

Similar to experiments in E coli lysates, the M2F+K140E variant exhibits improved initial labeling rate of the protein (4° C. samples) and a greater retention of activity after 32° C. incubation when synthetic peptide is present (32° C., +peptide) (FIG. 58). Thus, the M2F+K140E shows improved dynamic range in the assay.

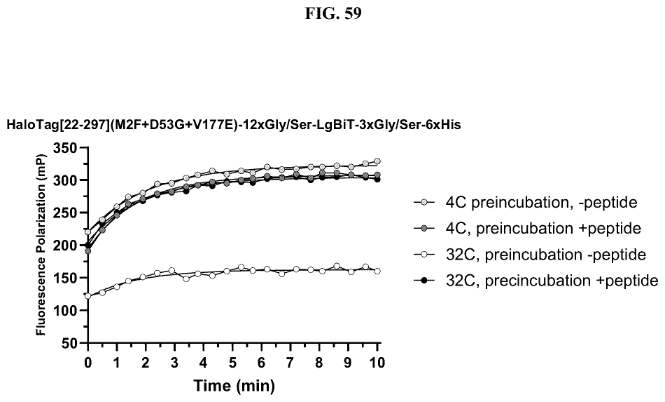

The M2F+D53G+V177E variant as a purified protein performs similarly to lysates, with a greater stability and initial activity relative to the M2F template (FIG. 59). However, it does have higher “background” labeling (-peptide) under these temperature challenge conditions, which limits its dynamic range or measured response to peptide. Higher temperature challenge conditions, such as 35° C., fit the optimal dynamic range of this variant.

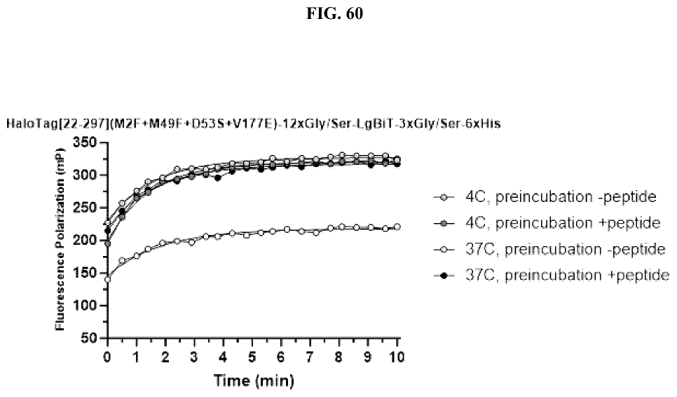

The M2F+D53S+V177E is an example of a more highly stabilized variant, which loses less than half of its activity at a higher 37° C. temperature challenge (FIG. 60). M2F+D53S+V177E still maintains a measurable stabilization and activity response to the presence of peptide, demonstrating that the stability of the protein can be tuned without compromising its functional as a complementation reporter.

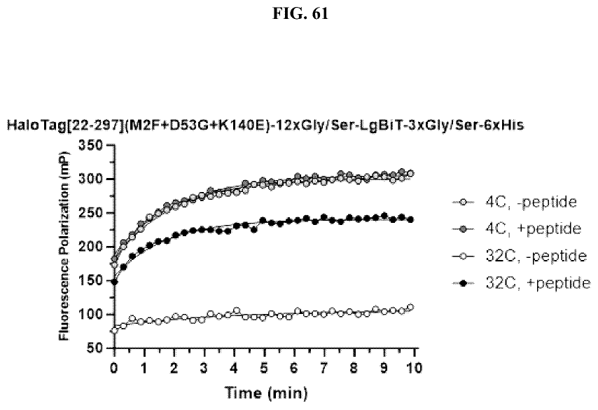

The M2F+D53G+K140E provides an improvement in response to peptide relative to the M2F+K140E double mutant (FIG. 61).

### Example 10

### Performance Evaluation in Mammalian Cells

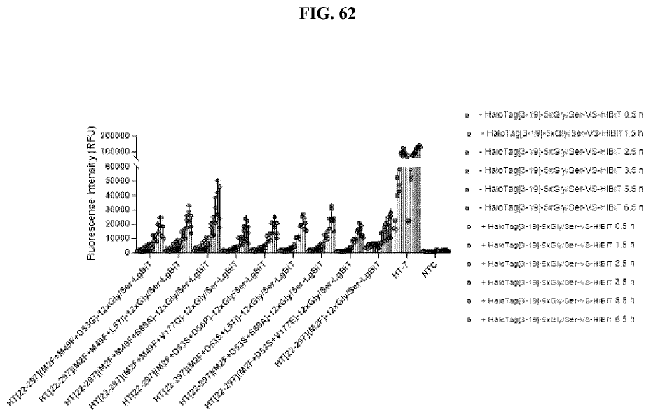

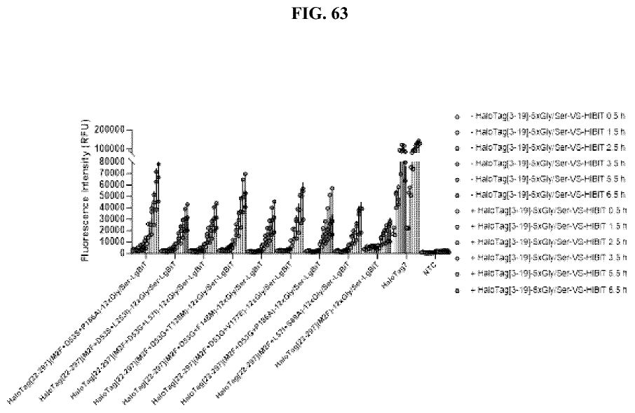

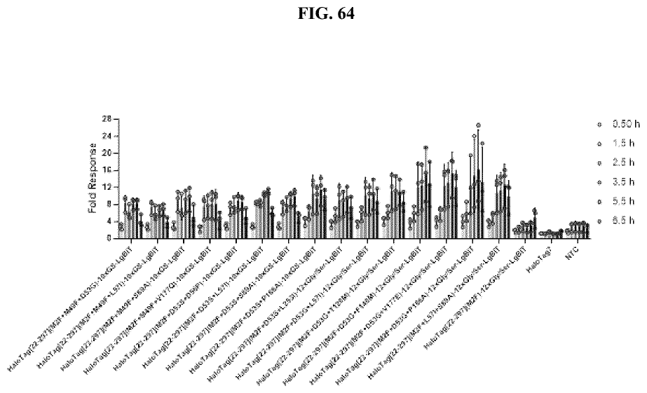

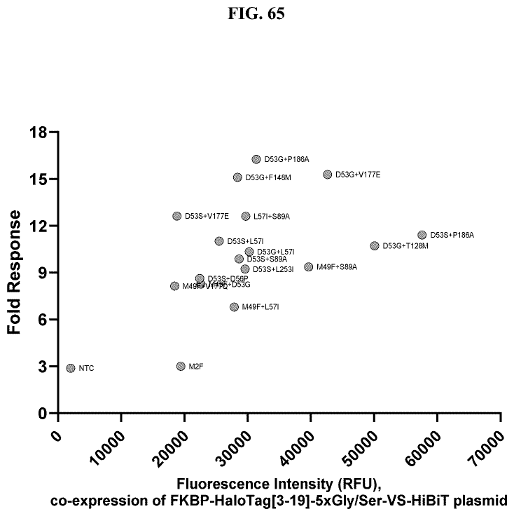

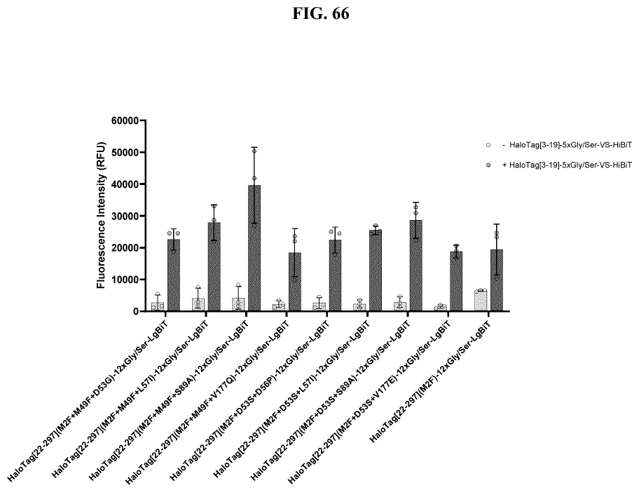

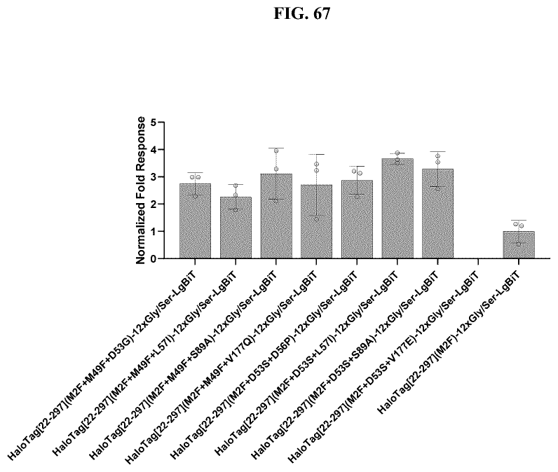

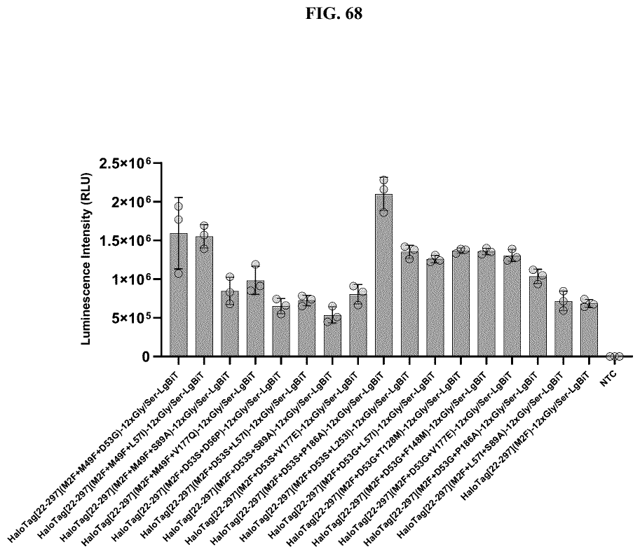

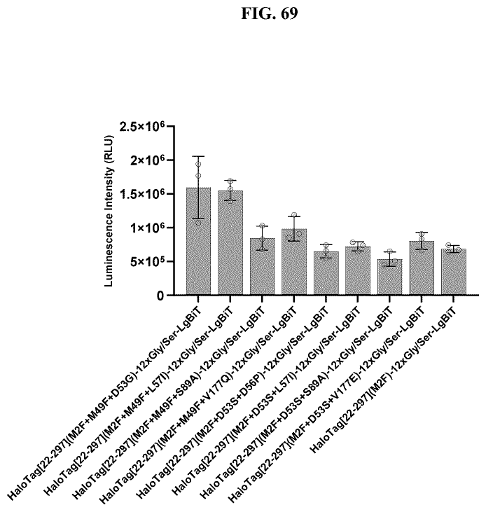

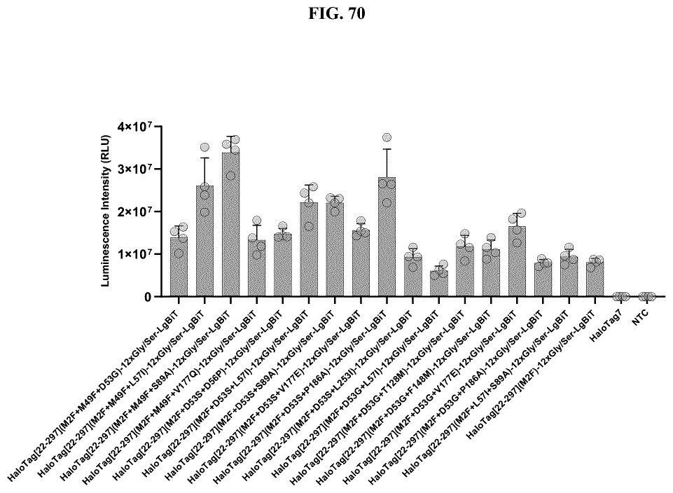

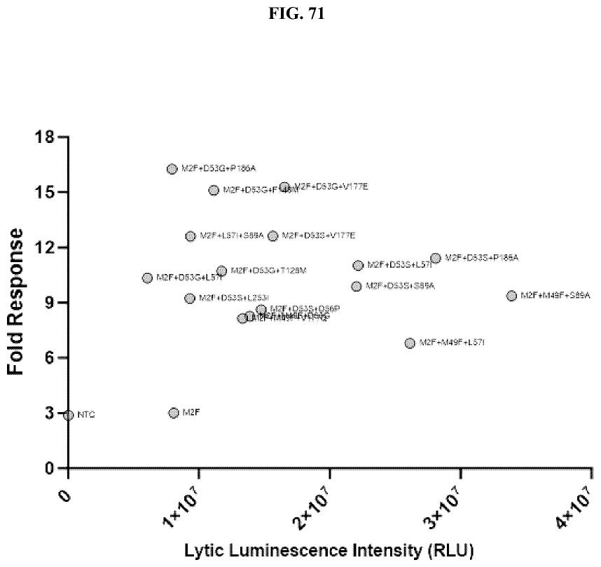

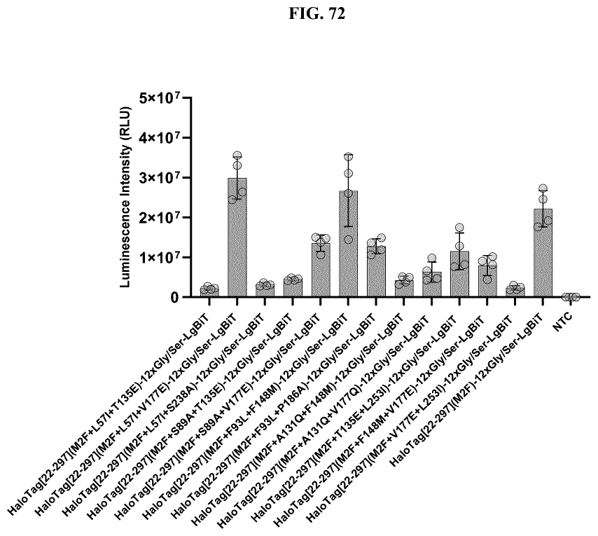

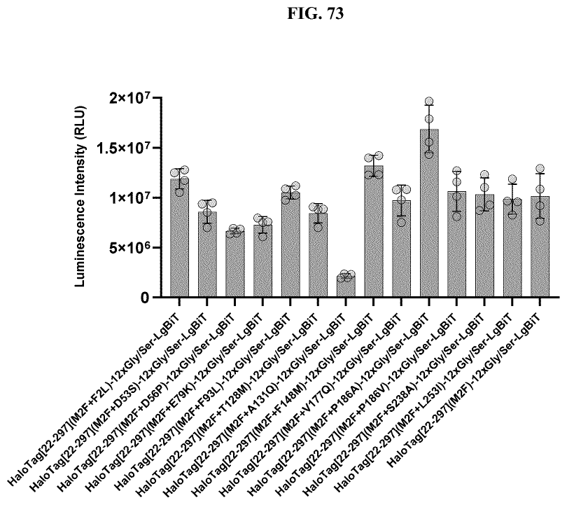

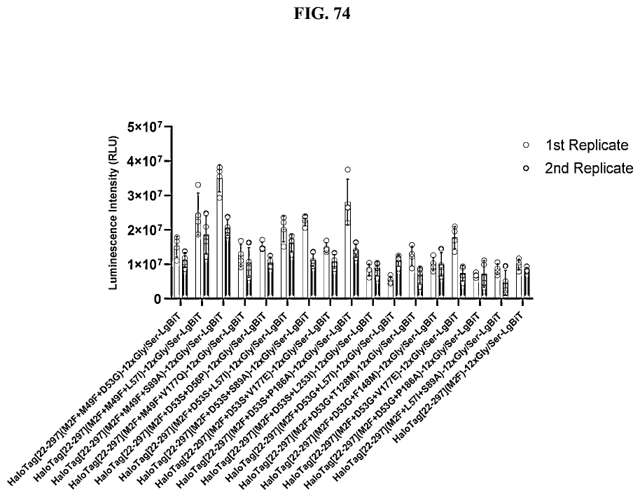

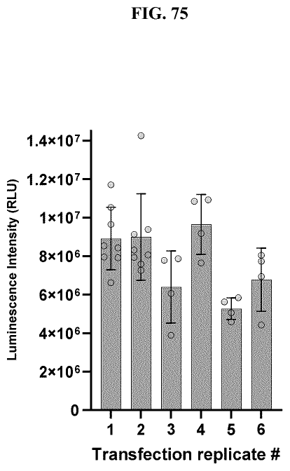

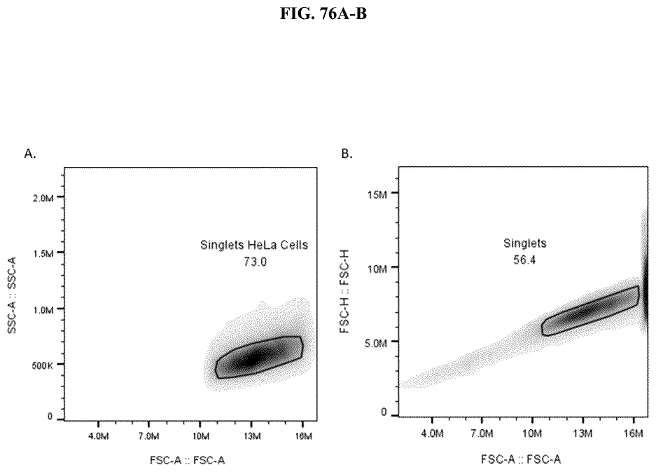

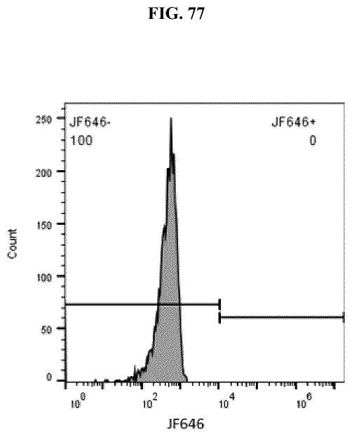

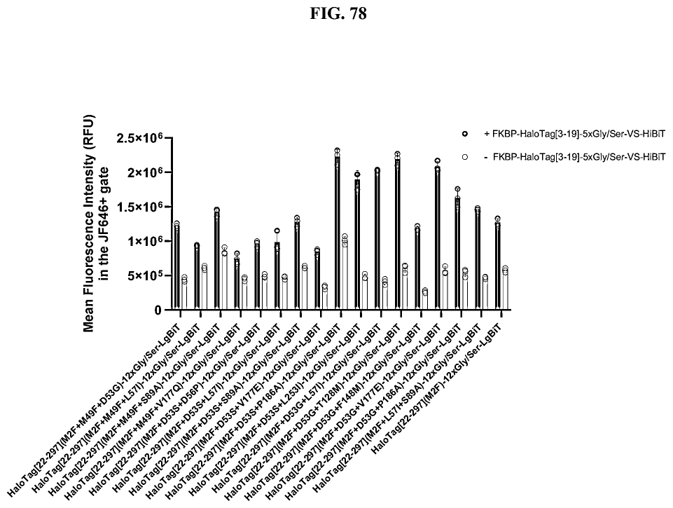

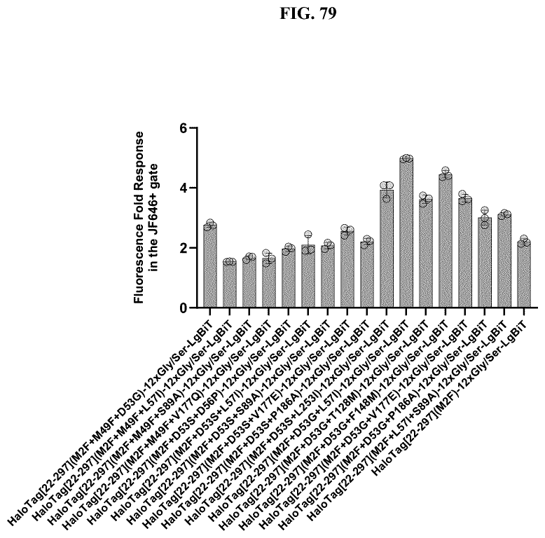

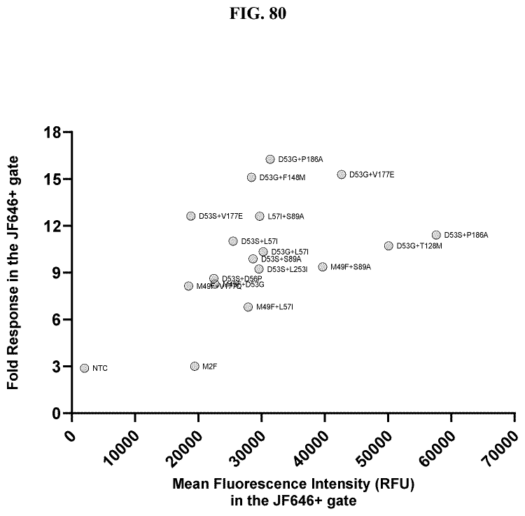

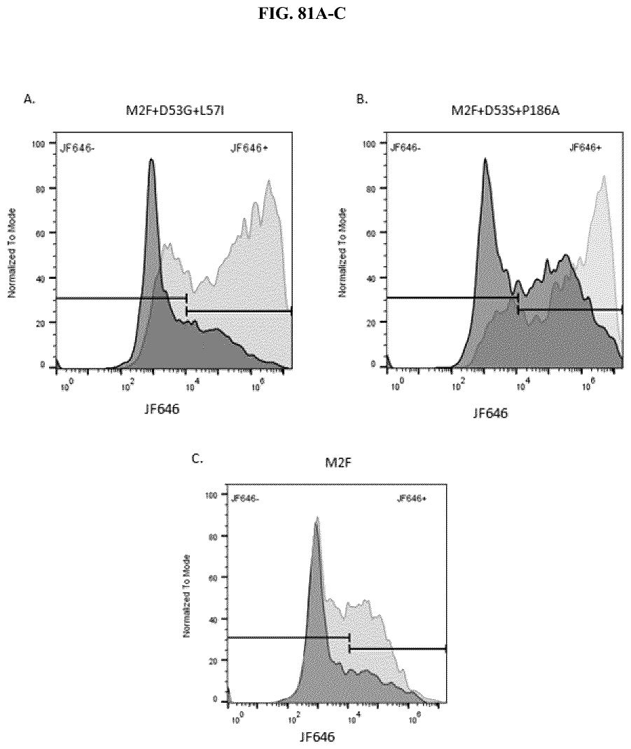

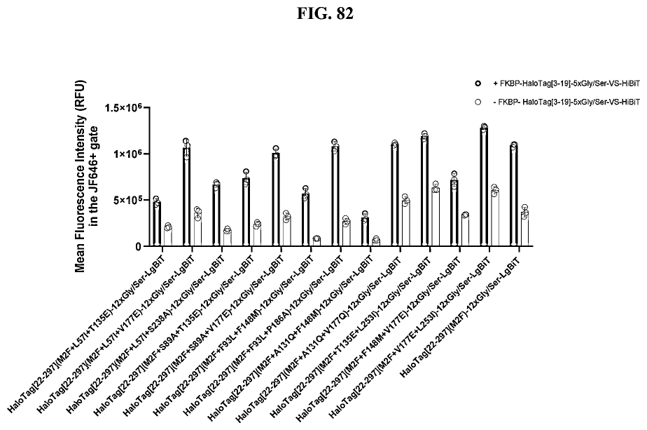

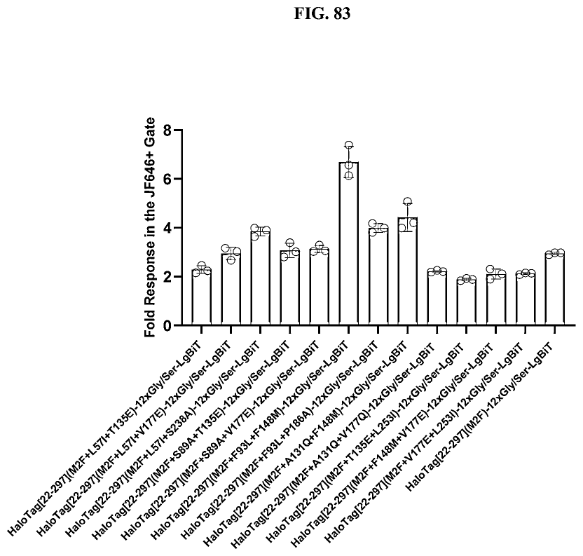

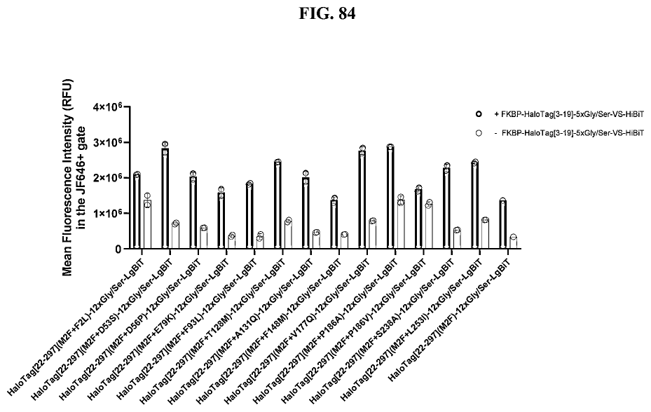

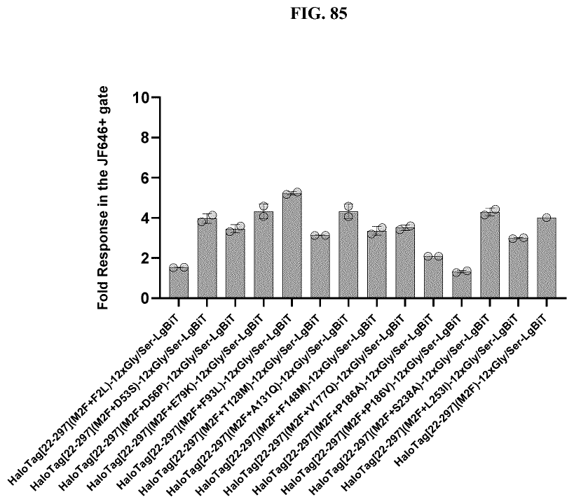

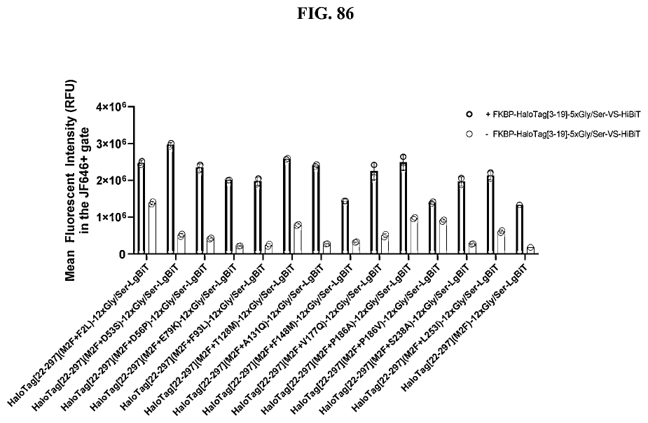

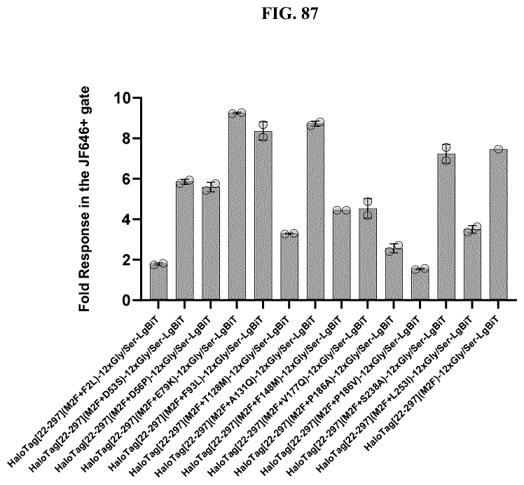

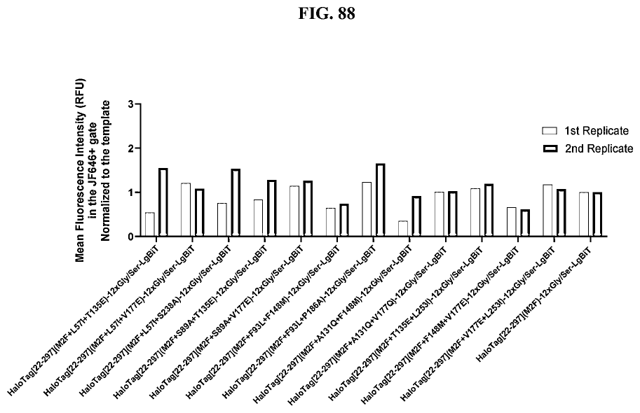

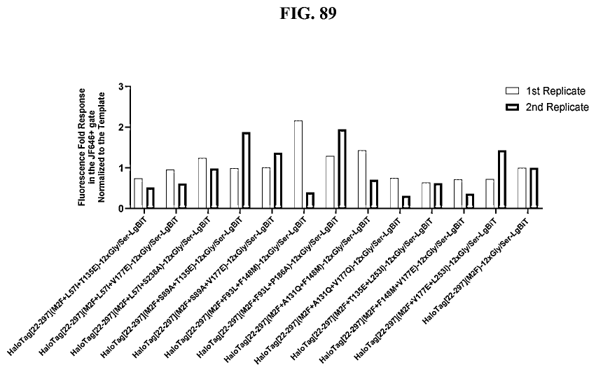

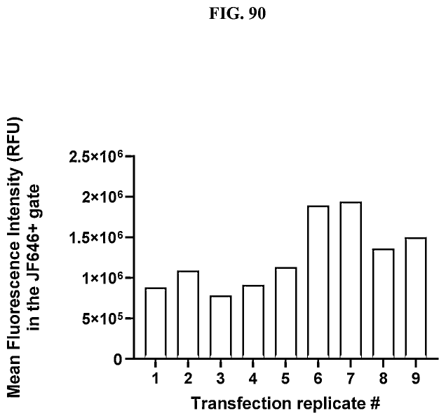

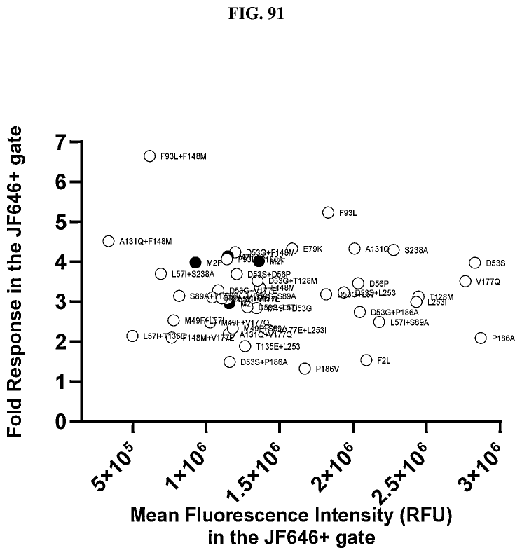

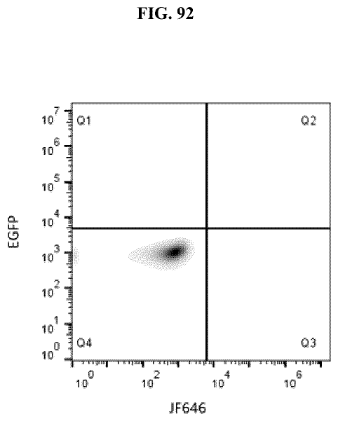

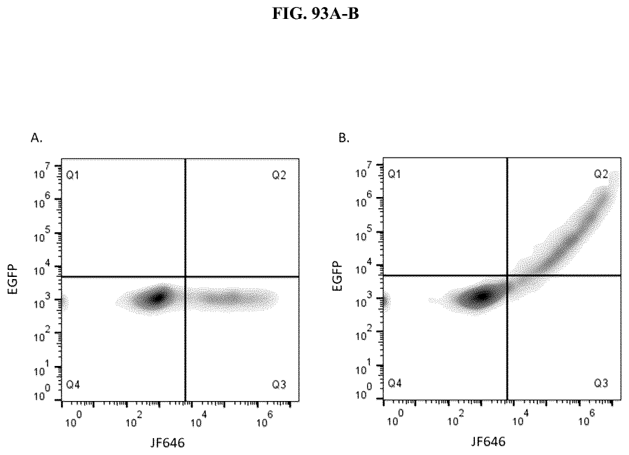

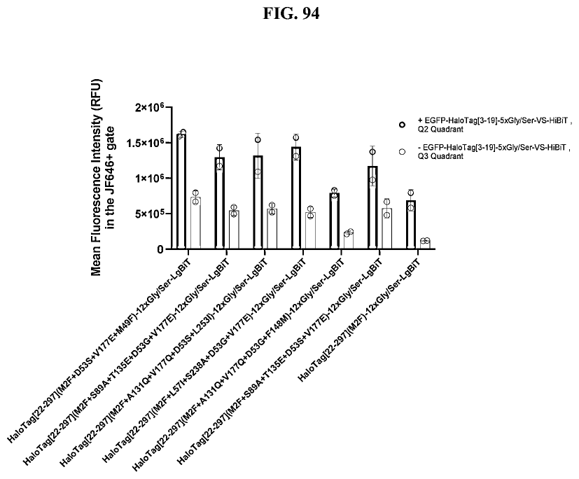

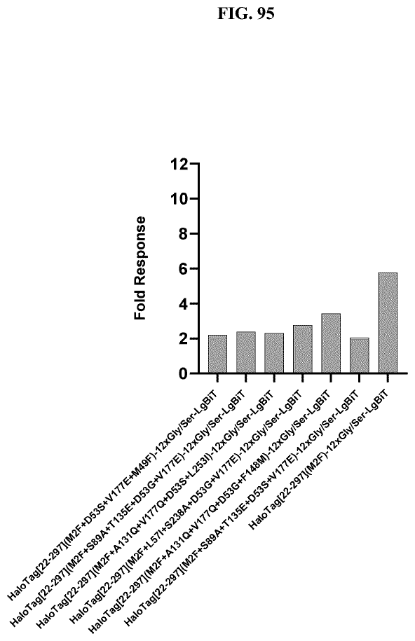

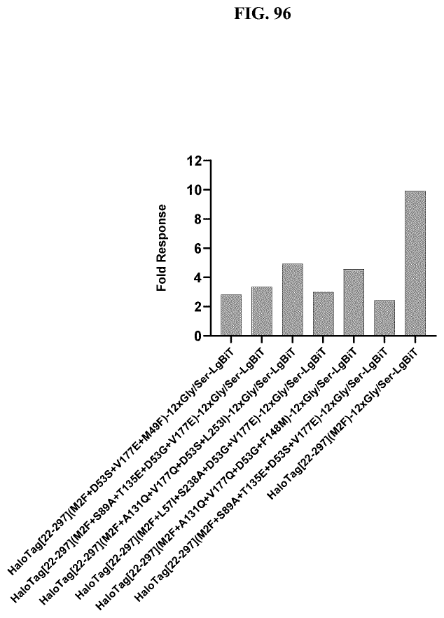

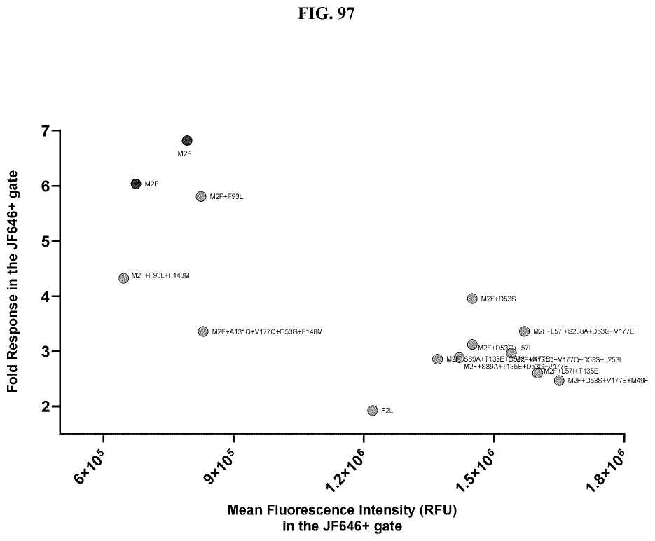

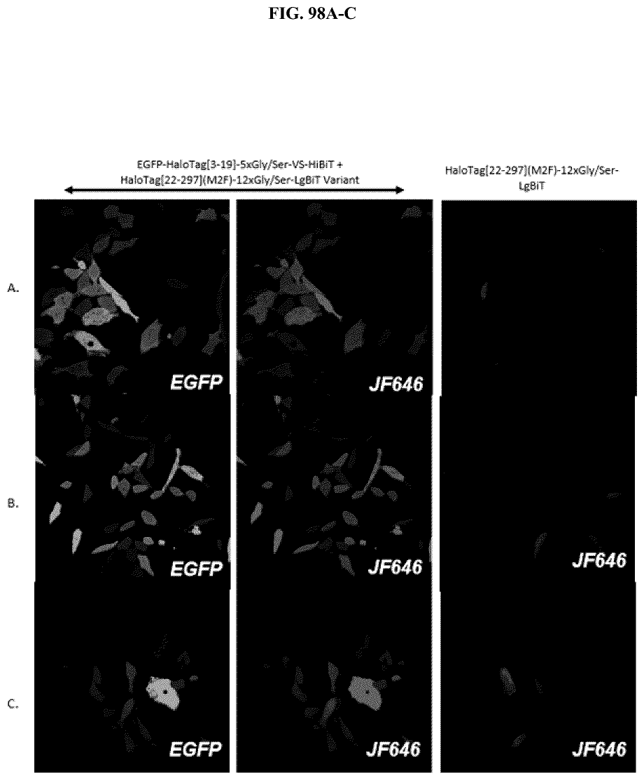

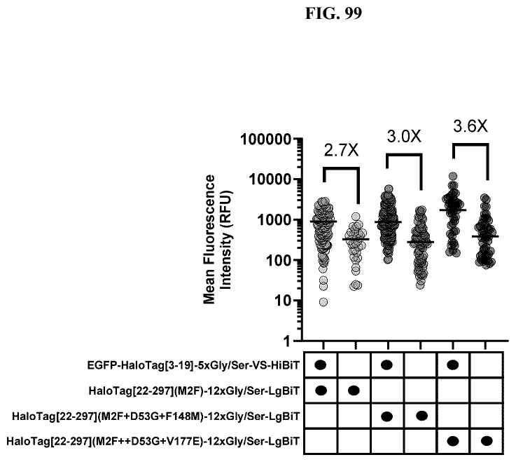

Experiments were conducted during development of embodiments herein to evaluate the performance of HaloTag[22-297](M2F)-12×Gly/Ser-LgBiT variants and the HaloTag[3-19]-5×Gly/Ser-VS-HiBiT tag upon overexpression in mammalian cells using plate assays (FIGS. 62-75), flow cytometry (FIGS. 76-97), and confocal imaging (FIGS. 98-99). In flow cytometry experiments, to assess the expression of HaloTag[3-19]-5×Gly/Ser-VS-HiBiT, a strategy where the enhanced Green Fluorescent Protein (EGFP) was fused to HaloTag[3-19]-5×Gly/Ser-VS-HiBiT was employed. This allowed for comparison of the far-red signal of the complemented complex in the far-red channel with the expression of HaloTag[3-19]-5×Gly/Ser-VS-HiBiT in the green channel. Additionally, this approach allowed for the measurement of HaloTag[3-19]-5×Gly/Ser-VS-HiBiT expression levels across different transfection experiments. The initial step of the gating strategy, involving the filtration of dead cells, cell debris, doubles, and cell aggregates, followed the same principles as described in Section B for single-color cytometry. However, further gating steps were necessary in the two-color cytometry assay to identify single-positive populations for each channel, as well as double-positive and double-negative populations.

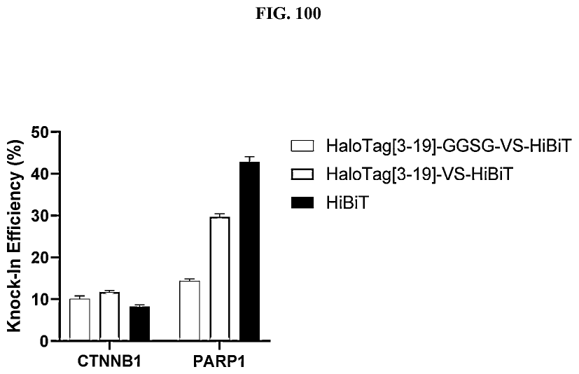

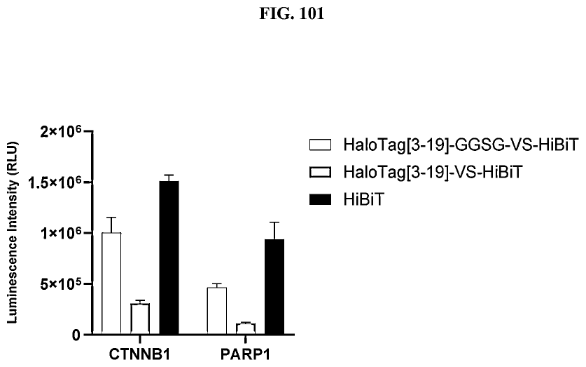

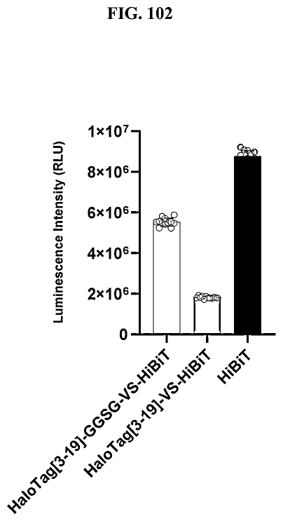

Experiments were conducted during development of embodiments herein to evaluate transiently transfected HaloTag[22-297](M2F)-12×Gly/Ser-LgBiT variant function in endogenously tagged PARP1-GGSG[HaloTag3-19]-GGSG-HiBiT in mammalian cells (FIGS. 100-102). The HaloTag[3-19]—VS-HiBiT variant with the two 4GS linkers exhibits significantly higher luminescent activity compared to the variant without linkers. These findings highlight the role of linker length in facilitating the complementation of the complex. The 4GS linker was retained for subsequent experiments.

To assess the fluorescent intensity and fold response of the endogenously expressed HaloTag[3-19]—VS-HiBiT variants in conjunction with overexpressed HaloTag[22-297](M2F)-12×Gly/Ser-LgBiT variants, transient transfections of the PARP-1 CRISPR cell line were conducted, which was tagged with the HaloTag[3-19]—VS-HiBiT variant containing 4GS linkers. Different concentrations of HaloTag[22-297](M2F)-12×Gly/Ser-LgBiT variants DNA were utilized to determine the optimal amount for efficient transfection of the PARP-1 CRISPR cell line. Furthermore, the effects of employing two distinct promoters were explored, namely the strong CMV promoter and the weak TK promoter, for the overexpression of the HaloTag[22-297](M2F)-12×Gly/Ser-LgBiT variants. This evaluation allowed for investigation of the influence of varying expression levels of the HaloTag[22-297](M2F)-12×Gly/Ser-LgBiT variants on Fluorescent intensity and the fold response.

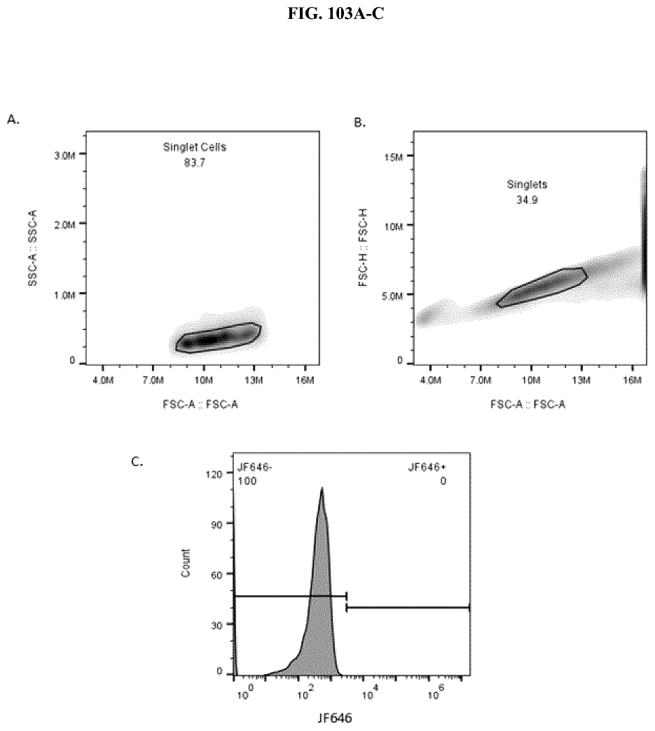

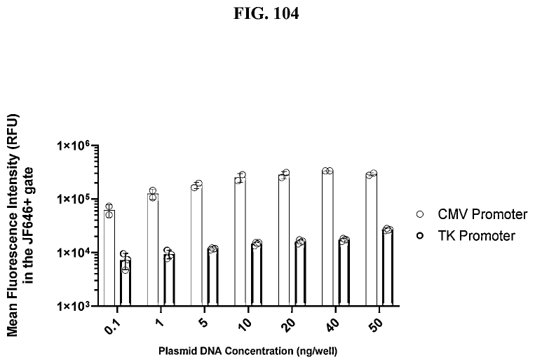

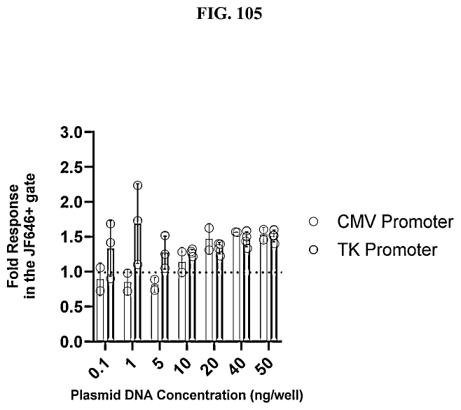

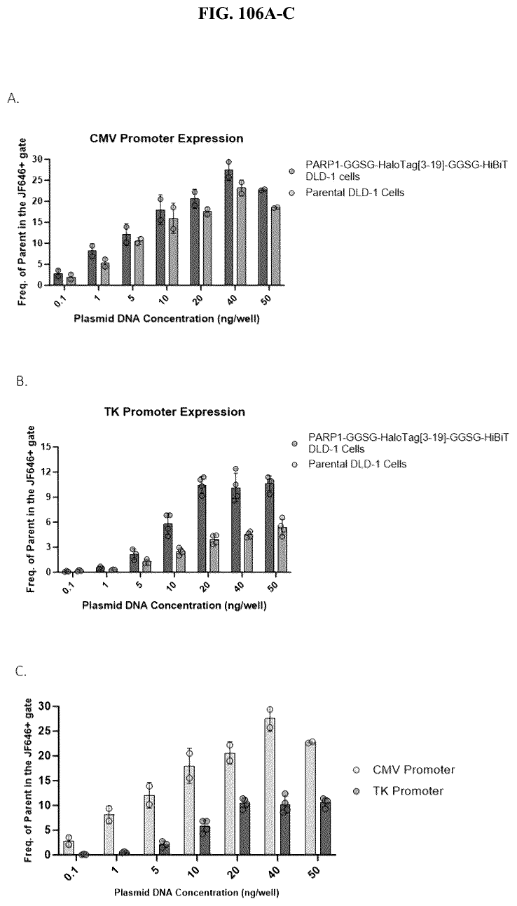

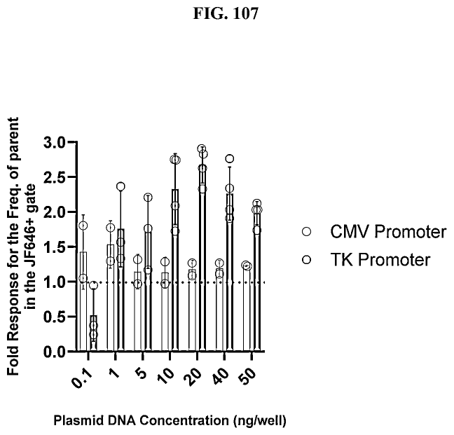

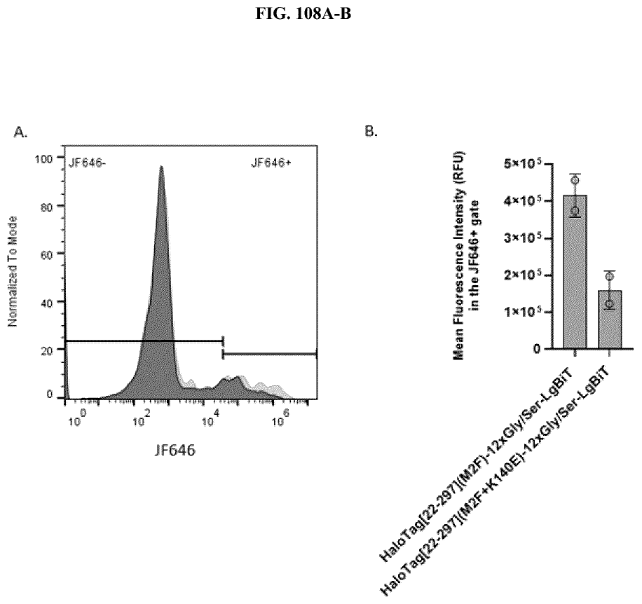

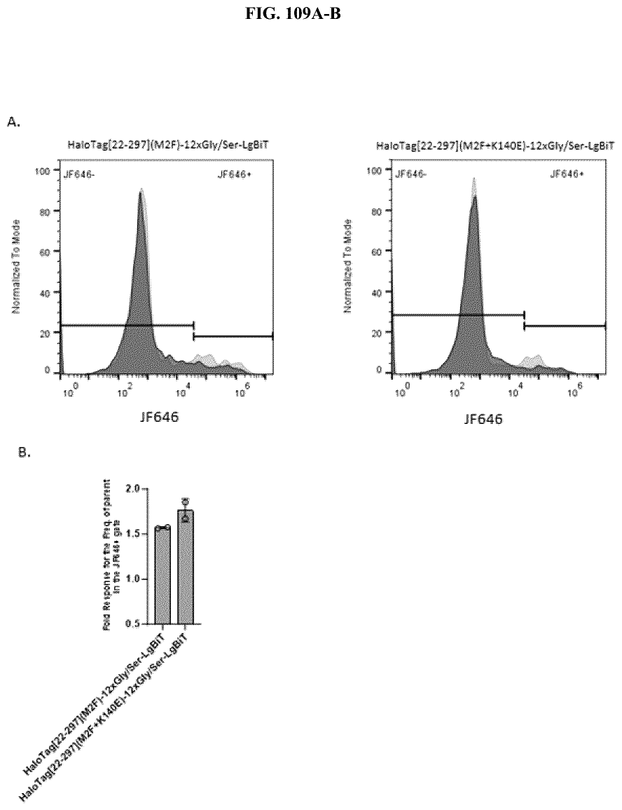

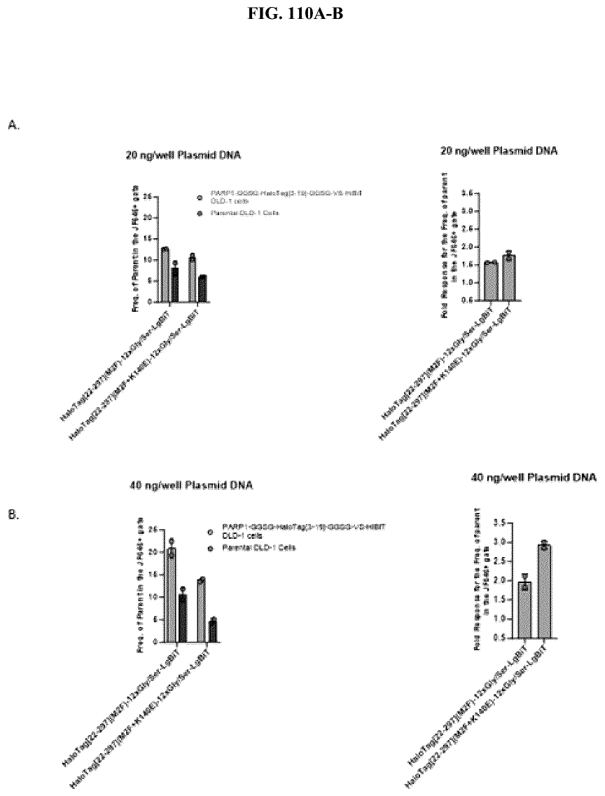

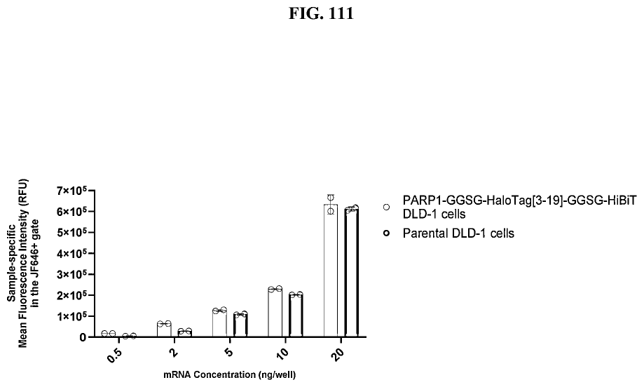

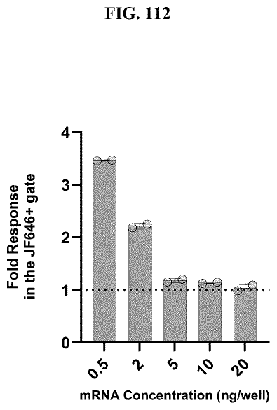

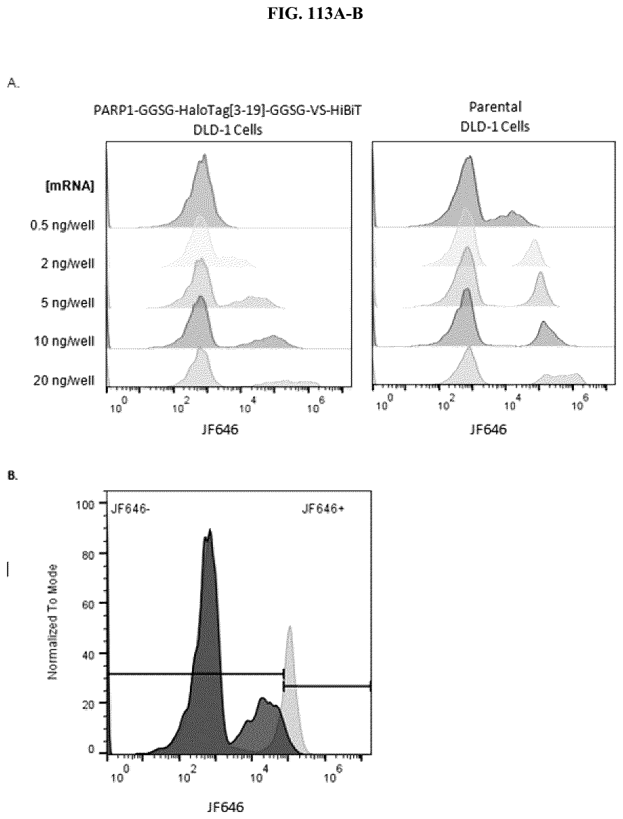

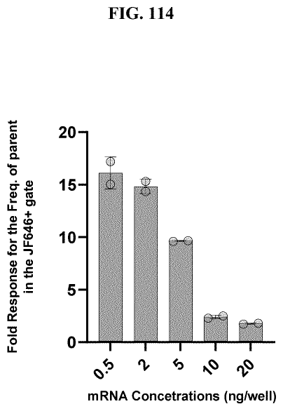

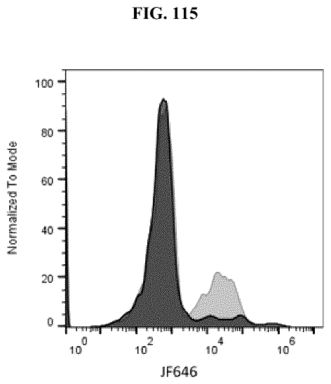

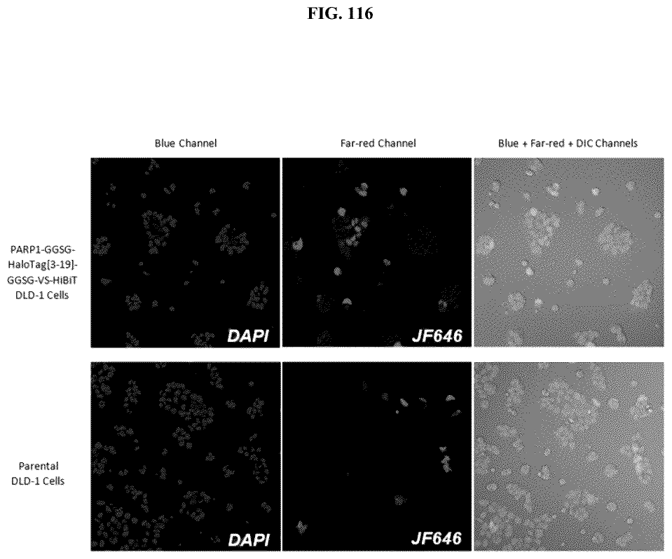

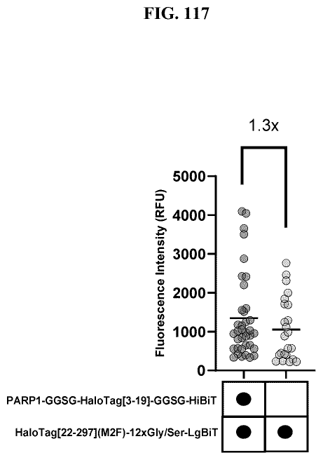

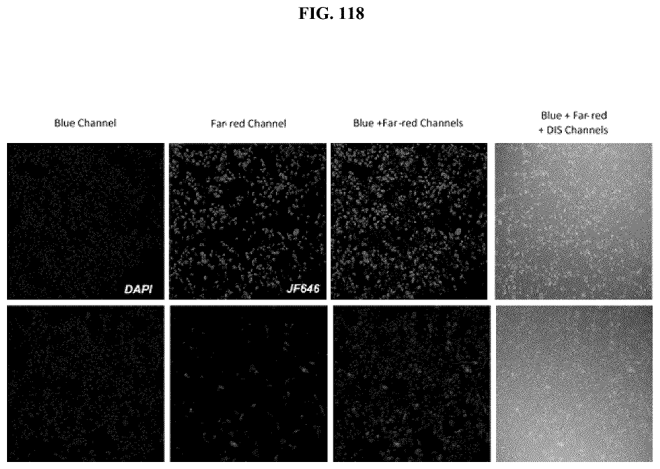

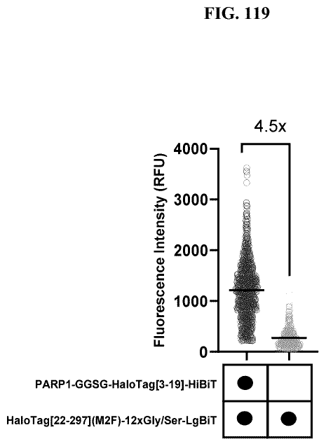

Experiments were conducted during development of embodiments herein to evaluate the performance of HaloTag[22-297](M2F)-12×Gly/Ser-LgBiT variants and the HaloTag[3-19]-5×Gly/Ser-VS-HiBiT tag upon overexpression in mammalian cells using flow cytometry (FIGS. 103-115) and confocal imaging (FIGS. 116-119).

A variable used to assess the fold response of the system in flow cytometry experiments was the comparison of fold response based on the percentage of cells in the JF646 positive gate relative to the total cells in the positive and negative JF646 populations. This variable is referred to as the frequency of the parent. Comparing this variable between CRISPR cells and parental cells provides insight into the enrichment of tagged CRISPR cells over parental cells within a specific gate. In the experimental phase, transient transfection of the PARP-1-tagged DLD-1 CRISPR cell line expressing GGSG-HaloTag[3-19]-GGSG-VS-HiBiT with HaloTag[22-297](M2F)-12×Gly/Ser-LgBiT variants mRNA was conducted. The primary aim was to investigate the potential benefits of using HaloTag[22-297](M2F)-12×Gly/Ser-LgBiT variants mRNA, as opposed to DNA, in terms of enhancing expression homogeneity and improving transfection efficiency. To achieve this objective, various concentrations of HaloTag[22-297](M2F)-12×Gly/Ser-LgBiT variants mRNA were applied to the CRISPR cell line for the evaluation of specific signals. Additionally, the parental DLD-1 cells were utilized as a control to measure the background signal and determine the concentration of HaloTag[22-297](M2F)-12×Gly/Ser-LgBiT variants mRNA that yields the highest fluorescent fold response.

### Example 11

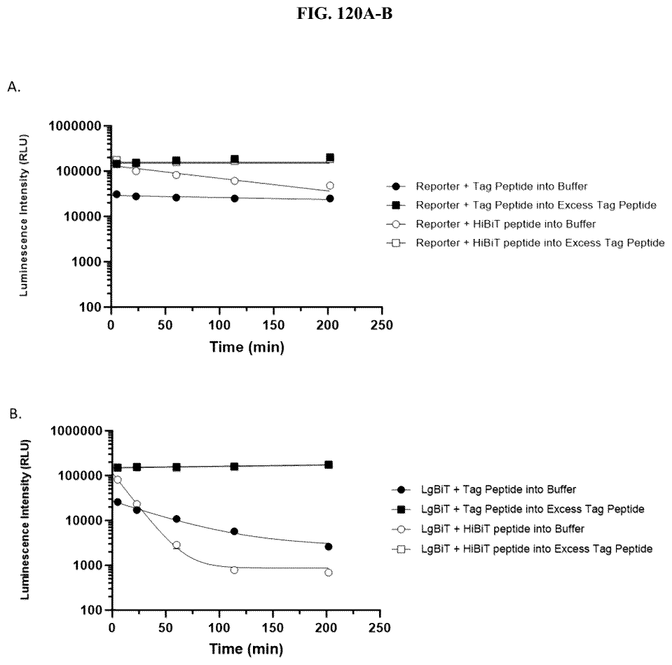

Experiments were conducted during development of embodiments herein to confirm data from biolayer interferometry (BLI) that shows exceptionally slow dissociation kinetics of the LgHT-LgBiT from the DualTag peptide (FIG. 120). It also shows that LgBiT alone has a faster dissociation from the DualTag peptide relative to the LgHT-LgBiT, indicating that additional contacts between the LgHT and DualTag sequences both contribute to the system's binding kinetics.

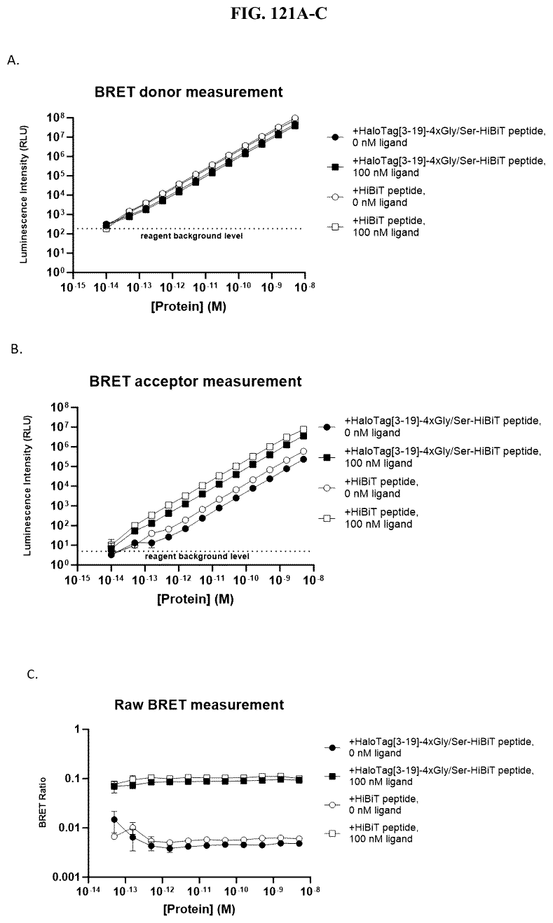

Experiments were conducted during development of embodiments herein to demonstrate the use of LgHT-LgBiT and the DualTag with NanoBRET (FIG. 121). It simultaneously measures the luminescence output from the LgBiT:HiBiT portion of the complex, which is dependent on the amount of complementation of the fragments, and the BRET ratio is directly related to the fraction of complexes bound to the HaloTag® NanoBRET® 618 Ligand. The use of these measurements to calculate the BRET ratio gives a measure of occupancy of the complex with HaloTag 618 ligand over a broad range of concentrations, allowing the LgHT activity to be monitored in both the presence or absence of complementation with HaloTag[3-19] portion of the DualTag peptide.

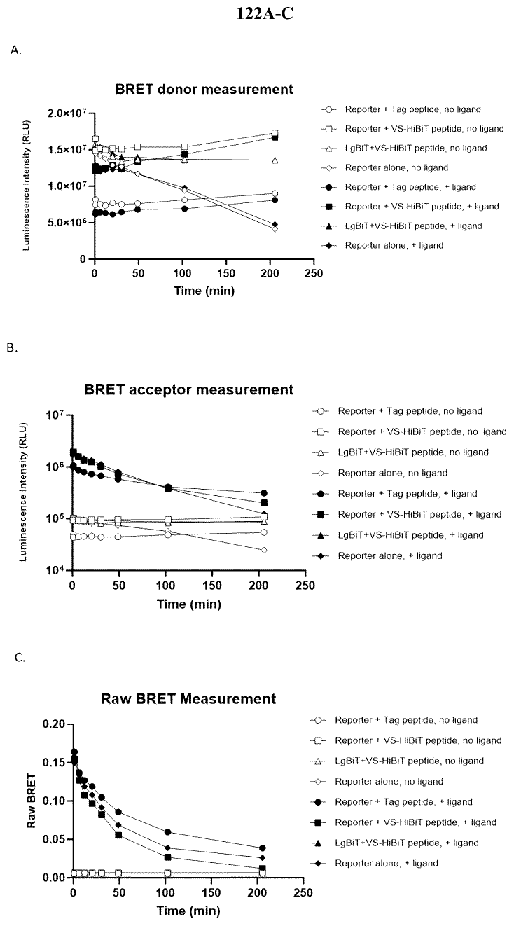

Experiments were conducted during development of embodiments herein to demonstrate the stabilization of the LgHT-LgBiT by the DualTag peptide (FIG. 122). The Raw BRET ratio, which indicates the amount of active LgHT-LgBiT remaining in the complex over time remains higher than the LgHT-LgBiT alone, showing that both its LgBiT luminescence and LgHT ligand binding activity remain intact for longer in the presence of DualTag peptide. The relatively stable luminescence donor signal over time that occurs as luminescence acceptor signal and BRET ratio decline indicates that the LgBiT in the LgHT-LgBiT fusion can retain activity even when the LgHT activity is lost.

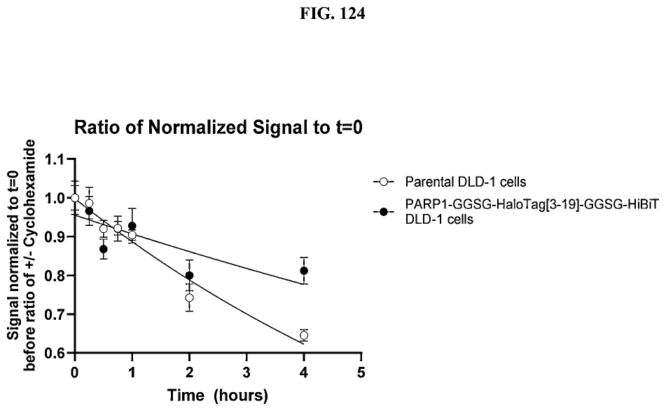

Experiments were conducted during development of embodiments herein to demonstrate that the level of active LgHT-LgBiT in maintained longer in cells also expressing a target protein with an endogenous DualTag using a Cycloheximide Pulse-Chase approach (FIG. 124). Alongside evidence from flow cytometry, it supports a model where the LgHT-LgBiT binds and is stabilized by the DualTag, which results in greater lifespan and therefore labeling of the active complex in cells.

### Example 12

### Labeling Kinetics of HaloTag[22-297]-12×Gly/Ser-LgBiT with Various HaloTag Ligands

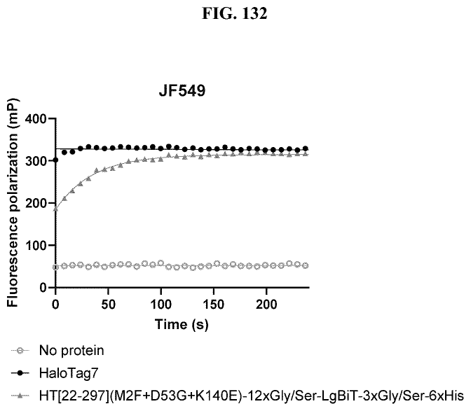

Experiments conducted during development of embodiments herein demonstrated that HaloTag[22-297](M2F+D53G+K140E)-12×Gly/Ser-LgBiT-3×Gly/Ser-6×His is functional with JF549 HaloTag ligand (FIG. 132).

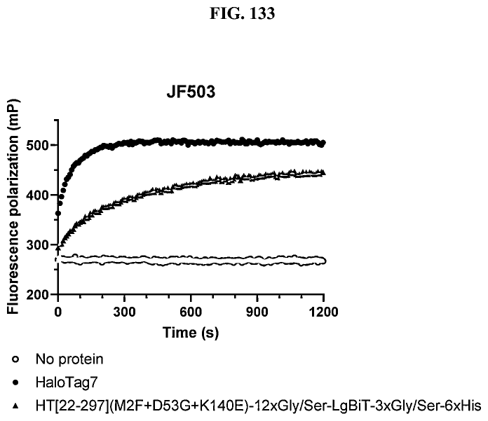

Experiments conducted during development of embodiments herein demonstrated that HaloTag[22-297](M2F+D53G+K140E)-12×Gly/Ser-LgBiT-3×Gly/Ser-6×His is functional with JF503 HaloTag ligand (FIG. 133).

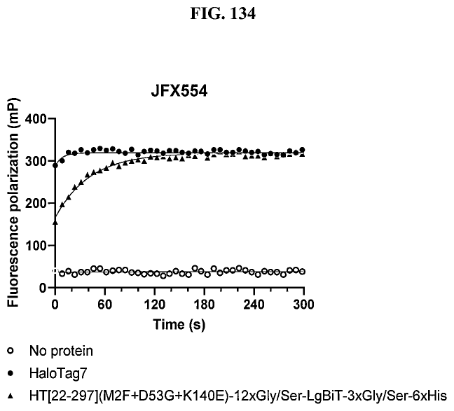

Experiments conducted during development of embodiments herein demonstrated that HaloTag[22-297](M2F+D53G+K140E)-12×Gly/Ser-LgBiT-3×Gly/Ser-6×His is functional with JFX554 HaloTag ligand (FIG. 134).

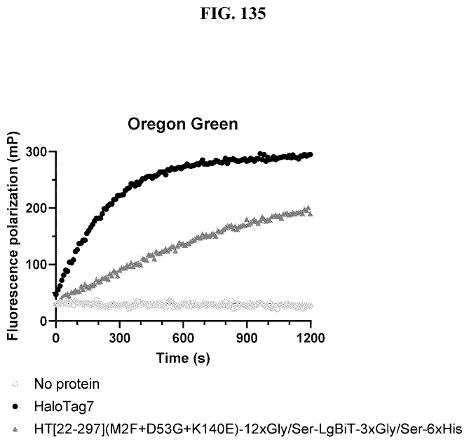

Experiments conducted during development of embodiments herein demonstrated that HaloTag[22-297](M2F+D53G+K140E)-12×Gly/Ser-LgBiT-3×Gly/Ser-6×His is functional with Oregon Green HaloTag ligand (FIG. 135).

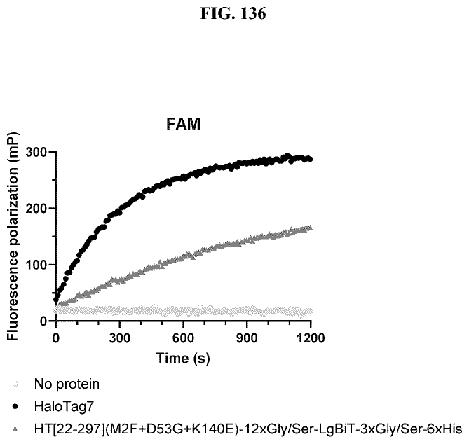

Experiments conducted during development of embodiments herein demonstrated that HaloTag[22-297](M2F+D53G+K140E)-12×Gly/Ser-LgBiT-3×Gly/Ser-6×His is functional with FAM HaloTag ligand (FIG. 136).

### Sequences

The following sequences are referred to herein, contained within the Sequence Listing filed herewith, and are within the scope of embodiments herein.
SEQ ID NO: 1—HaloTag®
SEQ ID NOS: 2-577—split HaloTag® (spHTs)
SEQ ID NOS: 578-1187—Exemplary peptide components of a modified dehalogenase complex (SmHTs)
SEQ ID NOS: 1888-3033—Exemplary polypeptide components of a modified dehalogenase complex (LgHTs)
SEQ ID NOS: 3034-3035—Exemplary SmHT consensus sequences
SEQ ID NO: 3036—β10 of NanoLuc
SEQ ID NO: 3037—Exemplary polypeptide component of a bioluminescent complex (LgBiT)
SEQ ID NO: 3038—Exemplary high-affinity peptide component of a bioluminescent complex (HiBiT)
SEQ ID NO: 3039—Exemplary low-affinity peptide component of a bioluminescent complex (SmBiT)
SEQ ID NO: 3040—Full length sequence of NanoTrip-β9-HiBiT
SEQ ID NO: 3041—Full length sequence of NanoTrip-β9-SmBiT)
SEQ ID NO: 3042—NanoLuc β1-5
SEQ ID NO: 3043—NanoLuc β1-6
SEQ ID NO: 3044—NanoLuc β1-7
SEQ ID NO: 3045—NanoLuc β1-8
SEQ ID NO: 3046—NanoLuc β6-10-like (Hi affinity)
SEQ ID NO: 3047—NanoLuc β7-10-like (Hi affinity)
SEQ ID NO: 3048—NanoLuc β8-10-like (Hi affinity)
SEQ ID NO: 3049—NanoLuc β9-10-like (Hi affinity)
SEQ ID NO: 3050—NanoLuc β6-10-like (Lo affinity)
SEQ ID NO: 3051—NanoLuc β7-10-like (Lo affinity)
SEQ ID NO: 3052—NanoLuc β8-10-like (Lo affinity)
SEQ ID NO: 3053—NanoLuc β9-10-like (Lo affinity)
SEQ ID NO: 3054—NanoLuc β6
SEQ ID NO: 3055—NanoLuc β7
SEQ ID NO: 3056—NanoLuc β8
SEQ ID NO: 3057—NanoLuc β9
SEQ ID NO: 3058—Exemplary high-affinity peptide component of a bioluminescent complex (HiBiT)
SEQ ID NO: 3059—Exemplary NanoLuc β9-like peptide
SEQ ID NO: 3060—Exemplary high-affinity peptide component of a bioluminescent complex (SmHiTrip10)
SEQ ID NOS: 3061, 3064-3066, and 3079-3091—Exemplary variant peptide components of a modified dehalogenase complex
SEQ ID NO: 3093—Exemplary high-affinity peptide component of a bioluminescent complex (HiBiT)
SEQ ID NOS: 3062-3063, 3067-3078, 3092, 3094-3109, and 4177-4181—Exemplary fusions of high-affinity peptide components of a bioluminescent complex and peptide components of a modified dehalogenase complex

TABLE 2
Exemplary peptide sequences

| SEQ | | |
|---|---|---|
| ID | | |
| NO | Description | Sequence |
| 3061 | HaloTag[3-19] | EIGTGFPFDPHYVEVLG |
| 3062 | HaloTag[3-19]-5xGly/Ser-VS-HiBiT | EIGTGFPFDPHYVEVLGGGSGGVSVSGWRLFKKIS |
| 3063 | VS-HiBiT-5xGly/Ser-HaloTag[3-19] | VSVSGWRLFKKISGGSGGEIGTGFPFDPHYVEVLG |
| 3064 | HaloTag[3-19] (I2R + G3K + G5R) | ERKTRFPFDPHYVEVLG |
| 3065 | HaloTag[3-19] (D9R + E14K + L16R) | EIGTGFPFRPHYVKVRG |
| 3066 | HaloTag[3-19] (I2R + G3K + G5R + G17R) | ERKTRFPFDPHYVEVLR |
| 3067 | HaloTag[3-19]-HiBiT | EIGTGFPFDPHYVEVLGVSGWRLFKKIS |
| 3068 | HaloTag[3-19] (G3D + G5E + P10E)-HiBIT | EIGTGFPFDPHYVEVLGVSGWRLFKKIS |
| 3069 | HaloTag[3-19] (D9R + E14K + L16R)-HiBiT | EIGTGFPFRPHYVKVRGVSGWRLFKKIS |
| 3070 | HaloTag[3-19] (I2R + G3K + G5R)-VS-HiBIT | ERKTRFPFDPHYVEVLGVSVSGWRLFKKIS |
| 3071 | HaloTag[3-19] (D9R + E14K + L16R)VS-HiBIT | EIGTGFPFRPHYVKVRGVSVSGWRLFKKIS |
| 3072 | HaloTag[3-19] (I2R + G3K+ G5R + G17R)-VS- | ERKTRFPFDPHYVEVLRVSVSGWRLFKKIS |
| | HiBiT | |
| 3073 | HaloTag[3-19] (I2V + G3S + T4E + G5D + D9E + | EVSEDFPFEPNYIEVLGVSVSGWRLFKKIS |
| | H11N + V13I)-VS-HiBiT | |
| 3074 | HaloTag[3-19] (I2R + G3S + T4S + G5T + D9E + | ERSSTFPFEEHYVEVLGVSVSGWRLFKKIS |
| | P10E)-VS-HiBiT | |
| 3075 | HaloTag[3-19] (G3S + T4S + G5D + D9K + P10S)- | EISSDFPFKSHYVEVLGVSVSGWRLFKKIS |
| | VS-HiBiT | |
| 3076 | HaloTag[3-19] | EKSTEFPFKDHFVRCLGVSVSGWRLFKKIS |
| | (I2K + G3S + G5E + D9K + P10D + Y12F + E14R + | |
| | V15C)-VS-HiBiT | |
| 3077 | HaloTag[3-19] | KFNDQWNYRANFLKIRRVSVSGWRLFKKIS |
| | (E1K + I2F + G3N + T4D + G5Q + F6W + P7N + | |
| | F8Y + D9R + P10A + H11N + Y12F + V13L + E14K + | |
| | V15I + L16R + G17R)-VS-HiBIT | |
| 3078 | HaloTag[3-19]-VS-HiBiT | EIGTGFPFDPHYVEVLGVSVSGWRLFKKIS |
| 3079 | HaloTag[3-19] | KFNDQWNYRANFLKIRR |
| | (E1K + I2F + G3N + T4D + G5Q + F6W + P7N + | |
| | F8Y + D9R + P10A + H11N + Y12F + V13L + | |
| | E14K + V15I + L16R + G17R) | |
| 3080 | HaloTag [4-19] (I2K) | KGTGFPFDPHYVEVLG |
| 3081 | HaloTag[3-19] (I2R + G3K + G5R + D9R + E14K + | ERKTRFPFRPHYVKVRR |
| | L16R + G17R) | |
| 3082 | HaloTag[3-19] | KFNDQFNFRANYVKVRR |
| | (E1K + I2F + G3N + T4D + G5Q + P7N + D9R + | |
| | P10A + H11N + E14K + L16R + G17R) | |
| 3083 | HaloTag[3-19] (D9R + E14K + L16R + G17R) | EIGTGFPFRPHYVKVRR |
| 3084 | HaloTag[3-19] (D9R + E14K + G17R) | EIGTGFPFRPHYVKVLR |
| 3085 | HaloTag[3-19] (D9R + E14R) | EIGTGFPFRPHYVRVLG |
| 3086 | HaloTag[3-19] (D9R) | EIGTGFPFRPHYVEVLG |
| 3087 | HaloTag[3-19] (I2F + T4D + G5Q) | EFGDQFPFDPHYVEVLG |
| 3088 | HaloTag[3-19] (G3N + T4D + H11N) | EINDGFPFDPNYVEVLG |
| 3089 | HaloTag[3-19] (F6W + F8Y) | EIGTGWPYDPHYVEVLG |
| 3090 | HaloTag[3-19] (G5E + P7Q + P10E) | EIGTEFQFDEHYVEVLG |
| 3091 | HaloTag[3-19] (G3D + G5E + F6W + F8Y + P10E + | EIDTEWPYDEHYIEILG |
| | V13I + V15I) | |
| 3092 | HaloTag[3-19]-4xGly/Ser-VS-HiBiT | EIGTGFPFDPHYVEVLGGGSGVSVSGWRLFKKIS |
| 3093 | Biotin-VS-HiBiT | VSVSGWRLFKKIS |
| 3094 | Biotin-HaloTag[3-19] (I2R)-4xGly/Ser-VS-HiBiT | ERGTGFPFDPHYVEVLGGGSGVSVSGWRLFKKIS |
| 3095 | Biotin-HaloTag[3-19] (G3K)-4xGly/Ser-VS-HiBIT | EIKTGFPFDPHYVEVLGGGSGVSVSGWRLFKKIS |
| 3096 | Biotin-HaloTag[3-19] (G5R)-4xGly/Ser-VS-HiBiT | EIGTRFPFDPHYVEVLGGGSGVSVSGWRLFKKIS |
| 3097 | Biotin-HaloTag[3-19] (D9R)-4xGly/Ser-VS-HiBiT | EIGTGFPFRPHYVEVLGGGSGVSVSGWRLFKKIS |
| 3098 | Biotin-HaloTag[3-19] (E14K)-4xGly/Ser-VS-HiBIT | EIGTGFPFDPHYVKVLGGGSGVSVSGWRLFKKIS |
| 3099 | Biotin-HaloTag[3-19] (L16R)-4xGly/Ser-VS-HiBiT | EIGTGFPFDPHYVEVRGGGSGVSVSGWRLFKKIS |
| 3100 | Biotin-HaloTag[3-19] (G17R)-4xGly/Ser-VS-HiBiT | EIGTGFPFDPHYVEVLRGGSGVSVSGWRLFKKIS |
| 3101 | Biotin-HaloTag[3-19] (I2R + G3K)-4xGly/Ser-VS- | ERKTGFPFDPHYVEVLGGGSGVSVSGWRLFKKIS |
| | HiBiT | |
| 3102 | Biotin-HaloTag[3-19] (I2R + G5R)-4xGly/Ser-VS- | ERGTRFPFDPHYVEVLGGGSGVSVSGWRLFKKIS |
| | HiBiT | |
| 3103 | Biotin-HaloTag[3-19] (D9R + E14K)-4xGly/Ser-VS- | EIGTGFPFRPHYVKVLGGGSGVSVSGWRLFKKIS |
| | HiBiT | |
| 3104 | Biotin-HaloTag[3-19] (D9R + L16R)-4xGly/Ser-VS- | EIGTGFPFRPHYVEVRGGGSGVSVSGWRLFKKIS |
| | HiBiT | |
| 3105 | Biotin-HaloTag[3-19] (I2R + D9R)-4xGly/Ser-VS- | ERGTGFPFRPHYVEVLGGGSGVSVSGWRLFKKIS |
| | HiBiT | |
| 3106 | Biotin-HaloTag[3-19] (I2R + G17R)-4xGly/Ser-VS- | ERGTGFPFDPHYVEVLRGGSGVSVSGWRLFKKIS |
| | HiBiT | |
| 3107 | Biotin-HaloTag[3-19] (G3K + E14K)-4xGly/Ser-VS- | EIKTGFPFDPHYVKVLGGGSGVSVSGWRLFKKIS |
| | HiBiT | |
| 3108 | Biotin-HaloTag[3-19] (G3K + L16R)-4xGly/Ser-VS- | EIKTGFPFDPHYVEVRGGGSGVSVSGWRLFKKIS |
| | HiBiT | |
| 3109 | Biotin-HaloTag[3-19] (G5R + G17R)-4xGly/Ser-VS- | EIGTRFPFDPHYVEVLRGGSGVSVSGWRLFKKIS |
| | HiBiT | |
| 4177 | Biotin-HaloTag[3-19] (I2R)-4xGly/Ser-VS-HiBiT | ERGTGFPFDPHYVEVLGGGSGVSVSGWRLFRRIS |
| | (K8R + K9R) | |
| 4178 | Biotin-HaloTag[3-19] (I2R)-VS-HiBiT | ERGTGFPFDPHYVEVLGVSVSGWRLFKKIS |
| 4179 | Biotin-HaloTag[3-19] (I2R)-4xGly/Ser-HiBiT | ERGTGFPFDPHYVEVLGGGSGVSGWRLFKKIS |
| 4180 | Biotin-VS-HiBiT-5xGly/Ser-HaloTag[3-19] (I2R) | VSVSGWRLFKKISGGSGGERGTGFPFDPHYVEVLG |
| 4181 | VS-HiBiT-HaloTag[3-19] (I2R) | VSVSGWRLFKKISERGTGFPFDPHYVEVLG |

TABLE 3
SEQ ID NOS: 3110-4064-Exemplary dual detector variants

| | SEQ |
|---|---|
| Mutant | ID NO |
| M2L + M49F | 3110 |
| M2L + D53S | 3111 |
| M2L + D53G | 3112 |
| M2L + D56P | 3113 |
| M2L + L57I | 3114 |
| M2L + E79K | 3115 |
| M2L + S89A | 3116 |
| M2L + F93L | 3117 |
| M2L + T128M | 3118 |
| M2L + A131Q | 3119 |
| M2L + T135E | 3120 |
| M2L + F148M | 3121 |
| M2L + V177E | 3122 |
| M2L + V177Q | 3123 |
| M2L + P186A | 3124 |
| M2L + P186V | 3125 |
| M2L + S238A | 3126 |
| M2L + L253I | 3127 |
| M2F + M49F + D53S | 3128 |
| M2F + M49F + D53G | 3129 |
| M2F + M49F + D56P | 3130 |
| M2F + M49F + L57I | 3131 |
| M2F + M49F + E79K | 3132 |
| M2F + M49F + S89A | 3133 |
| M2F + M49F + F93L | 3134 |
| M2F + M49F + T128M | 3135 |
| M2F + M49F + A131Q | 3136 |
| M2F + M49F + T135E | 3137 |
| M2F + M49F + F148M | 3138 |
| M2F + M49F + V177E | 3139 |
| M2F + M49F + V177Q | 3140 |
| M2F + M49F + P186A | 3141 |
| M2F + M49F + P186V | 3142 |
| M2F + M49F + S238A | 3143 |
| M2F + M49F + L253I | 3144 |
| M2F + D53S + D56P | 3145 |
| M2F + D53S + L57I | 3146 |
| M2F + D53S + E79K | 3147 |
| M2F + D53S + S89A | 3148 |
| M2F + D53S + F93L | 3149 |
| M2F + D53S + T128M | 3150 |
| M2F + D53S + A131Q | 3151 |
| M2F + D53S + T135E | 3152 |
| M2F + D53S + F148M | 3153 |
| M2F + D53S + V177E | 3154 |
| M2F + D53S + V177Q | 3155 |
| M2F + D53S + P186A | 3156 |
| M2F + D53S + P186V | 3157 |
| M2F + D53S + S238A | 3158 |
| M2F + D53S + L253I | 3159 |
| M2F + D53G + D56P | 3160 |
| M2F + D53G + L57I | 3161 |
| M2F + D53G + E79K | 3162 |
| M2F + D53G + S89A | 3163 |
| M2F + D53G + T128M | 3164 |
| M2F + D53G + A131Q | 3165 |
| M2F + D53G + T135E | 3166 |
| M2F + D53G + F148M | 3167 |
| M2F + D53G + V177E | 3168 |
| M2F + D53G + V177Q | 3169 |
| M2F + D53G + P186A | 3170 |
| M2F + D53G + P186V | 3171 |
| M2F + D53G + S238A | 3172 |
| M2F + D53G + L253I | 3173 |
| M2F + D56P + L57I | 3174 |
| M2F + D56P + E79K | 3175 |
| M2F + D56P + S89A | 3176 |
| M2F + D56P + F93L | 3177 |
| M2F + D56P + T128M | 3178 |
| M2F + D56P + A131Q | 3179 |
| M2F + D56P + T135E | 3180 |
| M2F + D56P + F148M | 3181 |
| M2F + D56P + V177E | 3182 |
| M2F + D56P + V177Q | 3183 |
| M2F + D56P + P186A | 3184 |
| M2F + D56P + P186V | 3185 |
| M2F + D56P + S238A | 3186 |
| M2F + D56P + L253I | 3187 |
| M2F + L571 + E79K | 3188 |
| M2F + L571 + S89A | 3189 |
| M2F + L571 + F93L | 3190 |
| M2F + L571 + T128M | 3191 |
| M2F + L571 + A131Q | 3192 |
| M2F + L57I + T135E | 3193 |
| M2F + L57I + F148M | 3194 |
| M2F + L57I + V177E | 3195 |
| M2F + L57I + V177Q | 3196 |
| M2F + L57I + P186A | 3197 |
| M2F + L57I + P186V | 3198 |
| M2F + L57I + S238A | 3199 |
| M2F + L57I + L253I | 3200 |
| M2F + E79K + S89A | 3201 |
| M2F + E79K + F93L | 3202 |
| M2F + E79K + T128M | 3203 |
| M2F + E79K + A131Q | 3204 |
| M2F + E79K + T135E | 3205 |
| M2F + E79K + F148M | 3206 |
| M2F + E79K + V177E | 3207 |
| M2F + E79K + V177Q | 3208 |
| M2F + E79K + P186A | 3209 |
| M2F + E79K + P186V | 3210 |
| M2F + E79K + S238A | 3211 |
| M2F + E79K + L253I | 3212 |
| M2F + S89A + F93L | 3213 |
| M2F + S89A + T128M | 3214 |
| M2F + S89A + A131Q | 3215 |
| M2F + S89A + T135E | 3216 |
| M2F + S89A + F148M | 3217 |
| M2F + S89A + V177E | 3218 |
| M2F + S89A + V177Q | 3219 |
| M2F + S89A + P186A | 3220 |
| M2F + S89A + P186V | 3221 |
| M2F + S89A + S238A | 3222 |
| M2F + S89A + L253I | 3223 |
| M2F + F93L + T128M | 3224 |
| M2F + F93L + A131Q | 3225 |
| M2F + F93L + T135E | 3226 |
| M2F + F93L + F148M | 3227 |
| M2F + F93L + V177E | 3228 |
| M2F + F93L + V177Q | 3229 |
| M2F + F93L + P186A | 3230 |
| M2F + F93L + P186V | 3231 |
| M2F + F93L + S238A | 3232 |
| M2F + F93L + L253I | 3233 |
| M2F + T128M + A131Q | 3234 |
| M2F + T128M + T135E | 3235 |
| M2F + T128M + F148M | 3236 |
| M2F + T128M + V177E | 3237 |
| M2F + T128M + V177Q | 3238 |
| M2F + T128M + P186A | 3239 |
| M2F + T128M + P186V | 3240 |
| M2F + T128M + S238A | 3241 |
| M2F + T128M + L253I | 3242 |
| M2F + A131Q + T135E | 3243 |
| M2F + A131Q + F148M | 3244 |
| M2F + A131Q + V177E | 3245 |
| M2F + A131Q + V177Q | 3246 |
| M2F + A131Q + P186A | 3247 |
| M2F + A131Q + P186V | 3248 |
| M2F + A131Q + S238A | 3249 |
| M2F + A131Q + L253I | 3250 |
| M2F + T135E + F148M | 3251 |
| M2F + T135E + V177E | 3252 |
| M2F + T135E + V177Q | 3253 |
| M2F + T135E + P186A | 3254 |
| M2F + T135E + P186V | 3255 |
| M2F + T135E + S238A | 3256 |
| M2F + T135E + L253I | 3257 |
| M2F + F148M + V177E | 3258 |
| M2F + F148M + V177Q | 3259 |
| M2F + F148M + P186A | 3260 |
| M2F + F148M + P186V | 3261 |
| M2F + F148M + S238A | 3262 |
| M2F + F148M + L253I | 3263 |
| M2F + V177E + P186A | 3264 |
| M2F + V177E + P186V | 3265 |
| M2F + V177E + S238A | 3266 |
| M2F + V177E + L253I | 3267 |
| M2F + V177Q + P186A | 3268 |
| M2F + V177Q + P186V | 3269 |
| M2F + V177Q + S238A | 3270 |
| M2F + V177Q + L253I | 3271 |
| M2F + P186A + S238A | 3272 |
| M2F + P186A + L253I | 3273 |
| M2F + P186V + S238A | 3274 |
| M2F + P186V + L253I | 3275 |
| M2F + S238A + L253I | 3276 |
| M2F + D56P + M49F + D53G | 3277 |
| M2F + D56P + D53G + L57I | 3278 |
| M2F + D56P + D53G + T128M | 3279 |
| M2F + D56P + D53G + F148M | 3280 |
| M2F + D56P + D53G + V177E | 3281 |
| M2F + D56P + D53G + P186A | 3282 |
| M2F + L57I + M49F + D53G | 3283 |
| M2F + L57I + D53G + T128M | 3284 |
| M2F + L57I + D53G + F148M | 3285 |
| M2F + L57I + D53G + V177E | 3286 |
| M2F + L57I + D53G + P186A | 3287 |
| M2F + S89A + M49F + D53G | 3288 |
| M2F + S89A + D53G + L57I | 3289 |
| M2F + S89A + D53G + T128M | 3290 |
| M2F + S89A + D53G + F148M | 3291 |
| M2F + S89A + D53G + V177E | 3292 |
| M2F + S89A + D53G + P186A | 3293 |
| M2F + V177E + M49F + D53G | 3294 |
| M2F + V177E + D53G + T128M | 3295 |
| M2F + V177E + D53G + F148M | 3296 |
| M2F + V177E + D53G + P186A | 3297 |
| M2F + P186A + M49F + D53G | 3298 |
| M2F + P186A + D53G + T128M | 3299 |
| M2F + P186A + D53G + F148M | 3300 |
| M2F + L253I + M49F + D53G | 3301 |
| M2F + L253I + D53G + L57I | 3302 |
| M2F + L253I + D53G + T128M | 3303 |
| M2F + L253I + D53G + F148M | 3304 |
| M2F + L253I + D53G + V177E | 3305 |
| M2F + L253I + D53G + P186A | 3306 |
| M2F + D53S + D56P + M49F | 3307 |
| M2F + D53S + D56P + L57I | 3308 |
| M2F + D53S + D56P + T128M | 3309 |
| M2F + D53S + D56P + F148M | 3310 |
| M2F + D53S + D56P + V177E | 3311 |
| M2F + D53S + D56P + P186A | 3312 |
| M2F + D53S + L57I + M49F | 3313 |
| M2F + D53S + L57I + T128M | 3314 |
| M2F + D53S + L57I + F148M | 3315 |
| M2F + D53S + L57I + V177E | 3316 |
| M2F + D53S + L57I + P186A | 3317 |
| M2F + D53S + S89A + M49F | 3318 |
| M2F + D53S + S89A + L57I | 3319 |
| M2F + D53S + S89A + T128M | 3320 |
| M2F + D53S + S89A + F148M | 3321 |
| M2F + D53S + S89A + V177E | 3322 |
| M2F + D53S + S89A + P186A | 3323 |
| M2F + D53S + V177E + M49F | 3324 |
| M2F + D53S + V177E + T128M | 3325 |
| M2F + D53S + V177E + F148M | 3326 |
| M2F + D53S + V177E + P186A | 3327 |
| M2F + D53S + P186A + M49F | 3328 |
| M2F + D53S + P186A + T128M | 3329 |
| M2F + D53S + P186A + F148M | 3330 |
| M2F + D53S + L253I + M49F | 3331 |
| M2F + D53S + L253I + L57I | 3332 |
| M2F + D53S + L253I + T128M | 3333 |
| M2F + D53S + L253I + F148M | 3334 |
| M2F + D53S + L253I + V177E | 3335 |
| M2F + D53S + L253I + P186A | 3336 |
| M2F + D53G + L57I + M49F + S89A | 3337 |
| M2F + D53G + L57I + M49F + V177Q | 3338 |
| M2F + D53G + T128M + M49F + L57I | 3339 |
| M2F + D53G + T128M + M49F + S89A | 3340 |
| M2F + D53G + T128M + M49F + V177Q | 3341 |
| M2F + D53G + F148M + M49F + L57I | 3342 |
| M2F + D53G + F148M + M49F + S89A | 3343 |
| M2F + D53G + F148M + M49F + V177Q | 3344 |
| M2F + D53G + V177E + M49F + L57I | 3345 |
| M2F + D53G + V177E + M49F + S89A | 3346 |
| M2F + D53G + P186A + M49F + L57I | 3347 |
| M2F + D53G + P186A + M49F + S89A | 3348 |
| M2F + D53G + P186A + M49F + V177Q | 3349 |
| M2F + D53S + D56P + M49F + L57I | 3350 |
| M2F + D53S + D56P + M49F + S89A | 3351 |
| M2F + D53S + D56P + M49F + V177Q | 3352 |
| M2F + D53S + L57I + M49F + S89A | 3353 |
| M2F + D53S + L57I + M49F + V177Q | 3354 |
| M2F + D53S + S89A + M49F + V177Q | 3355 |
| M2F + D53S + V177E + M49F + L57I | 3356 |
| M2F + D53S + V177E + M49F + S89A | 3357 |
| M2F + D53S + P186A + M49F + L57I | 3358 |
| M2F + D53S + P186A + M49F + S89A | 3359 |
| M2F + D53S + P186A + M49F + V177Q | 3360 |
| M2F + D53S + L253I + M49F + L57I | 3361 |
| M2F + D53S + L253I + M49F + S89A | 3362 |
| M2F + D53S + L253I + M49F + V177Q | 3363 |
| M2F + L57I + S89A + M49F + V177Q | 3364 |
| M2F + L57I + S89A + D53G + T128M | 3365 |
| M2F + L57I + S89A + D53G + F148M | 3366 |
| M2F + L57I + S89A + D53G + V177E | 3367 |
| M2F + L57I + S89A + D53G + P186A | 3368 |
| M2F + L57I + S89A + D53S + D56P | 3369 |
| M2F + L57I + S89A + D53S + V177E | 3370 |
| M2F + L57I + S89A + D53S + P186A | 3371 |
| M2F + L57I + S89A + D53S + L253I | 3372 |
| M2F + L57I + V177E + M49F + S89A | 3373 |
| M2F + L57I + V177E + D53G + T128M | 3374 |
| M2F + L57I + V177E + D53G + F148M | 3375 |
| M2F + L57I + V177E + D53G + P186A | 3376 |
| M2F + L57I + V177E + D53S + D56P | 3377 |
| M2F + L57I + V177E + D53S + P186A | 3378 |
| M2F + L57I + V177E + D53S + L253I | 3379 |
| M2F + L57I + T135E + M49F + D53G | 3380 |
| M2F + L57I + T135E + M49F + S89A | 3381 |
| M2F + L57I + T135E + M49F + V177Q | 3382 |
| M2F + L57I + T135E + D53G + T128M | 3383 |
| M2F + L57I + T135E + D53G + F148M | 3384 |
| M2F + L57I + T135E + D53G + V177E | 3385 |
| M2F + L57I + T135E + D53G + P186A | 3386 |
| M2F + L57I + T135E + D53S + D56P | 3387 |
| M2F + L57I + T135E + D53S + S89A | 3388 |
| M2F + L57I + T135E + D53S + V177E | 3389 |
| M2F + L57I + T135E + D53S + P186A | 3390 |
| M2F + L57I + T135E + D53S + L253I | 3391 |
| M2F + L57I + S238A + M49F + D53G | 3392 |
| M2F + L57I + S238A + M49F + S89A | 3393 |
| M2F + L57I + S238A + M49F + V177Q | 3394 |
| M2F + L57I + S238A + D53G + T128M | 3395 |
| M2F + L57I + S238A + D53G + F148M | 3396 |
| M2F + L57I + S238A + D53G + V177E | 3397 |
| M2F + L57I + S238A + D53G + P186A | 3398 |
| M2F + L57I + S238A + D53S + D56P | 3399 |
| M2F + L57I + S238A + D53S + S89A | 3400 |
| M2F + L57I + S238A + D53S + V177E | 3401 |
| M2F + L57I + S238A + D53S + P186A | 3402 |
| M2F + L57I + S238A + D53S + L253 | 3403 |
| M2F + S89A + T135E + M49F + D53G | 3404 |
| M2F + S89A + T135E + M49F + V177Q | 3405 |
| M2F + S89A + T135E + D53G + L57I | 3406 |
| M2F + S89A + T135E + D53G + T128M | 3407 |
| M2F + S89A + T135E + D53G + F148M | 3408 |
| M2F + S89A + T135E + D53G + V177E | 3409 |
| M2F + S89A + T135E + D53G + P186A | 3410 |
| M2F + S89A + T135E + D53S + D56P | 3411 |
| M2F + S89A + T135E + D53S + V177E | 3412 |
| M2F + S89A + T135E + D53S + P186A | 3413 |
| M2F + S89A + T135E + D53S + L253 | 3414 |
| M2F + S89A + T135E + L57I + V177E | 3415 |
| M2F + S89A + T135E + L57I + S238A | 3416 |
| M2F + S89A + V177E + D53G + T128M | 3417 |
| M2F + S89A + V177E + D53G + F148M | 3418 |
| M2F + S89A + V177E + D53G + P186A | 3419 |
| M2F + S89A + V177E + D53S + D56P | 3420 |
| M2F + S89A + V177E + D53S + P186A | 3421 |
| M2F + S89A + V177E + D53S + L253I | 3422 |
| M2F + S89A + V177E + L57I + S238A | 3423 |
| M2F + F93L + F148M + M49F + D53G | 3424 |
| M2F + F93L + F148M + M49F + L57I | 3425 |
| M2F + F93L + F148M + M49F + S89A | 3426 |
| M2F + F93L + F148M + M49F + V177Q | 3427 |
| M2F + F93L + F148M + D53G + L57 | 3428 |
| M2F + F93L + F148M + D53G + T128M | 3429 |
| M2F + F93L + F148M + D53G + V177E | 3430 |
| M2F + F93L + F148M + D53G + P186A | 3431 |
| M2F + F93L + F148M + D53S + D56P | 3432 |
| M2F + F93L + F148M + D53S + L57I | 3433 |
| M2F + F93L + F148M + D53S + S89A | 3434 |
| M2F + F93L + F148M + D53S + V177E | 3435 |
| M2F + F93L + F148M + D53S + P186A | 3436 |
| M2F + F93L + F148M + D53S + L253 | 3437 |
| M2F + F93L + F148M + L57I + S89A | 3438 |
| M2F + F93L + F148M + L57I + V177E | 3439 |
| M2F + F93L + F148M + L57I + T135E | 3440 |
| M2F + F93L + F148M + L57I + S238A | 3441 |
| M2F + F93L + F148M + S89A + T135E | 3442 |
| M2F + F93L + F148M + S89A + V177E | 3443 |
| M2F + F93L + P186A + M49F + D53G | 3444 |
| M2F + F93L + P186A + M49F + L57I | 3445 |
| M2F + F93L + P186A + M49F + S89A | 3446 |
| M2F + F93L + P186A + M49F + V177Q | 3447 |
| M2F + F93L + P186A + D53G + L57I | 3448 |
| M2F + F93L + P186A + D53G + T128M | 3449 |
| M2F + F93L + P186A + D53G + V177E | 3450 |
| M2F + F93L + P186A + D53S + D56P | 3451 |
| M2F + F93L + P186A + D53S + L57I | 3452 |
| M2F + F93L + P186A + D53S + S89A | 3453 |
| M2F + F93L + P186A + D53S + V177E | 3454 |
| M2F + F93L + P186A + D53S + L253I | 3455 |
| M2F + F93L + P186A + L57I + S89A | 3456 |
| M2F + F93L + P186A + L57I + V177E | 3457 |
| M2F + F93L + P186A + L57I + T135E | 3458 |
| M2F + F93L + P186A + L57I + S238A | 3459 |
| M2F + F93L + P186A + S89A + T135E | 3460 |
| M2F + F93L + P186A + S89A + V177E | 3461 |
| M2F + T135E + L253I + M49F + D53G | 3462 |
| M2F + T135E + L253I + M49F + L57I | 3463 |
| M2F + T135E + L253I + M49F + S89A | 3464 |
| M2F + T135E + L253I + M49F + V177Q | 3465 |
| M2F + T135E + L253I + D53G + L57 | 3466 |
| M2F + T135E + L253I + D53G + T128M | 3467 |
| M2F + T135E + L253I + D53G + F148M | 3468 |
| M2F + T135E + L253I + D53G + V177E | 3469 |
| M2F + T135E + L253I + D53G + P186A | 3470 |
| M2F + T135E + L253I + D53S + D56P | 3471 |
| M2F + T135E + L253I + D53S + V177E | 3472 |
| M2F + T135E + L253I + D53S + P186A | 3473 |
| M2F + T135E + L253I + L57I + S89A | 3474 |
| M2F + T135E + L253I + L57I + V177E | 3475 |
| M2F + T135E + L253I + L57I + S238A | 3476 |
| M2F + T135E + L253I + S89A + V177E | 3477 |
| M2F + T135E + L253I + F93L + F148M | 3478 |
| M2F + T135E + L253I + F93L + P186A | 3479 |
| M2F + F148M + V177E + M49F + D53G | 3480 |
| M2F + F148M + V177E + M49F + L57I | 3481 |
| M2F + F148M + V177E + M49F + S89A | 3482 |
| M2F + F148M + V177E + D53G + T128M | 3483 |
| M2F + F148M + V177E + D53G + P186A | 3484 |
| M2F + F148M + V177E + D53S + D56P | 3485 |
| M2F + F148M + V177E + D53S + L57I | 3486 |
| M2F + F148M + V177E + D53S + S89A | 3487 |
| M2F + F148M + V177E + D53S + P186A | 3488 |
| M2F + F148M + V177E + D53S + L253I | 3489 |
| M2F + F148M + V177E + L57I + S89A | 3490 |
| M2F + F148M + V177E + L57I + T135E | 3491 |
| M2F + F148M + V177E + L57I + S238A | 3492 |
| M2F + F148M + V177E + S89A + T135E | 3493 |
| M2F + F148M + V177E + F93L + P186A | 3494 |
| M2F + F148M + V177E + T135E + L253 | 3495 |
| M2F + A131Q + F148M + M49F + D53G | 3496 |
| M2F + A131Q + F148M + M49F + L57I | 3497 |
| M2F + A131Q + F148M + M49F + S89A | 3498 |
| M2F + A131Q + F148M + M49F + V177Q | 3499 |
| M2F + A131Q + F148M + D53G + L57I | 3500 |
| M2F + A131Q + F148M + D53G + T128M | 3501 |
| M2F + A131Q + F148M + D53G + V177E | 3502 |
| M2F + A131Q + F148M + D53G + P186A | 3503 |
| M2F + A131Q + F148M + D53S + D56P | 3504 |
| M2F + A131Q + F148M + D53S + L57I | 3505 |
| M2F + A131Q + F148M + D53S + S89A | 3506 |
| M2F + A131Q + F148M + D53S + V177E | 3507 |
| M2F + A131Q + F148M + D53S + P186A | 3508 |
| M2F + A131Q + F148M + D53S + L253 | 3509 |
| M2F + A131Q + F148M + L57I + S89A | 3510 |
| M2F + A131Q + F148M + L57I + V177E | 3511 |
| M2F + A131Q + F148M + L57I + T135E | 3512 |
| M2F + A131Q + F148M + L57I + S238A | 3513 |
| M2F + A131Q + F148M + S89A + T135E | 3514 |
| M2F + A131Q + F148M + S89A + V177E | 3515 |
| M2F + A131Q + F148M + F93L + P186A | 3516 |
| M2F + A131Q + F148M + T135E + L253I | 3517 |
| M2F + A131Q + F148M + V177Q + M49F + D53G | 3518 |
| M2F + A131Q + V177Q + M49F + L57I | 3519 |
| M2F + A131Q + V177Q + M49F + S89A | 3520 |
| M2F + A131Q + V177Q + D53G + L57I | 3521 |
| M2F + A131Q + V177Q + D53G + T128M | 3522 |
| M2F + A131Q + V177Q + D53G + F148M | 3523 |
| M2F + A131Q + V177Q + D53G | 3524 |
| M2F + A131Q + V177Q + D53G + P186A | 3525 |
| M2F + A131Q + V177Q + D53S + D56P | 3526 |
| M2F + A131Q + V177Q + D53S + L57I | 3527 |
| M2F + A131Q + V177Q + D53S + S89A | 3528 |
| M2F + A131Q + V177Q + D53S | 3529 |
| M2F + A131Q + V177Q + D53S + P186A | 3530 |
| M2F + A131Q + V177Q + D53S + L253I | 3531 |
| M2F + A131Q + V177Q + L57I + S89A | 3532 |
| M2F + A131Q + V177Q + L57I | 3533 |
| M2F + A131Q + V177Q + L57I + T135E | 3534 |
| M2F + A131Q + V177Q + L57I + S238A | 3535 |
| M2F + A131Q + V177Q + S89A + T135E | 3536 |
| M2F + A131Q + V177Q + S89A | 3537 |
| M2F + A131Q + V177Q + F93L + F148M | 3538 |
| M2F + A131Q + V177Q + F93L + P186A | 3539 |
| M2F + A131Q + V177Q + T135E + L253I | 3540 |
| M2F + A131Q + V177Q + F148M | 3541 |
| M2F + V177E + L253I + M49F + D53G | 3542 |
| M2F + V177E + L253I + M49F + L57I | 3543 |
| M2F + V177E + L253I + M49F + S89A | 3544 |
| M2F + V177E + L253I + M49F | 3545 |
| M2F + V177E + L253I + D53G + L57I | 3546 |
| M2F + V177E + L253I + D53G + T128M | 3547 |
| M2F + V177E + L253I + D53G + F148M | 3548 |
| M2F + V177E + L253I + D53G + P186A | 3549 |
| M2F + V177E + L253I + D53S + D56P | 3550 |
| M2F + V177E + L253I + D53S + P186A | 3551 |
| M2F + V177E + L253I + L57I + S89A | 3552 |
| M2F + V177E + L253I + L57I + S238A | 3553 |
| M2F + V177E + L253I + F93L + F148M | 3554 |
| M2F + V177E + L253I + F93L + P186A | 3555 |
| M2F + V177E + L253I + A131Q + F148M | 3556 |
| M2F + V177E + L253I + A131Q | 3557 |
| M2F + I112V | 3558 |
| M2F + P114T | 3559 |
| M2F + I115V | 3560 |
| M2F + P116G | 3561 |
| M2F + T117D | 3562 |
| M2F + E127K | 3563 |
| M2F + Q130R | 3564 |
| M2F + A131R | 3565 |
| M2F + K140E | 3566 |
| M2F + F148I | 3567 |
| M2F + F148L | 3568 |
| M2F + P154R | 3569 |
| M2F + P176A | 3570 |
| M2F + F185W | 3571 |
| M2F + A192G | 3572 |
| M2F + V202A | 3573 |
| M2F + I112V + P114T | 3574 |
| M2F + I112V + I115V | 3575 |
| M2F + I112V + P116G | 3576 |
| M2F + I112V + T117D | 3577 |
| M2F + I112V + E127K | 3578 |
| M2F + I112V + Q130R | 3579 |
| M2F + I112V + A131R | 3580 |
| M2F + I112V + K140E | 3581 |
| M2F + I112V + F148 | 3582 |
| M2F + I112V + F148L | 3583 |
| M2F + I112V + P154R | 3584 |
| M2F + I112V + P176A | 3585 |
| M2F + I112V + F185W | 3586 |
| M2F + I112V + A192G | 3587 |
| M2F + I112V + V202A | 3588 |
| M2F + P114T + I112V | 3589 |
| M2F + P114T + I115V | 3590 |
| M2F + P114T + P116G | 3591 |
| M2F + P114T + T117D | 3592 |
| M2F + P114T + E127K | 3593 |
| M2F + P114T + Q130R | 3594 |
| M2F + P114T + A131R | 3595 |
| M2F + P114T + K140E | 3596 |
| M2F + P114T + F148I | 3597 |
| M2F + P114T + F148L | 3598 |
| M2F + P114T + P154R | 3599 |
| M2F + P114T + P176A | 3600 |
| M2F + P114T + F185W | 3601 |
| M2F + P114T + A192G | 3602 |
| M2F + P114T + V202A | 3603 |
| M2F + I115V + I112V | 3604 |
| M2F + I115V + P114T | 3605 |
| M2F + I115V + P116G | 3606 |
| M2F + I115V + T117D | 3607 |
| M2F + I115V + E127K | 3608 |
| M2F + I115V + Q130R | 3609 |
| M2F + I115V + A131R | 3610 |
| M2F + I115V + K140E | 3611 |
| M2F + I115V + F148I | 3612 |
| M2F + I115V + F148L | 3613 |
| M2F + I115V + P154R | 3614 |
| M2F + I115V + P176A | 3615 |
| M2F + I115V + F185W | 3616 |
| M2F + I115V + A192G | 3617 |
| M2F + I115V + V202A | 3618 |
| M2F + P116G + I112V | 3619 |
| M2F + P116G + P114T | 3620 |
| M2F + P116G + I115V | 3621 |
| M2F + P116G + T117D | 3622 |
| M2F + P116G + E127K | 3623 |
| M2F + P116G + Q130R | 3624 |
| M2F + P116G + A131R | 3625 |
| M2F + P116G + K140E | 3626 |
| M2F + P116G + F148 | 3627 |
| M2F + P116G + F148L | 3628 |
| M2F + P116G + P154R | 3629 |
| M2F + P116G + P176A | 3630 |
| M2F + P116G + F185W | 3631 |
| M2F + P116G + A192G | 3632 |
| M2F + P116G + V202A | 3633 |
| M2F + T117D + I112V | 3634 |
| M2F + T117D + P114T | 3635 |
| M2F + T117D + I115V | 3636 |
| M2F + T117D + P116G | 3637 |
| M2F + T117D + E127K | 3638 |
| M2F + T117D + Q130R | 3639 |
| M2F + T117D + A131R | 3640 |
| M2F + T117D + K140E | 3641 |
| M2L + D53G + I112V | 3642 |
| M2L + D53G + P114T | 3643 |
| M2L + D53G + I115V | 3644 |
| M2L + D53G + P116G | 3645 |
| M2L + D53G + T117D | 3646 |
| M2L + D53G + E127K | 3647 |
| M2L + D53G + Q130R | 3648 |
| M2L + D53G + A131R | 3649 |
| M2L + D53G + K140E | 3650 |
| M2L + D53G + F148I | 3651 |
| M2L + D53G + F148L | 3652 |
| M2L + D53G + P154R | 3653 |
| M2L + D53G + P176A | 3654 |
| M2L + D53G + F185W | 3655 |
| M2L + D53G + A192G | 3656 |
| M2L + D53G + V202A | 3657 |
| M2L + D53G + I112V + P114T | 3658 |
| M2L + D53G + I112V + I115V | 3659 |
| M2L + D53G + I112V + P116G | 3660 |
| M2L + D53G + I112V + T117D | 3661 |
| M2L + D53G + I112V + E127K | 3662 |
| M2L + D53G + I112V + Q130R | 3663 |
| M2L + D53G + I112V + A131R | 3664 |
| M2L + D53G + I112V + K140E | 3665 |
| M2L + D53G + I112V + F148 | 3666 |
| M2L + D53G + I112V + F148L | 3667 |
| M2L + D53G + I112V + P154R | 3668 |
| M2L + D53G + I112V + P176A | 3669 |
| M2L + D53G + I112V + F185W | 3670 |
| M2L + D53G + I112V + A192G | 3671 |
| M2L + D53G + I112V + V202A | 3672 |
| M2L + D53G + P114T + I112V | 3673 |
| M2L + D53G + P114T + I115V | 3674 |
| M2L + D53G + P114T + P116G | 3675 |
| M2L + D53G + P114T + T117D | 3676 |
| M2L + D53G + P114T + E127K | 3677 |
| M2L + D53G + P114T + Q130R | 3678 |
| M2L + D53G + P114T + A131R | 3679 |
| M2L + D53G + P114T + K140E | 3680 |
| M2L + D53G + P114T + F148I | 3681 |
| M2L + D53G + P114T + F148L | 3682 |
| M2L + D53G + P114T + P154R | 3683 |
| M2L + D53G + P114T + P176A | 3684 |
| M2L + D53G + P114T + F185W | 3685 |
| M2L + D53G + P114T + A192G | 3686 |
| M2L + D53G + P114T + V202A | 3687 |
| M2L + D53G + I115V + I112V | 3688 |
| M2L + D53G + I115V + P114T | 3689 |
| M2L + D53G + I115V + P116G | 3690 |
| M2L + D53G + I115V + T117D | 3691 |
| M2L + D53G + I115V + E127K | 3692 |
| M2L + D53G + I115V + Q130R | 3693 |
| M2L + D53G + I115V + A131R | 3694 |
| M2L + D53G + I115V + K140E | 3695 |
| M2L + D53G + I115V + F148 | 3696 |
| M2L + D53G + I115V + F148L | 3697 |
| M2L + D53G + I115V + P154R | 3698 |
| M2L + D53G + I115V + P176A | 3699 |
| M2L + D53G + I115V + F185W | 3700 |
| M2L + D53G + I115V + A192G | 3701 |
| M2L + D53G + I115V + V202A | 3702 |
| M2L + D53G + P116G + I112V | 3703 |
| M2L + D53G + P116G + P114T | 3704 |
| M2L + D53G + P116G + I115V | 3705 |
| M2L + D53G + P116G + T117D | 3706 |
| M2L + D53G + P116G + E127K | 3707 |
| M2L + D53G + P116G + Q130R | 3708 |
| M2L + D53G + P116G + A131R | 3709 |
| M2L + D53G + P116G + K140E | 3710 |
| M2L + D53G + P116G + F148 | 3711 |
| M2L + D53G + P116G + F148L | 3712 |
| M2L + D53G + P116G + P154R | 3713 |
| M2L + D53G + P116G + P176A | 3714 |
| M2L + D53G + P116G + F185W | 3715 |
| M2L + D53G + P116G + A192G | 3716 |
| M2L + D53G + P116G + V202A | 3717 |
| M2L + D53G + T117D + I112V | 3718 |
| M2L + D53G + T117D + P114T | 3719 |
| M2L + D53G + T117D + I115V | 3720 |
| M2L + D53G + T117D + P116G | 3721 |
| M2L + D53G + T117D + E127K | 3722 |
| M2L + D53G + T117D + Q130R | 3723 |
| M2L + D53G + T117D + A131R | 3724 |
| M2L + D53G + T117D + K140E | 3725 |
| M2F + T117D + F148I | 3726 |
| M2F + T117D + F148L | 3727 |
| M2F + T117D + P154R | 3728 |
| M2F + T117D + P176A | 3729 |
| M2F + T117D + F185W | 3730 |
| M2F + T117D + A192G | 3731 |
| M2F + T117D + V202A | 3732 |
| M2F + E127K + I112V | 3733 |
| M2F + E127K + P114T | 3734 |
| M2F + E127K + I115V | 3735 |
| M2F + E127K + P116G | 3736 |
| M2F + E127K + T117D | 3737 |
| M2F + E127K + Q130R | 3738 |
| M2F + E127K + A131R | 3739 |
| M2F + E127K + K140E | 3740 |
| M2F + E127K + F148I | 3741 |
| M2F + E127K + F148L | 3742 |
| M2F + E127K + P154R | 3743 |
| M2F + E127K + P176A | 3744 |
| M2F + E127K + F185W | 3745 |
| M2F + E127K + A192G | 3746 |
| M2F + E127K + V202A | 3747 |
| M2F + Q130R + I112V | 3748 |
| M2F + Q130R + P114T | 3749 |
| M2F + Q130R + I115V | 3750 |
| M2F + Q130R + P116G | 3751 |
| M2F + Q130R + T117D | 3752 |
| M2F + Q130R + E127K | 3753 |
| M2F + Q130R + A131R | 3754 |
| M2F + Q130R + K140E | 3755 |
| M2F + Q130R + F148 | 3756 |
| M2F + Q130R + F148L | 3757 |
| M2F + Q130R + P154R | 3758 |
| M2F + Q130R + P176A | 3759 |
| M2F + Q130R + F185W | 3760 |
| M2F + Q130R + A192G | 3761 |
| M2F + Q130R + V202A | 3762 |
| M2F + A131R + I112V | 3763 |
| M2F + A131R + P114T | 3764 |
| M2F + A131R + I115V | 3765 |
| M2F + A131R + P116G | 3766 |
| M2F + A131R + T117D | 3767 |
| M2F + A131R + E127K | 3768 |
| M2F + A131R + Q130R | 3769 |
| M2F + A131R + K140E | 3770 |
| M2F + A131R + F148 | 3771 |
| M2F + A131R + F148L | 3772 |
| M2F + A131R + P154R | 3773 |
| M2F + A131R + P176A | 3774 |
| M2F + A131R + F185W | 3775 |
| M2F + A131R + A192G | 3776 |
| M2F + A131R + V202A | 3777 |
| M2F + K140E + I112V | 3778 |
| M2F + K140E + P114T | 3779 |
| M2F + K140E + I115V | 3780 |
| M2F + K140E + P116G | 3781 |
| M2F + K140E + T117D | 3782 |
| M2F + K140E + E127K | 3783 |
| M2F + K140E + Q130R | 3784 |
| M2F + K140E + A131R | 3785 |
| M2F + K140E + F1481 | 3786 |
| M2F + K140E + F148L | 3787 |
| M2F + K140E + P154R | 3788 |
| M2F + K140E + P176A | 3789 |
| M2F + K140E + F185W | 3790 |
| M2F + K140E + A192G | 3791 |
| M2F + K140E + V202A | 3792 |
| M2F + F148I + I112V | 3793 |
| M2F + F148I + P114T | 3794 |
| M2F + F148I + I115V | 3795 |
| M2F + F148I + P116G | 3796 |
| M2F + F148I + T117D | 3797 |
| M2F + F148I + E127K | 3798 |
| M2F + F148I + Q130R | 3799 |
| M2F + F148I + A131R | 3800 |
| M2F + F148I + K140E | 3801 |
| M2F + F148I + P154R | 3802 |
| M2F + F148I + P176A | 3803 |
| M2F + F148I + F185W | 3804 |
| M2F + F148I + A192G | 3805 |
| M2F + F148I + V202A | 3806 |
| M2F + F148L + I112V | 3807 |
| M2F + F148L + P114T | 3808 |
| M2F + F148L + I115V | 3809 |
| M2L + D53G + T117D + F148 | 3810 |
| M2L + D53G + T117D + F148L | 3811 |
| M2L + D53G + T117D + P154R | 3812 |
| M2L + D53G + T117D + P176A | 3813 |
| M2L + D53G + T117D + F185W | 3814 |
| M2L + D53G + T117D + A192G | 3815 |
| M2L + D53G + T117D + V202A | 3816 |
| M2L + D53G + E127K + I112V | 3817 |
| M2L + D53G + E127K + P114T | 3818 |
| M2L + D53G + E127K + I115V | 3819 |
| M2L + D53G + E127K + P116G | 3820 |
| M2L + D53G + E127K + T117D | 3821 |
| M2L + D53G + E127K + Q130R | 3822 |
| M2L + D53G + E127K + A131R | 3823 |
| M2L + D53G + E127K + K140E | 3824 |
| M2L + D53G + E127K + F148 | 3825 |
| M2L + D53G + E127K + F148L | 3826 |
| M2L + D53G + E127K + P154R | 3827 |
| M2L + D53G + E127K + P176A | 3828 |
| M2L + D53G + E127K + F185W | 3829 |
| M2L + D53G + E127K + A192G | 3830 |
| M2L + D53G + E127K + V202A | 3831 |
| M2L + D53G + Q130R + I112V | 3832 |
| M2L + D53G + Q130R + P114T | 3833 |
| M2L + D53G + Q130R + I115V | 3834 |
| M2L + D53G + Q130R + P116G | 3835 |
| M2L + D53G + Q130R + T117D | 3836 |
| M2L + D53G + Q130R + E127K | 3837 |
| M2L + D53G + Q130R + A131R | 3838 |
| M2L + D53G + Q130R + K140E | 3839 |
| M2L + D53G + Q130R + F148 | 3840 |
| M2L + D53G + Q130R + F148L | 3841 |
| M2L + D53G + Q130R + P154R | 3842 |
| M2L + D53G + Q130R + P176A | 3843 |
| M2L + D53G + Q130R + F185W | 3844 |
| M2L + D53G + Q130R + A192G | 3845 |
| M2L + D53G + Q130R + V202A | 3846 |
| M2L + D53G + A131R + I112V | 3847 |
| M2L + D53G + A131R + P114T | 3848 |
| M2L + D53G + A131R + I115V | 3849 |
| M2L + D53G + A131R + P116G | 3850 |
| M2L + D53G + A131R + T117D | 3851 |
| M2L + D53G + A131R + E127K | 3852 |
| M2L + D53G + A131R + Q130R | 3853 |
| M2L + D53G + A131R + K140E | 3854 |
| M2L + D53G + A131R + F148I | 3855 |
| M2L + D53G + A131R + F148L | 3856 |
| M2L + D53G + A131R + P154R | 3857 |
| M2L + D53G + A131R + P176A | 3858 |
| M2L + D53G + A131R + F185W | 3859 |
| M2L + D53G + A131R + A192G | 3860 |
| M2L + D53G + A131R + V202A | 3861 |
| M2L + D53G + K140E + I112V | 3862 |
| M2L + D53G + K140E + P114T | 3863 |
| M2L + D53G + K140E + I115V | 3864 |
| M2L + D53G + K140E + P116G | 3865 |
| M2L + D53G + K140E + T117D | 3866 |
| M2L + D53G + K140E + E127K | 3867 |
| M2L + D53G + K140E + Q130R | 3868 |
| M2L + D53G + K140E + A131R | 3869 |
| M2L + D53G + K140E + F148I | 3870 |
| M2L + D53G + K140E + F148L | 3871 |
| M2L + D53G + K140E + P154R | 3872 |
| M2L + D53G + K140E + P176A | 3873 |
| M2L + D53G + K140E + F185W | 3874 |
| M2L + D53G + K140E + A192G | 3875 |
| M2L + D53G + K140E + V202A | 3876 |
| M2L + D53G + F1481 + I112V | 3877 |
| M2L + D53G + F1481 + P114T | 3878 |
| M2L + D53G + F1481 + I115V | 3879 |
| M2L + D53G + F1481 + P116G | 3880 |
| M2L + D53G + F1481 + T117D | 3881 |
| M2L + D53G + F1481 + E127K | 3882 |
| M2L + D53G + F1481 + Q130R | 3883 |
| M2L + D53G + F1481 + A131R | 3884 |
| M2L + D53G + F1481 + K140E | 3885 |
| M2L + D53G + F1481 + P154R | 3886 |
| M2L + D53G + F1481 + P176A | 3887 |
| M2L + D53G + F1481 + F185W | 3888 |
| M2L + D53G + F1481 + A192G | 3889 |
| M2L + D53G + F1481 + V202A | 3890 |
| M2L + D53G + F148L + I112V | 3891 |
| M2L + D53G + F148L + P114T | 3892 |
| M2L + D53G + F148L + I115V | 3893 |
| M2F + D53G + V7E | 3894 |
| M2F + D53G + E79N | 3895 |
| M2F + D53G + I84V | 3896 |
| M2F + D53G + F93L | 3897 |
| M2F + D53G + L201I | 3898 |
| M2F + D53G + E203D | 3899 |
| M2F + D53G + S264A | 3900 |
| M2F + D53G + V7E + S264A | 3901 |
| M2F + D53G + L201I + V7E | 3902 |
| M2F + D53G + L201I + E79N | 3903 |
| M2F + D53G + L201I + I84V | 3904 |
| M2F + D53G + L201I + F93L | 3905 |
| M2F + D53G + L201I + P116G | 3906 |
| M2F + D53G + L201I + T117D | 3907 |
| M2F + D53G + L201I + A131R | 3908 |
| M2F + D53G + L201I + K140E | 3909 |
| M2F + D53G + E203D + V7E | 3910 |
| M2F + D53G + E203D + E79N | 3911 |
| M2F + D53G + E203D + I84V | 3912 |
| M2F + D53G + E203D + F93L | 3913 |
| M2F + D53G + E203D + P116G | 3914 |
| M2F + D53G + E203D + T117D | 3915 |
| M2F + D53G + E203D + A131R | 3916 |
| M2F + D53G + L201I + E203D + V7E | 3917 |
| M2F + D53G + L201I + E203D + E79N | 3918 |
| M2F + D53G + L201I + E203D + I84V | 3919 |
| M2F + D53G + L201I + E203D + F93L | 3920 |
| M2F + D53G + L201I + E203D + P116G | 3921 |
| M2F + D53G + L201I + E203D + T117D | 3922 |
| M2F + D53G + L201I + E203D + A131R | 3923 |
| M2F + D53G + L201I + I84V + F93L + K140E | 3924 |
| M2F + D53G + L201I + I84V + F93L + P116G | 3925 |
| M2F + D53G + L201I + A131R + P116G | 3926 |
| M2F + D53G + I84V + F93L + K140E | 3927 |
| M2F + D53G + I84V + F93L + P116G | 3928 |
| M2F + D53G + A131R + P116G | 3929 |
| M2F + K140E + V7E | 3930 |
| M2F + K140E + P37S | 3931 |
| M2F + K140E + D53G + V177A | 3932 |
| M2F + K140E + D53G + E79K | 3933 |
| M2F + K140E + G58E | 3934 |
| M2F + K140E + P116S | 3935 |
| M2F + K140E + T135D | 3936 |
| M2F + K140E + E165H | 3937 |
| M2F + K140E + L174R | 3938 |
| M2F + K140E + V177D | 3939 |
| M2F + K140E + V177Q + S238A | 3940 |
| M2F + K140E + V177A | 3941 |
| M2F + K140E + W183Y | 3942 |
| M2F + K140E + A196P | 3943 |
| M2F + K140E + L201I | 3944 |
| M2F + K140E + E203D | 3945 |
| M2F + K140E + K243T | 3946 |
| M2F + K140E + S264A | 3947 |
| M2F + K140E + K97Q | 3948 |
| M2F + D53G + L57I + V177E + S238A | 3949 |
| Q145H + P154R | 3950 |
| K140E + Q145H + P154R | 3951 |
| D53G + Q145H + P154R | 3952 |
| D53G + K140E + Q145H + P154R | 3953 |
| M2F + D53G + K140E + V7E | 3954 |
| M2F + D53G + K140E + P37E | 3955 |
| M2F + D53G + K140E + P37G | 3956 |
| M2F + D53G + K140E + P37S | 3957 |
| M2F + D53G + K140E + G58A | 3958 |
| M2F + D53G + K140E + G58P | 3959 |
| M2F + D53G + K140E + G58S | 3960 |
| M2F + D53G + K140E + G58D | 3961 |
| M2F + D53G + K140E + G58E | 3962 |
| M2F + D53G + K140E + M68V | 3963 |
| M2F + D53G + K140E + E73Q | 3964 |
| M2F + D53G + K140E + E79N | 3965 |
| M2F + D53G + K140E + E79D | 3966 |
| M2F + D53G + K140E + E79K | 3967 |
| M2F + D53G + K140E + K97R | 3968 |
| M2F + D53G + K140E + K104V | 3969 |
| M2F + D53G + K140E + P116S | 3970 |
| M2F + D53G + K140E + P116A | 3971 |
| M2F + D53G + K140E + P116G | 3972 |
| M2F + D53G + K140E + T117D | 3973 |
| M2F + D53G + K140E + T117S | 3974 |
| M2F + D53G + K140E + E123A | 3975 |
| M2F + D53G + K140E + E127S | 3976 |
| M2F + D53G + K140E + E127K | 3977 |
| M2F + D53G + K140E + E127Q | 3978 |
| M2F + D53G + K140E + A131R | 3979 |
| M2F + D53G + K140E + A131S | 3980 |
| M2F + D53G + K140S | 3981 |
| M2F + D53G + K140E + E163D | 3982 |
| M2F + D53G + K140E + D167A | 3983 |
| M2F + D53G + K140E + D167Q | 3984 |
| M2F + D53G + K140E + L174R | 3985 |
| M2F + D53G + K140E + N175S | 3986 |
| M2F + D53G + K140E + A192D | 3987 |
| M2F + D53G + K140E + A192G | 3988 |
| M2F + D53G + K140E + L201I | 3989 |
| M2F + D53G + K140E + E203D | 3990 |
| M2F + D53G + K140E + E203Q | 3991 |
| M2F + D53G + K140E + E203N | 3992 |
| M2F + D53G + K140E + H210R | 3993 |
| M2F + D53G + K140E + R234E | 3994 |
| M2F + D53G + K140E + K243T | 3995 |
| M2F + D53G + K140E + K243R | 3996 |
| M2F + D53G + K140E + D260Q | 3997 |
| M2F + D53G + K140E + D260G | 3998 |
| M2F + D53G + K140E + S264A | 3999 |
| M2F + D53G + K140E + S264E | 4000 |
| M2F + D53G + K140E + S264K | 4001 |
| M2F + D53G + K140E + E265Q | 4002 |
| M2F + D53G + K140E + A267V | 4003 |
| M2F + D53G + K140E + A267R | 4004 |
| M2F + D53G + K140E + L273Q | 4005 |
| M2F + D53G + K140E + L273H | 4006 |
| M2F + D53G + K140E + 1275T | 4007 |
| M2F + D53G + K140E + S276E | 4008 |
| M2F + D53G + K140E + S276P | 4009 |
| M2F + M49F + D53G + V177E | 4010 |
| M2F + V7E + T117D + A131R + S271K | 4011 |
| M2F + S271K | 4012 |
| M2F + A200K + W220F | 4013 |
| M2F + M68V + A131R + W220F + S271K | 4014 |
| M2F + V7E + V177E + W220F + S271K | 4015 |
| M2F + M68V + T117D + A200K | 4016 |
| M2F + V7E + T117D + V177E + A200K | 4017 |
| M2F + W220F | 4018 |
| M2F + V7E + M49F + D53G + M68V + S271K | 4019 |
| M2F + M49F + D53G + T117D | 4020 |
| M2F + V7E + M49F + D53G + A200K | 4021 |
| M2F + V7E + M49F + D53G + M68V + T117D + V177E | 4022 |
| M2F + V7E + A200K + W220F | 4023 |
| M2F + V7E + T117D + A200K + S271K | 4024 |
| M2F + V7E + M68V + T117D + A131R + A200K + W220F | 4025 |
| M2F + T117D + A131R + V177E + A200K + S271K | 4026 |
| M2F + M68V + A131R + V177E + S271K | 4027 |
| M2F + M68V | 4028 |
| M2F + M49F + D53G + M68V + T117D + S271K | 4029 |
| M2F + T117D + A131R + V177E + W220F | 4030 |
| M2F + V177E + A200K + W220F + S271K | 4031 |
| M2F + M68V + A200K + S271K | 4032 |
| M2F + V7E + T117D + A131R + W220F | 4033 |
| M2F + V7E + M68V + V177E + W220F + S271K | 4034 |
| M2F + M49F + D53G + M68V + A131R + S271K | 4035 |
| M2F + M68V + A200K + W220F + S271K | 4036 |
| M2F + V7E | 4037 |
| M2F + V7E + M49F + D53G + T117D + V177E + A200K | 4038 |
| M2F + V7E + M68V + T117D + V177E + A200K + S271K | 4039 |
| M2F + M68V + T117D + A131R | 4040 |
| M2F + M68V + T117D + W220F | 4041 |
| M2F + V177E + A200K | 4042 |
| M2F + M68V + T117D + A131R + A200K + W220F + S271K | 4043 |
| M2F + V7E + M49F + D53G + T117D + A131R + A200K + W220F | 4044 |
| M2F + V7E + M49F + D53G + M68V + A131R + V177E + W220F | 4045 |
| M2F + M68V + T117D + A131R + W220F | 4046 |
| M2F + T117D + A131R + V177E + W220F + S271K | 4047 |
| M2F + M49F + D53G + A131R | 4048 |
| M2F + V7E + M49F + D53G + A131R + V177E + S271K | 4049 |
| M2F + V7E + T117D + A131R + V177E | 4050 |
| M2F + M68V + V177E + W220F + S271K | 4051 |
| M2F + M49F + D53G + T117D + A131R + A200K + W220F + S271K | 4052 |
| M2F + V7E + M68V + T117D + W220F | 4053 |
| M2F + T117D + A200K | 4054 |
| M2F + M68V + T117D + A131R + V177E + A200K + W220F | 4055 |
| M2F + V7E + A131R | 4056 |
| M2F + T117D + S271K | 4057 |
| M2F + V7E + M49F + D53G + V177E + A200K | 4058 |
| M2F + M49F + D53G + M68V + A131R + A200K + W220F + S271K | 4059 |
| M2F + V7E + M68V + A200K + W220F + S271K | 4060 |
| M2F + V7E + M68V + A131R + A200K + S271K | 4061 |
| M2F + M49F + D53G + T117D + V177E + A200K + W220F | 4062 |
| M2F + V177E + W220F + S271K | 4063 |
| M2F + M68V + A200K | 4064 |

TABLE 4
Additional exemplary constructs

| SEQ ID NO | Construct |
|---|---|
| 4065 | FKBP-15xGly/Ser-HiBiT |
| 4066 | 6xHis-HaloTag7 |
| 4067 | 6xHis-3xGly/Ser-FKBP-15xGly/Ser-HaloTag[3-19] |
| 4068 | 6xHis-3xGly/Ser-FKBP-15xGly/Ser-HaloTag[3-19]-5xGly/Ser-VS-HiBiT |
| 4069 | HaloTag[22-297](M2F)-6xGly/Ser-6xHis |
| 4070 | HaloTag[22-297](M2F)-12xGly/Ser-LgBiT |
| 4071 | LgBiT-10xGly/Ser-HaloTag[22-297](M2F) |
| 4072 | HaloTag[22-297](M2F + M49F + D53G)-12xGly/Ser-LgBiT |
| 4073 | HaloTag[22-297](M2F + M49F + L57I)-12xGly/Ser-LgBiT |
| 4074 | HaloTag[22-297](M2F + M49F + S89A)-12xGly/Ser-LgBiT |
| 4075 | HaloTag[22-297](M2F + D53S + D56P)-12xGly/Ser-LgBiT |
| 4076 | HaloTag[22-297](M2F + D53G + V177E)-12xGly/Ser-LgBiT |
| 4077 | HaloTag[22-297](M2L)-12xGly/Ser-LgBiT |
| 4078 | HaloTag[22-297](M2F + D53S)-12xGly/Ser-LgBiT |
| 4079 | HaloTag[22-297](M2F + D56P)-12xGly/Ser-LgBiT |
| 4080 | HaloTag[22-297](M2F + E79K)-12xGly/Ser-LgBiT |
| 4081 | HaloTag[22-297](M2F + F93L)-12xGly/Ser-LgBiT |
| 4082 | HaloTag[22-297](M2F + T128M)-12xGly/Ser-LgBiT |
| 4083 | HaloTag[22-297](M2F + A131Q)-12xGly/Ser-LgBiT |
| 4084 | HaloTag[22-297](M2F + F148M)-12xGly/Ser-LgBiT |
| 4085 | HaloTag[22-297](M2F + V177Q)-12xGly/Ser-LgBiT |
| 4086 | HaloTag[22-297](M2F + P186A)-12xGly/Ser-LgBiT |
| 4087 | HaloTag[22-297](M2F + P186V)-12xGly/Ser-LgBiT |
| 4088 | HaloTag[22-297](M2F + S238A)-12xGly/Ser-LgBiT |
| 4089 | HaloTag[22-297](M2F + L253I)-12xGly/Ser-LgBiT |
| 4090 | EGFP-15xGly/Ser-HT[3-19]-10xGly/Ser-VSHiBiT |
| 4091 | HaloTag[22-297](M2F)-12xGly/Ser-LgBiT-3xGly/Ser-6xHis |
| 4092 | HaloTag[22-297](M2F + D53G + F148M)-12xGly/Ser-LgBiT-3xGly/Ser-6xHis |
| 4093 | HaloTag[22-297](M2F + D53G + V177E)-12xGly/Ser-LgBiT-3xGly/Ser-6xHis |
| 4094 | HaloTag[22-297](M2F + F148M + V177E)-12xGly/Ser-LgBiT-3xGly/Ser-6xHis |
| 4095 | 6xHis-3xGly/Ser-FKBP-15xGly/Ser-HaloTag[3-19]-VS-HiBiT |
| 4096 | 6xHis-3xGly/Ser-FKBP-15xGly/Ser-HaloTag[3-19]-2xGly/Ser-VS-HiBiT |
| 4097 | 6xHis-3xGly/Ser-FKBP-15xGly/Ser-HaloTag[3-19]-4xGly/Ser-VS-HiBiT |
| 4098 | 6xHis-3xGly/Ser-FKBP-15xGly/Ser-HaloTag[3-19]-6xGly/Ser-VS-HiBiT |
| 4099 | 6xHis-3xGly/Ser-FKBP-15xGly/Ser-HaloTag[3-19]-8xGly/Ser-VS-HiBiT |
| 4100 | 6xHis-3xGly/Ser-FKBP-15xGly/Ser-HaloTag[3-19]-10xGly/Ser-VS-HiBiT |
| 4101 | 6xHis-3xGly/Ser-FKBP-15xGly/Ser-HaloTag[3-19]-12xGly/Ser-VS-HiBiT |
| 4102 | 6xHis-3xGly/Ser-FKBP-15xGly/Ser-HaloTag[3-19]-15xGly/Ser-VS-HiBiT |
| 4103 | 6xHis-3xGly/Ser-FKBP-15xGly/Ser-VS-HiBiT-HaloTag[3-19] |
| 4104 | 6xHis-3xGly/Ser-FKBP-15xGly/Ser-VS-HiBiT-2xGly/Ser-HaloTag[3-19] |
| 4105 | 6xHis-3xGly/Ser-FKBP-15xGly/Ser-VS-HiBiT-4xGly/Ser-HaloTag[3-19] |
| 4106 | 6xHis-3xGly/Ser-FKBP-15xGly/Ser-VS-HiBiT-6xGly/Ser-HaloTag[3-19] |
| 4107 | 6xHis-3xGly/Ser-FKBP-15xGly/Ser-VS-HiBiT-8xGly/Ser-HaloTag[3-19] |
| 4108 | 6xHis-3xGly/Ser-FKBP-15xGly/Ser-VS-HiBiT-12xGly/Ser-HaloTag[3-19] |
| 4109 | 6xHis-3xGly/Ser-FKBP-15xGly/Ser-HaloTag[3-19]-AP-VS-HiBiT |
| 4110 | 6xHis-3xGly/Ser-FKBP-15xGly/Ser-HaloTag[3-19]-KP-VS-HiBiT |
| 4111 | 6xHis-3xGly/Ser-FKBP-15xGly/Ser-HaloTag[3-19]-EP-VS-HiBiT |
| 4112 | 6xHis-3xGly/Ser-FKBP-15xGly/Ser-HaloTag[3-19]-PAPAP-VS-HiBiT |
| 4113 | 6xHis-3xGly/Ser-FKBP-15xGly/Ser-HaloTag[3-19]-PKPKP-VS-HiBiT |
| 4114 | 6xHis-3xGly/Ser-FKBP-15xGly/Ser-HaloTag[3-19]-PEPEP-VS-HiBiT |
| 4115 | 6xHis-3xGly/Ser-FKBP-HaloTag[3-19]-VS-HiBiT |
| 4116 | 6xHis-3xGly/Ser-FKBP-4xGly/Ser-HaloTag[3-19]-VS-HiBiT |
| 4117 | 6xHis-3xGly/Ser-FKBP-8xGly/Ser-HaloTag[3-19]-VS-HiBiT |
| 4118 | 6xHis-3xGly/Ser-FKBP-15xGly/Ser-HaloTag[3-19]-VTING-VS-HiBiT |
| 4119 | 6xHis-3xGly/Ser-FKBP-15xGly/Ser-HaloTag[4-19]-VS-HiBiT |
| 4120 | 6xHis-3xGly/Ser-FKBP-15xGly/Ser-HaloTag[8-19]-VS-HiBiT |
| 4121 | 6xHis-3xGly/Ser-FKBP-15xGly/Ser-HaloTag[3-18]-VS-HiBiT |
| 4122 | 6xHis-3xGly/Ser-FKBP-15xGly/Ser-HaloTag[4-18]-VS-HiBiT |
| 4123 | 6xHis-3xGly/Ser-FKBP-15xGly/Ser-HaloTag[3-17]-VS-HiBiT |
| 4124 | 6xHis-3xGly/Ser-FKBP-15xGly/Ser-HaloTag[4-17]-VS-HiBiT |
| 4125 | 6xHis-3xGly/Ser-FKBP-15xGly/Ser-HaloTag[8-17]-VS-HiBiT |
| 4126 | 6xHis-3xGly/Ser-FKBP-15xGly/Ser-HaloTag[3-19]-VS-HiBiT(K8R + K9R) |
| | 6xHis-3xGly/Ser-FKBP-15xGly/Ser-HaloTag[3-19]-4xGly/Ser-VS- |
| 4127 | HiBiT(K8R + K9R) |
| 4128 | 6xHis-3xGly/Ser-FKBP-15xGly/Ser-VS-HiBiT(K8R + K9R)-HaloTag[3-19] |
| 4129 | 6xHis-3xGly/Ser-FKBP-15xGly/Ser-VS-HiBiT(K8R + K9R)-4xGly/Ser-HaloTag[3-19] |
| 4130 | 6xHis-3xGly/Ser-FKBP-15xGly/Ser-VS-HiBiT(K8R + K9R)-12xGly/Ser-HaloTag[3-19] |
| 4131 | LgBiT |
| 4132 | HaloTag[22-297](M2F + D53S + V177E + M49F)-12xGly/Ser-LgBiT-3xGly/Ser-6XHis |
| 4133 | Aga2-15xGly/Ser-4xGly/Ser-HaloTag[3-19]-4xGly/Ser-VS-HiBiT |
| 4134 | HaloTag[3-19]-VS-HiBiT-15xGly/Ser-FKBP-5xGly/Ser-6xHis |
| 4135 | HaloTag[3-19]-4xGly/Ser-VS-HiBiT-15xGly/Ser-FKBP-5xGly/Ser-6xHis |
| 4136 | HaloTag[3-19]-6xGly/Ser-VS-HiBiT-15xGly/Ser-FKBP-5xGly/Ser-6xHis |
| 4137 | HaloTag[3-19]-8xGly/Ser-VS-HiBiT-15xGly/Ser-FKBP-5xGly/Ser-6xHis |
| 4138 | HaloTag[3-19]-10xGly/Ser-VS-HiBiT-15xGly/Ser-FKBP-5xGly/Ser-6xHis |
| 4139 | HaloTag[3-19]-12xGly/Ser-VS-HiBiT-15xGly/Ser-FKBP-5xGly/Ser-6xHis |
| 4140 | HaloTag[3-19]-15xGly/Ser-VS-HiBiT-15xGly/Ser-FKBP-5xGly/Ser-6xHis |
| 4141 | VS-HiBiT-HaloTag[3-19]-FKBP-5xGly/Ser-6xHis |
| 4142 | VS-HiBiT-2xGly/Ser-HaloTag[3-19]-FKBP-5xGly/Ser-6xHis |
| 4143 | VS-HiBiT-4xGly/Ser-HaloTag[3-19]-FKBP-5xGly/Ser-6xHis |
| 4144 | VS-HiBiT-6xGly/Ser-HaloTag[3-19]-FKBP-5xGly/Ser-6xHis |
| 4145 | VS-HiBiT-8xGly/Ser-HaloTag[3-19]-FKBP-5xGly/Ser-6xHis |
| 4146 | VS-HiBiT-10xGly/Ser-HaloTag[3-19]-FKBP-5xGly/Ser-6xHis |
| 4147 | HaloTag[3-19]-AP-VS-HiBiT-15xGly/Ser-FKBP-5xGly/Ser-6xHis |
| 4148 | HaloTag[3-19]-KP-VS-HiBiT-15xGly/Ser-FKBP-5xGly/Ser-6xHis |
| 4149 | HaloTag[3-19]-EP-VS-HiBiT-15xGly/Ser-FKBP-5xGly/Ser-6xHis |
| 4150 | HaloTag[3-19]-PAPAP-VS-HiBiT-15xGly/Ser-FKBP-5xGly/Ser-6xHis |
| 4151 | HaloTag[3-19]-PKPKP-VS-HiBiT-15xGly/Ser-FKBP-5xGly/Ser-6xHis |
| 4152 | HaloTag[3-19]-3XEAAAK-VS-HiBiT-15xGly/Ser-FKBP-5xGly/Ser-6xHis |
| 4153 | HaloTag[3-19]-VS-HiBiT-FKBP-5xGly/Ser-6xHis |
| 4154 | HaloTag[3-19]-VS-HiBiT-4xGly/Ser-FKBP-5xGly/Ser-6xHis |
| 4155 | HaloTag[3-19]-VS-HiBiT-8xGly/Ser-FKBP-5xGly/Ser-6xHis |
| 4156 | HaloTag[3-19]-VTING-VS-HiBiT-15xGly/Ser-FKBP-5xGly/Ser-6xHis |
| 4157 | HaloTag[3-19]-ING-VS-HiBiT-15xGly/Ser-FKBP-5xGly/Ser-6xHis |
| 4158 | HaloTag[4-19]-VS-HiBiT-15xGly/Ser-FKBP-5xGly/Ser-6xHis |
| 4159 | HaloTag[8-19]-VS-HiBiT-15xGly/Ser-FKBP-5xGly/Ser-6xHis |
| 4160 | HaloTag[3-18]-VS-HiBiT-15xGly/Ser-FKBP-5xGly/Ser-6xHis |
| 4161 | HaloTag[4-18]-VS-HiBiT-15xGly/Ser-FKBP-5xGly/Ser-6xHis |
| 4162 | HaloTag[8-18]-VS-HiBiT-15xGly/Ser-FKBP-5xGly/Ser-6xHis |
| 4163 | HaloTag[3-17]-VS-HiBiT-15xGly/Ser-FKBP-5xGly/Ser-6xHis |
| 4164 | HaloTag[4-17]-VS-HiBiT-15xGly/Ser-FKBP-5xGly/Ser-6xHis |
| 4165 | HaloTag[8-17]-VS-HiBiT-15xGly/Ser-FKBP-5xGly/Ser-6xHis |
| 4166 | HaloTag[3-19]-VS-HiBiT(K8R + K9R)-15xGly/Ser-FKBP-5xGly/Ser-6xHis |
| 4167 | HaloTag[3-19]-4xGly/Ser-VS-HiBiT(K8R + K9R)-FKBP-5xGly/Ser-6xHis |
| 4168 | VS-HiBiT(K8R + K9R)-HaloTag[3-19]-FKBP-5xGly/Ser-6xHis |
| 4169 | VS-HiBiT(K8R + K9R)-4xGly/Ser-HaloTag[3-19]-FKBP-5xGly/Ser-6xHis |
| 4170 | VS-HiBiT(K8R + K9R)-10xGly/Ser-HaloTag[3-19]-FKBP-5xGly/Ser-6xHis |
| 4171 | Aga2-15xGly/Ser-EGFP-4xGly/Ser-HaloTag[3-19]-4xGly/Ser-NVSGWRLFKKISN |
| 4172 | Aga2-15xGly/Ser-EGFP-4xGly/Ser-HaloTag[3-19]-4xGly/Ser-NVTGYRLFKKISN |
| 4173 | Aga2-15xGly/Ser-EGFP-4xGly/Ser-HaloTag[3-19]-4xGly/Ser-VTGYRLFEKIS |
| 4174 | Aga2-15xGly/Ser-EGFP-4xGly/Ser-HaloTag[3-19]-4xGly/Ser-VS-HiBiT |
| 4175 | HaloTag[22-297](M2F + K140E)-12xGly/Ser-LgBiT-3xGly/Ser-6xHis |
| 4176 | HaloTag[22-297](M2F + D53G + K140E)-12xGly/Ser-LgBiT-3xGly/Ser-6xHis |

1. A dual-reporter system comprising:
(a) a tandem peptide tag comprising (i) a peptide component of a bioluminescent complex fused to (ii) a peptide component of a modified dehalogenase complex; and
(b) a tandem polypeptide reporter comprising (i) a polypeptide component of the bioluminescent complex and (ii) a polypeptide component of the modified dehalogenase complex;
wherein the peptide component and the polypeptide component of the bioluminescent complex are capable of interacting to form the bioluminescent complex, and wherein the bioluminescent complex is capable of producing bioluminescence in the presence of a substrate for the bioluminescent complex; and
wherein the peptide component and the polypeptide component of the modified dehalogenase complex are capable of interacting to form the modified dehalogenase complex, and wherein the modified dehalogenase complex is capable of forming a covalent bond with a haloalkyl ligand.

2. The system of claim 1, wherein the tandem polypeptide reporter exhibits high affinity for the tandem peptide tag.

3. The system of claim 2, wherein the peptide component of the bioluminescent complex exhibits high affinity for the polypeptide component of the bioluminescent complex.

4. The system of claim 3, wherein the high affinity is a K_{d} less than 1 μM.

5. The system of claim 4, wherein the high affinity is a K_{d} less than 1 nM.

6. The system of claim 1, wherein the peptide component of the bioluminescent complex has at least 70% identity with SEQ ID NO: 3038.

7. The system of claim 6, wherein the peptide component of the bioluminescent complex has 100% identity with SEQ ID NO: 3038.

8. The system of claim 1, wherein the polypeptide component of the bioluminescent complex has at least 70% identity with SEQ ID NO: 3037.

9. The system of claim 8, wherein the polypeptide component of the bioluminescent complex has 100% identity with SEQ ID NO: 3037.

10. The system of claim 1, wherein the peptide component of the modified dehalogenase complex has at least 70% identity with SEQ ID NO: 3034.

11. The system of claim 10, wherein the peptide component of the modified dehalogenase complex has 100% identity with SEQ ID NO: 3034.

12. The system of claim 1, wherein the polypeptide component of the modified dehalogenase complex has at least 70% identity with SEQ ID NO: 1188.

13. The system of claim 12, wherein the polypeptide component of the modified dehalogenase complex has 100% identity with SEQ ID NO: 1188.

14. The system of claim 1, wherein the tandem peptide tag has at least 70% identity to one of SEQ ID NOS: 3062-3063, 3067-3078, 3092, 3094-3109, or 4177-4181.

15. The system of claim 14, wherein the tandem peptide tag has 100% identity to one of SEQ ID NOS: 3062-3063, 3067-3078, 3092, 3094-3109, or 4177-4181.

16. The system of claim 1, wherein the tandem polypeptide reporter has at least 70% identity to one of SEQ ID NOS: 3110-4064.

17. The system of claim 16, wherein the tandem polypeptide reporter has 100% identity to one of SEQ ID NOS: 3110-4064.

18. The system of claim 1, further comprising the substrate for the bioluminescent complex.

19. The system of claim 1, further comprising the haloalkyl ligand.

20. The system of claim 19, wherein the haloalkyl ligand comprises a haloalkane moiety linked to a fluorophore.

21. The system of claim 20, wherein the fluorophore has an excitation spectrum that overlaps with an emission spectrum of the bioluminescent complex and the substrate.

22. The system of claim 1, wherein the tandem peptide tag is linked to a target element.

23. (canceled)

24. A tandem peptide tag comprising a peptide component of bioluminescent complex fused to a peptide component of modified dehalogenase complex;
wherein the peptide component of the bioluminescent complex is capable of interacting with a polypeptide component of the bioluminescent complex to form the bioluminescent complex, and wherein the bioluminescent complex is capable of producing bioluminescence in the presence of a substrate for the bioluminescent complex; and
wherein the peptide component of the modified dehalogenase complex is capable of interacting with a polypeptide component of the modified dehalogenase complex to form the modified dehalogenase complex, and wherein the modified dehalogenase complex is capable of forming a covalent bond with a haloalkyl ligand.

25-43. (canceled)

44. A tandem polypeptide reporter comprising a polypeptide component of the bioluminescent complex and a polypeptide component of the modified dehalogenase complex;
wherein the polypeptide component of the bioluminescent complex is capable of interacting with a peptide component of the bioluminescent complex to form the bioluminescent complex, and wherein the bioluminescent complex is capable of producing bioluminescence in the presence of a substrate for the bioluminescent complex; and
wherein the polypeptide component of the modified dehalogenase complex is capable of interacting with a peptide component of the modified dehalogenase complex to form the modified dehalogenase complex, and wherein the modified dehalogenase complex is capable of forming a covalent bond with a haloalkyl ligand.

45-60. (canceled)

61. A method of detecting a component of interest in a system, the method comprising:
(a) linking the component of interest to a tandem peptide tag comprising a peptide component of bioluminescent complex fused to a peptide component of modified dehalogenase complex, wherein the peptide component of the bioluminescent complex is capable of interacting with a polypeptide component of the bioluminescent complex to form the bioluminescent complex, wherein the bioluminescent complex is capable of producing bioluminescence in the presence of a substrate for the bioluminescent complex, wherein the peptide component of the modified dehalogenase complex is capable of interacting with a polypeptide component of the modified dehalogenase complex to form the modified dehalogenase complex, and wherein the modified dehalogenase complex is capable of forming a covalent bond with a haloalkyl ligand;
(b) contacting the component of interest linked to the tandem peptide tag with a polypeptide component of a modified dehalogenase complex under condition such that the modified dehalogenase complex is formed;
(c) contacting the modified dehalogenase complex with a haloalkyl ligand comprising a haloalkane linked to a fluorophore; and
(d) detecting fluorescence.

62. A method of detecting a component of interest using a system of claim 1, the method comprising:
(a) linking the component of interest to the tandem peptide tag comprising a peptide component of bioluminescent complex fused to a peptide component of modified dehalogenase complex, wherein the peptide component of the bioluminescent complex is capable of interacting with a polypeptide component of the bioluminescent complex to form the bioluminescent complex, wherein the bioluminescent complex is capable of producing bioluminescence in the presence of a substrate for the bioluminescent complex, wherein the peptide component of the modified dehalogenase complex is capable of interacting with a polypeptide component of the modified dehalogenase complex to form the modified dehalogenase complex, and wherein the modified dehalogenase complex is capable of forming a covalent bond with a haloalkyl ligand;
(b) contacting the component of interest linked to the tandem peptide tag with a polypeptide component of a bioluminescent complex under condition such that the bioluminescent complex is formed;
(c) contacting the bioluminescent complex with a substrate for the bioluminescent complex; and
(d) detecting bioluminescence.

63. A method of detecting a component of interest in a system, the method comprising:
(a) linking the component of interest to a tandem peptide tag comprising a peptide component of bioluminescent complex fused to a peptide component of modified dehalogenase complex, wherein the peptide component of the bioluminescent complex is capable of interacting with a polypeptide component of the bioluminescent complex to form the bioluminescent complex, wherein the bioluminescent complex is capable of producing bioluminescence in the presence of a substrate for the bioluminescent complex, wherein the peptide component of the modified dehalogenase complex is capable of interacting with a polypeptide component of the modified dehalogenase complex to form the modified dehalogenase complex, and wherein the modified dehalogenase complex is capable of forming a covalent bond with a haloalkyl ligand;
(b) contacting the component of interest linked to the tandem peptide tag with a tandem polypeptide reporter comprising (i) the polypeptide component of a bioluminescent complex and (ii) the polypeptide component of a modified dehalogenase complex under condition such that the bioluminescent complex and modified dehalogenase complexes are formed;
(c) contacting the bioluminescent complex with a substrate for the bioluminescent complex and/or a haloalkyl ligand comprising a haloalkane linked to a fluorophore; and
(d) detecting bioluminescence and/or fluorescence.

64. A method of detecting a component of interest in a system, the method comprising:
(a) linking the component of interest to a tandem peptide tag comprising a peptide component of bioluminescent complex fused to a peptide component of modified dehalogenase complex, wherein the peptide component of the bioluminescent complex is capable of interacting with a polypeptide component of the bioluminescent complex to form the bioluminescent complex, wherein the bioluminescent complex is capable of producing bioluminescence in the presence of a substrate for the bioluminescent complex, wherein the peptide component of the modified dehalogenase complex is capable of interacting with a polypeptide component of the modified dehalogenase complex to form the modified dehalogenase complex, and wherein the modified dehalogenase complex is capable of forming a covalent bond with a haloalkyl ligand;
(b) contacting the component of interest linked to the tandem peptide tag with independent polypeptides comprising (i) the polypeptide component of a bioluminescent complex and (ii) the polypeptide component of a modified dehalogenase complex under condition such that the bioluminescent complex and modified dehalogenase complexes are formed;
(c) contacting the bioluminescent complex with a substrate for the bioluminescent complex and/or a haloalkyl ligand comprising a haloalkane linked to a fluorophore; and
(d) detecting bioluminescence and/or fluorescence.
